(19) United States
(12) Patent Application Publication
(10) Pub. No.: US 2019/0144852 A1
(43) Pub. Date: May 16, 2019

(54) Combinatorial Metabolic Engineering Using a CRISPR System

(71) Applicant: The Board of Trustees of the University of Illinois, Urbana, IL (US)

(72) Inventors: Huimin Zhao, Champaign, IL (US); Jiazhang Lian, Urbana, IL (US)

(21) Appl. No.: 16/189,683

(22) Filed: Nov. 13, 2018

Related U.S. Application Data
(60) Provisional application No. 62/585,533, filed on Nov. 13, 2017.

(51) Int. Cl. C12N 15/10; C12N 15/113; C12N 15/81

(52) U.S. Cl. CPC C12N 15/1037; C12N 15/113; C12N 15/81; C12N 2310/20; C12N 15/1079; C12N 15/1086; C12N 15/1058

(57) ABSTRACT

The present disclosure provides a combinatorial metabolic engineering system based on an orthogonal tri-functional CRISPR system that combines transcriptional activation, transcriptional interference, and gene deletion (CRISPR-AID). This strategy enables perturbation of the metabolic and regulatory networks in a modular, parallel, and high throughput manner. The present disclosure further provides a multi-functional genome-wide CRISPR (MAGIC) system for high throughput genotype-phenotype mapping.

### CROSS-REFERENCE TO RELATED APPLICATIONS

This application claims priority to U.S. Provisional Patent Application No. 62/585,533, filed Nov. 13, 2017, the disclosure of which is hereby incorporated by cross-reference in its entirety.

### STATEMENT REGARDING FEDERALLY SPONSORED RESEARCH OR DEVELOPMENT

This application was made with United States government support awarded by U.S. Department of Energy (DE-SC0018260). The United States government has certain rights in this invention.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file is 219 kilobytes in size, and titled 18-1731_SequenceListing_ST25.txt.

### BACKGROUND

### Field

The present disclosure provides systems, compositions, and methods for targeted genome engineering based on an orthogonal tri-functional CRISPR system that combines transcriptional activation, transcriptional interference, and gene deletion (CRISPR-AID). The present disclosure further provides a multi-functional genome-wide CRISPR (MAGIC) system and method for high throughput genotype-phenotype mapping.

### Description of the Related Art

Microbial cell factories have been increasingly engineered to produce fuels, chemicals, and pharmaceuticals using various renewable feedstocks (Nielsen, J., et al., Cell 164: 1185-1197 (2016); Du, J., et al., J. Ind. Microbiol. Biotechnol. 38:873-890 (2011)). However, microorganisms have evolved robust metabolic and regulatory networks to survive and grow in specific environments rather than to synthesize the products of industrial interest. Therefore, metabolic engineering of the producing microorganisms is required to rewire the cellular metabolism, i.e. to enhance the supply of the precursor metabolites (Lian, J. & Zhao, H., J., Ind. Microbiol. Biotechnol. 42: 437-451 (2015); Lian, J., et al., Metab. Eng. 24:139-149 (2014); Lian, J., et al., Metab. Eng. 23:92-99 (2014)), to maximize fermentation titer, yield, and productivity for commercially viable processes. To perturb the extensive regulation and complex interactions between metabolic pathways, researchers often need to modify multiple metabolic engineering targets with different modes of regulation, such as to increase expression of genes encoding rate-limiting enzymes, decrease expression of essential genes, and remove expression of competing pathways (Nielsen, J., et al., Cell 164:1185-1197 (2016)). Researchers should be able to control a full spectrum of expression profiles for multiple genes of interest simultaneously. Unfortunately, such rewiring of cellular metabolism is often carried out sequentially and with low throughput, which is largely due to the lack of facile and multiplex genome engineering tools. Homologous recombination based gene replacement is commonly used for genome engineering of the producing microorganisms, but suffers from low efficiency and throughput and is labor and time intensive (Hegemann, J. H., et al., Methods Mol. Biol. 313:129-144 (2006)). Consequently, genome engineering targets are mainly tested individually or in a few combinations. However, due to the limited knowledge on the regulation of cellular metabolism, it is highly desirable to test more metabolic engineering targets in combinations, particularly for those with synergistic interactions. Therefore, development of a combinatorial metabolic engineering strategy to modify the host genome in a modular, parallel, and high throughput manner will be critical to the optimization of microbial cell factories.

Additionally, functional profiling of genotype-phenotype relationships has broad applications in both fundamental biology and biotechnology, such as to decipher the genetic determinants of microbial pathogenesis and construct cell factories with maximal production of the desired metabolites (Si, T., et al., Biotechnol. Adv. 33:1420-1432, (2015)). Nevertheless, the understanding of the complexity of cellular network is rather limited. For example, in the most well-studied eukaryote Saccharomyces cerevisiae, about 1000 genes are included in the most advanced genome-scale metabolic models, while there are more than 6000 genes in the yeast genome (Lian, J., et al., Metab. Eng., (2018); Nielsen, J. & Keasling, J. D., Cell 164:1185-1197, (2016)). In other words, most of the genes have not been clearly mapped into biological pathways or phenotypic traits. Therefore, the identification of genetic determinants particularly for those that work synergistically remains the biggest challenge for understanding and engineering complex phenotypes.

There have been no reports on the development of a multi-functional genome-scale CRISPR system. In other words, the genotypic diversity created by exiting methods is not comprehensive, as both upregulation and downregulation of multiple targets are generally required to engineer the desired phenotype.

### BRIEF SUMMARY

The present disclosure relates to a system for targeted genome engineering and methods for altering the expression of genes and interrogating the function of genes.

One aspect of the disclosure provides a system for targeted genome engineering, the system comprising one or more vectors comprising: (i) a first single guide RNA (sgRNA) that is capable of binding a target nucleic acid and binding a first nuclease-deficient RNA-guided DNA endonuclease protein; (ii) a second sgRNA that is capable of binding a target nucleic acid and binding a second nuclease-deficient RNA-guided DNA endonuclease protein; (iii) a third sgRNA that is capable of binding a target nucleic acid and binding a catalytically-active RNA-guided DNA endonuclease protein; (iv) a polynucleotide encoding a first nuclease-deficient RNA-guided DNA endonuclease protein that binds to the first sgRNA and causes transcriptional activation; (v) a polynucleotide encoding a second nuclease-deficient RNA-guided DNA endonuclease protein that binds to the second sgRNA and causes transcriptional interference; and (vi) a polynucleotide encoding a catalytically active RNA-guided DNA endonuclease protein that binds to the third sgRNA and causes a double-stranded nucleic acid break and causes gene deletion. In some embodiments, components (i), (ii), (iii), (iv), (v), and (vi) of the system for targeted genome engineering are located on the same or different vectors of the system.

In some embodiments of the disclosure, the catalytically active RNA-guided DNA endonuclease protein is CRISPR associated protein (Cas9). In other embodiments, the Cas9 is from Streptococcus pyogenes (SpCas9), Neisseria meningitides (NmCas9), Streptococcus thermophiles (St1Cas9), or Staphylococcus aureus (SaCas9).

In some embodiments of the disclosure, the system for targeted genome engineering comprises one or more vectors that are plasmids or viral vectors.

In some embodiments of the disclosure, the system for targeted genome engineering comprises a first nuclease-deficient RNA-guided DNA endonuclease protein that is functional only when bound to the first sgRNA; a second nuclease-deficient RNA-guided DNA endonuclease protein that is functional only when bound to the second sgRNA; and a catalytically active RNA-guided DNA endonuclease protein that is functional only when bound to the third sgRNA.

In other embodiments of the disclosure, the system for targeted genome engineering does not utilize synthetic CRISPR-repressible promoters or synthetic CRISPR-activatable promoters.

In some embodiments of the disclosure, all of the sgRNAs of the system for targeted genome engineering are expressed in an expression cassette comprising a type II promoter or a type III promoter.

Another aspect of the disclosure provides a polynucleotide comprising a nucleotide sequence encoding a Cpf1 nuclease-deficient RNA-guided DNA endonuclease protein operably linked to at least one VP64-p65AD (VP) activator domain. In some embodiments of the disclosure, the Cpf1 protein is from Lachnospiraceae bacterium or Acidaminococcus sp. In other embodiments of the disclosure, the Cpf1 nuclease-deficient RNA-guided DNA endonuclease protein comprises the sequence of amino acids set forth in SEQ ID NO:573 or at least 95% sequence identity to the sequence set forth in SEQ ID NO:573. In yet other embodiments of the disclosure, the polynucleotide encodes the sequence of amino acids set forth in SEQ ID NO:574 or at least 95% sequence identity to the sequence set forth in SEQ ID NO:574.

Yet another aspect of the present disclosure provides a polynucleotide comprising a nucleotide sequence encoding a Cas9 RNA-guided DNA endonuclease protein operably linked to more than one repression domain. In some embodiments of the disclosure, the Cas9 protein is from Streptococcus pyogenes, Neisseria meningitides, Streptococcus thermophiles, or Staphylococcus aureus. In other embodiments of the disclosure, the Cas9 RNA-guided DNA endonuclease protein comprises the sequence of amino acids set forth in SEQ ID NO:575 or at least 95% sequence identity to the sequence set forth in SEQ ID NO:575. In yet other embodiments of the disclosure, the polynucleotide encodes the sequence of amino acids set forth in SEQ ID NO:576 or at least 95% sequence identity to the sequence set forth in SEQ ID NO:576.

In some embodiments of the disclosure, the polynucleotide comprises a nucleotide sequence encoding a dSpCas9 protein operably linked to the C-terminal end to a RD11 repression domain, wherein a RD5 repression domain is operably linked to the C-terminal end of the RD11 domain, wherein a RD2 repression domain is operably linked to the C-terminal end of the RD5 domain. In other embodiments of the disclosure, the at least one repression domain is operably linked to the N-terminal and/or C-terminal ends of the nuclease-deficient RNA-guided DNA endonuclease protein, or operably linked in tandem at the C-terminal end of the nuclease-deficient RNA-guided DNA endonuclease protein.

Yet another aspect of the present disclosure provides a method of altering the expression of gene products, the method comprising: introducing into a cell the system of targeted genome engineering described above, wherein the expression of at least one gene product is increased, the expression of at least one gene product is decreased, and the expression of at least one gene products is deleted relative to a cell that has not been transformed with the system for targeted genome engineering.

In some embodiments of the present disclosure, the method of altering the expression of gene products further comprises selecting for successfully transformed cells by applying selective pressure.

In some embodiments of the present disclosure, the method occurs in vivo or in vitro.

In some embodiments of the present disclosure, the cell involved in the method of altering the expression of gene products is a eukaryotic cell. In other embodiments of the present disclosure, the cell is a yeast cell. In yet other embodiments, the yeast cell is Saccharomyces cerevisiae.

In some embodiments of the present disclosure, the at least one gene product is a protein involved in the mevalonate pathway. In other embodiments of the present disclosure, the expression of HMG1 is increased, the expression of ERGS is decreased, and the expression of ROX1 is deleted.

In some embodiments of the present disclosure, the method of altering the expression of gene products further comprises increasing production of an isoprenoid in the cell. In other embodiments, the isoprenoid is β-carotene.

In some embodiments of the present disclosure, the method of altering the expression of gene products further comprises increasing expression of a surface protein on the cell. In some embodiments, the expression of PDI1 is increased, the expression of MNN9 is decreased, and the expression of PMR1 is deleted. In other embodiments, the method further comprises increasing EGII display levels and cellulase activity.

Yet another aspect of the present disclosure provides a method of identifying the genetic basis of one or more phenotypes of cells, the method comprising: (i) preparing three genome-scale sgRNA expressing plasmid libraries from oligonucleotides wherein the first genome-scale sgRNA expressing plasmid library is for upregulating genes of the cells, wherein the second genome-scale sgRNA expressing plasmid library is for downregulating genes of the cells, and the third genome-scale sgRNA expressing plasmid library is for deleting genes of the cells; (ii) transforming the three genome-scale sgRNA expressing plasmid libraries into the cells; (iii) introducing into the cells a polynucleotide encoding a first nuclease-deficient RNA-guided DNA endonuclease protein that binds to the sgRNA of the first genome-scale sgRNA expressing plasmid library and causes transcriptional activation of genes of a cell, a polynucleotide encoding a second nuclease-deficient RNA-guided DNA endonuclease protein that binds to the sgRNA of the second genome-scale sgRNA expressing plasmid library and causes transcriptional repression of genes of the cells, and a polynucleotide encoding a catalytically active RNA-guided DNA endonuclease protein that binds to a the sgRNA of the third genome-scale sgRNA expressing plasmid library and causes double-stranded nucleic acid breaks and gene deletion of genes of a cell; (iv) isolating transformed cells with one or more phenotypes; and (v) determining the genomic loci of the DNA molecule that causes the one or more phenotypes.

In some embodiments of the disclosure, the cell is a yeast cell. In other embodiments, the cell is a eukaryotic cell.

In some embodiments of the method of identifying the genetic basis of one or more phenotypes of a cell, the phenotype is furfural tolerance or yeast surface display of recombinant proteins.

Therefore, provided herein are orthogonal and generally applicable tri-functional CRISPR systems comprising CRISPRa, CRISPRi, and CRISPRd (CRISPR-AID) for metabolic engineering of eukaryotic and prokaryotic cells, both in vitro and in vivo. Due to the modular and multiplex advantages of the CRISPR system, CRISPR-AID can be used for combinatorial optimization of various metabolic engineering targets and exploration of the synergistic interactions among transcriptional activation, transcriptional interference, and gene deletion in S. cerevisiae.

As further described herein, the tri-functional CRISPR system can be combined with array-synthesized oligo pools to create a multi-functional genome-wide CRISPR (MAGIC) system. While most existing methods for genome-scale engineering are limited to a single mode of genomic alteration (i.e., overexpression, repression, or deletion), the MAGIC system can be used for high throughput genotype-phenotype mapping to identify novel genetic determinants of complex phenotypes, particularly those with synergistic interactions when regulated to different expression levels.

Additional features and advantages are described herein, and will be apparent from the following Detailed Description, Drawings and the claims.

### BRIEF DESCRIPTION OF THE DRAWINGS

The features, objects and advantages other than those set forth above will become more readily apparent when consideration is given to the detailed description below. Such detailed description makes reference to the following drawings, wherein:

FIG. 1A-1B illustrates the design of CRISPR-AID for combinatorial metabolic engineering. FIG. 1A shows a schematic of cell factories for sustainable production of fuels, chemicals, and drugs from renewable resources. FIG. 1B shows a schematic of development of CRISPR-AID using three orthogonal CRISPR proteins, one nuclease-deficient CRISPR protein fused with an activation domain for CRISPRa, another nuclease-deficient mutant fused with a repression domain for CRISPRi, and a third catalytically active CRISPR protein for CRISPRd. FIG. 1C shows a schematic CISPR-AID enabled combinatorial metabolic engineering by exploring all the possible gRNA combinations to construct optimal cell factories.

FIG. 2A-2D illustrates construction of a reporter strain for CRISPR-AID. FIG. 2A is a graph showing fluorescence intensities of mVenus and mCherry of the reporter strain. FIG. 2B is a graph showing strain CT for CRISPRa, with dSpCad9-VPR (Sg6) for the activation of CYC1p included as a positive control. The expression level of mCherry was increased more than 5-fold. FIG. 2C is a graph showing strain CT for CRISPRi, with dSpCad9-MXI1 (Sg1) for the interference of TEF1p included as a positive control. The expression level of mVenus was decreased around 10-fold. FIG. 2D illustrates strain CT for CRISPRd, with SpCas9 (Sg11) for the deletion of ADE2 gene included as a positive control. Error bars represent the mean±s.d. of biological quadruplicates.

FIG. 3 is a graph showing the deletion efficiency of orthogonal CRISPR proteins for CRISPR-AID. The orthogonality was tested by co-transforming the CRISPR proteins (SpCas9, St1Cas9, SaCas9, and LbCpf1) and gRNAs (Sg10, Sg64, Sg95, and Sg122) with different origins and evaluating ADE2 deletion efficiency.

FIG. 4A-4E illustrates optimization of CRISPRa by testing all the combinations (FIG. 4A) of 4 nuclease-deficient CRISPR proteins, including dSpCas9 (FIG. 4B), dSaCas9 (FIG. 4C), dSt1Cas9 (FIG. 4D), and dLbCpf1 (FIG. 4E), and 3 activation domains (V, VP, and VPR) with different levels of strength, dSpCas9-VPR and dLbCpf1-VP were found to be the optimal combinations with the strongest activation and highest degree of flexibility in gRNA design. Error bars represent the mean±s.d. of biological quadruplicates.

FIG. 5A-5C illustrates optimization of CRISPRi by repression domain engineering. FIG. 5A is a schematic showing the workflow of repression domain engineering for optimal CRISPRi. Endogenous repression domains (RD1, RD2, RD3, RD4, RD5, RD6, RD7, RD8, RD9, RD10, and RD11) were tested individually for CRISPRi efficiency and then multiple repression domains were combined either in the form of N- and C-terminal tagged (2RD5, 2RD11, and 5RD11) or tandem repeat at the C-terminus (RD1152) for maximal CRISPRi efficiency. FIG. 5B is a graph and schematic showing enhanced CRISPRi efficiency using endogenous repression domains. The MXI1 repression domain was replaced with 11 well-characterized repression domains from S. cerevisiae. CRISPRi efficiency was quantified by normalizing the mVenus fluorescence intensities to those of dSpCas9-MXI1. FIG. 5C is a graph and schematic showing further enhanced CRISPRi efficiency using multiple repression domains. The mVenus fluorescence intensities were normalized to those without gRNA targeting sequences (SgH). Error bars represent the mean±s.d. of biological quadruplicates.

FIG. 6A-6B illustrates selection of appropriate nuclease-deficient CRISPR protein for CRISPRi. The CRISPRi efficiency using dSpCas9-MXI1 (FIG. 6A) and dLbCpf1-MXI1 (FIG. 6B) were systematically compared, with several gRNAs targeting both the promoter region (blocking transcriptional initiation; Sg1, Sg27 and Sg28 for dSpCas9-MXI1; Sg125 and Sg126 for dLbCpf1-MXI1) and coding region (blocking transcriptional elongation; Sg109, Sg110, Sg111, Sg112, Sg113, and Sg114 for dSpCas9-MXI1; Sg135, Sg136, and Sg137 for dLbCpf1-MXI1) included for analysis. Generally, more efficient CRISPRi was achieved when using dSpCas9-MXI1 and targeting the promoter region. Error bars represent the mean±s.d. of biological quadruplicates.

FIG. 7A-7B illustrates CRISPRi using the engineered repression domain for additional reporter strains. FIG. 7A is a graph and schematic showing the CRISPRi efficiency using dSpCas9-MXI1 and dSpCas9-RD1152 for strain CF targeting FBA1p. FIG. 7B is a graph and schematic showing the CRISPRi efficiency using dSpCas9-MXI1 and dSpCas9-RD1152 for strain CH targeting HHF2p. The CRISPRi efficiency was normalized to that achieved using dSpCas9-MXI1. Error bars represent the mean±s.d. of biological quadruplicates.

FIG. 8 is a graph showing the multiplex gRNA design for CRISPR-AID. PC: Individual gRNA cassette. Design I: expression of multiple gRNAs in a single cassette driven by a type III promoter (SNR52p) (SNR52p-gRNAa-Csy4-gRNAi-Csy4-gRNAd-SUP4t). Design II: expression of multiple gRNAs in multiple cassettes driven by a type III promoter (SNR52p) ([SNR52p-gRNAa-SUP4t]-[SNR52p-gRNAi-SUP4t]-[SNR52p-gRNAd-SUP4t]). Design III: expression of multiple gRNAs in a single cassette driven by a type II promoter (TEF1p) (TEF1p-Csy4-gRNAa-Csy4-gRNAi-Csy4-gRNAd-Csy4-CY1t). Plasmids containing only one gRNA cassette were included as positive controls (PC). Design I allowed the expression of no more than 2 gRNAs. Design II and Design III allowed the expression of full length multiple gRNAs with genome engineering efficiency comparable to those with one gRNA. Error bars represent the mean±s.d. of biological quadruplicates.

FIG. 9A-9C illustrates CRISPR-AID using the reporter yeast strain CT. By transforming the reporter strain with a single plasmid containing an array of 3 gRNAs, transcriptional activation of mCherry (FIG. 9A), transcriptional interference of mVenus (FIG. 9B), and deletion of an endogenous ADE2 gene (FIG. 9C) were achieved simultaneously with high efficiency. The inset in FIG. 9C shows a representative result of ADE2 deletion using CRISPR-AID. Error bars represent the mean±s.d. of biological quadruplicates.

FIG. 10A-10C illustrates CRISPR-AID for rational metabolic engineering. FIG. 10A is a schematic showing β-Carotene biosynthesis as a representative example of rational metabolic engineering. HMG1, ERG9, and ROX1 were chosen as the targets for CRISPRa, CRISPRi, and CRISPRd, respectively. FIG. 10B is a graph showing improved β-carotene production using single gRNA plasmids (A-pSg175, I-pSg172, and D-pSg186), a double gRNA plasmid (AI-pSg585), and a triple gRNA plasmid (AID-pSg239). The inset shows the yeast cultures before (SgH) and after (Sg239) CRISPR-AID engineering. FIG. 10C is a graph showing verification of CRISPRa (HMG1) and CRISPRi (ERG9) for transcriptional regulation using qPCR. Error bars represent the mean±s.d. of biological triplicates.

FIG. 11A-11E illustrates diagnostic PCR verification of the deletion of the targeted genes by CRISPRd. FIG. 11A shows diagnostic PCR verification of the deletion of ROX1 by CRISPRd. FIG. 11B shows diagnostic PCR verification of the deletion of PMR1 by CRISPRd. FIG. 11C shows diagnostic PCR verification of the deletion of PEP4 by CRISPRd. FIG. 11D shows diagnostic PCR verification of the deletion of VPS1 by CRISPRd. FIG. 11E shows diagnostic PCR verification of the deletion of YPS1 by CRISPRd.

FIG. 12A-12E illustrates CRISPR-AID for combinatorial metabolic engineering. FIG. 12A is a schematic showing yeast surface display of recombinant proteins as a representative example of combinatorial metabolic engineering. Protein folding and secretory machinery, protein super-glycosylation and other surface-displayed proteins, and degradation pathways were chosen as the targets for CRISPRa, CRISPRi, and CRISPRd, respectively. FIG. 12B is a graph showing combinatorial optimization of EGII display on the yeast surface. EGII activities of the FACS enriched optimal combination (AID-FACS16) and those with the corresponding single component (A-pSg221, I-pSg230, and D-pSg205) were measured. FIG. 12C is a graph showing verification of CRISPRa (PDI1) and CRISPRi (MNN9) for transcriptional regulation using qPCR. FIG. 12D is a graph showing the synergistic interactions among activated (PDI1), interfered (MNN9), and deleted (PMR1) metabolic engineering targets. EGII activities of the double mutants, including AI-pSg417, AD-pSg418, and ID-pSg419, were measured. FIG. 12E is a graph showing single-factor optimization of EGII display on the yeast surface. EGII activities of the strains with one gRNA (A-pSg218, I-pSg204, and D-pSg186) and the combination of the ones with the highest activities in each category (AID-pSg257) were measured. Error bars represent the mean±s.d. of biological triplicates.

FIG. 13 is a graph showing EGII activity with one gRNA. 14 CRISPRa, 17 CRISPRi, and 5 CRISPRd targets were chosen, most of which resulted in improved protein display level and EGII activity. Sg218 (ERO2), Sg204 (PMR1), and Sg186 (ROX1) worked the best for CRISPRa, CRISPRi, and CRISPRd, respectively. The gRNA plasmids were transformed into CEN-EGII and the resultant recombinant strains were cultured in SED-HIS-URA/G418 medium for ˜3 days for cellulase activity assays. Error bars represent the mean±s.d. of biological triplicates.

FIG. 14A-14B illustrates quantification of recombinant proteins displayed on yeast surface using immunostaining. FIG. 14A is a graph showing unstained and PE stained control yeast strain as analyzed by flow cytometry. FIG. 14B is a graph showing unstained and PE stained EGII-displaying strain as analyzed by flow cytometry.

FIG. 15A-15B illustrates FACS sorting of the EGII-displaying library. FIG. 15A illustrates FACS sorting profiles of the control yeast strain. FIG. 15B illustrates FACS sorting profiles of the EGII-displaying library. The gate P2 was set to collect yeast cells with top 1% of the highest fluorescence.

FIG. 16 is a graph showing EGII activity of the transformed library and the FACS sorted library. The library strains were cultured in SED-HIS-URA/G418 medium for ˜3 days for cellulase activity assays. Error bars represent the mean±s.d. of biological triplicates.

FIG. 17 is a graph showing EGII activity of the FACS sorted individual clones. 96 single clones with the highest fluorescence signals were sorted using FACS, and the plasmids were extracted and re-transformed into CEN-EGII strain with a fresh background. 26 yeast strains showing the highest PE fluorescence intensity after re-transformation were chosen for cellulase activity assays. FACS-Re16 and FACS-Re22 showed the highest EGII activity. Error bars represent the mean±s.d. of biological triplicates.

FIG. 18A-18B illustrates single factor optimization using CRISPR-AID. The top candidates from each category (A-pSg218, ERO1 activation; I-pSg204, PMR1 interference; and D-pSg186, ROX1 deletion) were combined (AID-pSg257) and characterized. Transcriptional regulation and genome editing were verified using qPCR and diagnostic PCR, respectively. FIG. 18A is a graph showing verification of CRISPRa (ERO1) and CRISPRi (PMR1) for transcriptional regulation using qPCR. Error bars represent the mean±s.d. of biological triplicates. FIG. 18B illustrates verification of the disruption of ROX1 in D (pSg186, 3 independent clones) and AID (pSg257, 2 independent clones) strains using diagnostic PCR.

FIG. 19A-19B illustrates CRISPRi using truncated gRNAs. FIG. 19A is a graph showing the effect of gRNA truncation on CRISPRi efficiency. FIG. 19B is a graph showing a comparison of CRISPRi efficiency using full length and truncated gRNAs. The full length (Sg1) and truncated (Sg27) gRNAs were transformed into dSpCas9-MXI1 containing yeast strain and resulted in comparable CRISPRi efficiency. Error bars represent the mean±s.d. of biological quadruplicates.

FIG. 20 is a graph showing CRISPRa using modular RNA scaffold. MS2 aptamer was included in Sg45, and the specific RNA binding protein (MS2) would recruit VP64 to activate the expression of mCherry under the control of CYC1p. CRISPRa efficiency was comparable with that achieved using dSpCas9-VPR. Error bars represent the mean±s.d. of biological quadruplicates.

FIG. 21 is a graph showing CRISPRi using engineered modular RNA scaffold. The fusion of an aptamer resulted in much lower CRISPRi efficiency, even though a repression domain was recruited through the specific RNA binding protein. The use of different aptamers and repression domains did not increase CRISPRi efficiency significantly. A much higher CRISPRi efficiency could be achieved using Sg27, without the inclusion of an aptamer and a repression domain. Notably, such high CRISPRi efficiency could only be achieved for a few cases when targeting the promoter region, if no repression domain was included. Error bars represent the mean±s.d. of biological quadruplicates.

FIG. 22 is a schematic showing the MAGIC pipeline for genome-wide mapping genotype-phenotype relationships. Guide sequences for genome-scale activation, interference, and deletion were synthesized as arrayed oligos on DNA chip and cloned into the corresponding gRNA expression plasmids using Golden-Gate Assembly. The MAGIC library was constructed by transforming the pooled plasmid libraries into the CRISPR-AID integrated yeast strain, and subject to growth enrichment under various conditions or high throughput screening. The enrichment and depletion of guide sequences were profiled using next generation sequencing. The MAGIC workflow can be iterated to better understand and engineer complex phenotypes.

FIG. 23A-23C illustrates score distribution of the designed guide sequences for the genome-scale activation (FIG. 23A), and interference (FIG. 23B), and deletion (FIG. 23C) libraries, respectively. Based on the score equation detailed in Example 6, the highest score for activation, interference, and deletion libraries are 3, 4, and 4, respectively. The dashed line represents the percentage of gRNAs with high scores (higher than 60% of the maximal score).

FIG. 24A-24I illustrates that iterative MAGIC enabled genome-wide understanding and engineering of furfural tolerance in yeast. FIG. 24A illustrates the MAGIC library screened in the first round under a furfural concentration of 5 mM. FIG. 24B is a graph showing the relative biomass accumulation of the top guide sequences under a furfiral concentration of 5 mM. FIG. 24C illustrates the MAGIC library screened in the second round under a furfural concentration of 10 mM. FIG. 24D is a graph showing the relative biomass accumulation of the top guide sequences under a furfiral concentration of 10 mM. FIG. 24E illustrates the MAGIC library screened in the third round under a furfural concentration of 15 mM. FIG. 24F is a graph showing the relative biomass accumulation of the top guide sequences under a furfiral concentration of 15 mM. The light grey dots represented the control guide sequences. FIG. 24G is a graph showing furfural tolerance of the engineered strains identified in each round of MAGIC screening, R1, R2, and R3. FIG. 24H is a graph showing verification of gain- and reduction-of-function mutations by qPCR. FIG. 24I is a graph showing synergistic interactions among targets (T) identified in different rounds of MAGIC screening. Error bars represent the mean±s.d. of biological triplicates.

FIG. 25 is a graph showing verification of the second round MAGIC screening identified targets when integrated into the X4 locus of R1 strain (SIZ1i). The strains were pre-cultured in SED medium until saturation and then inoculated into fresh SED medium supplemented with 12.5 mM furfural. The cell density was measured in 24h. Error bars represent the mean±s.d. of biological triplicates.

FIG. 26 is a graph showing verification of the third round MAGIC screening identified targets when integrated into the XI1 locus of the R2 strain (SIZ1i-NAT1a). The strains were pre-cultured in SED medium until saturation and then inoculated into fresh SED medium supplemented with 17.5 mM furfural. The cell density was measured in 36h. Error bars represent the mean±s.d. of biological triplicates.

FIG. 27A-27D illustrates the fermentation profiles of WT and the R3 strain in synthetic medium with or without the supplementation of 17.5 mM furfural (Ff). FIG. 27A is a graph showing cell density over time of the WT and the R3 strain in synthetic medium with or without the supplementation of 17.5 mM Ff. FIG. 27B is a graph showing glucose consumption over time of the WT and the R3 strain in synthetic medium with or without the supplementation of 17.5 mM Ff. FIG. 27C is a graph showing ethanol production over time of the WT and the R3 strain in synthetic medium with or without the supplementation of 17.5 mM Ff. FIG. 27D is a graph showing furfural and furfuryl alcohol (FfOH) concentration over time of the WT and the R3 strain in synthetic medium with or without the supplementation of 17.5 mM Ff. Error bars represent the mean±s.d. of biological triplicates.

FIG. 28A-28C illustrations the identification of genetic determinants of yeast surface display of recombinant proteins by MAGIC. FIG. 28A is a graph showing the 1^{st} round of MAGIC screening identified HOCld as the best target, followed by UBP3i and MNN9i, and the 2^{nd} round of MAGIC screening identified NUP157i and PDI1a as the top candidates that worked synergistically with HOCld to improve display levels of recombinant proteins on yeast surface. The cellulase activity of WT (bAID-EG), EG11 (HOC1d), EG12 (UBP3i), EG13 (MNN9i), EG21 (HOC1d-NUP157i), and EG22 (HOC1d-PDI1a) were measured and compared. FIG. 28B is a gel image confirming the deletion of HOC1 and interference of NUP157 in EG21 by diagnostic PCR. FIG. 28C is a graph confirming the deletion of HOC1 and interference of NUP157 in EG21 by qPCR. Error bars represent the mean±s.d. of biological triplicates.

FIG. 29 is a graph showing the comparison of the furfural tolerance of the engineered yeast strains obtained by two rounds of MAGIC and CHAnGE screening. The WT, R1 (SIZ1i), R2 (SIZ1i-NAT1a) and CHAnGE strain (SIZ1d-LCB3d)³ were pre-cultured in SED until saturation and then inoculated into fresh SED medium supplemented with 10 mM furfural with an initial OD of 0.05. The cell density was measured in 24h. Error bars represent the mean±s.d. of biological triplicates

FIG. 30A-30B illustrates characterization of the integration and gRNA expression efficiency of the pre-selected genomic loci. FIG. 30A is a graph showing the relative mCherry fluorescence intensities of eight colonies from each loci. FIG. 30B is a graph showing the relative mVenus fluorescence intensities of eight colonies from each loci. NC indicates the absence of any targeting gRNA; PC for CRISPRa includes a gRNA expression plasmid for mCherry activation, while PC for CRISPRi includes a gRNA expression plasmid for mVenus repression.

While the present methods and compositions are susceptible to various modifications and alternative forms, exemplary embodiments thereof are shown by way of example in the drawings and are herein described in detail. It should be understood, however, that the description of exemplary embodiments is not intended to limit the methods and compositions to the particular forms disclosed, but on the contrary, the intention is to cover all modifications, equivalents and alternatives falling within the spirit and scope of the methods and compositions as defined by the embodiments above and the claims below. Reference should therefore be made to the embodiments above and claims below for interpreting the scope of the methods and compositions.

### DETAILED DESCRIPTION

The system and methods now will be described more fully hereinafter with reference to the accompanying drawings, in which some, but not all embodiments of the methods and compositions are shown. Indeed, the methods and compositions can be embodied in many different forms and should not be construed as limited to the embodiments set forth herein; rather, these embodiments are provided so that this disclosure will satisfy applicable legal requirements.

Likewise, many modifications and other embodiments of the system and methods described herein will come to mind to one of skill in the art to which the systems and methods pertains having the benefit of the teachings presented in the foregoing descriptions and the associated drawings. Therefore, it is to be understood that the methods and compositions are not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art to which the systems and methods pertain.

Articles “a” and “an” are used herein to refer to one or to more than one (i.e. at least one) of the grammatical object of the article. By way of example, “an element” means at least one element and can include more than one element.

As used herein, the term “and/or” includes any and all combinations of one or more of the associated listed items. As used herein, the singular forms “a,” “an,” and “the” are intended to include the plural forms as well as the singular forms, unless the context clearly indicates otherwise.

The embodiments illustratively described herein suitably can be practiced in the absence of any element or elements, limitation or limitations that are not specifically disclosed herein. Thus, for example, in each instance herein any of the terms “comprising,” “consisting essentially of,” and “consisting of” may be replaced with either of the other two terms, while retaining their ordinary meanings.

The term “about” in association with a numerical value means that the numerical value can vary plus or minus by 5% or less of the numerical value.

CRISPR-CAS9 System

The Clustered Regularly Interspersed Short Palindromic Repeats/CRISPR-associated (CRISPR/Cas) system includes recently identified types of sequence-specific nucleases. CRISPR/Cas molecules are components of a prokaryotic adaptive immune system that is functionally analogous to eukaryotic RNA interference, using RNA base pairing to direct DNA or RNA cleavage. Directing DNA double stranded breaks requires two components: the Cas9 protein, which functions as an endonuclease, and CRISPR RNA (crRNA) and tracer RNA (tracrRNA) sequences that aid in directing the Cas9/RNA complex to target DNA sequence. The modification of a single targeting RNA can be sufficient to alter the nucleotide target of a Cas9 protein. In some cases, crRNA and tracrRNA can be engineered as a single cr/tracrRNA hybrid to direct Cas9 cleavage activity. The CRISPR/Cas system can be used in bacteria, yeast, humans, and zebrafish.

CRISPR-AID System

Designing an optimal microbial cell factory often requires overexpression, knock-down, and knock-out of multiple gene targets. Unfortunately, such rewiring of cellular metabolism is often carried out sequentially and with low throughput. A combinatorial metabolic engineering strategy based on a tri-functional CRISPR system is described herein that combines orthogonal proteins for transcriptional activation, transcriptional interference, and gene deletion (CRISPR-AID) in eukaryotic and prokaryotic cells (e.g., mammalian, bacterial, yeast cells)

CRISPR-AID, a tri-functional CRISPR system combining transcriptional activation (CRISPRa), transcriptional interference (CRISPRi), and gene deletion (CRISPRd), for combinatorial metabolic engineering is provided herein. The systems enable the exploration of the gain- and loss-of-function combinations that work synergistically to improve the desired phenotypes. CRISPR-AID not only includes three modes of genome engineering (gene activation, gene interference, and gene deletion), but also has different mechanisms of genome modulation than, for example, RNAi and offers several advantages. For example, down-regulation using CRISPRi or RNAi is required for the modulation of essential genes, while CRISPRd enables more stable and in many cases significant phenotypes when targeting non-essential genes; CRISPRa is less biased for overexpression of large genes during large scale combinatorial optimization; CRISPRi blocks transcription in the nucleus while RNAi affects mRNA stability and translation, and CRISPRi is generally found to have higher repression efficiency in many situations. Using CRISPR-AID, different modes of genomic modifications (i.e. activation, interference, and deletion) can be introduced via gRNAs on a plasmid or other delivery method. Combinatorial metabolic engineering can be achieved by testing all the possible gRNA combinations. All the combinations of the metabolic engineering targets of the metabolic and regulatory network related to a desired phenotype can be explored.

One embodiment provides a system for targeted genome engineering, the system comprising one or more vectors comprising: (i) a first single guide RNA (sgRNA) that is capable of binding a target nucleic acid and binding a first nuclease-deficient RNA-guided DNA endonuclease protein; (ii) a second sgRNA that is capable of binding a target nucleic acid and binding a second nuclease-deficient RNA-guided DNA endonuclease protein; (iii) a third sgRNA that is capable of binding a target nucleic acid and binding a catalytically-active RNA-guided DNA endonuclease protein; (iv) a polynucleotide encoding a first nuclease-deficient RNA-guided DNA endonuclease protein that binds to the first sgRNA and causes transcriptional activation; (v) a polynucleotide encoding a second nuclease-deficient RNA-guided DNA endonuclease protein that binds to the second sgRNA and causes transcriptional interference; and (vi) a polynucleotide encoding a catalytically active RNA-guided DNA endonuclease protein that binds to the third sgRNA and causes a double-stranded nucleic acid break and causes gene deletion.

The system for targeted genome engineering can comprise more than one first single guide RNA (sgRNA) (e.g., 2, 3, 4, 5, 10, or more) that are capable of binding a target nucleic acid sequence and binding a first nuclease-deficient RNA-guided DNA endonuclease protein; more than one second sgRNA (e.g., 2, 3, 4, 5, 10, or more) that are capable of binding a target nucleic acid sequence and binding a second nuclease-deficient RNA-guided DNA endonuclease protein; more than one third sgRNA (e.g., 2, 3, 4, 5, 10, or more) that is capable of binding a target nucleic acid and binding a catalytically-active RNA-guided DNA endonuclease protein; a polynucleotide encoding a first nuclease-deficient RNA-guided DNA endonuclease protein that binds to the first group of sgRNA and causes transcriptional activation; a polynucleotide encoding a second nuclease-deficient RNA-guided DNA endonuclease protein that binds to the second group of sgRNA and causes transcriptional interference; and a polynucleotide encoding a catalytically active RNA-guided DNA endonuclease protein that binds to the third group of sgRNA and causes a double-stranded nucleic acid break and causes gene deletion.

The single guide RNA (sgRNA) capable of directing nuclease-deficient RNA-guided DNA endonuclease mediated transcriptional activation of target DNA, the sgRNA capable of causing transcriptional interference, and the sgRNA that capable of directing catalytically active RNA-guided DNA endonuclease mediated gene deletion or knock-out of target DNA can each target a different target nucleic acid.

As used herein, the term “targeted genome engineering” refers to a type of genetic engineering in which DNA is inserted, deleted, modified, modulated or replaced in the genome of a living organism or cell. Targeted genome engineering can involve integrating nucleic acids into or deleting nucleic acids from genomic DNA at a target site of interest in order to manipulate (e.g., increase, decrease, knockout, activate, interfere with) the expression of one or more genes. Targeted genome engineering can also involve recruiting RNA polymerase to or repressing RNA polymerase at a target site of interest in the genomic DNA in order to activate or repress expression of one or more genes.

Several aspects of the disclosure relate to vector systems comprising one or more vectors, or vectors as such. Vectors can be designed for expression of nuclease deficient RNA-guided DNA endonucleases, catalytically active RNA-guided DNA endonucleases, and polynucleotides (e.g. nucleic acid transcripts, proteins, or enzymes) in prokaryotic or eukaryotic cells. For example, nuclease deficient RNA-guided DNA endonucleases, catalytically active RNA-guided DNA endonucleases or polynucleotides can be expressed in bacterial cells such as Escherichia coli, insect cells (using baculovirus expression vectors), yeast cells, or mammalian cells. Suitable cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

A vector or expression vector is a replicon, such as a plasmid, phage, or cosmid, to which another nucleic acid segment can be attached so as to bring about the replication of the attached segment. A vector is capable of transferring polynucleotides (e.g. gene sequences) to target cells.

Expression refers to the process by which a polynucleotide is transcribed from a DNA template (such as into a sgRNA, tRNA or mRNA) and/or the process by which a transcribed mRNA is subsequently translated into peptides, polypeptides, or proteins. Transcripts and encoded polypeptides can be collectively referred to as “gene product.” A polypeptide is a linear polymer of amino acids that are linked by peptide bonds. If the polynucleotide is derived from genomic DNA, expression may include splicing of the mRNA in a eukaryotic cell.

Many suitable expression vectors and features thereof are known in the art. Expression vectors can contain, without limitation, a centromeric (CEN) sequence, an autonomous replication sequence (ARS), a promoter, an origin of replication, and a marker gene (e.g., auxotrophic, antibiotic, or other selectable markers). Examples of expression vectors may include plasmids, yeast artificial chromosomes, 2μπι plasmids, yeast integrative plasmids, yeast replicative plasmids, shuttle vectors, and episomal plasmids. Examples of vectors that can be used with the CRISPR-AID and CRISPR-MAGIC systems include, for example, BsaI-free pRS423, and those described in Table 1 and Table 2.

One or more vectors can be plasmids or viral vectors. In other embodiments, the viral vector is a lentivirus vector, an adenovirus vector, or an adeno-associated vector (AAV).

In some embodiments, a vector is a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include, but are not limited to, pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, a recombinant mammalian expression vector is capable of directing expression of a nucleic acid in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

Vectors can be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A. respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

A promoter is any nucleic acid sequence that regulates the initiation of transcription for a particular polypeptide-encoding nucleic acid under its control. A promoter minimally includes the genetic elements necessary for the initiation of transcription (e.g., RNA polymerase Ill-mediated transcription), and can further include one or more genetic regulatory elements that serve to specify the prerequisite conditions for transcriptional initiation. Promoter means a cis-acting DNA sequence, generally 80-120 base pairs long and located upstream of the initiation site of a gene, to which RNA polymerase may bind and initiate correct transcription. There can be associated additional transcription regulatory sequences which provide on/off regulation of transcription and/or which enhance (increase) expression of the downstream coding sequence. A coding sequence is the part of a gene or cDNA which codes for the amino acid sequence of a protein, or for a functional RNA such as a tRNA or rRNA.

A promoter can be encoded by an endogenous genome of a cell, or it can be introduced as part of a recombinantly engineered polynucleotide. A promoter sequence can be taken from one species and used to drive expression of a gene in a cell of a different species. A promoter sequence can also be artificially designed for a particular mode of expression in a particular species, through random mutation or rational design. In recombinant engineering applications, specific promoters are used to express a recombinant gene under a desired set of physiological or temporal conditions or to modulate the amount of expression of a recombinant nucleic acid. Promoters used in the systems described herein include, for example, type II promoters (e.g., TEF1p, GPDp, PGK1p, and HXT7p) and type III promoters (SNR52p, PROp, and TYRp).

Regulatory elements are promoters, enhancers, internal ribosomal entry sites (IRES), and other expression control elements (e.g. transcription termination signals, such as polyadenylation signals and poly-U sequences). Such regulatory elements are described, for example, in Goeddel, (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g. liver, pancreas), or particular cell types (e.g. lymphocytes). Regulatory elements can also direct expression in a temporal-dependent manner, such as in a cell-cycle dependent or developmental stage-dependent manner, which may or may not also be tissue or cell-type specific. In some embodiments, a vector for expressing gRNAs and/or RNA-guided DNA endonuclease proteins comprises one or more pol III promoters, one or more pol II promoters, one or more pol I promoters, or combinations thereof. Examples of pol III promoters include, but are not limited to, U6 and H1 promoters.

Regulatory elements also include enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

Reporter yeast strains can be used in the systems and methods described herein. Reporter yeast stains can be transformed with one or more reporter plasmids containing gRNAs for transcriptional activation, interference, and deletion. Reporter plasmids can be used for observing the function of genetic elements, and contain a reporter or marker gene (e.g., luciferase or GFP) that offers a read-out of the activity of the genetic element. For example, a promoter of interest could be engineered upstream of the luciferase gene to determine the level of transcription driven by that promoter. The reporter plasmids can be linearized before transformation into a yeast cell. The purpose of linearization of the reporter plasmids is to integrate them into the genome. To demonstrate the CRISPR-AID system in yeast, a reporter yeast strain can be used comprising mCherry driven by a medium-strength promoter CYC1p for CRISPRa (transcriptional activation), mVenus driven by a strong promoter TEF1p for CRISPRi (transcriptional interference), and ADE2, an endogenous gene whose disruption results in the formation of red colonies in adenine deficient synthetic medium, for CRISPRd (gene deletion).

Transcriptional activation or activate refers to activation of gene expression, which can include, but is not limited to, increasing the levels of gene products or initiating gene expression of a previously inactive gene. Robust and controllable systems for activation of native gene expression have been pursued for multiple applications in gene therapy, regenerative medicine, and synthetic biology. These systems, rather than introducing heterologous genes that are expressed from constitutive or tunable promoters, use proteins that regulate transcription of genes in their natural chromosomal context. When activated, the amount of a gene product or gene expression can be increased by about 2, 3, 4, 5, 10, 15, 20, 25, 30, 35, 40, 45, 50 fold or more.

Transcriptional interference refers to the suppressive, direct, and in cis influence of one transcription process by a secondary transcriptional process. Transcriptional interference can be achieved by either blocking transcriptional initiation (i.e. binding to the promoter region) or transcriptional elongation (i.e. binding to the coding sequences). The result of transcriptional interference is that the amount of a gene product or gene expression is decreased by about 2, 3, 4, 5, 10, 15, 20, 25, 30, 35, 40, 45, 50 fold or more.

As used herein, the terms “gene deletion” or “knockout” refers to a genetic technique in which a gene is made inoperative. That is, a gene product is not expressed. Knocking out two genes simultaneously results in a double knockout. Similarly, triple knockout (TKO) and quadruple knockouts (QKO) are used to describe three or four knocked out genes, respectively. Heterozygous knockouts refer to when only one of the two gene copies (alleles) is knocked out, and homozygous knockouts refer to when both gene copies are knocked out. Therefore, the expression of at least one gene product is altered (e.g., increased, decreased, knocked out, deleted, or activated) using the targeted genome engineering systems described herein, relative to an unaltered cell. In an embodiment, the expression of one or more gene products are increased, the expression of one or more gene produces are decreased, and the expression of one or more gene products are knocked out by at least three separately-acting RNA-guided DNA endonucleases.

Endonucleases

A nuclease protein is a non-specific endonuclease. It is directed to a specific DNA target by a gRNA, where it causes a double-strand break. Nuclease-deficient RNA-guided DNA endonucleases can cause transcriptional activation or transcriptional interference. There are many versions of RNA-guided DNA endonucleases isolated from different bacteria.

Each RNA-guided DNA endonuclease binds to its target sequence only in the presence of a protospacer adjacent motif (PAM), on the non-targeted DNA strand. Therefore, the locations in a genome that can be targeted by different RNA-guided DNA endonuclease can be dictated by locations of PAM sequences. A catalytically-active RNA-guided DNA endonuclease cuts 3-4 nucleotides upstream of the PAM sequence. Recognition of the PAM sequence by a RNA-guided DNA endonuclease protein is thought to destabilize the adjacent DNA sequence, allowing interrogation of the sequence by the sgRNA, and allowing the sgRNA-DNA pairing when a matching sequence is present. Exemplary protospacers and PAM motifs the can be used of the systems and methods described herein are listed in Table 2. The three independent RNA-guided DNA endonuclease proteins of the tri-functional systems described herein can have protospacer adjacent motif (PAM) sequences and gRNA scaffold sequences that are different from each other.

RNA-guided DNA endonucleases isolated from different bacterial species recognize different PAM sequences. For example, the SpCas9 nuclease cuts upstream of the PAM sequence 5′-NGG-3′ (where “N” can be any nucleotide base), while the PAM sequence 5′-NNGRR(N)-3′ is required for SaCas9 (from Staphylococcus aureus) to target a DNA region for editing. While the PAM sequence itself is necessary for cleavage, it is not included in the single guide RNA sequence. A nuclease-deficient RNA-guided DNA endonuclease protein is directed by RNA base pairing to target DNA, but is not capable of cleaving the phosphodiester bond within a polynucleotide chain. Thus, a nuclease-deficient RNA-guided endonuclease protein can be used to specifically target any region of the genome without causing cleavage. RNA-guided DNA endonucleases (e.g., Cas9) are rendered nuclease-deficient by amino acid point mutations. For example, the H840A and D10A mutations in the HHN-nuclease domain and RuvC1 domain, respectively, in the Cas9 from Streptococcus pyogenes inactivate cleavage activity, but do not prevent binding of the RNA-guided DNA endonuclease. Additionally, an E832A mutation in the Cpf1 protein from Lachnospiraceae bacterium ND2006 inactivates cleavage activity, but does not prevent binding. Nuclease-deficient RNA-guided DNA endonuclease proteins include, but are not limited to, nuclease-deficient Cas9 from Streptococcus pyogenes (dSpCas9), nuclease-deficient Cas9 from Staphylococcus aureus (dSaCas9), nuclease-deficient Cas9 from Streptococcus thermophiles (dSt1Cas9), nuclease-deficient Cpf1 from Lachnospiraceae bacterium ND2006 (dLbCpf1), and nuclease-deficient Cpf1 from Acidaminococcus sp. BV3L6 (AsCpf1). Nuclease-deficient RNA-guided DNA endonuclease proteins can be fused with various effector domains (e.g., transcriptional activators, repression domains, or fluorescent proteins). Transcriptional activation or interference can be achieved by fusing an activation or repression domain to a nuclease-deficient CRISPR protein (e.g., Cas9, Cpf1).

A nuclease-deficient RNA-guided DNA endonuclease protein can be operably linked to at least one activation domain to form a nuclease-deficient RNA-guided DNA endonuclease that causes transcriptional activation. As used here, the term “activation domain” refers to a transcription factor that increases transcription of the gene that it targets. Activation domains can be derived from a transcription factor protein. Activation domains can contain amino acid compositions rich in acidic amino acids, hydrophobic amino acids, prolines, glutamines, or hydroxylated amino acids. Alpha helix structural motifs can also be common in activation domains. Activation domains contain about 5 amino acids to about 200 amino acids (La Russa, M. F., et al., Mol. Cell. Biol. 35:3800-3809 (2015); Maeder, M. I., et al., Nat. Methods 10:977-979 (2013); Qi, I.S., et al., Cell 152:1173-1183 (2013); Gilbert, L. A., et al., Cell 159:647-661 (2014); Zalatan, J. G., et al., Cell 160:339-350 (2015); Chavez A., et al., Nat. Methods 12:326-8 (2015)).

Two DNA sequences are operably linked if the nature of the linkage does not interfere with the ability of the sequences to affect their normal functions relative to each other. For instance, a promoter region would be operably linked to a coding sequence of the protein if the promoter were capable of effecting transcription of that coding sequence.

A nuclease-deficient RNA-guided DNA endonuclease protein can be, for example dSpCas9, dSaCas9, dSt1Cas9, or dLbCpf1 and an activation domain can be, for example, VP64 (V), VP64-p65AD (VP), VP64-p65AD-Rta (VPR), or GAL4-AD. A nuclease-deficient RNA-guided DNA endonuclease protein can be, for example, dLbCpf1 and a one activation domain can be, for example, VP64-p65AD (VP).

A nuclease-deficient RNA-guided DNA endonuclease protein can be operably linked to at least one repression domain to form a nuclease-deficient RNA-guided DNA endonuclease protein that causes transcriptional interference. A repression domain is a transcription factor that decreases transcription of the gene that it targets. (La Russa, M. F., et al., Mol. Cell. Biol. 35:3800-3809 (2015); Maeder, M. I., et al., Nat. Methods 10:977-979 (2013); Qi, I. S., et al., Cell 152:1173-1183 (2013); Gilbert, L. A., et al., Cell 159:647-661 (2014); Zalatan, J. G., et al., Cell 160:339-350 (2015)). Like activation domains, repression domains can vary in length and amino acid sequence, and do not have significant sequence homology with one another. Repression domains can have amino acid compositions rich in alanines, prolines, and charged amino acids. Repression domains can contain about 5 amino acids to about 200 amino acids. A repression domain can be small (e.g., about 5 to 200 amino acids, about 5 to 150 amino acids, about 10 to 100 amino acids, about 20 to 80 amino acids, about 10 to 50 amino acids) while demonstrating strong transcriptional repression.

A nuclease-deficient RNA-guided DNA endonuclease protein can be operably linked multiple repression domains (e.g., 2, 3, 4, 5, 6, 7, 8, 9, 10 or more repression domains) to form a nuclease-deficient RNA-guided DNA endonuclease protein that causes transcriptional interference.

Examples of nuclease-deficient RNA-guided DNA endonuclease protein that cause transcriptional interference include dSpCas9, dSaCas9, dSt1Cas9, or dLbCpf1. Examples of repression domains include MXI1, RD1 (TUP1), RD2, RD3, RD4, RD5 (MIG1), RD6, RD7, RD8, RD9, RD10, RD11 (UME6), or KRAB or combinations thereof. Furthermore, there are several mammalian transcription factors (e.g., p53, Erg-1, C/EBPc) that can function as both activation domains and repression domains.

A catalytically active RNA-guided DNA endonuclease protein is an RNA-guided DNA endonuclease protein that is directed by RNA base pairing and capable of cleaving a phosphodiester bond within a polynucleotide chain. Catalytically active RNA-guided DNA endonuclease proteins include, for example, Cas9 from Streptococcus pyogenes (SpCas9), Neisseria meningitides (NmCas9), Streptococcus thermophiles (St1Cas9), and Staphylococcus aureus (SaCas9) and Cpf1 from Lachnospiraceae bacterium ND2006 (LbCpf1) and Acidaminococcus sp. BV3L6 (AsCpf1).

As used herein, the term “target DNA” refers to chromosomal DNA. Target DNA includes nucleic acids that can be activated, repressed, deleted, knocked-out, or interfered with. For example, target DNA can include protein coding sequences and promoter sequences. Target DNA can be about 18 nucleotides to about 25 nucleotides in length. Target DNA for CRISPRa can be, for example, about 250 base pairs upstream of the coding sequences or about 200 base pairs upstream of the transcription starting site (TSS). Target DNA for CRISPRa can be, for example, about 23 base pairs (e.g., 21, 22, 23, 24, or 25 base pairs) in length. Target DNA for CRISPRi can be, for example, about 100 base pairs to about 150 base pairs upstream of the coding sequences or 50 base pairs to about 100 base pairs upstream of the TSS. Target DNA for CRISPRa can be, for example, about 20 base pairs (e.g., 18, 19, 20, 21, or 22 base pairs) in length. Target DNA for CRISPRd can be, for example, about 21 base pairs (e.g., 19, 20, 21, 22 or 23 base pairs) in length. Most organisms have the same genomic DNA in every cell, but only certain genes are active in each cell to allow for cell function and differentiation within the body. The genome of an organism (encoded by the genomic DNA) is the (biological) information of heredity which is passed from one generation of organism to the next.

A system described herein can further comprise one or more additional sgRNA molecules that are capable of binding a target nucleic acid and a catalytically-active RNA-guided DNA endonuclease protein that causes a double-stranded nucleic acid break of one or more additional target nucleic acid molecules. In this aspect, the genome can be cut at several different sites (e.g., 1, 2, 3, 4, 5, 6, 7, 8, 9, or 10 sites) at or near the same time, and the homology directed repair donor included in the sgRNA expression plasmid can be inserted into those one or more sites (Bao, Z., et al., 2015, ACS Synth. Biol., 5:585-594).

The systems described herein can utilize orthogonal RNA-guided DNA endonuclease proteins. Orthogonal refers to ligand-protein pairs, whereby the RNA-guided DNA endonuclease protein is only functional when in the presence of its cognate gRNA pair. For example, a nuclease-deficient RNA-guided DNA endonuclease protein (e.g., dSpCas9, dSaCas9, dSt1Cas9, and dLbCpf1) is functional only when bound to a sgRNA ortholog. A catalytically active RNA-guided DNA endonuclease protein (e.g., Cas9) can be functional only when bound to a sgRNA ortholog. The gRNA structure sequences as well as the PAM sequences are different, both of which endow the activity of the CRISPR proteins described in Table 7 to be orthogonal.

A nuclease-deficient RNA-guided DNA endonuclease or catalytically active RNA-guided DNA endonuclease, can be expressed from an expression cassette. An expression cassette is a distinct component of vector DNA comprising a gene and regulatory elements to be expressed by a transformed or transfected cell, whereby the expression cassette directs the cell to make RNA and protein. Different expression cassettes can be transformed or transfected into different organisms including bacteria, yeast, plants, and mammalian cells as long as the correct regulatory element sequences are used.

Once a target DNA and RNA-guided DNA endonuclease have been selected, the next step is to design a specific guide RNA sequence. Several software tools exist for designing an optimal guide with minimum off-target effects and maximum on-target efficiency. Examples include Synthego Design Tool, Desktop Genetics, Benchling, and MIT CRISPR Designer.

sgRNA

As used herein, “single guide RNA” (the terms “single guide RNA,” “guide RNA (gRNA),” and “sgRNA” may be used interchangeably herein) refers to a single RNA species capable of directing catalytically active RNA-guided DNA endonuclease mediated single stranded or double stranded cleavage of target DNA; capable of directing nuclease-deficient RNA-guided DNA endonuclease mediated transcriptional activation of target DNA; capable of directing nuclease-deficient RNA-guided DNA endonuclease mediated transcriptional interferences of target DNA. Single-stranded gRNA sequences are transcribed from double-stranded DNA sequences inside the cell.

A guide RNA is a specific RNA sequence that recognizes a target DNA region of interest and directs a RNA-guided DNA endonuclease there for editing. A gRNA has at least two regions. First, a crispr RNA (crRNA) or spacer sequence, which is a nucleotide sequence complementary to the target DNA, and second a tracr RNA, which serves as a binding scaffold for the RNA-guided DNA endonuclease. The gRNA sequence that is complementary to the target DNA is known as the protospacer. The crRNA and tracr RNA can exist as one molecule or as two separate molecules, as they are in nature. gRNA and sgRNA as used herein refer to a single molecule comprising at least a crRNA region and a tracr RNA region or two separate molecules wherein the first comprises the crRNA region and the second comprises a tracr RNA region. The crRNA region of the gRNA is a customizable component that enables specificity in every CRISPR reaction. A guide RNA used in the systems and methods can also comprise an endoribonuclease recognition site (e.g., Csy4) for multiplex processing of gRNAs. If an endoribonuclease recognition site is introduced between neighboring gRNA sequences, more than one gRNA can be transcribed in a single expression cassette.

A guide RNA used in the systems and methods are short, single-stranded polynucleotide molecules about 20 nucleotides to about 300 nucleotides in length. The spacer sequence (targeting sequence) that hybridizes to a complementary region of the target DNA of interest can be about 20-30 nucleotides in length.

A sgRNA capable of directing nuclease-deficient RNA-guided DNA endonuclease mediated transcriptional activation of target DNA can be about 43 nucleotides (e.g., about 40, 41, 42, 43, 44, 45, or 46 nucleotides) in length. A sgRNA can guide a nuclease-deficient RNA-guided DNA endonuclease near the promoter or enhancer regions of a gene to activate transcription (e.g., about 250 bp upstream of the coding sequences or about 200 bp upstream of the TSS). The activation domain(s) of the nuclease-deficient RNA-guided DNA endonuclease recruits RNA polymerase to activate the expression of the target gene.

A sgRNA capable of directing nuclease-deficient RNA-guided DNA endonuclease mediated transcriptional interference of target DNA can be about 96 nucleotides (e.g., about 91, 92, 93, 94, 95, 96, 97, 98, 99, or 100 nucleotides) in length. A sgRNA can guide a nuclease-deficient RNA-guided DNA endonuclease near the promoter or enhancer regions of a gene to interfere with transcription (e.g., about 100-150 bp upstream of the coding sequence or 50-100 bp upstream of TSS). The repression domain(s) of the nuclease-deficient RNA-guided DNA endonuclease interferes with the binding of the RNA polymerase, which in turn represses transcription of the target gene.

A sgRNA capable of directing catalytically-active RNA-guided DNA endonuclease mediated gene deletion of target DNA can be can be about 248 nucleotides (e.g., 236, 237, 238, 239, 240, 241, 242, 243, 244, 245, 246, 247, 248, 249, 250, 251, 252, 253, 254, 255, 256, 257, 258, 259, or 260 nucleotides) in length. A sgRNA can guide a catalytically active RNA-guided DNA endonuclease to the coding sequence of a gene. The sgRNA used to direct gene deletion can include DNA donor sequences for homology-directed repair.

sgRNAs can be synthetically generated or by making the sgRNA in vivo or in in vitro, starting from a DNA template.

One method of making sgRNAs comprises expressing the sgRNA sequence in cells from a transformed or transfected plasmid. The sgRNA sequence is cloned into a plasmid vector, which is then introduced into cells. The cells use their normal RNA polymerase enzyme to transcribe the genetic information in the newly introduced DNA to generate the sgRNA.

sgRNA can also be made by in vitro transcription (IVT). sgRNA is transcribed from a corresponding DNA sequence outside the cell. A DNA template is designed that contains the guide sequence and an additional RNA polymerase promoter site upstream of the sgRNA sequence. The sgRNA is then transcribed using commercially available kits with reagents and recombinant RNA polymerase.

sgRNAs can also be synthetically generated. Synthetically generated sgRNAs can be chemically modified to prevent degradation of the molecule within the cell.

Exemplary oligonucleotides that can be used to synthesize gRNAs of the systems described herein are listed in Table 4 and Table 5.

A sgRNA can target a regulatory element (e.g., a promoter, enhancer, or other regulatory element) in the target genome. A sgRNA can also target a coding sequence in the target genome.

The sgRNAs of the system and methods described herein can also be truncated (e.g., comprising 12-16 nucleotide targeting sequences). For example, Sg27 gRNAs is a truncated version of the full length Sg1. The sgRNA can be unmodified or modified. For example, modified sgRNAs can comprise one or more 2′-O-methyl and/or 2′-O-methyl phosphorothioate nucleotides.

A first single guide RNA (sgRNA) that is capable of binding a target nucleic acid sequence and binding a first nuclease-deficient RNA-guided DNA endonuclease protein; a second sgRNA that is capable of binding a target nucleic acid sequence and binding a second nuclease-deficient RNA-guided DNA endonuclease protein; a third sgRNA that is capable of binding a target nucleic acid sequence and binding a catalytically active RNA-guided DNA endonuclease protein; a polynucleotide encoding a first nuclease-deficient RNA-guided DNA endonuclease protein that binds to the first sgRNA and causes transcriptional activation; a polynucleotide encoding a second nuclease-deficient RNA-guided DNA endonuclease protein that binds to the second sgRNA and causes transcriptional interference; and a polynucleotide encoding a catalytically active RNA-guided DNA endonuclease protein that binds to the third sgRNA and causes a double-stranded nucleic acid break and causes gene deletion can be located on the same or different vectors of the system.

The three sgRNAs or three pools of sgRNAs that can be used in the systems and methods herein are orthogonal to each other, meaning that the first sgRNA or first pool of sgRNAs are only be recognized by the nuclease-deficient RNA-guided DNA endonuclease capable of causing transcriptional activation; the second sgRNA or second pool of sgRNAs can only be recognized by the nuclease-deficient RNA-guided DNA endonuclease capable of causing transcriptional interference; and, the third sgRNA or third pool of sgRNAs can only be recognized by the catalytically active RNA-guided DNA endonuclease capable of causing gene deletion.

sgRNAs are not particularly limited and can be any sgRNA. A sgRNA that is capable of binding a nuclease-deficient RNA-guided DNA endonuclease protein that causes transcriptional activation can be, for example, sg6, sg149, sg150, sg155, sg156, sg157, sg175, sg221, or sg218. A sgRNA that is capable of binding a nuclease-deficient RNA-guided DNA endonuclease protein that causes transcriptional interference can be, for example, sg1, sg27, sg28, sg112, sg113, sg114, sg172, sg120, sg121, sg230, or sg204. A sgRNA that is capable of binding a catalytically active RNA-guided DNA endonuclease protein that causes a double-stranded nucleic acid break and causes gene deletion can be, for example, sg11, sg186, sg205, sg265, sg266, or sg267.

sgRNA that is capable of binding a target nucleic acid sequence and binding a nuclease-deficient RNA-guided DNA endonuclease protein that causes transcriptional interference can be expressed in an expression cassette comprising a type II promoter or a type III promoter.

One or more vectors that express sgRNA and/or RNA-guided DNA endonuclease proteins can further comprise a polynucleotide encoding for a marker protein. The marker protein can be, for example, an antibiotic resistance protein or a florescence protein for easier monitoring of genome integration and expression, and to label or track particular cells.

A polynucleotide encoding a marker protein can be expressed on a separate vector from a vector that expresses sgRNA and/or RNA-guided DNA endonuclease proteins.

A marker protein is a protein encoded by a gene that when introduced into a cell (prokaryotic or eukaryotic) confers a trait suitable for artificial selection. Marker proteins are used in laboratory, molecular biology, and genetic engineering applications to indicate the success of a transformation, a transfection or other procedure meant to introduce foreign DNA into a cell. Marker proteins include, but are not limited to, proteins that confer resistance to antibiotics, herbicides, or other compounds, which would be lethal to cells, organelles or tissues not expressing the resistance gene or allele. Selection of transformants is accomplished by growing the cells or tissues under selective pressure, i.e., on media containing the antibiotic, herbicide or other compound. If the marker protein is a “lethal” marker, cells which express the marker protein will live, while cells lacking the marker protein will die. If the marker protein is “non-lethal,” transformants (i.e., cells expressing the selectable marker) will be identifiable by some means from non-transformants, but both transformants and non-transformants will live in the presence of the selection pressure.

Selective pressure refers to the influence exerted by some factor (such as an antibiotic, heat, light, pressure, or a marker protein) on natural selection to promote one group of organisms or cells over another. In the case of antibiotic resistance, applying antibiotics cause a selective pressure by killing susceptible cells, allowing antibiotic-resistant cells to survive and multiply.

Selective pressure can be applied by contacting the cells with an antibiotic and selecting the cells that survive. The antibiotic can be, for example, kanamycin, puromycin, spectinomycin, streptomycin, ampicillin, carbenicillin, bleomycin, erythromycin, polymyxin B, tetracycline, or chloramphenicol.

In some embodiments, the systems and methods do not utilize synthetic CRISPR-repressible promoters (e.g., CRP-a) or synthetic CRISPR-activatable promoters (e.g., CAP). Synthetic CRISPR-repressible or CRISPR-activatable promoters are designed for CRISPRa and CRISPRi in mammalian cells (Kiani, S., et al., 2015, Nat. Methods, 12:1051-1054). A repressible promoter can express genes constitutively unless they are switched off by a repressor (e.g., protein or small molecule). An activatable promoter, or inducible promoter, can express genes only when an activator (e.g., protein or small molecule) is present.

Polynucleotides of the Systems

Also provided are examples of polynucleotides useful in the systems and methods described herein.

The terms “polynucleotide,” “nucleotide,” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. Nucleic acid molecule means a single- or double-stranded linear polynucleotide containing either deoxyribonucleotides or ribonucleotides that are linked by 3′-5′-phosphodiester bonds. A nucleic acid construct is a nucleic acid molecule which is isolated from a naturally occurring gene or which has been modified to contain segments of nucleic acid which are combined and juxtaposed in a manner which would not otherwise exist in nature. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), single guide RNA (sgRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

A recombinant nucleic acid molecule, for instance a recombinant DNA molecule, is a novel nucleic acid sequence formed in vitro through the ligation of two or more nonhomologous DNA molecules (for example a recombinant plasmid containing one or more inserts of foreign DNA cloned into at least one cloning site).

Homology refers to the similarity between two nucleic acid sequences. Homology among DNA, RNA, or proteins is typically inferred from their nucleotide or amino acid sequence similarity. Significant similarity is strong evidence that two sequences are related by evolutionary changes from a common ancestral sequence. Alignments of multiple sequences are used to indicate which regions of each sequence are homologous. The term “percent homology” is used herein to mean “sequence similarity.” The percentage of identical nucleic acids or residues (percent identity) or the percentage of nucleic acids residues conserved with similar physicochemical properties (percent similarity), e.g. leucine and isoleucine, is used to quantify the homology.

Complement or complementary sequence means a sequence of nucleotides which forms a hydrogen-bonded duplex with another sequence of nucleotides according to Watson-Crick base-pairing rules. For example, the complementary base sequence for 5′-AAGGCT-3′ is 3′-TTCCGA-5′. Downstream refers to a relative position in DNA or RNA and is the region towards the 3′ end of a strand. Upstream means on the 5′ side of any site in DNA or RNA.

As described herein, “sequence identity” is related to sequence homology. Homology comparisons may be conducted by eye or using sequence comparison programs. These commercially available computer programs may calculate percent (%) homology between two or more sequences and may also calculate the sequence identity shared by two or more amino acid or nucleic acid sequences. Sequence homologies may be generated by any of a number of computer programs known in the art, for example BLAST or FASTA.

Percentage (%) sequence identify can be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Ungapped alignments are performed only over a relatively short number of residues. Although this is a very simple and consistent method, it fails to take into consideration that, for example, in an otherwise identical pair of sequences, one insertion or deletion may cause the following amino acid residues to be put out of alignment, thus potentially resulting in a large reduction in percent homology when a global alignment is performed. Therefore, most sequence comparison methods are designed to produce optimal alignments that take into consideration possible insertions and deletions without unduly penalizing the overall homology or identity score. This is achieved by inserting “gaps” in the sequence alignment to try to maximize local homology or identity.

A polynucleotide can comprise a nucleotide sequence encoding a nuclear localization sequence (NLS). A NLS is an amino acid sequence that tags a protein for import into the cell nucleus by nuclear transport. Typically, this signal consists of one or more short sequences of positively charged lysines or arginines exposed on the protein surface. Different nuclear localized proteins may share the same NLS. A NLS can be added to the C-terminus, N-terminus, or both termini of an RNA-guided DNA endonuclease protein (e.g., NLS-protein, protein-NLS, or NLS-protein-NLS) to ensure nuclease activity in the cell. A NLS sequence can comprise, for example, the sequence of amino acids set forth in SEQ ID NO: 577 (PKKKRKV) or SEQ ID NO:578 (KRPAATKKAGQAKKKKK).

A polynucleotide can also comprise a nucleotide sequence encoding a polypeptide linker sequence. Linkers are short (e.g., about 3 to 20 amino acids) polypeptide sequences that can be used to operably link protein domains. Linkers can comprise flexible amino acid residues (e.g., glycine or serine) in order to permit adjacent protein domains to move freely related to one another. A linker sequence can comprise, for example, the sequence of amino acids set forth in SEQ ID NO:579 (GSSKLSGGGSGGSGS), SEQ ID NO:580 (GGGSGGSGS), or SEQ ID NO:581 (GGGSGGSGSKLGGSGGS).

For example, a polynucleotide can comprise a nucleotide sequence encoding a Cpf1 nuclease-deficient RNA-guided DNA endonuclease protein operably linked to at least one VP64-p65AD (VP) activator domain. A Cpf1 protein can be, for example, from Lachnospiraceae bacterium or Acidaminococcus sp.

An activator domain can be operably linked to the N-terminal and/or C-terminal ends of a nuclease-deficient RNA-guided DNA endonuclease protein, or operably linked in tandem at the N-terminal and/or C-terminal ends of a nuclease-deficient RNA-guided DNA endonuclease protein.

A Cpf1 nuclease-deficient RNA-guided DNA endonuclease protein can be linked at the N-terminal and C-terminal ends to a NLS polypeptide (e.g., NLS-dLbCpf1-NLS). A Cpf1 nuclease-deficient RNA-guided DNA endonuclease protein can comprise a NLS polypeptide operably linked to the N-terminal end of the Cpf1 protein, which is operably linked at the C-terminal end to a NLS polypeptide, which is operably linked at the C-terminal end to at least one VP64-p65AD (VP) activator (e.g., NLS-dLbCpf1-NLS-VP). The NLS polypeptides of the Cpf1 nuclease-deficient RNA-guided DNA endonuclease protein can be the same or different NLS polypeptides.

A Cpf1 nuclease-deficient RNA-guided DNA endonuclease protein can comprise the sequence of amino acids set forth in SEQ ID NO:573 or at least 70%, 75%, 80%, 85%, 90%, 95%, 96%, 97%, or 98% sequence identity to the sequence set forth in SEQ ID NO:573. A Cpf1 nuclease-deficient RNA-guided DNA endonuclease protein can be operably linked to at least one VP64-p65AD (VP) activator domain, which can comprise the sequence of amino acids set forth in SEQ ID NO:574 or at least 70%, 75%, 80%, 85%, 90%, 95%, 96%, 97%, or 98% sequence identity to the sequence set forth in SEQ ID NO:574. A polynucleotide encoding a Cpf1 nuclease-deficient RNA-guided DNA endonuclease protein operably linked to at least one VP64-p65AD (VP) activator domain can comprise the sequence of nucleic acids set forth in SEQ ID NO:662, or at least 70%, 75%, 80%, 85%, 90%, 95%, 96%, 97%,or 98% sequence identity to the sequence of nucleic acids set forth in SEQ ID NO:662.

Another polynucleotide can comprise a nucleotide sequence encoding a Cas9 nuclease-deficient RNA-guided DNA endonuclease protein operably linked to more than one repression domain. A Cas9 RNA-guided DNA endonuclease protein can be from, for example, Streptococcus pyogenes, Neisseria meningitides, Streptococcus thermophiles, or Staphylococcus aureus. A Cas9 nuclease-deficient RNA-guided DNA endonuclease protein can be operably linked to, for example, a RD1 (TUP1), RD2, RD3, RD4, RD5 (MIG1), RD6, RD7, RD8, RD9, RD10, or RD11 (UME6) repression domain, or combinations thereof.

A polynucleotide can comprise a nucleotide sequence encoding a dSpCas9 protein operably linked to the C-terminal end to a RD11 repression domain, wherein a RD5 repression domain is operably linked to the C-terminal end of the RD11 domain, wherein a RD2 repression domain is operably linked to the C-terminal end of the RD5 domain.

A repression domain can be operably linked to the N-terminal and/or C-terminal ends of a nuclease-deficient RNA-guided DNA endonuclease protein, or operably linked in tandem at the N-terminal and/or C-terminal ends of a nuclease-deficient RNA-guided DNA endonuclease protein.

A Cas9 RNA-guided DNA endonuclease protein can be linked at the N-terminal and C-terminal ends to a NLS polypeptide (e.g., NLS-dLbCpf1-NLS). A Cas9 RNA-guided DNA endonuclease protein can comprise a NLS polypeptide operably linked to the N-terminal end of the Cas9 protein, which is operably linked at the C-terminal end to a NLS polypeptide, which is operably linked at the C-terminal end via a linker to a RD11 polypeptide, which is linked at the C-terminal end via a linker to a RD5 polypeptide, which is linked at the C-terminal end via a linker to a RD2 polypeptide. The NLS polypeptides of the Cas9 RNA-guided DNA endonuclease protein can be the same or different NLS polypeptides.

A Cas9 nuclease-deficient RNA-guided DNA endonuclease protein can comprise the sequence of amino acids set forth in SEQ ID NO:575 or at least 95% sequence identity to the sequence set forth in SEQ ID NO:575. A polynucleotide comprising a nucleotide sequence encoding a Cas9 nuclease-deficient RNA-guided DNA endonuclease protein operably linked to more than one repression domain can comprise the sequence of amino acids set forth in SEQ ID NO:575 or at least 70%, 75%, 80%, 85%, 90%, 95%, 96%, 97%, or 98% sequence identity to the sequence set forth in SEQ ID NO:575. A polynucleotide encoding a Cas9 nuclease-deficient RNA-guided DNA endonuclease protein operably linked to more than one repression domain can comprise the sequence of nucleic acids set forth in SEQ ID NO:743, or at least 70%, 75%, 80%, 85%, 90%, 95%, 96%, 97%, or 98% sequence identity to the sequence of nucleic acids set forth in SEQ ID NO:743.

Methods of Altering Gene Expression Via CRISPR-AID

Methods of altering the expression of gene products are provided herein. The methods comprise introducing into a cell a system for targeted genome engineering as described herein; wherein the expression of at least one gene product (e.g., about 1, 2, 3, 4, 5, 10, or more) is increased, the expression of at least one gene product (e.g., about 1, 2, 3, 4, 5, 10, or more) is decreased, and the expression of at least one gene product (e.g., about 1, 2, 3, 4, 5, 10, or more) is deleted relative to a cell that has not been transformed or transfected with the system for targeted genome engineering.

The methods can further comprise selecting for successfully transformed or transfected cells by applying selective pressure (e.g., culturing cells in the presence of selective media).

One or more vectors of a system described herein can further comprise a polynucleotide encoding for a marker protein such as an antibiotic resistance protein or a florescence protein.

Transformation or transfection is the directed modification of the genome of a cell by introducing recombinant DNA from another cell of a different genotype, leading to its uptake and integration into the subject cell's genome. In bacteria, the recombinant DNA is not typically integrated into the bacterial chromosome, but instead replicates autonomously as a plasmid. A vector can be introduced into cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.). It will be appreciated by those skilled in the art that the design of the expression vector can depend on such factors as the choice of the host cell to be transformed, the level of expression desired, etc.

Methods for transforming or transfecting a cell with an expression vector may differ depending upon the species of the desired cell. For example, yeast cells may be transformed by lithium acetate treatment (which may further include carrier DNA and PEG treatment) (the LiAc/SS carrier and DNA/PEG method) or electroporation. Mammalian cells can be transfected via liposome-mediated transfection, using non-liposomal transfection agents (e.g., polymers and lipids), or by electroporation. These methods are included for illustrative purposes and are in no way intended to be limiting or comprehensive. Routine experimentation through means well known in the art may be used to determine whether a particular expression vector or transformation method is suited for a given host cell. Furthermore, reagents and vectors suitable for many different host microorganisms are commercially available and/or well known in the art.

Any gene product pathway, combination of pathways, operon, group of related genes, or groups of unrelated genes can be targeted using systems described herein.

The method can occur in vivo or in vitro. The cell can be a eukaryotic cell or a prokaryotic cell. Eukaryotic cells include mammalian cells (e.g., mouse, human, dog, monkey), insect cells (e.g., bee, fruit fly) plant cells, algae cells, and fungal cells (e.g., yeast). The cell can be a yeast cell such as Saccharomyces cerevisiae.

The at least one gene product can be, for example, a protein involved in the mevalonate pathway, either directly or indirectly. Proteins involved in the mevalonate pathway include, but are not limited to, acetoacetyl-CoA thiolase, HMG-CoA synthase, HMG-CoA reductase (HMG-1), mevalonate-5-kinase, mevalonate-3-kinase, mevalonate-3-phosphate-5-kinase, phosphomevalonate kinase, mevalonate-5-pyrophosphate decarboxylase, and sopentenyl pyrophosphate isomerase, ERG9, ROX1, ARP6, SER33, YJL064w, and YPL062w.

A system for genome engineering can simultaneously cause an increase in expression of HMG1, a decrease in expression of ERG9, and the deletion of expression of ROX1. Simultaneously refers to occurring, operating, or done at or about the same time.

A system for genome engineering can, for example, causes increased production of an isoprenoid in a cell. Isoprenoid refers to the class of naturally occurring organic compounds derived from terpene. Examples of isoprenoids include, but are not limited to, carotene, phytol, retinol (vitamin A), tocopherol (vitamin E), dolichols, squalene, ginsenosides, and taxol. In some embodiments, the isoprenoid is β-carotene. In other embodiments, the production of β-carotene is increased by at least 1 fold, 1.5 fold, 2 fold, 2.5 fold, 3 fold, 3.5 fold, 4 fold, 4.5 fold, or 5 fold.

The systems for genome engineering described herein can increase expression of a surface protein on a cell. The expression of PDI1 can be increased, the expression of MNN9 can be decreased, and the expression of PMR1 can be deleted, all simultaneously. In other embodiments, EGII display levels and cellulase activity are increased. Any combination of genes can be targeted by the systems described herein.

Multi-Functional Genome-Wide CRISPR (MAGIC)

Also provided are methods of identifying the genetic basis of one or phenotypes of a host cell using the orthogonal CRISPR-AID system described above. A method of identifying the genetic basis of one or more phenotypes of cells, the method comprising: (i) preparing three genome-scale sgRNA expressing plasmid libraries from oligonucleotides wherein the first genome-scale sgRNA expressing plasmid library is for upregulating genes of the cells, wherein the second genome-scale sgRNA expressing plasmid library is for downregulating genes of the cells, and the third genome-scale sgRNA expressing plasmid library is for deleting genes of the cells; (ii) transforming the three genome-scale sgRNA expressing plasmid libraries into the cells; (iii) introducing into the cells (e.g., by transformation or transfection) a polynucleotide encoding a first nuclease-deficient RNA-guided DNA endonuclease protein that binds to the sgRNA of the first genome-scale sgRNA expressing plasmid library and causes transcriptional activation of genes of the cells, a polynucleotide encoding a second nuclease-deficient RNA-guided DNA endonuclease protein that binds to the sgRNA of the second genome-scale sgRNA expressing plasmid library and causes transcriptional repression of genes of a cell, and a polynucleotide encoding a catalytically active RNA-guided DNA endonuclease protein that binds to a the sgRNA of the third genome-scale sgRNA expressing plasmid library and causes double-stranded nucleic acid breaks and gene deletion of genes of the cells; (iv) isolating transformed cells with one or more phenotypes; and (v) determining the genomic loci of the DNA molecule that causes the one or more phenotypes.

The MAGIC system can comprise more than one sgRNA capable of directing nuclease-deficient RNA-guided DNA endonuclease mediated transcriptional activation of target DNA, more than one sgRNA capable of directing nuclease-deficient RNA-guided DNA endonuclease mediated transcriptional interference of target DNA, and more than one capable of sgRNA capable of directing catalytically active RNA-guided DNA endonuclease mediated gene deletion of target DNA.

A library of sgRNA is a plurality of sgRNAs that are capable of targeting a plurality of genomic loci in a population of cells.

A genome-scale sgRNA expressing plasmid library is a library of sgRNA that can perturb all the genes in a cell at once. For example, a genome-scale sgRNA expressing plasmid library in Saccharomyces cerevisiae can perturb the more than 6000 genes in the yeast genome. A method of identifying the genetic basis of one or more phenotypes of cells can also be performed with a sgRNA expressing plasmid library that is less than genome-scale, for example, 100 genes, 200 genes, 300 genes, 400 genes, 500 genes, 1000 genes, or more.

The first, second, and third genome-scale sgRNA expressing plasmid libraries used in the method of identifying the genetic basis of one or more phenotypes of cells can each target the same genes, either on a genome-scale or less than genome-scale.

Additionally, the first, second, and third sgRNA expressing plasmid libraries can be transformed or transfected into the cell all at once or separately.

Genome-scale sgRNA expressing plasmid libraries can be prepared, for example, by the methods described below in Example 6. In particular, a genome-scale sgRNA expressing plasmid library can be prepared by extracting ORF and RNA coding sequences and their promoter sequences from a genome database of interest (e.g., the Saccharomyces genome database; yeastgenome.org). The promoter sequences, entire sequences, and coding sequences can be used for the design of activation, interference, and deletion guide sequences, respectively. The desired region sequences can be given to the CHOPCHOP program to generate all possible guide sequences. All the generated guide sequences can be ranked according to the binding efficiency, off-target effects, binding position, and the DNA synthesis and cloning considerations. For each gene, the top 3, top 4, top 5, top 6, top 7, top 8, top 9, or top 10 sequences with the highest scores can be selected for transcription activation, transcription repression, and gene deletion or knock-out libraries, respectively.

Adapters containing priming sequences and a restriction enzyme site (e.g., BsaI sites) can be added (by ligation or PCR) to both ends of each oligonucleotide for PCR amplification and Golden Gate assembly. An adapter is a short, chemically synthesized, single-stranded or double-stranded oligonucleotide that can be ligated to the ends of other DNA or RNA molecules and used for library preparation with Next Generation Sequencing (NGS) platforms. Adapters can include platform-specific sequences for fragment recognition by particular sequencer platforms. Adapters can also contain single or dual sample indexes depending on the number of libraries combined for sequencing together and the level of accuracy needed. Sample indexes can permit multiple samples to be sequenced together on the same instrument.

The unique priming sequences allow the construction of each library independently. Next, plasmids (e.g., bacterial plasmids) can be constructed containing the optimal activation, interference, and deletion guide sequences. Each of the plasmid libraries can then be transformed using standard high-efficiency transformation methods (e.g., the LiAc/SS carrier DNA/PEG method) into cells (e.g., yeast cells, mammalian cells, and insect cells) and optionally grown under selective pressure.

Genomic loci associated with certain phenotypes (e.g., yeast surface display of recombinant proteins) can be identified by undergoing multiple rounds of MAGIC screening and confirming that certain genomic loci are associated with certain phenotypes using diagnostic PCR and qPCR. The methods described in Example 7 and Example 8 can be used to determine the genomic loci of the DNA molecule that causes the phenotype. NGS can be used to conduct genotype-phenotype mapping and identify the genetic determinants (genotypes) of complex phenotypes (e.g., furfural tolerance and yeast surface display of recombinant proteins, intracellular accumulation of S-adenosyl-S-methionine, and glucose repression).

Genomic loci refer to a fixed position on a chromosome, like the position of a gene or a marker.

The term “cell” includes progeny thereof. It is also understood that all progeny may not be precisely identical, such as in DNA content, due to deliberate or inadvertent mutation. Variant progeny that have the same function or biological property of interest, as screened for in the original cell, are included.

A phenotype can be any phenotype, for example, furfural tolerance or yeast surface display of recombinant proteins. A phenotype is any observable characteristic or functional effect that can be measured in an assay such as changes in cell growth, proliferation, morphology, increase in protein expression, decrease in protein expression, lack of protein expression, enzyme function, signal transduction, expression patterns, downstream expression patterns, reporter gene activation, hormone release, growth factor release, neurotransmitter release, ligand binding, apoptosis, and product formation. Such assays include, but are not limited to, transformation assays, changes in proliferation, anchorage dependence, growth factor dependence, foci formation, growth in soft agar, tumor proliferation in nude mice, and tumor vascularization in nude mice; apoptosis assays, e.g., DNA laddering and cell death, expression of genes involved in apoptosis; signal transduction assays, e.g., changes in intracellular calcium, cAMP, cGMP, IP3, changes in hormone and neurotransmitter release; receptor assays, e.g., estrogen receptor and cell growth; growth factor assays, e.g., EPO, hypoxia and erythrocyte colony forming units assays; enzyme product assays, e.g., FAD-2 induced oil desaturation; transcription assays, e.g., reporter gene assays; and protein production assays, e.g., VEGF ELISAs. A candidate gene is “associated with” a selected phenotype if modulation of gene expression (e.g., increase in gene expression, decrease in gene expression, or knock out of gene expression) of the candidate gene causes a change in the selected phenotype.

As used herein, the term subject refers to any animal classified as a mammal, including humans, mice, rats, domestic and farm animals, non-human primates, and zoo, sport or pet animals, such as dogs, horses, cats, and cows.

The practice of the present systems and methods employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

The terminology used herein is for the purpose of exemplifying particular embodiments only and is not intended to limit the scope of the methods and compositions as disclosed herein. Any method and material similar or equivalent to those described herein can be used in the practice of the methods and compositions as disclosed herein and only exemplary methods, devices, and materials are described herein.

The methods and compositions now will be exemplified for the benefit of the artisan by the following non-limiting examples that depict some of the embodiments by and in which the methods and compositions can be practiced.

### Example 1: Design of CRISPR-AID for Combinatorial Metabolic Engineering

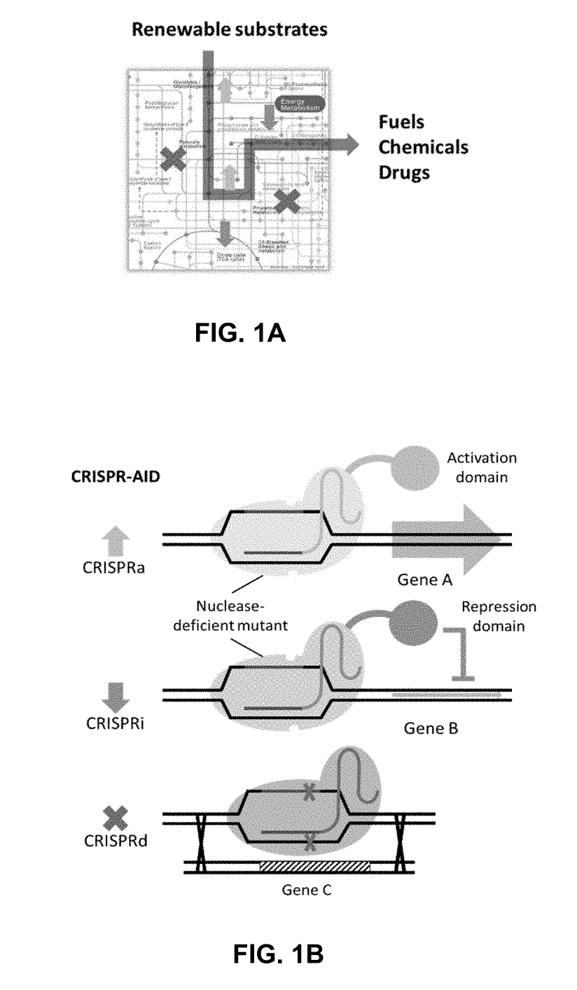

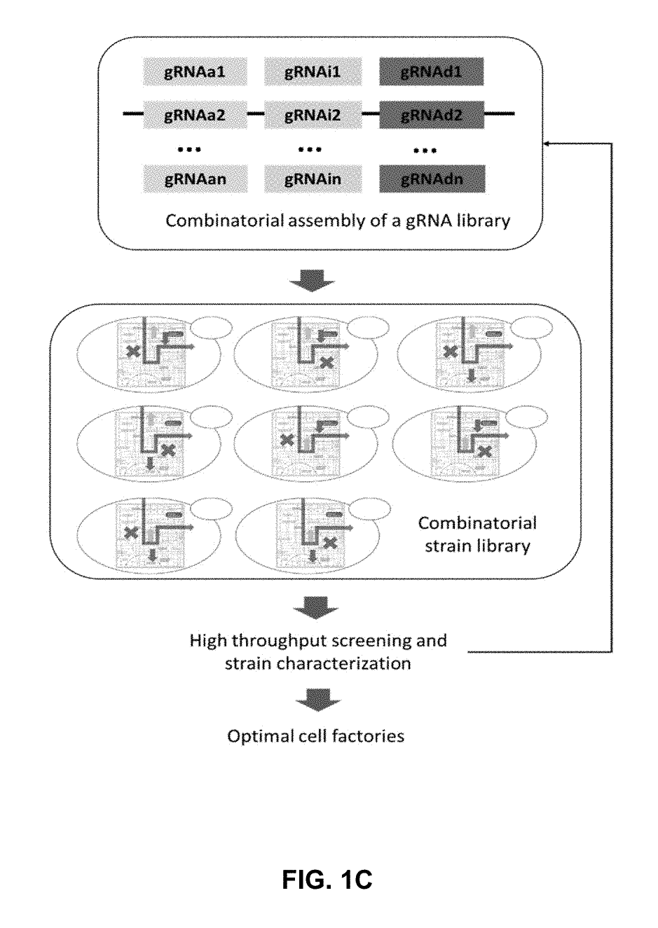

To construct optimal cell factories using combinatorial metabolic engineering, a synthetic biology toolkit that enables different modes of genetic manipulation of multiple targets in the metabolic and regulatory network, including increased expression, decreased expression, and zero expression, in a modular, parallel and high throughput manner was needed (FIG. 1A). A tri-functional CRISPR-AID system using three orthogonal CRISPR proteins was developed (FIG. 1B), one nuclease-deficient CRISPR protein fused with an activation domain for transcriptional activation (CRISPRa), a second nuclease-deficient CRISPR protein fused with a repression domain for transcriptional interference (CRISPRi), and a third catalytically active CRISPR protein for gene deletion (CRISPRd). For metabolic engineering of complex phenotypes, such as stress tolerance and production of recombinant proteins, numerous metabolic engineering targets can be identified. Since the host genome can be manipulated in a modular and high throughput manner via plasmid-borne gRNAs, CRISPR-AID enables combinatorial optimization of various metabolic engineering targets. In conjugation with high throughput screening, the combination of the activated, interfered, and deleted metabolic engineering targets that work synergistically to yield the optimal phenotype can be determined (FIG. 1C). If necessary, the process can be repeated iteratively.

### Example 2: Construction and Optimization of the CRISPR-AID System

To enable fast evaluation of orthogonal genome editing and transcriptional regulation, a reporter yeast strain was constructed: mCherry driven by a medium-strength promoter CYC1p for CRISPRa, mVenus driven by a strong promoter TEF1p for CRISPRi, and ADE2, an endogenous gene whose disruption would result in the formation of red colonies in adenine deficient synthetic medium, for CRISPRd.

Strains and Cultivation Conditions.

E. coli strain DH5a was used to maintain and amplify plasmids and recombinant strains were cultured at 37° C. in Luria broth medium containing 100 μg mL⁻¹ ampicillin. S. cerevisiae CEN.PK2-1C strain (EUROSCARF, Frankfurt, Germany) was used as the host for homologous recombination based cloning, recombinant protein expression and surface display, and β-carotene production. Yeast strains were cultivated in complex medium consisting of 2% peptone and 1% yeast extract supplemented with 2% glucose (YPD). Recombinant strains were grown on synthetic complete medium consisting of 0.17% yeast nitrogen base, 0.5% ammonium sulfate, and the appropriate amino acid drop out mix, supplemented with 2% glucose (SCD). When necessary, 200 μg mL⁻¹ G418 (KSE Scientific, Durham, N.C.) was supplemented to the growth media. Ammonium sulfate was replaced with 0.1% mono-sodium glutamate (SED), when G418 was used in synthetic medium. All restriction enzymes, Q5 polymerase, and the E. coli-S. cerevisiae shuttle vectors were purchased from New England Biolabs (Ipswich, Mass.). All chemicals were purchased from Sigma-Aldrich (St. Louis, Mo.) unless otherwise specified.

Plasmid and Strain Construction.

Recombinant plasmids were constructed using restriction digestion/ligation, Gibson Assembly, Golden-Gate Assembly, or the yeast homologous recombination based DNA assembler method (Shao, Z., et al., Nucleic Acids Res. 37:e16 (2009)). All the recombinant plasmids and gRNA plasmids used in this study were listed in Table 1 and Table 2, respectively.

TABLE 1
Plasmids used in this study

| Plasmids | Genotype | Reference |
|---|---|---|
| pRS406 | Integrative vector with URA3 marker | |
| pH1 | pRS425-PDC1p-eGFP-ADH1t | Lian, J., et al., ACS |
| | | Synth. Biol. 4: 332-341 |
| | | (2015); Lian, J., |
| | | et al., ACS |
| | | Synth. Biol. 5: 689-697 |
| | | (2016) |
| pH3 | pRS425-ENO2p-eGFP-CYC1t-TPI1p | Lian, J., et al., ACS |
| | | Synth. Biol. 4: 332-341 |
| | | (2015); Lian, J., |
| | | et al., ACS |
| | | Synth. Biol. 5: 689-697 |
| | | (2016) |
| pH4 | pRS425-TPI11p-eGFP-TPI1t-TEF1p | Lian, J., et al., ACS |
| | | Synth. Biol. 4: 332-341 |
| | | (2015); Lian, J., |
| | | et al., ACS |
| | | Synth. Biol. 5: 689-697 |
| | | (2016) |
| pH5 | pRS425-TEF1p-eGFP-TEF1t | Lian, J., et al., ACS |
| | | Synth. Biol. 4: 332-341 |
| | | (2015); Lian, J., |
| | | et al., ACS |
| | | Synth. Biol. 5: 689-697 |
| | | (2016) |
| pH6 | pRS425-TEF1t-PGK1p-BamHI-HXT7t | Lian, J., et al., ACS |
| | | Synth. Biol. 4: 332-341 |
| | | (2015); Lian, J., |
| | | et al., ACS |
| | | Synth. Biol. 5: 689-697 |
| | | (2016) |
| p41K-CEN-Delta | pRS-KanMX-Delta1-PmeI-CEN/ARS-PmeI-Delta2 | Du, J., et al., |
| | | Nucleic Acids Res. |
| | | 40: e142 (2012) |
| pcDNA-NMdCas9-VPR | Harboring dNmCas9-VPR | Bao, Z., et al., ACS |
| | | Synth. Biol. (2017) |
| pcDNA-SPdCas9-VPR | Harboring dSpCas9-VPR | Bao, Z., et al., ACS |
| | | Synth. Biol. (2017) |
| M-ST1n-VPR | Harboring dSt1Cas9-VPR | Addgene (Chavez, A., |
| | | et al., Nat. |
| | | Methods 12: 326-328 |
| | | (2015)) |
| AAV_NLS-dSaCas9- | Harboring dSaCas9-VPR | Addgene (Kiani, S., |
| NLS-VPR | | et al., Nat. |
| | | Methods 12: 1051-1054 |
| | | (2015)) |
| pCR | Harboring SpSgRNA scaffold in BsaI-free pRS423 | Bao, Z., et al., ACS |
| | | Synth. Biol. 4: 585-594 |
| | | (2015) |
| pCT | Harboring SpCas9 | Bao, Z., et al., ACS |
| | | Synth. Biol. 4: 585-594 |
| | | (2015) |
| pTDH3-dCas9-Mxi1 | Harboring TDH3p-dSpCas9-MXI1-ADH1t | Gilbert, L. A., et |
| | | al., Cell 154: 442-451 |
| | | (2013) |
| pSimpleII-U6-tracr-U6- | Harboring NmCas9 and NmSgRNA scaffold | Addgene (Hou, Z., |
| BsmBI-NLS-NmCas9- | | et al., Proc. Natl. |
| HA-NLS(s) | | Acad. Sci. U.S.A. |
| | | 110: 15644-15649 |
| | | (2013)) |
| MSP1673 | Harboring St1Cas9 and St1SgRNA scaffold | Addgene |
| | | (Kleinstiver, B. P., |
| | | et al., Nature |
| | | 523: 481-485 |
| | | (2015)) |
| BPK2139 | Harboring SaCas9 | Addgene |
| | | (Kleinstiver, B. P., |
| | | et al., Nature |
| | | 523: 481-485 |
| | | (2015)) |
| pcDNA3.1-hAsCpf1 | Harboring AsCpf1 | Addgene (Zetsche, B., |
| | | et al., Cell |
| | | 163: 759-771 |
| | | (2015)) |
| pcDNA3.1-hLbCpf1 | Harboring LbCpf1 | Addgene (Zetsche, B., |
| | | et al., Cell |
| | | 163: 759-771 |
| | | (2015)) |
| VVT1 | Harboring SaSgRNA scaffold | Addgene |
| | | (Kleinstiver, B. P., |
| | | et al., Nature |
| | | 523: 481-485 |
| | | (2015)) |
| pJZC588 | SgRNA with 2x MS2 (wt+f6) | Addgene (Zalatan, J. G., |
| | | et al., Cell |
| | | 160: 339-350 |
| | | (2015)) |
| pJZC603 | SgRNA with 2x PP7 | Addgene (Zalatan, J. G., |
| | | et al., Cell |
| | | 160: 339-350 |
| | | (2015)) |
| pJZC620 | Harboring dCas9, MCP-VP64, and PCP-VP64 | Addgene (Zalatan, J. G., |
| | | et al., Cell |
| | | 160: 339-350 |
| | | (2015)) |
| YIplac211-YB/E/I | Yeast integrative vector with URA3 marker and | Euroscarf |
| | CrtYB, CrtE, and CrtI expression cassettes | (Verwaal, R., et |
| | | al., Appl. Environ. |
| | | Microbiol. |
| | | 73: 4342-4350 |
| | | (2007)) |
| YIplac128-I | Yeast integrative vector with LEU2 marker and CrtI | Euroscarf |
| | expression cassettes | (Verwaal, R., et |
| | | al., Appl. Environ. |
| | | Microbiol. |
| | | 73: 4342-4350 |
| | | (2007)) |
| p406-CT | pRS406-CYC1p-mCherry-TEF1t-TEF1p-mVenus- | This study |
| | PGK1t | |
| p406-CF | pRS406-CYC1p-mCherry-TEF1t-FBA1p-mVenus- | This study |
| | PGK1t | |
| p406-CH | pRS406-CYC1p-mCherry-TEF1t-HHF2p-mVenus- | This study |
| | PGK1t | |
| p406-CR1 | pRS406-CYC1p-mCherry-TEF1t-REV1p-mVenus- | This study |
| | PGK1t | |
| p406-CR2 | pRS406-CYC1p-mCherry-TEF1t-RNR2p-mVenus- | This study |
| | PGK1t | |
| p406-YD-EGII | pRS406-TEF1p-prepro-HisTag-EGII-GS-cSAG1- | This study |
| | PGK1t | |
| pH5-SpCas9 | pRS425-TEF1p-NLS-SpCas9-NLS-TEF1t | This study |
| pH5-NmCas9 | pRS425-TEF1p-NLS-NmCas9-NLS-TEF1t | This study |
| pH5-St1Cas9 | pRS425-TEF1p-St1Cas9-NLS-TEF1t | This study |
| pH5-SaCas9 | pRS425-TEF1p-SaCas9-NLS-TEF1t | This study |
| pH5-AsCpf1 | pRS425-TEF1p-AsCpf1-NLS-TEF1t | This study |
| pH5-LbCpf1 | pRS425-TEF1p-LbCpf1-NLS-TEF1t | This study |
| pSgH | pRS423*(BsaI-free)-SNR52p-BsaI-BsaI-SUP4t | This study |
| pSpSgH | pRS423*(BsaI-free)-SNR52p-BsaI-BsaI-SpSgRNA- | This study |
| | SUP4t | |
| pNmSgH | pRS423*(BsaI-free)-SNR52p-BsaI-BsaI-NmSgRNA- | This study |
| | SUP4t | |
| pSt1SgH | pRS423*(BsaI-free)-SNR52p-BsaI-BsaI-St1SgRNA- | This study |
| | SUP4t | |
| pSaSgH | pRS423*(BsaI-free)-SNR52p-BsaI-BsaI-SaSgRNA- | This study |
| | SUP4t | |
| pRS423-H5 | pRS423-TEF1p-eGFP-TEF1t | This study |
| pH5-NLS-St1Cas9 | pRS425-TEF1p-NLS-St1Cas9-NLS-TEF1t | This study |
| pH5-NLS-SaCas9 | pRS425-TEF1p-NLS-SaCas9-NLS-TEF1t | This study |
| pH5-NLS-AsCpf1 | pRS425-TEF1p-NLS-AsCpf1-NLS-TEF1t | This study |
| pH5-NLS-LbCpf1 | pRS425-TEF1p-NLS-LbCpf1-NLS-TEF1t | This study |
| pTDH3-dLbCpf1-MXI1 | pTDH3p-dLbCpf1-MXI1-ADH1t | This study |
| pTDH3-dLbCpf1-V | pTDH3p-dLbCpf1-V-ADH1t | This study |
| pTDH3-dLbCpf1-VP | pTDH3p-dLbCpf1-VP-ADH1t | This study |
| pTDH3-dLbCpf1-VPR | pTDH3p-dLbCpf1-VPR-ADH1t | This study |
| pH6-dSpCas9-V | pRS425-PGK1p-dSpCas9-V-HXT7t | This study |
| pH6-dSpCas9-VP | pRS425-PGK1p-dSpCas9-VP-HXT7t | This study |
| pH6-dSpCas9-VPR | pRS425-PGK1p-dSpCas9-VPR-HXT7t | This study |
| pH6-dSt1Cas9-V | pRS425-PGK1p-dSt1Cas9-V-HXT7t | This study |
| pH6-dSt1Cas9-VP | pRS425-PGK1p-dSt1Cas9-VP-HXT7t | This study |
| pH6-dSt1Cas9-VPR | pRS425-PGK1p-dSt1Cas9-VPR-HXT7t | This study |
| pH6-dSaCas9-V | pRS425-PGK1p-dSaCas9-V-HXT7t | This study |
| pH6-dSaCas9-VP | pRS425-PGK1p-dSaCas9-VP-HXT7t | This study |
| pH6-dSaCas9-VPR | pRS425-PGK1p-dSaCas9-VPR-HXT7t | This study |
| pTDH3-dSpCas9-RD1 | pTDH3p-dSpCas9-RD1-ADH1t | This study |
| pTDH3-dSpCas9-RD2 | pTDH3p-dSpCas9-RD2-ADH1t | This study |
| pTDH3-dSpCas9-RD3 | pTDH3p-dSpCas9-RD3-ADH1t | This study |
| pTDH3-dSpCas9-RD4 | pTDH3p-dSpCas9-RD4-ADH1t | This study |
| pTDH3-dSpCas9-RD5 | pTDH3p-dSpCas9-RD5-ADH1t | This study |
| pTDH3-dSpCas9-RD6 | pTDH3p-dSpCas9-RD6-ADH1t | This study |
| pTDH3-dSpCas9-RD7 | pTDH3p-dSpCas9-RD7-ADH1t | This study |
| pTDH3-dSpCas9-RD8 | pTDH3p-dSpCas9-RD8-ADH1t | This study |
| pTDH3-dSpCas9-RD9 | pTDH3p-dSpCas9-RD9-ADH1t | This study |
| pTDH3-dSpCas9-RD10 | pTDH3p-dSpCas9-RD10-ADH1t | This study |
| pTDH3-dSpCas9-RD11 | pTDH3p-dSpCas9-RD11-ADH1t | This study |
| pTDH3-RD2-dSpCas9- | pTDH3p-RD2-dSpCas9-RDS-ADH1t | This study |
| RD5 | | |
| pTDH3-RD2-dSpCas9- | pTDH3p-RD2-dSpCas9-RD11-ADH1t | This study |
| RD11 | | |
| pTDH3-RD5-dSpCas9- | pTDH3p-RD5-dSpCas9-RD11-ADH1t | This study |
| RD11 | | |
| pTDH3-dSpCas9- | pTDH3p-dSpCas9-RD11-RD5-RD2-ADH1t | This study |
| RD1152 | | |
| pH4-dSpCas9-RD1152 | pRS425-TPI1p-dSpCas9-RD11-RD5-RD2-TPI1t- | This study |
| | TEF1p | |
| pH3-Csy4 | pRS425-ENO2p-Csy4-PGK1t-TPI1p | This study |
| pAID6 | p41K-CEN-Delta-TDH3p-dLbCpf1-VP-ADH1t- | This study |
| | ENO2p-Csy4-PGK1t-TPI1p-dSpCas9-RD11-RD5- | |
| | RD2-TPI1t-TEF1p-SaCas9-NLS-TEF1t | |
| pSpMS2SgH | pRS423*-SNR52p-BsaI-BsaI-SpSgRNA-MS2-SUP4t | This study |
| pSpPP7SgH | pRS423*-SNR52p-BsaI-BsaI-SpSgRNA-PP7-SUP4t | This study |
| pSpComSgH | pRS423*-SNR52p-BsaI-BsaI-SpSgRNA-Com-SUP4t | This study |
| pH1-PP7-MXI1 | pRS425-PDC1p-PP7-MXI1-ADH1t | This study |
| pH1-PP7-RD2 | pRS425-PDC1p-PP7-RD2-ADH1t | This study |
| pH1-PP7-RD4 | pRS425-PDC1p-PP7-RD4-ADH1t | This study |
| pH1-Com-MXI1 | pRS425-PDC1p-Com-MXI1-ADH1t | This study |
| pH1-Com-RD2 | pRS425-PDC1p-Com-RD2-ADH1t | This study |
| pH1-Com-RD4 | pRS425-PDC1p-Com-RD4-ADH1t | This study |
| pH4-MS2-VP64 | pRS425-TPI1p-MS2-VP64-TPI1t-TEF1p | This study |

TABLE 2
gRNA plasmids constructed in this study

| | | | | | | | SEQ | |
|---|---|---|---|---|---|---|---|---|
| | | | | | | | ID | |
| Plasmid | Cas9 | Target | AID | Position | Strand | Protospacer | NO: | PAM |
| pSg1 | Sp | TEF1p | i | −115 to −134 | t | ttgatatttaagttattaaa | 01 | tgg |
| pSg6 | Sp | CYC1p | a | −183 to −202 | t | actttagtgctgacacatac | 02 | agg |
| pSg10 | Sp | ADE2 | d | 157 to 177 | nt | gatatcaagaggattggaaa | 03 | agg |
| pSg11 | Sp | | Same as psg10, except that 100 bp hr donor | | | | | |
| | | | was integrated (HI-CRISPR) | | | | | |
| pSg12 | Nm | ADE2 | d | 394 tp 413 | t | acgtccctattgaatgttgg | 04 | aagagatt |
| pSg13 | Nm | ADE2 | d | 826 to 845 | t | aactctggacattataccat | 05 | tgatgctt |
| pSg14 | St1 | ADE2 | d | 548 to 567 | t | aaaaatgggcaccatttact | 06 | aaagaat |
| pSg15 | St1 | ADE2 | d | 622 to 641 | t | ccaattgtagagactatcca | 07 | caagga |
| pSg27 | Sp | TEF1p | i | −115 to −128 | t | tttaagttattaaa | 08 | tgg |
| pSg28 | Sp | TEF1p | i | −125 to −138 | nt | taaatatcaatggg | 09 | agg |
| pSg29 | Nm | ADE2 | d | 871 to 890 | t | gaagctcatttgagatcaat | 10 | attggatt |
| pSg30 | St1 | ADE2 | d | 466 to 485 | t | ggaagaggtaacttcgttgt | 11 | aaagaat |
| pSg31 | Sa | ADE2 | d | 833 to 855 | nt | gcaagcatcaatggtataatgtc | 12 | cagagt |
| pSg32 | Nm | ADE2 | d | 473 to 496 | t | gtaacttcgttgtaaagaataagg | 13 | aaatgatt |
| pSg33 | Sp | CYC1p | a | −183 to −196 | t | gtgctgacacatac | 14 | agg |
| pSg35 | Sp | TEF1p | i | −115 to −134 | t | gatatttaagttattaaa | 15 | tgg |
| pSg36 | Sp | TEF1p | i | −115 to −134 | t | tatttaagttattaaa | 16 | tgg |
| pSg37 | Sp | TEF1p | i | −115 to −134 | t | atttaagttattaaa | 17 | tgg |
| pSg38 | Sp | TEF1p | i | −115 to −134 | t | ttaagttattaaa | 18 | tgg |
| pSg39 | Sp | TEF1p | i | −115 to −134 | t | taagttattaaa | 19 | tgg |
| pSg40 | Sp | TEF1p | i | −115 to −134 | t | agttattaaa | 20 | tgg |
| pSg45 | SpMS2 | CYC1p | a | | The same as Sg33 | | | |
| pSg46 | SpPP7 | TEF1p | i | | The same as Sg27 | | | |
| pSg55 | Sp | REV1p | a | −250 to −269 | t | gaaaaaagtagcta | 21 | agg |
| pSg56 | Sp | RNR2p | a | −242 to −261 | t | ccgtaccataccct | 22 | tgg |
| pSg64 | St1 | ADE2 | d | 621 to 640 | nt | ggatagtctctacaattggg | 23 | taagaaa |
| pSg65 | Sa | CYC1p | a | −217 to −239 | t | tccgccaggcgtgtatatatagc | 24 | gtggat |
| pSg66 | Sa | RNR2p | a | −203 to −225 | t | aacgaagcaggaaatgagagaat | 25 | gagagt |
| pSg68 | As | ADE2 | d | 155 to 177 | nt | gatatcaagaggattggaaaagg | 26 | tttc |
| pSg69 | Lb | ADE2 | d | 155 to 177 | nt | gatatcaagaggattggaaaagg | 27 | tttc |
| pSg87 | Sa | RNR2p | a | −203 to −223 | t | cgaagcaggaaatgagagaat | 28 | gagagt |
| pSg88 | Sa | RNR2p | a | −219 to −239 | nt | cttcgttcatttcgagtttcc | 29 | aagggt |
| pSg89 | Sa | RNR2p | a | −384 to −404 | t | cagacctccctgcgagcgggc | 30 | atgggt |
| pSg90 | Sa | CYC1p | a | −217 to −237 | t | cgccaggcgtgtatatatagc | 31 | gtggat |
| pSg91 | Sa | CYC1p | a | −277 to −297 | t | tcatttggcgagcgttggttg | 32 | gtggat |
| pSg92 | Sa | CYC1p | a | −337 to −357 | t | gatctttccggtctctttggc | 33 | gtggat |
| pSg93 | Sa | ADE2d | d | 367 to 387 | nt | ggcttgttccacaggaacact | 34 | ttgggt |
| pSg94 | Sa | ADE2d | d | 438 to 458 | nt | gccaaagtcctcgacttcaag | 35 | acgaat |
| pSg95 | Sa | ADE2d | d | 695 to 715 | nt | acaacttcgccttaagttgaa | 36 | cggagt |
| pSg109 | Sp | TEF1p | i | 1 to −19 | t | tctaagttttaattacaaaa | 37 | tgg |
| pSg110 | Sp | mVenus | i | 3 to 22 | t | ggaattcgtgagcaagggcg | 38 | tgg |
| pSg111 | Sp | mVenus | i | 21 to 40 | t | cgaggagctgttcaccgggg | 39 | cgg |
| pSg112 | Sp | mVenus | i | 38 to 57 | nt | gaccaggatgggcaccaccc | 40 | agg |
| pSg113 | Sp | mVenus | i | 54 to 73 | nt | cgtcgccgtccagctcgacc | 41 | ggg |
| pSg114 | Sp | mVenus | i | 140 to 159 | nt | ggtggtgcagatcagcttca | 42 | tgg |
| pSg115 | Sp | FBA1p | i | 1 to −19 | t | caagtaatacatattcaaaa | 43 | tgg |
| pSg116 | Sp | FBA1p | i | −4 to −23 | nt | gaatatgtattacttggtta | 44 | tgg |
| pSg117 | Sp | FBA1p | i | −48 to −67 | t | aagaacagaagaataacgca | 45 | agg |
| pSg118 | Sp | FBA1p | i | −145 to −164 | t | ttatccctcatgttgtctaa | 46 | cgg |
| pSg119 | Sp | HHF2p | i | 1 to −19 | t | caatcaatacaataaaataa | 47 | tgg |
| pSg120 | Sp | HHF2p | i | −29 to −48 | nt | tactcttttgaacaagatgt | 48 | agg |
| pSg121 | Sp | HHF2p | i | −107 to −120 | t | ataagtatattaggatgagg | 49 | cgg |
| pSg122 | Lb | ADE2 | d | 219 to 241 | nt | gtgtaggaacatcaacatgctca | 50 | ttta |
| pSg123 | Lb | ADE2 | d | 282 to 304 | t | cccttctccagaaacaatcagat | 51 | ttta |
| pSg124 | Lb | ADE2 | d | 430 to 452 | t | ccattcgtcttgaagtcgaggac | 52 | tttt |
| pSg125 | Lb | TEF1 | i | −101 to −123 | t | agttattaaatggtcttcaattt | 53 | ttta |
| pSg126 | Lb | TEF1 | i | −118 to −140 | nt | ataacttaaatatcaatgggagg | 54 | ttta |
| pSg127 | St1 | RNR2 | a | −210 to −229 | t | aatgaacgaagcaggaaatg | 55 | agagaat |
| pSg128 | St1 | RNR2 | a | −308 to −327 | t | gcgtgttgttgctgctgaca | 56 | aaagaaa |
| pSg131 | SpCom | TEF1 | i | | The same as Sg27 | | | |
| pSg135 | Lb | TEF1 | i | −33 to −55 | t | cttcttgctcattagaaagaaag | 57 | ttta |
| pSg136 | Lb | TEF1 | i | −5 to −27 | nt | taattaaaacttagattagattg | 58 | tttg |
| pSg137 | Lb | mVenus | i | 51 to 73 | nt | cgtcgccgtccagctcgaccagg | 59 | ttta |
| pSg138 | St1 | RNR2 | a | −277 to −296 | t | tttcttagcaaagcaaagga | 60 | ggggaa |
| pSg139 | St1 | RNR2 | a | −220 to −239 | t | ggaaactcgaaatgaacgaa | 61 | gcagga |
| pSg140 | St1 | RNR2 | a | −274 to −293 | t | cttagcaaagcaaaggaggg | 62 | gaagca |
| pSg141 | St1 | RNR2 | a | −164 to −183 | t | atagcggtagtgtttgcgcg | 63 | ttacca |
| pSg142 | St1 | CYC1 | a | −327 to −346 | nt | gtaaaccccggccaaagaga | 64 | ccggaa |
| pSg143 | St1 | CYC1 | a | −226 to −245 | nt | acacgcctggcggatctgct | 65 | cgagga |
| pSg144 | St1 | CYC1 | a | −383 to −402 | t | acctgaatctaaaattcccg | 66 | ggagca |
| pSg145 | Sa | ADE2 | d | The same as Sg95, but with 100 bp HR (HI-CRISPR) | | | | |
| pSg146 | St1 | CYC1 | a | −319 to −338 | t | gccggggtttacggacgatg | 67 | gcagaa |
| pSg147 | St1 | REV1 | a | −247 to −266 | t | gacggaaaaaagtagctaag | 68 | gaagaa |
| pSg148 | St1 | REV1 | a | −383 to −402 | nt | caaagcattcaattcaaatg | 69 | aaagaa |
| pSg149 | Lb | RNR2 | a | −239 to −261 | nt | caagggtatggtacggtgctatc | 70 | tttc |
| pSg150 | Lb | RNR2 | a | −309 to −331 | nt | tcagcagcaacaacacgctacgc | 71 | tttg |
| pSg155 | Lb | CYC1 | a | −306 to −328 | t | cggacgatggcagaagaccaaag | 72 | ttta |
| pSg156 | Lb | CYC1 | a | −269 to −291 | t | gcgagcgttggttggtggatcaa | 73 | tttg |
| pSg157 | Lb | CYC1 | a | −174 to −196 | t | gtgctgacacatacaggcatata | 74 | ttta |
| pSg163 | AID6 | Sg156-Sg112-Sg145 | | | | | | |
| pSg172 | Sp | ERG9 | i | −87 to −106 | t | ataaatggaaagttaggaca | 75 | ggg |
| pSg175 | Lb | HMG1 | a | −228 to −250 | t | cggctatgaaaagctgttgttcg | 76 | tttt |
| pSg186 | Sa | ROX1 | d | 68 to 88 | t | actaccacaggatcttaatag | 77 | acgaat |
| pSg194 | Lb | PEX5 | a | −182 to −204 | nt | catattcgaagcttacaatcgag | 78 | ttta |
| pSg195 | Lb | PEX5 | a | −296 to −318 | t | taccagcaatcagctgactaaca | 79 | ttta |
| pSg196 | Lb | PTI1 | a | −259 to −281 | t | ttgctcttacccgactctgaaga | 80 | ttta |
| pSg197 | Lb | PTI1 | a | −174 to −196 | nt | gcaagacctcaaacaatcgtact | 81 | tttc |
| pSg198 | Sp | SED1 | i | −165 to −187 | t | gctggggtagaactagagta | 82 | agg |
| pSg199 | Sp | SED1 | i | −127 to −146 | nt | ttatatgacagttcaaaaga | 83 | ggg |
| pSg200 | Sp | SED1 | i | 101 to 120 | nt | ggaagtggagatggaagagg | 84 | agg |
| pSg201 | Sp | YCH1 | i | −169 to −188 | t | ctacatgcaaacgacaaata | 85 | cgg |
| pSg202 | Sp | YCH1 | i | −61 to −80 | nt | gctgaaaactgtatgtgcgg | 86 | agg |
| pSg203 | Sp | YCH1 | i | 43 to 62 | nt | atccaacgatgcaattcagt | 87 | cgg |
| pSg204 | Sp | PMR1 | i | −107 to −126 | nt | aaatgggaatggaaagaacg | 88 | ggg |
| pSg205 | Sa | PMR1 | d | 683 to 703 | nt | atctctcagaaatcggtacaa | 89 | ttgaat |
| pSg217 | Lb | CCW12 | a | −242 to −264 | t | caacaactatctgcgataactca | 90 | tttg |
| pSg218 | Lb | ERO1 | a | −221 to −243 | nt | cagggtcttctataagagaaacc | 91 | tttc |
| pSg219 | Lb | HAC1 | a | −266 to −288 | nt | agccctacttaatgctgagccac | 92 | tttt |
| pSg220 | Lb | KAR2 | a | −214 to −236 | t | gctatgttagctgcaactttcta | 93 | tttt |
| pSg221 | Lb | PDI1 | a | −275 to −297 | t | gaaacacgtgtcctgaaaattat | 94 | tttc |
| pSg222 | Lb | SEC1 | a | −235 to −257 | t | aaaatcatcgaatagccgatcga | 95 | ttta |
| pSg223 | Lb | SLY1 | a | −217 to −239 | t | ccagtcactatcatcatcatcat | 96 | tttt |
| pSg224 | Lb | SSO1 | a | −256 to −278 | nt | acgggcaaaaactggattctccc | 97 | ttta |
| pSg225 | Lb | SSO2 | a | −234 to −256 | t | tgtcttacgagccgggtaccaag | 98 | ttta |
| pSg226 | Lb | UBI4 | a | −231 to −253 | t | caggggcgatgccacttatcagt | 99 | tttt |
| pSg227 | Sp | OCH1 | i | −134 to −153 | nt | ggattggcgagaaataatgt | 100 | cgg |
| pSg228 | Sp | OCH1 | i | −113 to −132 | nt | gcagatggggagagagaatg | 101 | tgg |
| pSg229 | Sp | OCH1 | i | 20 to 39 | nt | tttccttgtagcgatcaggt | 102 | ggg |
| pSg230 | SP | MNN9 | i | −112 to −131 | nt | gaaataacgggtcccaagag | 103 | cgg |
| pSg231 | Sp | MNN9 | i | 27 to 46 | nt | cccacgggttctttcttagg | 104 | cgg |
| pSg239 | AID | Sg175-Sg172-Sg186 | | | | | | |
| pSg257 | AID | Sg218-Sg204-Sg186 | | | | | | |
| pSg260 | Sp | PMR1 | i | −129 to −148 | nt | gcgagcaaacactattatga | 105 | tgg |
| pSg261 | Sp | PMR1 | i | 86 to 105 | nt | agaagggcttggtttcgaaa | 106 | ggg |
| pSg262 | Sp | KEX2 | i | −116 to −135 | nt | caaaacgggatatttaagcc | 107 | agg |
| pSg263 | Sp | KEX2 | i | −76 to −95 | nt | agccgaatgaatgaaatatg | 108 | tgg |
| pSg264 | Sp | KEX2 | i | 56 to 75 | nt | ttgttgtgatgatacaagag | 109 | cgg |
| pSg265 | Sa | PEP4 | d | 821 to 841 | t | ttgaaggtatcggtttaggcg | 110 | acgagt |
| pSg266 | Sa | VPS8 | d | 470 to 490 | t | tatgcatttggaacttgaacg | 111 | tagggt |
| pSg267 | Sa | YPS1 | d | 1190 to 1210 | nt | atacgtaataccctatcctgg | 112 | aagagt |

| | | |
|---|---|---|
| FACS16 | AID | Sg221-Sg230-Sg205 (the same as FACS22) |
| pSg417 | AI | Sg221-Sg230-SgH |
| pSg418 | AD | Sg221-SgH-Sg205 |
| pSg419 | ID | SgH-Sg230-Sg205 |
| pSg585 | AI | Sg175-Sg172-SgH |

Oligonucleotides used for gene amplification, pathway assembly, diagnostic PCR verification, and qPCR analysis were listed in Table 3.

TABLE 3
Oligonucleotides used in this study.

| Oligos | Sequences (5′-3′) | SEQ ID NO. | Applications |
|---|---|---|---|
| CT-F1 | ctcactatagggcgaattgggtaccctcgagaatttttttggaa | 113 | Construct p406-CT |
| | aaccaag | | (Gibson) |
| CT-R1 | gttatcctcctcgcccttgctcaccattattaatttagtgtgtgtatt | 114 | |
| | tg | | |
| CT-F2 | cacaaatacacacactaaattaataatggtgagcaagggcga | 115 | |
| | ggag | | |
| CT-R2 | gcctgttgctatcgataccgtcgacatagcgccgatcaaagta | 116 | |
| | tag | | |
| CT-F3 | tcggcgctatgtcgacggtatcgatagcaacaggcgcgttgg | 117 | |
| | ac | | |
| CT-R3 | ctaaagggaacaaaagctggagctccaggaagaatacactat | 118 | |
| | actg | | |
| CF-F | cgctatgtcgac tgggtcattacgtaaataatgatag | 119 | p406-CF (ligation) |
| CF-R | ctcacgaattccat tttgaatatgtattacttggttatg | 120 | |
| CH-F | cgctatgtcgac gttttgacaccgagccatagc | 121 | p406-CH (ligation) |
| CH-R | ctcacgaattccat tattttattgtattgattgttg | 122 | |
| CR1-F | cgctatgtcgac catccacatattttaatcac | 123 | p406-CR1 (ligation) |
| CR1-R | ctcacgaattccat cgctggatatgcctagaaatg | 124 | |
| CR2-F | cgctatgtcgac aactatgcgaaatccggagcaac | 125 | p406-CR2 (ligation) |
| CR2-R | ctcacgaattccat ggtaattggacaaataaatac | 126 | |
| NmCas9-F | gttcgcggatcc atggtgcctaagaagaagagaaag | 127 | pH5-NmCas9 |
| NmCas9-R | cacccgctcgag ttaatccagcttctttttcttcg | 128 | (ligation) |
| St1Cas9-F | gttcgcggatcc atgagcgacctggtgctgggcctg | 129 | pH5-St1 Cas9 |
| St1Cas9-R | cacccgctcgag tcacaccttcctcttcttcttgg | 130 | (ligation) |
| SaCas9-F | gacatgccatggggaaacggaactacatcctg | 131 | pH5-SaCas9 |
| SaCas9-R | gaacgcgtcgacttacttgtcatcgtcatccttg | 132 | (ligation) |
| AsCpf1-F | gttcgcggatcc atgacacagttcgagggctttac | 133 | pH5-As (Lb) Cpf1 |
| LbCpf1-F | gttcgcggatcc atgagcaagctggagaagtttacaaactg | 134 | (ligation) |
| Cpf1-R | cacccgctcgag tca ctttttcttttttgcctggcc | 135 | |
| SgH-F | ccactacgtgctcgagtctttgaaaagataatg | 136 | pSgH (ligation) |
| SgH-R | Gcagggagctcagacataaaaaacaaaaaaa | 137 | |
| | ggagacctcggtctccgatcatttatctacactgc | | |
| SpSgH-F | ctccgcagtgaaagataaatgatcggagaccgaggtctccgt | 138 | pSpSgH (ligation) |
| | tttagagctagaaatagc | | |
| SpSgH-R | cagacataaaaaacaaaaaaa | 139 | |
| | ggatcaaaaaagcaccgactcggtg | | |
| NmSgH-F | ctccgcagtgaaagataaatgatcggagaccgaggtctccgt | 140 | pNmSgH (ligation) |
| | tgtagctccctactcat | | |
| NmSgH-R | cagacataaaaaacaaaaaaa | 141 | |
| | ggatctaaacgatgccccttaaagc | | |
| St1SgH-F | ctccgcagtgaaagataaatgatcggagaccgaggtctccgt | 142 | pSt1SgH (ligation) |
| | ttttgtactctcagaaat | | |
| St1SgH-R | cagacataaaaaacaaaaaaa | 143 | |
| | ggatcaaaaaaacaccctgccataaaatg | | |
| SaSgH-F | ctccgcagtgaaagataaatgatcggagaccgaggtctccgt | 144 | pSaSgH (ligation) |
| | taagtactctgtaata | | |
| SaSgH-R | cagacataaaaaacaaaaaaa | 145 | |
| | ggatcaaaaaaatctcgccaacaag | | |
| NLS-BamHI-F | gatccatgcctccaaaaaagaagagaaaggtcggtagtggtt | 146 | Insert N-terminal |
| | ctg | | NLS at BamHI or |
| NLS-BamHI-R | gatccagaaccactaccgacctttctcttcttttttggaggcatg | 147 | NcoI site |
| NLS-NcoI-F | catgggccctccaaaaaagaagagaaaggtcggtagtggttc | 148 | |
| | ttc | | |
| NLS-NcoI-R | catggaagaaccactaccgacctttctcttcttttttggagggcc | 149 | |
| ADE2-KO-F | atggattctagaacagttggtatattaggagggggacaatttcg | 150 | linear donor for |
| | tacgctgcaggtcgac | | ADE2 deletion |
| ADE2-KO-R | ttacttgttttctagataagcttcgtaaccgacagtttctgcatag | 151 | |
| | gccactagtggatc | | |
| Csy4-F | gttggaagatctatg ggtgatcattatctggatattc | 152 | pH3-Csy4 (ligation) |
| Csy4-R | cacccgctcgag tta aaaccagggcacgaaac | 153 | |
| dCas9-AD-F | actttttacaacaaatataaaacaGatggactacaaagaccat | 154 | pH6-dSp/St1-Cas9- |
| | gacggtg | | V/VP/VPR (Gibson) |
| dCas9-V-R | gaattaataaaagtgttcgcaaaggatctcacagcaaggctga | 155 | |
| | gaaatccatatc | | |
| dCas9-VP-R | gaattaataaaagtgttcgcaaaggatctcataacatatcgaga | 156 | |
| | tcgaaatc | | |
| dCas9-VPR-R | gaattaataaaagtgttcgcaaaggatctcaagaagcgtagtc | 157 | |
| | cggaacgtc | | |
| dSaCas9-AD-F | actttttacaacaaatataaaacagatggccccaaagaagaag | 158 | pH6-dSaCas9- |
| | cggaag | | V/VP/VPR (Gibson) |
| dSaCas9-V-R | gaattaataaaagtgttcgcaaaggatccagcatgtccaggtc | 159 | |
| | gaaatcatcaag | | |
| dSaCas9-VPR-R | gaattaataaaagtgttcgcaaaggatctcaaaacagagatgt | 160 | |
| | gtcgaagatg | | |
| dLbCpf1-F1 | ccgccaccatggct cctccaaaaaagaagagaaag | 161 | dLbCpf1 |
| dLbCpf1-R1 | caccacgatatacagcagattgcgctcgcccctagcgatgcc | 162 | OE-PCR |
| | gatcacataggggttatc | | |
| dLbCpf1-F2 | ctgaagcacgacgataacccctatgtgatcggcatcgctagg | 163 | |
| | ggcgagcgcaatctgctg | | |
| dLbCpf1-R2 | ccgccgaagcttctttttcttttttgcctggccgg | 164 | |
| RD1-F | agttccaagcttggcggcagcggcggcagc | 165 | Amplification of |
| | atgactgccagcgtttcgaatac | | RD1/RD2/ |
| RD1-R | cacccgctcgag tta aggtggttgctgttgttgaagttg | 166 | RD3/RD4 |
| RD2-F | agttccaagcttggcggcagcggcggcagc | 167 | |
| | tacgaagaagagatcaagcac | | |
| RD2-R | cacccgctcgag tta cgcaactggaacagatgcagatg | 168 | |
| RD3-F | agttccaagcttggcggcagcggcggcagc | 169 | |
| | gctagtttgcaccaggatcac | | |
| RD3-R | cacccgctcgag tta agatttgtgtaactcaacgtc | 170 | |
| RD5-F | agttccaagcttggcggcagcggcggcagc | 171 | Amplification of |
| | gattcacaagttcaagaactg | | RD5/RD6 |
| RD5-R | cacccgctcgag tcagtccatgtgtgggaaggg | 172 | |
| RD6-F | agttccaagcttggcggcagcggcggcagc | 173 | |
| | actagtggtacgaatttgcac | | |
| RD6-R | Same as RD5-R | | |
| RD7-F | agttccaagcttggcggcagcggcggcagc | 174 | Amplification of |
| | atggtaatcttcaaagaacg | | RD7/RD8/ |
| RD7-R | cacccgctcgag tta gataagtggcggtaatattg | 175 | RD9 |
| RD8-F | Same as RD7-F | | |
| RD8-R | cacccgctcgag tta agatttgttattttctgcaatttg | 176 | |
| RD9-F | agttccaagcttggcggcagcggcggcagc | 177 | |
| | ttctgtcaagttttcgtaacaaag | | |
| RD9-R | cacccgctcgag ttaaacttttaggccattgac | 178 | |
| RD10-F | agttccaagcttggcggcagcggcggcagc | 179 | Amplification of |
| | tgtgtagtgaacttgcaaaac | | RD10 |
| RD10-R | cacccgctcgag tta atcacggaggtatctcaaccg | 180 | |
| RD11-F | agttccaagcttggcggcagcggcggcagc | 181 | Amplification of |
| | aattctgcatcttcatctac | | RD11 |
| RD11-R | cacccgctcgag tta tgtagaattgttgctttcgaaaatg | 182 | |
| N-RD-F | ccgccaccatggct cccaagaaaaagcgcaaggtag | 183 | Insert RD2 and RD5 |
| N-RD2-R | gaggagccatggacgcaactggaacagatgcagatg | 184 | at N-terminus |
| N-RD5-R | gaggagccatggagtccatgtgtgggaagggcaacg | 185 | |
| 3gRNA-F1 | nnnnnggtctccggactctttgaaaagataatgtatg | 186 | Assemble three |
| 3gRNA-R1 | nnnnnggtctcccggacttgcatgcctgcagggagctc | 187 | gRNA cassettes into |
| 3gRNA-F2 | nnnnnggtctcctccgtctttgaaaagataatgtatg | 188 | a single plasmid |
| 3gRNA-R2 | nnnnnggtctccctggcttgcatgcctgcagggagctc | 189 | using Golden-Gate |
| 3gRNA-F3 | nnnnnggtctccccagtctttgaaaagataatgtatg | 190 | Assembly |
| 3gRNA-R3 | nnnnnggtctcccaaccttgcatgcctgcagggagctc | 191 | |
| ReFu-F1 | ggttgagtgttgttccagtttggaacaagagtc | 192 | Assemble CRISPR |
| ReFu-R1N | catgccggtagaggtgtggtcaataagag | 193 | protein cassettes and |
| ReFu-F2N | agctttggacttcttcgccagaggtttg | 194 | Csy4 cassette into a |
| ReFu-R2N | gcttggtgccacttgtcacatacaattc | 195 | single plasmid using |
| ReFu-F3 | cctgcagggtgtcgacgctgcgggtatagaaag | 196 | DNA assembler |
| ReFu-R3 | ctgccctttatattccctgttacagcagccgagc | 197 | |
| ReFu-F4 | gcggccgctatatctaggaacccatcaggttg | 198 | |
| ReFu-R4 | gattgctatgctttctttctaatgagcaagaag | 199 | |
| ReFu-F5 | ccgcggatagcttcaaaatgtttctactc | 200 | |
| ReFu-R5 | gggtttcgccacctctgacttgagcgtc | 201 | |
| SpMS2H-R | cagacataaaaaacaaaaaaa ggatc | 202 | pSpMS2SgH |
| | gggaagactccccagtgactg | | |
| SpPP7H-R | cagacataaaaaacaaaaaaa ggatc | 203 | pSpPP7SgH |
| | gggaactgctgcgtaagggtttc | | |
| SpComH-R | cagacataaaaaacaaaaaaa ggatc | 204 | pSpComSgH |
| | gatgctcgcaggcattcaggcaccgactcggtgc | | |
| PCP-MXI1-F1 | gttcgcggatcc atgcccaaaaagaaaagaaaagtg | 205 | pH1-PCP- |
| PCP-MXI1-R1 | tcttgggagctccctc ggagccacggcccagcg | 206 | MXI1(ligation) |
| MCP-VP64-F | gttggaagatct atgcccaaaaagaaaagaaaagtg | 207 | pH4-MCP-VP64 |
| MCP-VP64-R | cacccgctcgag tcagttgatgagcatgtccagatc | 208 | (ligation) |
| ROX1-Conf-F | tattctgttcagacagggacc | 209 | Verification and |
| ROX1-Conf-R | gatagctgttcgagcttgacac | 210 | sequencing primers |
| PMR1-Conf-F | catctaacgaggccaacaatag | 211 | for CRISPRd |
| PMR1-Conf-R | atataagctatacaagaggctg | 212 | |
| PEP4-conf-F | cgatcatgaagcttcatcaagc | 213 | |
| PEP4-conf-R | ctctccaattcggcgacttgac | 214 | |
| VPS8-conf-F | acgagaccggaaatatagagtg | 215 | |
| VPS8-conf-R | caggagaatggctagcggactg | 216 | |
| YPS1-conf-F | cgacttgaacgttaccgggttg | 217 | |
| YPS1-conf-R | tcagatggacagtccattgcgc | 218 | |
| qHMG1-F | agaagtggacggtgatttgag | 219 | Quantitative PCR |
| qHMG1-R | catggcaccttgtggttcta | 220 | analysis primers |
| qERG9-F | cttctggcccaaggaaatct | 221 | |
| qERG9-R | gacgaggtggtttatacagtcc | 222 | |
| qPDI1-F | gtcaacgacccaaagaagga | 223 | |
| qPDI1-R | tggcgtaggtatcagctagt | 224 | |
| qMNN9-F | ggagaaggaaagacacgcttta | 225 | |
| qMNN9-R | ccaagaagtgtgaggtcctatg | 226 | |
| qERO1-F | ttgctctgttgatgtcgtagag | 227 | |
| qERO1-R | tcatccgcttccttcattgtat | 228 | |
| qPMR1-F | ccttagcggttgctgctatt | 229 | |
| qPMR1-R | accttctcacgatggctttac | 230 | |
| qALG9-F | ccgttgccatgttgttgtatg | 231 | |
| qALG9-R | gccaggaaattgtacgctaaac | 232 | |

Oligonucleotides and gBLOCKs (IDT, Coralville, Iowa, USA) used for gRNA construction were listed in Table 4 and Table 5, respectively. Yeast plasmids were isolated using a Zymoprep Yeast Plasmid Miniprep II Kit (Zymo Research, Irvine, Calif.) and amplified in E. coli for verification by both restriction digestion and DNA sequencing.

TABLE 4
Oligos used to construct gRNAs

| Oligos | Sequences (5′-3′) | SEQ ID NO: |
|---|---|---|
| pSg1-F | gatcttgatatttaagttattaaa | 233 |
| pSg1-R | aaactttaataacttaaatatcaa | 234 |
| pSg6-F | gatc actttagtgctgacacatac | 235 |
| pSg6-R | aaac gtatgtgtcagcactaaagt | 236 |
| pSg10-F | gatc gatatcaagaggattggaaa | 237 |
| pSg10-R | aaactttccaatcctcttgatatc | 238 |
| pSg12-F | gatc acgtccctattgaatgttgg | 239 |
| pSg12-R | caacccaacattcaatagggacgt | 240 |
| pSg13-F | gatc aactctggacattataccat | 241 |
| pSg13-R | caacatggtataatgtccagagtt | 242 |
| pSg14-F | gatc aaaaatgggcaccatttact | 243 |
| pSg14-R | aaacagtaaatggtgccca | 244 |
| pSg15-F | gatc ccaattgtagagactatcca | 245 |
| pSg15-R | aaactggatagtctctacaattgg | 246 |
| pSg27-F | gatc tttaagttattaaa | 247 |
| pSg27-R | aaactttaataacttaaa | 248 |
| pSg28-F | gatc taaatatcaatggg | 249 |
| pSg28-R | aaac cccattgatattta | 250 |
| pSg29-F | gatc gaagctcatttgagatcaat | 251 |
| pSg29-R | caac attgatctcaaatgagcttc | 252 |
| pSg30-F | gatc ggaagaggtaacttcgttgt | 253 |
| pSg30-R | aaac acaacgaagttacctcttcc | 254 |
| pSg31-F | gatc gcaagcatcaatggtataatgtc | 255 |
| pSg31-R | aaacgacattataccattgatgcttgc | 256 |
| pSg32-F | gatcgtaacttcgttgtaaagaataagg | 257 |
| pSg32-R | caacccttattctttacaacgaagttac | 258 |
| pSg33-F | gatcgtgctgacacatac | 259 |
| pSg33-R | aaacgtatgtgtcagcac | 260 |
| pSg35-F | gatc gatatttaagttattaaa | 261 |
| pSg35-R | tttaataacttaaatatc | 262 |
| pSg36-F | gatc tatttaagttattaaa | 263 |
| pSg36-R | aaac tttaataacttaaata | 264 |
| pSg37-F | gatc atttaagttattaaa | 265 |
| pSg37-R | aaac tttaataacttaaat | 266 |
| pSg38-F | gatc ttaagttattaaa | 267 |
| pSg38-R | aaac tttaataacttaa | 268 |
| pSg39-F | gatc taagttattaaa | 269 |
| pSg39-R | aaac tttaataactta | 270 |
| pSg40-F | gatc agttattaaa | 271 |
| pSg40-R | aaac tttaataact | 272 |
| pSg55-F | gatc gaaaaaagtagcta | 273 |
| pSg55-R | aaac tagctacttttttc | 274 |
| pSg56-F | gatc ccgtaccataccct | 275 |
| pSg56-R | aaac agggtatggtacgg | 276 |
| pSg64-F | gatc ggatagtctctacaattggg | 277 |
| pSg64-R | aaac cccaattgtagagactatcc | 278 |
| pSg65-F | gatctccgccaggcgtgtatatatagc | 279 |
| pSg65-R | aaacgctatatatacacgcctggcgga | 280 |
| pSg66-F | gatcaacgaagcaggaaatgagagaat | 281 |
| pSg66-R | aaacattctctcatttcctgcttcgtt | 282 |
| pSg68-F | gatctaatttctactcttgtagatgatatcaagaggattggaaaagg | 283 |
| pSg68-R | aaaaccttttccaatcctcttgatatcatctacaagagtagaaatta | 284 |
| pSg69-F | gatcaatttctactaagtgtagatgatatcaagaggattggaaaagg | 285 |
| pSg69-R | aaaaccttttccaatcctcttgatatcatctacacttagtagaaatt | 286 |
| pSg87-F | gatccgaagcaggaaatgagagaat | 287 |
| pSg87-R | aaacattctctcatttcctgcttcg | 288 |
| pSg88-F | gatccttcgttcatttcgagtttcc | 289 |
| pSg88-R | aaacggaaactcgaaatgaacgaag | 290 |
| pSg89-F | gatccagacctccctgcgagcgggc | 291 |
| pSg89-R | aaacgcccgctcgcagggaggtctg | 292 |
| pSg90-F | gatccgccaggcgtgtatatatagc | 293 |
| pSg90-R | aaacgctatatatacacgcctggcg | 294 |
| pSg91-F | gatctcatttggcgagcgttggttg | 295 |
| pSg91-R | aaaccaaccaacgctcgccaaatga | 296 |
| pSg92-F | gatcgatctttccggtctctttggc | 297 |
| pSg92-R | aaacgccaaagagaccggaaagatc | 298 |
| pSg93-F | gatcggcttgttccacaggaacact | 299 |
| pSg93-R | aaacagtgttcctgtggaacaagcc | 300 |
| pSg94-F | gatcgccaaagtcctcgacttcaag | 301 |
| pSg94-R | aaaccttgaagtcgaggactttggc | 302 |
| pSg95-F | gatcacaacttcgccttaagttgaa | 303 |
| pSg95-R | aaacttcaacttaaggcgaagttgt | 304 |
| pSg109-F | gatctctaagttttaattacaaaa | 305 |
| pSg109-R | aaacttttgtaattaaaacttaga | 306 |
| pSg110-F | gatcggaattcgtgagcaagggcg | 307 |
| pSg110-R | aaaccgcccttgctcacgaattcc | 308 |
| pSg111-F | gatccgaggagctgttcaccgggg | 309 |
| pSg111-R | aaacccccggtgaacagctcctcg | 310 |
| pSg112-F | gatcgaccaggatgggcaccaccc | 311 |
| pSg112-R | aaacgggtggtgcccatcctggtc | 312 |
| pSg113-F | gatccgtcgccgtccagctcgacc | 313 |
| pSg113-R | aaacggtcgagctggacggcgacg | 314 |
| pSg114-F | gatcggtggtgcagatcagcttca | 315 |
| pSg114-R | aaactgaagctgatctgcaccacc | 316 |
| pSg115-F | gatccaagtaatacatattcaaaa | 317 |
| pSg115-R | aaacttttgaatatgtattacttg | 318 |
| pSg116-F | gatcgaatatgtattacttggtta | 319 |
| pSg116-R | aaactaaccaagtaatacatattc | 320 |
| pSg117-F | gatcaagaacagaagaataacgca | 321 |
| pSg117-R | aaactgcgttattcttctgttctt | 322 |
| pSg118-F | gatcttatccctcatgttgtctaa | 323 |
| pSg118-R | aaacttagacaacatgagggataa | 324 |
| pSg119-F | gatccaatcaatacaataaaataa | 325 |
| pSg119-R | aaacttattttattgtattgattg | 326 |
| pSg120-F | gatctactcttttgaacaagatgt | 327 |
| pSg120-R | aaacacatcttgttcaaaagagta | 328 |
| pSg121-F | gatcataagtatattaggatgagg | 329 |
| pSg121-R | aaaccctcatcctaatatacttat | 330 |
| pSg122-F | gatcaatttctactaagtgtagat gtgtaggaacatcaacatgctca | 331 |
| pSg122-R | aaaatgagcatgttgatgttcctacac atctacacttagtagaaatt | 332 |
| pSg123-F | gatcaatttctactaagtgtagat cccttctccagaaacaatcagat | 333 |
| pSg123-R | aaaaatctgattgtttctggagaaggg atctacacttagtagaaatt | 334 |
| pSg124-F | gatcaatttctactaagtgtagat ccattcgtcttgaagtcgaggac | 335 |
| pSg124-R | aaaagtcctcgacttcaagacgaatgg atctacacttagtagaaatt | 336 |
| pSg125-F | gatcaatttctactaagtgtagat agttattaaatggtcttcaattt | 337 |
| pSg125-R | aaaa aaattgaagaccatttaataact atctacacttagtagaaatt | 338 |
| pSg126-F | gatcaatttctactaagtgtagat ataacttaaatatcaatgggagg | 339 |
| pSg126-R | aaaa cctcccattgatatttaagttat atctacacttagtagaaatt | 340 |
| pSg127-F | gatcaatgaacgaagcaggaaatg | 341 |
| pSg127-R | aaaccatttcctgcttcgttcatt | 342 |
| pSg128-F | gatcgcgtgttgttgctgctgaca | 343 |
| pSg128-R | aaactgtcagcagcaacaacacgc | 344 |
| pSg135-F | gatcaatttctactaagtgtagat cttcttgctcattagaaagaaag | 345 |
| pSg135-R | aaaa ctttctttctaatgagcaagaag atctacacttagtagaaatt | 346 |
| pSg136-F | gatcaatttctactaagtgtagat taattaaaacttagattagattg | 347 |
| pSg136-R | aaaa caatctaatctaagttttaatta atctacacttagtagaaatt | 348 |
| pSg137-F | gatcaatttctactaagtgtagat cgtcgccgtccagctcgaccagg | 349 |
| pSg137-R | aaaa cctggtcgagctggacggcgacg atctacacttagtagaaatt | 350 |
| pSg138-F | gatctttcttagcaaagcaaagga | 351 |
| pSg138-R | aaactcctttgctttgctaagaaa | 352 |
| pSg139-F | gatcggaaactcgaaatgaacgaa | 353 |
| pSg139-R | aaacttcgttcatttcgagtttcc | 354 |
| pSg140-F | gatccttagcaaagcaaaggaggg | 355 |
| pSg140-R | aaacccctcctttgctttgctaag | 356 |
| pSg141-F | gatcatagcggtagtgtttgcgcg | 357 |
| pSg141-R | aaaccgcgcaaacactaccgctat | 358 |
| pSg142-F | gatcgtaaaccccggccaaagaga | 359 |
| pSg142-R | aaactctctttggccggggtttac | 360 |
| pSg143-F | gatcacacgcctggcggatctgct | 361 |
| pSg143-R | aaacagcagatccgccaggcgtgt | 362 |
| pSg144-F | gatcacctgaatctaaaattcccg | 363 |
| pSg144-R | aaaccgggaattttagattcaggt | 364 |
| pSg146-F | gatcgccggggtttacggacgatg | 365 |
| pSg146-R | aaaccatcgtccgtaaaccccggc | 366 |
| pSg147-F | gatcgacggaaaaaagtagctaag | 367 |
| pSg147-R | aaaccttagctacttttttccgtc | 368 |
| pSg148-F | gatccaaagcattcaattcaaatg | 369 |
| pSg148-R | aaaccatttgaattgaatgctttg | 370 |
| pSg149-F | gatcaatttctactaagtgtagat caagggtatggtacggtgctatc | 371 |
| pSg149-R | aaaa gatagcaccgtaccatacccttg atctacacttagtagaaatt | 372 |
| pSg150-F | gatcaatttctactaagtgtagat tcagcagcaacaacacgctacgc | 373 |
| pSg150-R | aaaa gcgtagcgtgttgttgctgctga atctacacttagtagaaatt | 374 |
| Sg155-F | gatcaatttctactaagtgtagat cggacgatggcagaagaccaaag | 375 |
| Sg155-R | aaaa ctttggtcttctgccatcgtccg atctacacttagtagaaatt | 376 |
| Sg156-F | gatcaatttctactaagtgtagat gcgagcgttggttggtggatcaa | 377 |
| Sg156-R | aaaa ttgatccaccaaccaacgctcgc atctacacttagtagaaatt | 378 |
| Sg157-F | gatcaatttctactaagtgtagat gtgctgacacatacaggcatata | 379 |
| Sg157-R | aaaa tatatgcctgtatgtgtcagcac atctacacttagtagaaatt | 380 |
| pSg172-F | gatcataaatggaaagttaggaca | 381 |
| pSg172-R | aaactgtcctaactttccatttat | 382 |
| pSg175-F | gatcaatttctactaagtgtagatcggctatgaaaagctgttgttcg | 383 |
| pSg175-R | aaaacgaacaacagcttttcatagccgatctacacttagtagaaatt | 384 |
| pSg194-F | gatcaatttctactaagtgtagatcatattcgaagcttacaatcgag | 385 |
| pSg194-R | aaaactcgattgtaagcttcgaatatgatctacacttagtagaaatt | 386 |
| pSg195-F | gatcaatttctactaagtgtagattaccagcaatcagctgactaaca | 387 |
| pSg195-R | aaaatgttagtcagctgattgctggtaatctacacttagtagaaatt | 388 |
| pSg196-F | gatcaatttctactaagtgtagatttgctcttacccgactctgaaga | 389 |
| pSg196-R | aaaatcttcagagtcgggtaagagcaaatctacacttagtagaaatt | 390 |
| pSg197-F | gatcaatttctactaagtgtagatgcaagacctcaaacaatcgtact | 391 |
| pSg197-R | aaaaagtacgattgtttgaggtcttgcatctacacttagtagaaatt | 392 |
| pSg198-F | gatcgctggggtagaactagagta | 393 |
| pSg198-R | aaactactctagttctaccccagc | 394 |
| pSg199-F | gatcttatatgacagttcaaaaga | 395 |
| pSg199-R | aaactcttttgaactgtcatataa | 396 |
| pSg200-F | gatcggaagtggagatggaagagg | 397 |
| pSg200-R | aaaccctcttccatctccacttcc | 398 |
| pSg201-F | gatcctacatgcaaacgacaaata | 399 |
| pSg201-R | aaactatttgtcgtttgcatgtag | 400 |
| pSg202-F | gatcgctgaaaactgtatgtgcgg | 401 |
| pSg202-R | aaacccgcacatacag cagc | 402 |
| pSg203-F | gatcatccaacgatgcaattcagt | 403 |
| pSg203-R | aaacactgaattgcatcgttggat | 404 |
| pSg204-F | gatcaaatgggaatggaaagaacg | 405 |
| pSg204-R | aaaccgttctttccattcccattt | 406 |
| pSg217-F | gatcaatttctactaagtgtagatcaacaactatctgcgataactca | 407 |
| pSg217-R | aaaatgagttatcgcagatagttgttgatctacacttagtagaaatt | 408 |
| pSg218-F | gatcaatttctactaagtgtagatcagggtcttctataagagaaacc | 409 |
| pSg218-R | aaaaggtttctcttatagaagaccctgatctacacttagtagaaatt | 410 |
| pSg219-F | gatcaatttctactaagtgtagatagccctacttaatgctgagccac | 411 |
| pSg219-R | aaaagtggctcagcattaagtagggctatctacacttagtagaaatt | 412 |
| pSg220-F | gatcaatttctactaagtgtagatgctatgttagctgcaactttcta | 413 |
| pSg220-R | aaaatagaaagttgcagctaacatagcatctacacttagtagaaatt | 414 |
| pSg221-F | gatcaatttctactaagtgtagatgaaacacgtgtcctgaaaattat | 415 |
| pSg221-R | aaaaataattttcaggacacgtgtttcatctacacttagtagaaatt | 416 |
| pSg222-F | gatcaatttctactaagtgtagataaaatcatcgaatagccgatcga | 417 |
| pSg222-R | aaaatcgatcggctattcgatgattttatctacacttagtagaaatt | 418 |
| pSg223-F | gatcaatttctactaagtgtagatccagtcactatcatcatcatcat | 419 |
| pSg223-R | aaaaatgatgatgatgatagtgactggatctacacttagtagaaatt | 420 |
| pSg224-F | gatcaatttctactaagtgtagatacgggcaaaaactggattctccc | 421 |
| pSg224-R | aaaagggagaatccagtttttgcccgtatctacacttagtagaaatt | 422 |
| pSg225-F | gatcaatttctactaagtgtagattgtcttacgagccgggtaccaag | 423 |
| pSg225-R | aaaacttggtacccggctcgtaagacaatctacacttagtagaaatt | 424 |
| pSg226-F | gatcaatttctactaagtgtagatcaggggcgatgccacttatcagt | 425 |
| pSg226-R | aaaaactgataagtggcatcgcccctgatctacacttagtagaaatt | 426 |
| pSg227-F | gatcggattggcgagaaataatgt | 427 |
| pSg227-R | aaacacattatttctcgccaatcc | 428 |
| pSg228-F | gatcgcagatggggagagagaatg | 429 |
| pSg228-R | aaaccattctctctccccatctgc | 430 |
| pSg229-F | gatctttccttgtagcgatcaggt | 431 |
| pSg229-R | aaacacctgatcgctacaaggaaa | 432 |
| pSg230-F | gatcgaaataacgggtcccaagag | 433 |
| pSg230-R | aaacctcttgggacccgttatttc | 434 |
| pSg231-F | gatccccacgggttctttcttagg | 435 |
| pSg231-R | aaaccctaagaaagaacccgtggg | 436 |
| pSg260-F | gatcgcgagcaaacactattatga | 437 |
| pSg260-R | aaactcataatagtgtttgctcgc | 438 |
| pSg261-F | gatcagaagggcttggtttcgaaa | 439 |
| pSg261-R | aaactttcgaaaccaagcccttct | 440 |
| pSg262-F | gatccaaaacgggatatttaagcc | 441 |
| pSg262-R | aaacggcttaaatatcccgg | 442 |
| pSg263-F | gatcagccgaatgaatgaaatatg | 443 |
| pSg263-R | aaaccatatttcattcattcggct | 444 |
| pSg264-F | gatcttgttgtgatgatacaagag | 445 |
| pSg264-R | aaacctcttgtatcatcacaacaa | 446 |

TABLE 5
gBLOCKs used in this study

| | Sequences | SEQ ID NO: |
|---|---|---|
| Sg10 | ctttggtctccgatc | 447 |
| | aaattctcctgccaaacaaataagcaactccaatgaccacgttaatggct | |
| | aatcctcttgatatcgaaaaactagctgaaaaatgtgatgtgctaacgat | |
| | gatatcaagaggattggaaa | |
| | gtttggagacctttc | |
| Sg145 | ctttggtctccgatc | 448 |
| | tccacaaggacaatatttgtgacttatgttatgcgcctgctagagttccg | |
| | ggcagaaaatgcaatcaaatcttttcccggttgtggtatatttggtgtgg | |
| | acaacttcgccttaagttgaa | |
| | gtttggagacctttc | |
| Sg163 | gttcgcggatcc | 449 |
| | gttcactgcgtataggcagAATTTCTACTAAGTGTAGAT | |
| | gcgagcgttggttggtggatcaa | |
| | gttcactgccgtataggcaggaccaggatgggcaccacccGTTTTAGAG | |
| | CTAGAAATAGCAAGTTAAAATAAGGCTAGTCCGTT | |
| | ATCAACTTGAAAAAGTGGCACCGAGTCGGTGC | |
| | gttcactggtataggcagtccacaaggacaatatttgtgacttatgttatgcgcctg | |
| | ctagagttccgggcagaaaatgcaatcaaatcttttcccggttgtggtatatttggtgt | |
| | ggacaacttcgccttaagttgaaGTTTTAGTACTCTGGAAACAG | |
| | AATCTACTAAAACAAGGCAAAATGCCGTGTTTATC | |
| | TCGTCAACTTGTTGGCGAGA gttcactgccatgtataggcag | |
| | ctcgagcgggtg | |
| Sg186 | ctttggtctccgatc | 450 |
| | ctacacctaagattccaagacccaagaacgcatttattctgttcagacag | |
| | ggaccgctcaaggtgtggaaataccccataattcaaacatttctaaaatt | |
| | actaccacaggatcttaatag | |
| | gtttggagacctttc | |
| Sg205 | ctttggtctccgatc | 451 |
| | tacataaaacctcacaaacgatcgaaaaatcttcctttaacgatcagcct | |
| | cttgtatagcttatatgggtacattagtcaaggaaggtcatggtaagggt | |
| | atctctcagaaatcggtacaa | |
| | gtttggagacctttc | |
| Sg265 | ctttggtctccgatc | 452 |
| | cgatatcacttggttacctgttcgtcgtaaggcttactgggaagtcaagt | |
| | cgccgaattggagagccatggtgccgccatcgatactggtacttctttga | |
| | ttgaaggtatcggtttaggcg | |
| | gtttggagacctttc | |
| Sg266 | ctttggtctccgatc | 453 |
| | catatgttccgatggtactcatgtagctgcctcataccagaccggaaata | |
| | tagagtgaaacccacttctgaaccaacaaatggtatgaccccaacgcctg | |
| | tatgcatttggaacttgaacg | |
| | gtttggagacctttc | |
| Sg267 | ctttggtctccgatc | 454 |
| | ttgcgcacctagttcagtagcgatcatacttaccactgtttgaggtaaat | |
| | accaccaaaatcgaacactatttccatactatcatcagatggacagtcca | |
| | atacgtaataccctatcctgg | |
| | gtttggagacctttc | |

The gRNA targeting sequences were underlined, the gRNA scaffold sequences were shown in uppercase, and the Cys4 sites were dotted underlined.

Yeast strains were transformed using the LiAc/SS carrier DNA/PEG method, and transformants were selected on the appropriate agar plates. Recombinant yeast strains constructed in this study were listed in Table 6.

TABLE 6
Yeast Strains used in this study

| Strains | Genotype |
|---|---|
| CEN.PK2-1C | MATa; his3D1; leu2-3 112; ura3-52; trp1-289; MAL2-8c; SUC2 |
| CEN-iAID6 | CEN.PK2-1C-KanMX-TDH3p-dLbCpf1-VP64-p65-ADH1t-ENO2p-Csy4-PGK1t- |
| | TPI1p-dSpCas9-RD11-RD5-RD2-TPI1t-TEF1p-SaCas9-TEF1t |
| CT | CEN.PK2-1C-ura3::URA3-CYC1p-mCherry-TEF1t-TEF1p-mVenus-PGK1t |
| CF | CEN.PK2-1C-ura3::URA3-CYC1p-mCherry-TEF1t-FBA1p-mVenus-PGK1t |
| CH | CEN.PK2-1C-ura3::URA3-CYC1p-mCherry-TEF1t-HHF2p-mVenus-PGK1t |
| CR1 | CEN.PK2-1C-ura3::URA3-CYC1p-mCherry-TEF1t-REV1p-mVenus-PGK1t |
| CR2 | CEN.PK2-1C-ura3::URA3-CYC1p-mCherry-TEF1t-RNR2p-mVenus-PGK1t |
| CEN-Crt | CEN-iAID6-ura3::URA3-TDH3p-CrtYB-CYC1t-TDH3p-CrtE-CYC1t-TDH3p- |
| | CrtI-CYC1t |
| CEN-EGII | CEN-iAID6-ura3::URA3-TEF1p-prepro-HisTag-EGII-AGA1-PGK1t |

The reporter plasmid p406-CT was constructed by cloning each expression element including CYC1p, mCherry, TEF1t, TEF1p, mVenus, and PGK1t into pRS406 using Gibson Assembly. Other reporter plasmids were constructed by replacing TEF1p in p406-CT with FBA1p (strong promoter, p406-CF), HHF2p (strong promoter, p406-CH), REV1p (weak promoter, p406-CR1), and RNR2p (medium-strength promoter, p406-CR2), respectively. The reporter yeast strains were constructed by integrating EcoRV linearized reporter plasmids into the ura3 locus of the CEN.PK2-1C genome.

For the construction of individual gRNA expression plasmids, several helper plasmids (pSgH2, pSpSgH, pNmSgH, pSt1SgH, pSaSgH, pSpMS2SgH, pSpPP7SgH, and pSpComSgH) containing SNR52p, two BsaI sites, gRNA scaffold sequences, and SUP4t were constructed first based on a modified, BsaI-free pRS423 vector (Bao, Z., et al., ACS Synth. Biol. 4:585-594 (2015)). Then the targeting sequences were synthesized as short oligos, which were annealed and phosphorylated and cloned into the corresponding BsaI digested helper plasmids. To construct multiple gRNAs expression plasmids, the individual gRNA expression cassettes were pieced together using Golden-Gate Assembly (design II), or the gRNA arrays were synthesized as gBLOCKs and cloned into pRS423-H5 (design III) using restriction digestion/ligation.

CRISPR protein expression plasmids were constructed by cloning the PCR amplified fragments into pH1, pH3, pH4, pH5, and pH6 (Lian, J. & Zhao, H., ACS Synth. Biol. 4:332-341 (2015); Lian, J. & Zhao, H., ACS Synth. Biol. 5:689-697 (2016)) using BamHI/XhoI or NcoI/XhoI digestion and ligation. To clone additional NLS into the N-terminus of some CRISPR proteins, adapter (BamHI-NLS-BamHI or NcoI-NLS-NcoI) was inserted into the BamHI or NcoI site. The nuclease-deficient LbCpf1 (E832A) was created by overlap extension PCR and cloned into the NcoI/HindIII site of pTDH3-dSpCas9-MXI1 to construct pTDH3-dLbCpf1-MXI1. MXI1 fragment of pTDH3-dSpCas9-MXI1 and pTDH3-dLbCpf1-MXI1 was replaced by HindIII/XhoI digestion to construct dSpCas9 with different repression domains and dLbCpf1 with various activation domains, respectively. pAID6 was constructed by cloning each CRISPR-AID module (dLbCpf1-VP, Csy4, dSpCas9-RD1152, and SaCas9) into pRS41K-CEN-Delta using DNA Assembler. CEN-iAID6 was constructed by integrating PmeI digested pAID6 into the delta site and selection for G418 resistance. The successful integration of AID6 cassettes was verified by both diagnostic PCR and CRISPR functional assays.

The β-carotene producing strain (CEN-Crt) and Trichoderma reesei endoglucanase II (EGII)-displaying strain (CEN-EGII) were constructed by integrating StuI linearized YIplac211-YB/E/I (Verwaal, R., et al., Appl. Environ. Microbiol. 73:4342-4350 (2007)) and p406-YD-EGII (TEF1p-prepro-HisTag-EGII-AGA1-PGK1t) (Si, T., et al., Nat. Commun. 8:15187 (2017)), respectively, into the ura3 locus of CEN-iAID6 genome and selection on SED-URA/G418.

Fluorescence Intensity Measurement.

Recombinant yeast strains were pre-cultured in the corresponding selective medium for 2 days and then inoculated into the fresh synthetic media with an initial OD of 0.1. Mid-log phase yeast cells were diluted 5-fold in ddH₂O and mVenus and mCherry fluorescence signals were measured at 514 nm-528 nm and 587 nm-610 nm, respectively, using a Tecan Infinite M1000 PRO multimode reader (Tecan Trading AG, Switzerland). The fluorescence intensity (relative fluorescence units; RFU) was normalized to cell density that was determined by measuring the absorbance at 600 nm using the same microplate reader.

gRNA design.

gRNA for gene deletion (CRISPRd) was designed using Benchling CRISPR tool (benchling.com), and those with both high on-targeting score and off-targeting score were selected. For CRISPRa and CRISPRi, the gRNA binding position was equally important as the sequence itself. Based on previous studies (Gilbert, L. A., et al., Cell 159:647-661 (2014); Konermann, S., et al., Nature 517:583-588 (2015); Smith, J. D., et al., Genome Biol. 17:45 (2016)) and our empirical experience, ˜250 bp upstream of the coding sequences or ˜200 bp upstream of the transcription starting site (TSS) worked the best for CRISPRa; ˜100-150 bp upstream of the coding sequences or 50-100 bp upstream of TSS worked the best for CRISPRi by blocking transcriptional initiation and those targeting the non-template strand of the coding sequences worked the best for CRISPRi by blocking transcriptional elongation. Since on-targeting score and off-targeting score were not available for Cpf1, the following criteria were considered: GC contents between 35% and 65%, no polyT, no secondary structure, and minimal off-target effect (less than 12 bp match by BLAST to the yeast genome).

Quantitative PCR Analysis.

Mid-log phase yeast cells were collected and used to determine the relative expression levels via qPCR. Total RNAs were isolated using the RNeasy Mini Kit (QIAGEN, Valencia, Calif., USA) following the manufacturer's instructions. 1 μg of the RNA samples were then reversed transcribed into cDNA using the Transcriptor First Strand cDNA Synthesis Kit using oligo-dT primer (Roche, Indianapolis, Ind., USA). The qPCR experiments were carried out using SYBR Green-based method in the QuantStudio 7 Flex Real-Time PCR System (ThermoFisher Scientific).

Results

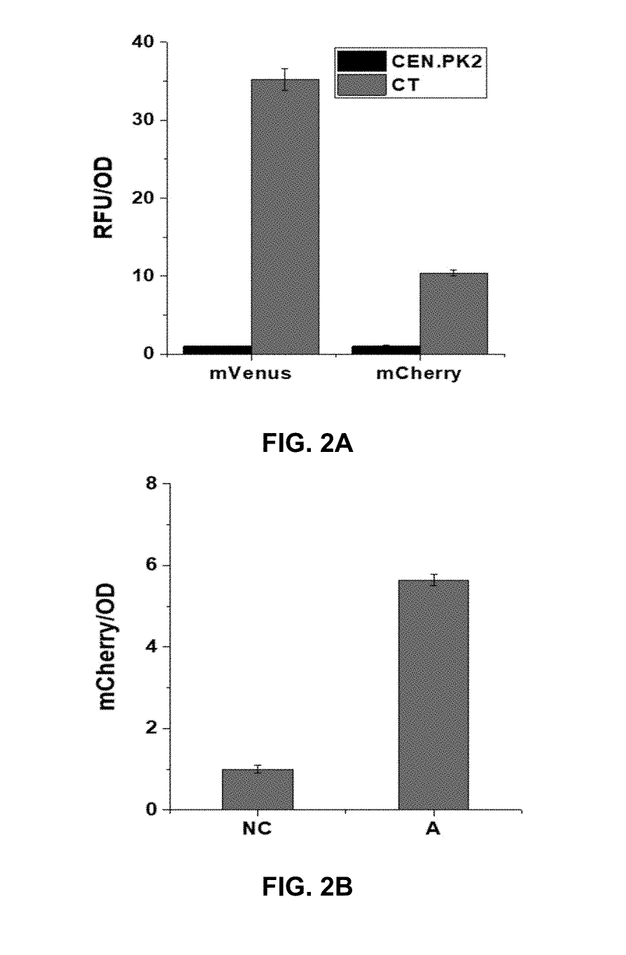

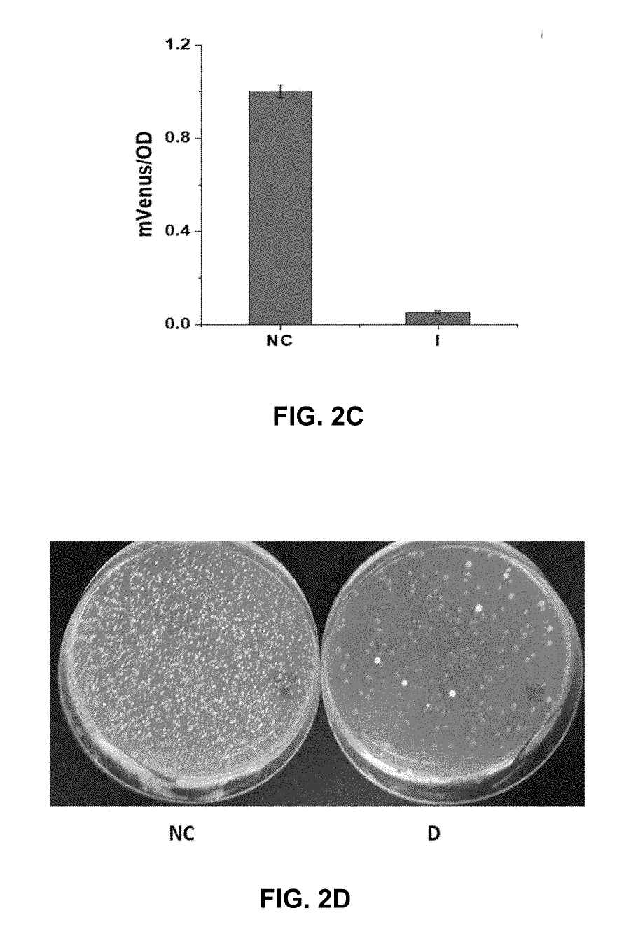

A CRISPR protein (SpCas9) has been well characterized for genome engineering in yeast (Zalatan, J. G., et al., Cell 160:339-350 (2015); Jakociunas, T., et al., Metab. Eng. 34:44-59 (2016); DiCarlo, J. E., et al., Nucleic Acids Res. 41:4336-4343 (2013); Bao, Z., et al., ACS Synth. Biol. 4:585-594 (2015); Lian, J., et al., Biotechnol. Bioeng. 113:2462-2473 (2016); Liu, Z., et al., ACS Synth. Biol. (2017); Gilbert, L. A., et al., Cell 154:442-451 (2013)), a number of CRISPR protein orthologs were characterized. dSpCas9-VPR (Chavez, A., et al., Nat. Methods 12:326-328 (2015)), dSpCas9-MXI1 (Gilbert, L. A., et al., Cell 154:442-451 (2013)), and SpCas9 (DiCarlo, J. E., et al., Nucleic Acids Res. 41:4336-4343 (2013); Bao, Z., et al., ACS Synth. Biol. 4:585-594 (2015)) were included as the positive controls for the optimization of CRISPR-AID modules. Strain CT was constructed by integrating CYC1p-mCherry-TEF1t and TEF1p-mVenus-PGK1t into the ura3 locus of the CEN.PK2 genome (FIG. 2A).When tested individually in the reporter yeast strain CT, more than 5-fold activation of mCherry expression (dSpCas9-VPR with Sg6) (FIG. 2B), around 10-fold interference of mVenus expression (dSpCas9-MXI1 with Sg1) (FIG. 2C), and nearly 100% deletion of ADE2 gene (SpCas9 with Sg11) (FIG. 2D) were obtained. The deletion of ADE2, shown as red colonies, was achieved with an efficiency of nearly 100%. Notably, CRISPRa (FIG. 2B), CRISPRi (FIG. 2C), and CRISPRd (FIG. 2D) were carried out individually.

Those functional CRISPR proteins were further optimized for transcriptional regulation by engineering the optimal effector domains. To develop the orthogonal tri-functional CRISPR system, at least three functional CRISPR proteins are needed. Thus, a few CRISPR protein orthologs in S. cerevisiae were characterized. Several CRISPR proteins (Table 7) including Cas9 from Streptococcus pyogenes (SpCas9) (Cong, L., et al., Science 339:819-823 (2013); Mali, P., et al., Science 339:823-826 (2013); Bao, Z., et al., ACS Synth. Biol. 4:585-594 (2015)), Neisseria meningitides (NmCas9) (Hou, Z., et al., Proc. Natl. Acad. Sci. U.S.A 110:15644-15649 (2013); Esvelt, K. M., et al., Nat. Methods 10:1116-1121 (2013)), Streptococcus thermophiles (St1Cas9) (Esvelt, K. M., et al., Nat. Methods 10:1116-1121 (2013); Kleinstiver, B. P., et al., Nature 523:481-485 (2015)), and Staphylococcus aureus (SaCas9) (Kleinstiver, B. P., et al., Nature 523:481-485 (2015); Ran, F. A., et al., Nature 520:186-191 (2015)) and Cpf1 (Zetsche, B., et al., Cell 163:759-771 (2015)) from Lachnospiraceae bacterium ND2006 (LbCpf1) and Acidaminococcus sp. BV3L6 (AsCpf1) have been characterized and found to be functional in mammalian cells.

TABLE 7
CRISPR protein orthologs

| | PAM |
|---|---|
| SpCas9 | 5′-guide-NGG3′ |
| NmCas9 | 5′-guide-NNNNGAAT3′ |
| St1Cas9 | 5′-guide-NNAGAAW3′ |
| SaCas9 | 5′-guide-NNGRRT3′ |
| AsCpf1 | 5′-TTTN-guide-3′ |
| LbCpf1 | 5′-TTTN-guide-3′ |

The gRNA structure sequences as well as the PAM sequences are different, both of which endow the activity of these CRISPR proteins to be orthogonal.

Therefore, the nuclease activities of these CRISPR proteins in yeast were characterized using ADE2 deletion as a reporter. Interestingly, although a single nuclear localization sequence (NLS) tag at the C-terminus was sufficient to target the CRISPR proteins to the nucleus of mammalian cells (Esvelt, K. M., et al., Nat. Methods 10:1116-1121 (2013); Kleinstiver, B. P., et al., Nature 523:481-485 (2015); Zetsche, B., et al., Cell 163:759-771 (2015)), it was found that dual-NLSs at both termini were required for nuclease activity of St1Cas9 and LbCpf1 in yeast (Table 8).

TABLE 8
Nuclease activity of CRISPR protein orthologs in yeast

| Nuclease | gRNA | Protein-NLS | NLS-Protein-NLS |
|---|---|---|---|
| SpCas9 | Sg10 | — | ~80% |
| NmCas9 | Sg12 | — | 0 |
| | Sg13 | — | 0 |
| | Sg29 | — | 0 |
| | Sg32 | — | 0 |
| St1Cas9 | Sg14 | 0 | ~62% |
| | Sg15 | 0 | 0 |
| | Sg30 | 0 | ~2.4% |
| | Sg64 | 0 | ~72% |
| SaCas9 | Sg31 | ~50% | ~46% |
| | Sg93 | ~27% | ~30% |
| | Sg94 | ~4.6% | ~5.2% |
| | Sg95 | ~77% | ~84% |
| AsCpf1 | Sg68 | 0 | ~0.2% |
| LbCpf1 | Sg69 | 0 | ~59% |
| | Sg122 | 0 | ~92% |
| | Sg123 | 0 | ~55% |
| | Sg124 | 0 | ~0.3% |

Nuclease activity was evaluated by co-transforming 500 ng CRISPR protein plasmid, 500 ng gRNA plasmids, and 500 ng linear DNA donor for the deletion of whole ADE2 coding sequences. The results represented an average of biological triplicates.

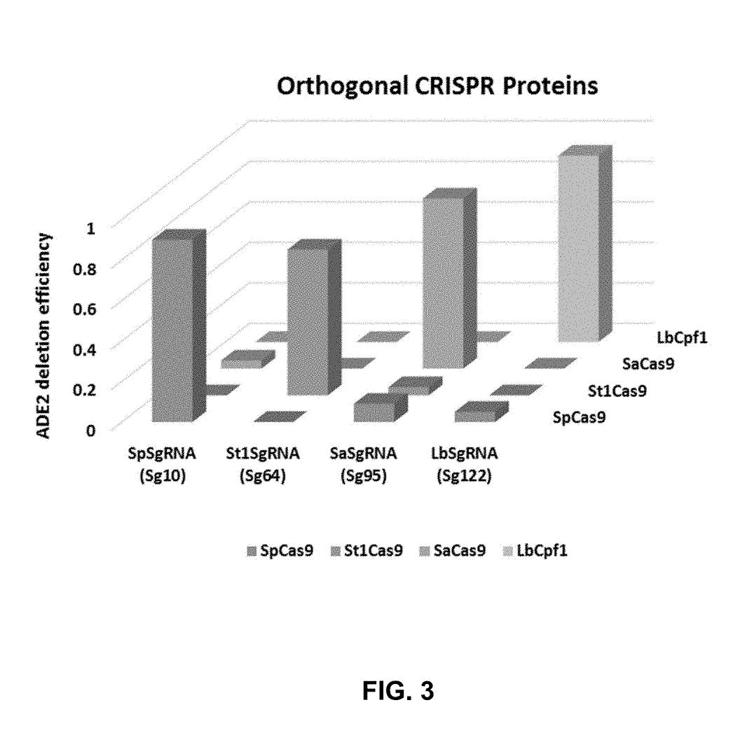

Nuclease activity for NmCas9 and AsCpf1 was not detectable under any conditions, probably due to different protein folding environments between yeast and mammalian cells. More than three CRISPR proteins (e.g., SpCas9, St1Cas9, SaCas9, LbCpf1) were found to be functional and orthogonal to each other, i.e. functional only when bound to their own cognate gRNAs (e.g., Sg10, Sg64, Sg95, and Sg122, respectively). In all cases, 500 ng linear donor DNA that resulted in the deletion of the whole ADE2 coding sequences was co-transformed as well. The CRISPR proteins were only functional when their cognate gRNAs were present. 1-2 red colonies might be found on selective agar plates, but not in a reproducible manner, due to the spontaneous homologous recombination between the genome and the linear donor. (FIG. 3).

To enable multiplex genome engineering, the previously developed HI-CRISPR design was followed (Bao, Z., et al., ACS Synth. Biol. 4:585-594 (2015)), where the homology donor sequences were integrated into the gRNA expression cassette. It was found that the stable maintenance of the homology donor resulted in a further increase in CRISPRd efficiency: from 80% with Sg10 (Table 8) to ˜98% with Sg11 (FIG. 2A-2D) for SpCas9 and from 77% (Table 8) with Sg95 to ˜95% with Sg145 for SaCas9. Therefore, SpCas9, SaCas9, St1Cas9, and LbCpf1 as well as their corresponding nuclease-deficient forms were chosen for further studies.

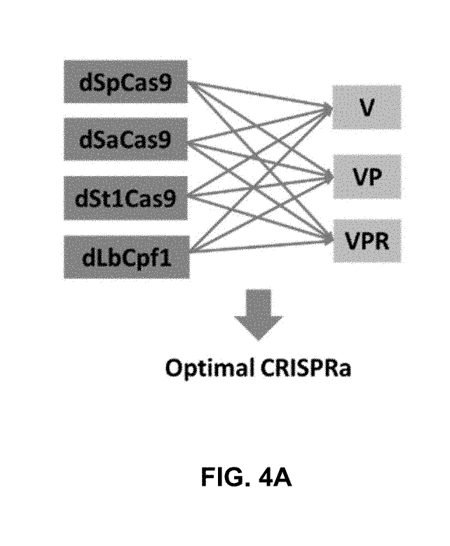

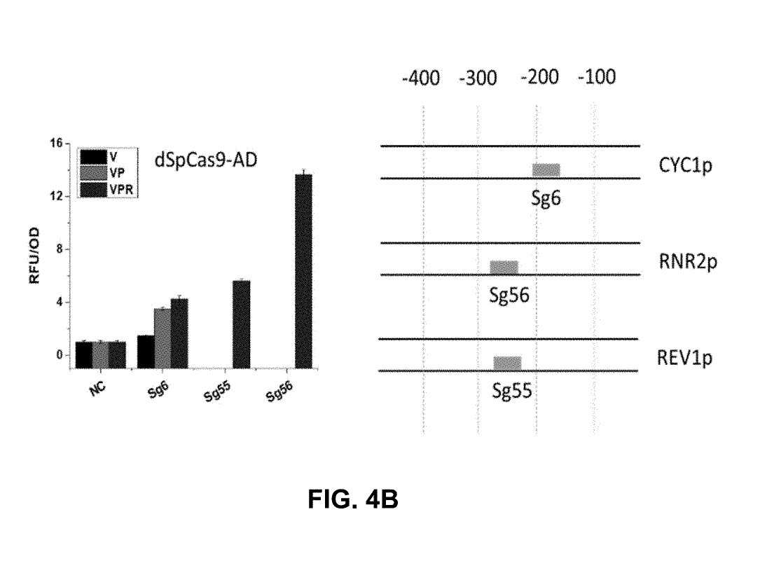

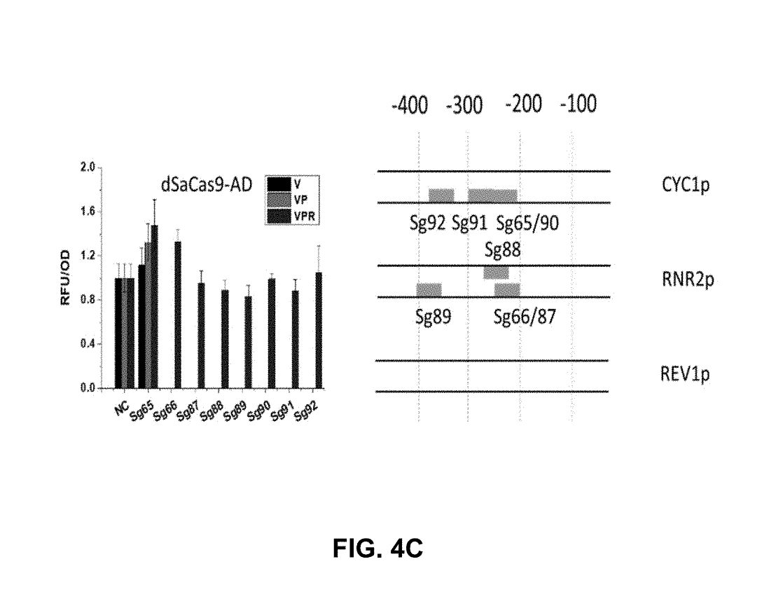

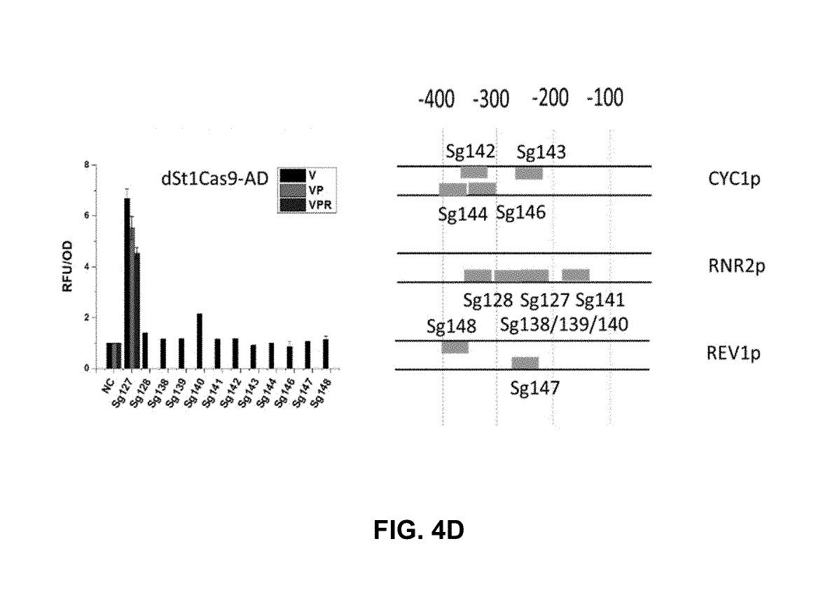

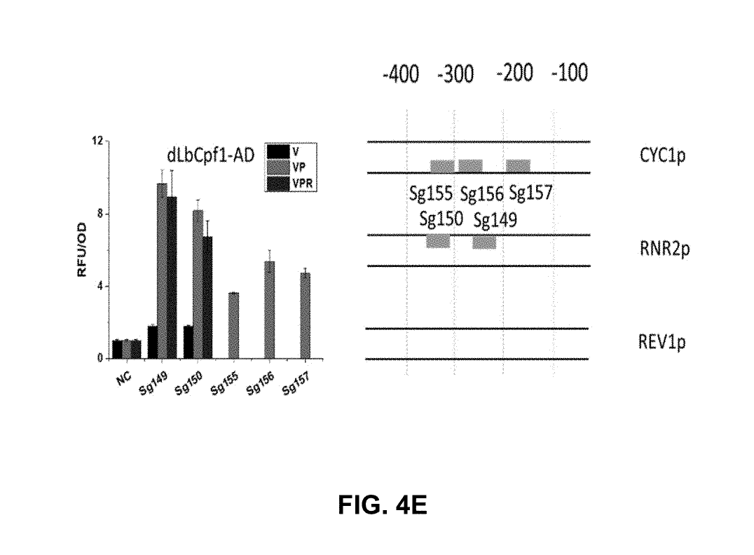

Next, the combination of the CRISPR proteins and the activation domains to achieve maximal CRISPRa was optimized. By testing all possible combinations (FIG. 4A) of 4 nuclease-deficient CRISPR proteins (dSpCas9, dSaCas9, dSt1Cas9, and dLbCpf1) and 3 activation domains (VP64 (V), VP64-p65AD (VP), and VP64-p65AD-Rta (VPR)) (Chavez, A., et al., Nat. Methods 12:326-328 (2015)), it was found that the optimal activation domain was CRISPR protein dependent: for dSpCas9, stronger activation domain resulted in more efficient CRISPRa (FIG. 4B); for dSt1Cas9, the order was completely reversed (FIG. 4D); while for dLbCpf1, the medium strength activation domain (VP) worked the best (FIG. 4E). Interestingly, although SaCas9 was functional for CRISPRd, only marginal activation was observed using dSaCas9 with various activation domains and several gRNAs targeting different regions of CYC1p and RNR2p (FIG. 4C). Since only 1 out of 12 gRNAs resulted in significant transcriptional activation (FIG. 4D), dSt1Cas9 was not further evaluated for practical metabolic engineering applications. Therefore, we chose dSpCas9 and dLbCpf1 as CRISPRa candidates.

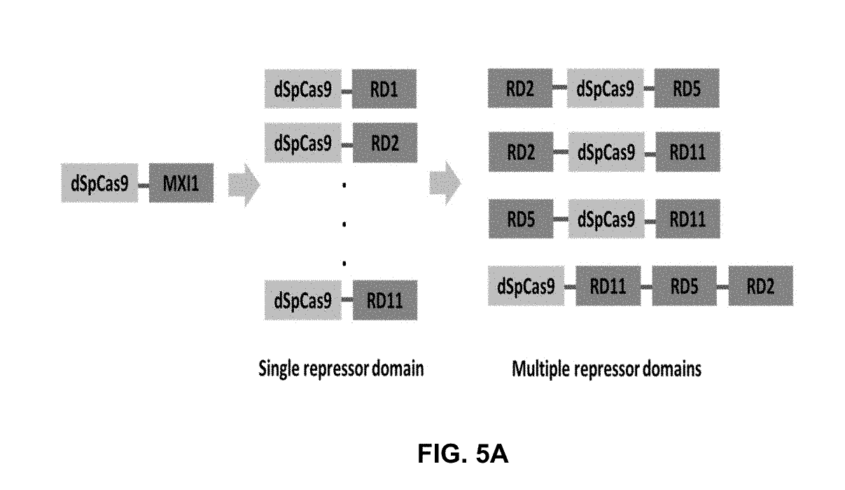

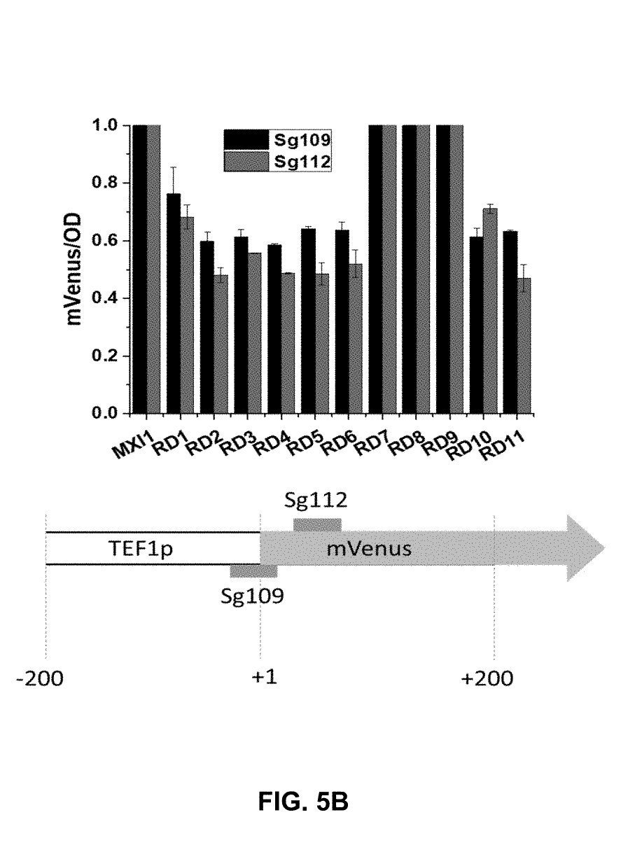

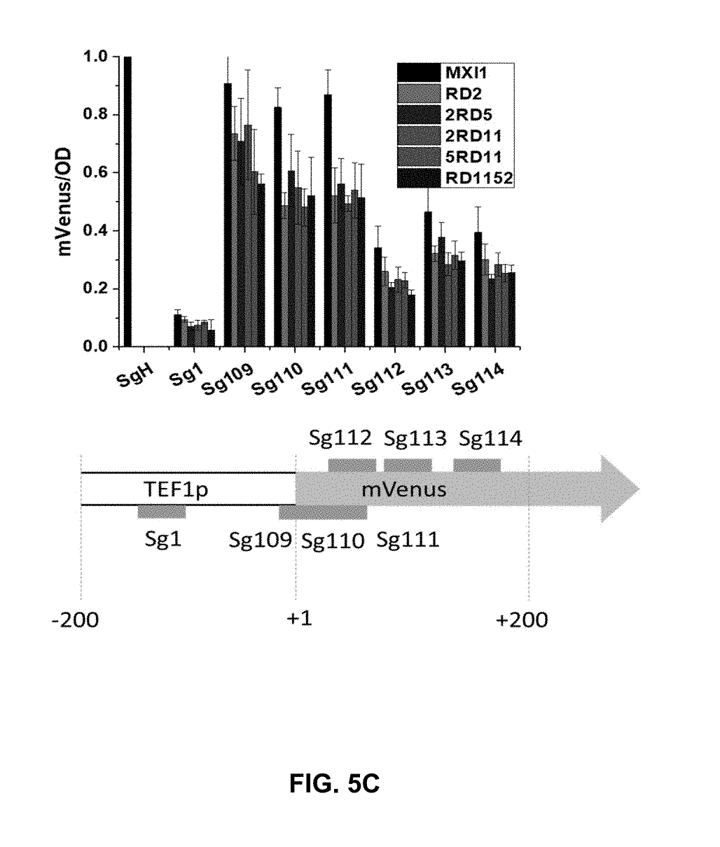

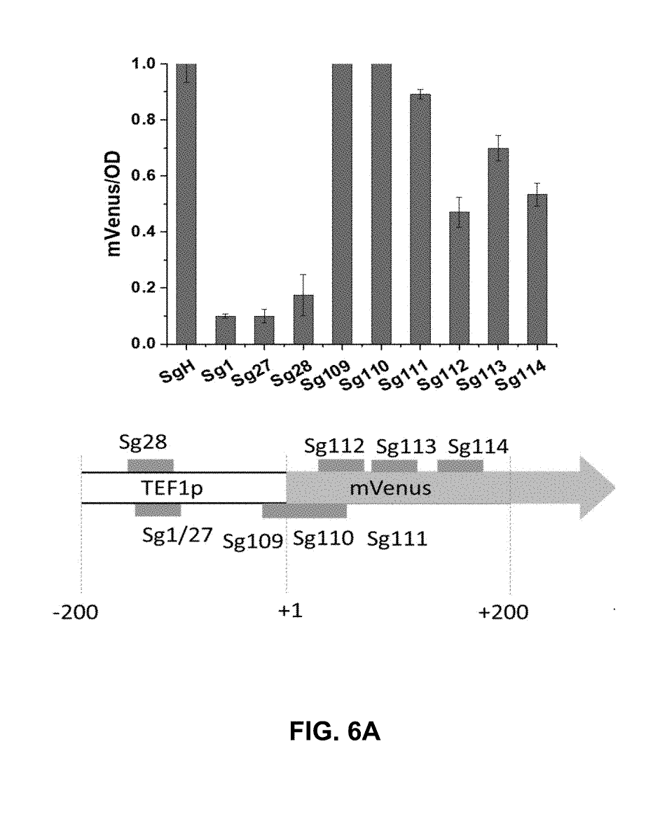

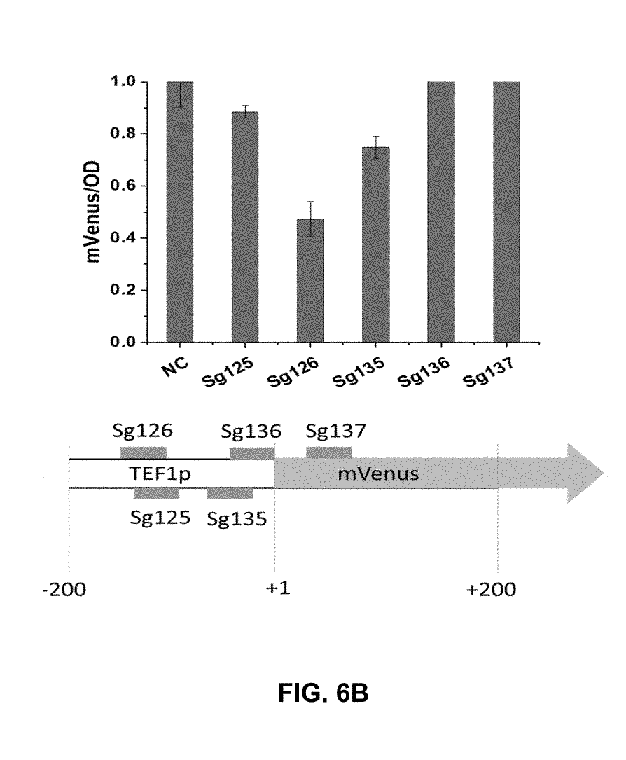

In previous studies, only one repression domain from mammalian cells (MXI1) has been reported and used for CRISPRi in yeast (Gilbert, L. A., et al., Cell 154:442-451 (2013)). Thus, the endogenous repression domain should work better to achieve maximal CRISPRi (FIG. 5A). CRISPRi can be achieved by either blocking transcriptional initiation (i.e. binding to the promoter region) or transcriptional elongation (i.e. binding to the coding sequences). Indeed, although dSpCas9-MXI1 could block transcriptional initiation efficiently, the CRISPRi efficiency to block transcriptional elongation was much lower (FIG. 5A-5C and FIG. 6A-6C). By replacing MXI1 with the native repression domains (Table 9), such as those from TUP1, MIG1, and UME6, the efficiency of CRISPRi was significantly improved.

TABLE 9
Repression domains for CRISPRi in yeast

| | Repressor | Domain (aa) | Function (From SGD) |
|---|---|---|---|
| RD1 | TUP1 | 1-200 | General transcription repressor that binds histones and is |
| RD2 | | 73-129 | involved in nucleosome positioning; forms repressor |
| RD3 | | 277-340 | complex with CYC8 |
| RD4 | | 73-340 | |
| RD5 | MIG1 | 481-504 | Transcription factor involved in glucose repression |
| RD6 | | 380-504 | |
| RD7 | CRT1 | 1-130 | Major transcriptional repressor of DNA-damage-regulated |
| RD8 | | 1-240 | genes |
| RD9 | | 709-811 | |
| RD10 | XTC1 | 75-100 | A direct transcriptional repressor |
| RD11 | UME6 | 508-594 | Represses transcription by recruiting conserved histone |
| | | | deacetylase RPD3 and chromatin-remodeling factor ISW2 |

Well-characterized repression domains were chosen. TUP1 (Edmondson, D. G., et al., Genes Dev. 10:1247-1259 (1996)); MIG1 (Ostling, J., et al., Mol. Cell. Biol. 16:753-761 (1996)); CRT1 (Zhang, Z. & Reese, J. C., Mol. Cell. Biol. 25:7399-7411 (2005)); XTC1 (Traven, A., et al., Nucleic Acids Res. 30:2358-2364 (2002)); UME6 (Kadosh, D. & Struhl, K., Cell 89:365-371 (1997)). The repression domain can be small while demonstrating strong transcriptional repression.

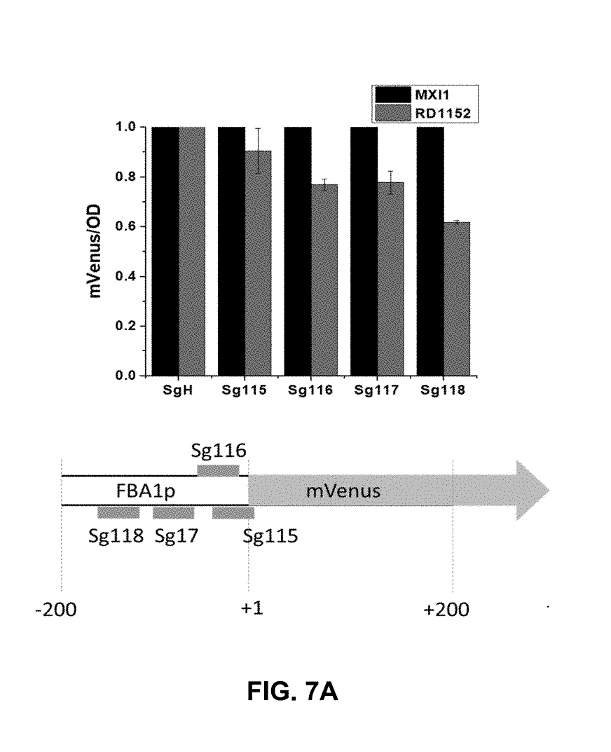

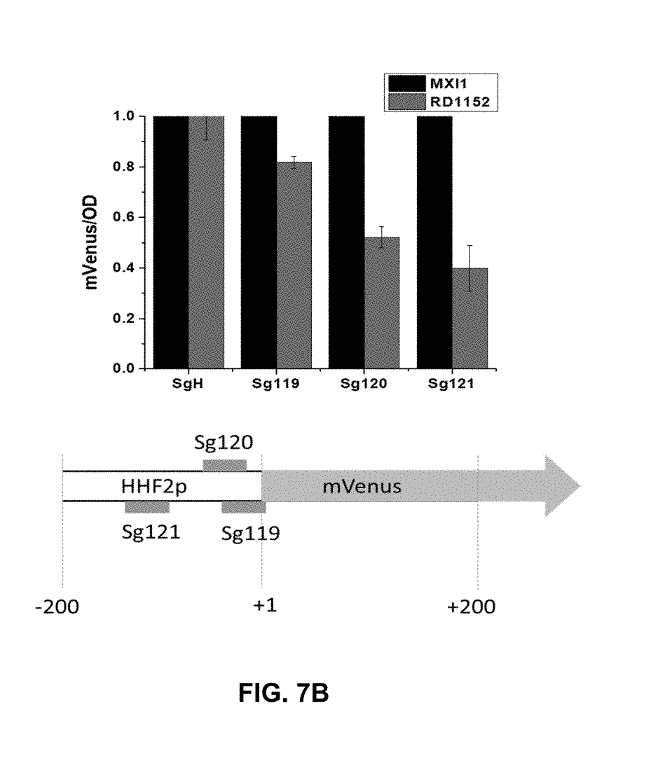

Among several repression domains, RD2, RD5, and RD11 worked the best when fused at the C-terminus of dSpCas9 for CRISPRi (FIG. 5B). Inspired by the design of strong activation domains for CRISPRa (Chavez, A., et al., Nat. Methods 12:326-328 (2015)), multiple repression domains together, either in the form of N- and C-terminal tagged or tandem repeat at the C-terminus, were combined to engineer an optimal repression domain for CRISPRi (FIG. 5A). It was found that the use of multiple repression domains further enhanced CRISPRi efficiency (FIG. 5C). More importantly, the engineered repression domain also improved CRISPRi efficiency when targeting other promoters, such as FBA1p and HHF2p (FIG. 7A-7B). dSpCas9-RD1152 (dSpCas9-RD11-RD5-RD2) demonstrated the highest CRISPRi efficiency and was chosen for further studies. Since dLbCpf1 was not efficient enough for CRISPRi (FIG. 6B), the optimal design of the tri-functional and orthogonal CRISPR-AID system was determined to be dLbCpf1-VP for CRISPRa, dSpCas9-RD1152 for CRISPRi, and SaCas9 for CRISPRd.

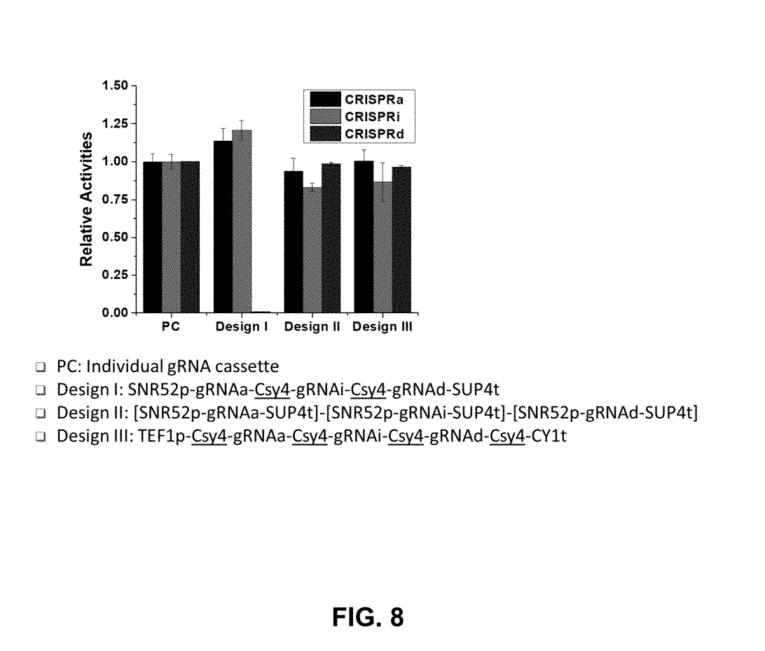

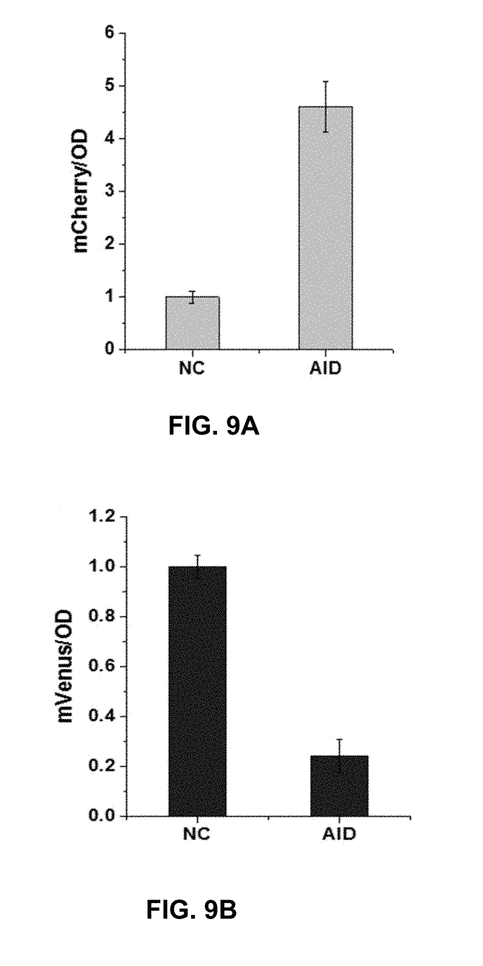

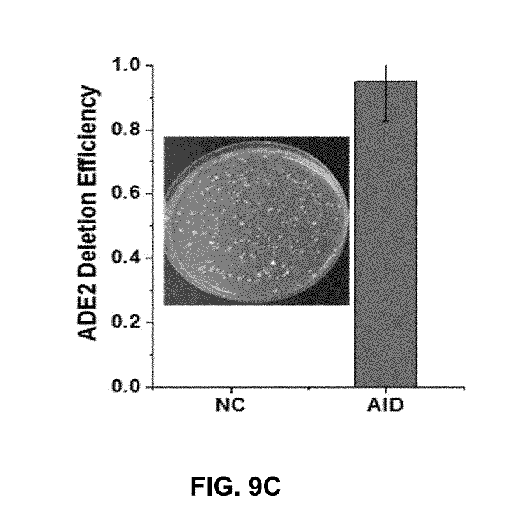

After optimization of the individual modules, all three CRISPR modules were assembled together and integrated them into the yeast genome for stable maintenance. In addition, an endoribonuclease (Csy4) module was included for multiplex processing of gRNAs. In this case, several gRNAs can be transcribed in a single expression cassette, if the Csy4 recognition sites are introduced between neighboring gRNA sequences. Firstly, an array of 3 gRNAs were cloned downstream of SNR52p (design I), a type III promoter commonly used for gRNA expression in yeast. Unfortunately, only the first two gRNAs were found to be functional (FIG. 8), probably due to the limited capability of the type III promoter to transcribe long sequences. Then, the expression of multiple gRNAs were tested as individual expression cassettes (design II) or using a type II promoter (TEF1p, design III). In both cases, all the three gRNAs were fully transcribed and the tri-functional CRISPR-AID was demonstrated in the reporter yeast strain CT (FIG. 8). As shown in FIG. 9A-9C, after introducing a single plasmid containing an array of gRNAs (pSg163, design III), the expression of mCherry was increased by 5-fold (FIG. 9A), the expression of mVenus was decreased by 5-fold (FIG. 9B), and the deletion of ADE2 was achieved with an efficiency higher than 95% (FIB. 9C). More importantly, comparable CRISPRa, CRISPRi, and CRISPRd efficiencies were obtained when the gRNAs were cloned individually or in the array format (FIG. 8). Notably, CRISPRi was demonstrated by targeting mVenus coding sequences (blocking transcriptional elongation) rather than targeting TEF1p (blocking transcriptional initiation), since the expression of SaCas9 and the gRNA array were both driven by TEF1p in our CRISPR-AID system. Otherwise, much higher CRISPRi efficiency could be expected by slightly modifying the design of the gRNA array.

Using the optimized CRISPR-AID system, 5-fold activation of a red fluorescent protein, 5-fold interference of a yellow fluorescent protein, and >95% deletion of an endogenous gene can be achieved simultaneously by transforming a single plasmid into yeast. This strategy enables perturbation of the metabolic and regulatory networks in a modular, parallel, and high throughput manner.

### Example 3: Rational Metabolic Engineering Using CRISPR-AID

After the proof-of-concept study, to confirm that CRISPR-AID can be stably maintained and used for metabolic engineering applications, CRISPR-AID was tested with a well-known phenotype, the production of β-carotene in yeast.

β-Carotene Production and Quantification.

β-Carotene producing strains with gRNAs were pre-cultured in SED-HIS-URA/G418 medium for approximately 2 days, inoculated into 5 mL fresh medium with an initial OD₆₀₀ of 0.1 in 14 mL culture tubes, and cultured under aerobic conditions (30° C., 250 rpm) for 5 days. The stationery phase yeast cells were collected by centrifuge at 13,000×g for 1 min and cell precipitates were resuspended in 1 mL of 3N HCl, boiled for 5 min, and then cooled in an ice-bath for 5 min. The lysed cells were washed with ddH₂O and resuspended in 400 μL acetone to extract β-carotene. The cell debris was removed by centrifuge and the β-carotene containing supernatant was analyzed for its absorbance at 454 nm. The production of β-carotene was normalized to the cell density.

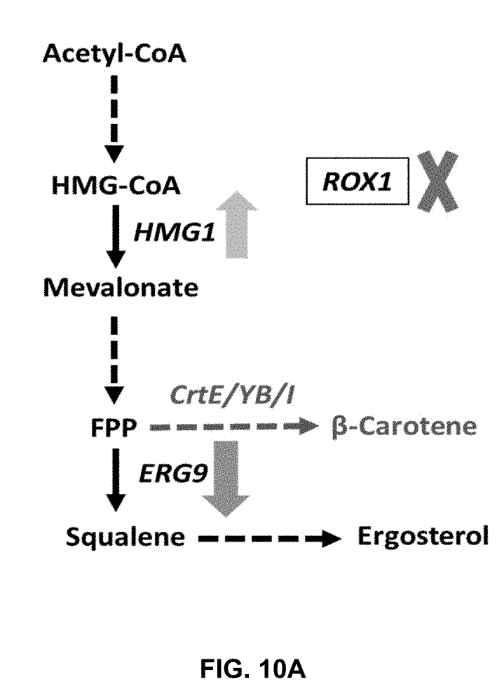

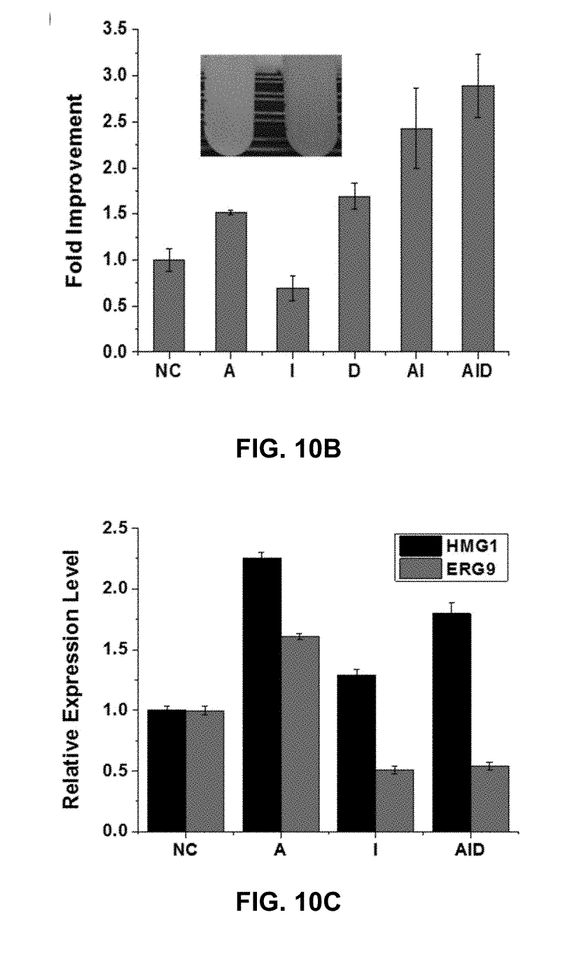

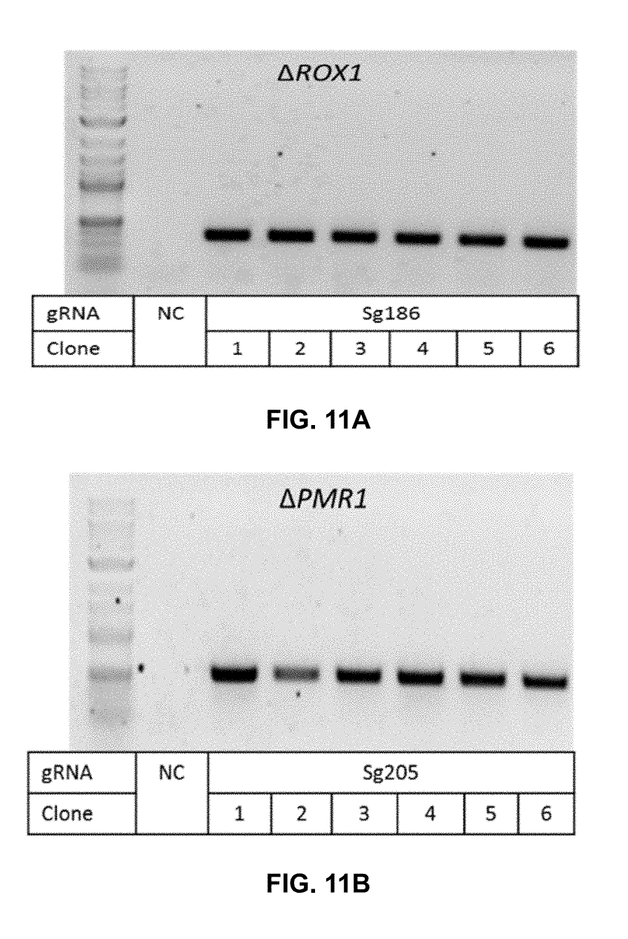

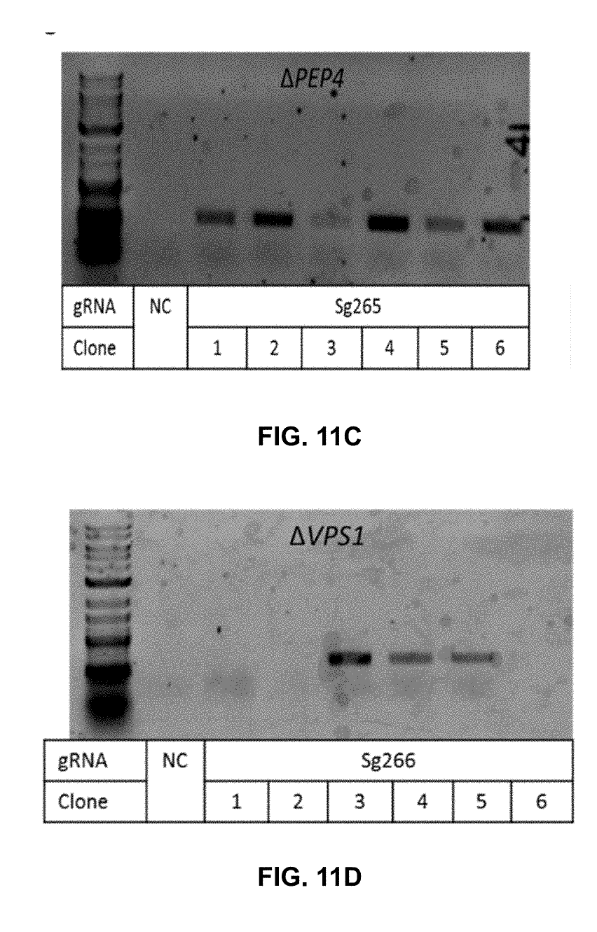

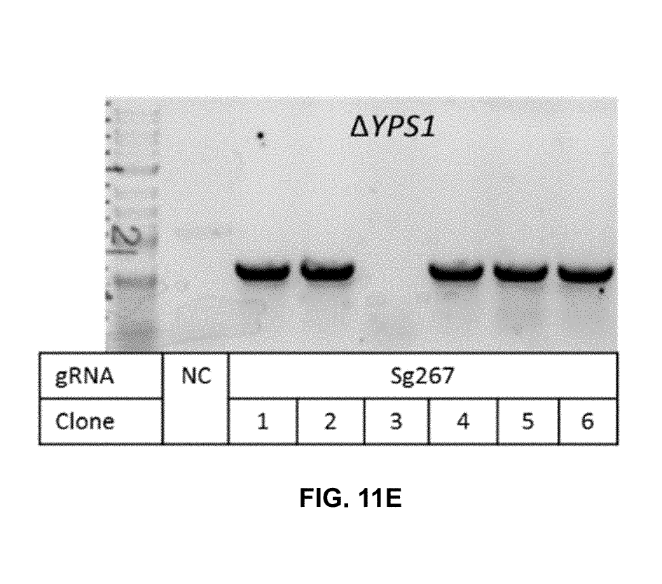

In previous studies, it has been found that overexpression of HMG1 (Xie, W., et al., Metab. Eng. 28:8-18 (2015); Verwaal, R., et al., Appl. Environ. Microbiol. 73:4342-4350 (2007)), encoding a rate-limiting enzyme of the mevalonate pathway, down-regulation of ERG9 (Xie, W., et al., Metab. Eng. 28:8-18 (2015)), an essential gene at the branching point of the (3-carotene biosynthesis and endogenous sterol biosynthesis, and the deletion of ROX1 (Ozaydin, B., et al., Metab. Eng. 15:174-183 (2013)), encoding a stress responsive transcriptional regulator, could significantly increase the production of β-carotene. Therefore, these three targets were selected for CRISPRa, CRISPRi, and CRISPRd, respectively (FIG. 10A). Indeed, it was found that a single gRNA resulted in around 1.7-fold improvement in β-carotene production, while the combination of three gRNAs further improved the production to 2.8-fold (FIG. 10B). After transformation of the corresponding gRNAs, single clones were picked up from the selection plates and cultured in liquid medium. Then genomic DNAs were extracted and subject to diagnostic PCR, with an amplicon only when the desired gene was disrupted. Quantitative PCR (qPCR) and diagnostic PCR further confirmed the enhanced expression of HMG1, down-regulation of ERG9 (FIG. 10C), and deletion of ROX1 (FIG. 11A). Notably, the overexpression of HMG1 resulted in increased expression of ERG9, probably due to the enhanced overall metabolic fluxes towards the mevalonate pathway. In addition, the repression of ERG9 lowered the production of β-carotene, probably due to impaired cell fitness. In other words, HMG1 up-regulation and ERG9 down-regulation should be combined to achieve high β-carotene production (FIG. 10B). Such a synergy between up-regulation of HMG1 and down-regulation of ERG9 was consistent with previous studies (Paradise, E. M., et al., Biotechnol. Bioeng. 100:371-378 (2008)).

Thus, the application of CRISPR-AID was used for rational metabolic engineering with β-carotene production as a case study, and demonstrated a 3-fold increase in β-carotene production in a single step.

### Example 4: CRISPR-AID for Combinatorial Metabolic Engineering

CRISPR-AID was also applied to combinatorial metabolic engineering.

Screening of EGII-Displaying Mutants and Cellulase Activity Assays.

After transforming the combinatorial gRNA library plasmids, the recombinant yeast strains (>10⁵ independent clones with more than 100-fold redundancy) were cultured at 30° C. for 3 days and then subject to immunostaining and flow cytometry analysis (Si, T., et al., Nat. Commun. 8:15187 (2017)). The primary and secondary antibodies were monoclonal mouse anti-histidine tag antibody (1:100 dilution, Bio-Rad, Raleigh, N.C.) and goat anti-mouse IgG (H+L) secondary antibody, Biotin-XX conjugate (1:100 dilution, ThermoFisher Scientific, Rockford, Ill.), respectively. The levels of biotin on the yeast surface were quantified using Streptavidin, R-phycoerythrin conjugate (1:100 dilution, ThermoFisher Scientific). The phycoerythrin (PE) fluorescence was analyzed with a LSR II Flow Cytometer (BD Biosciences, San Jose, Calif.). FACS experiments were performed on a BD FACS Aria III cell sorting system (BD Biosciences, San Jose, Calif.). In the first round of sorting, around 30,000 cells representing the top 1% highest fluorescence were collected. The second round of sorting collected 96 individual yeast cells with the highest fluorescence into a 96-well microplate. Then the plasmids were extracted and retransformed into the CEN-EGII strain with a fresh background, 26 of the retransformed yeast mutants conferred the highest PE fluorescence were further analyzed by the cellulase activity assay. Briefly, 400 μL yeast cells from overnight culture were washed twice with ddH₂O and resuspend in the same volume of 1% (w v⁻¹) carboxymethyl cellulose (CMC) solution (0.1 M sodium acetate, pH 5). After incubation at 30° C. for 16 h with vigorous shaking, the supernatant was analyzed using a modified DNS method (Gonçalves, C., et al., Anal. Methods 2:2046-2048 (2010)) to quantify the amount of the reducing sugars, which was normalized to the cell density to represent the EGII enzyme activity.

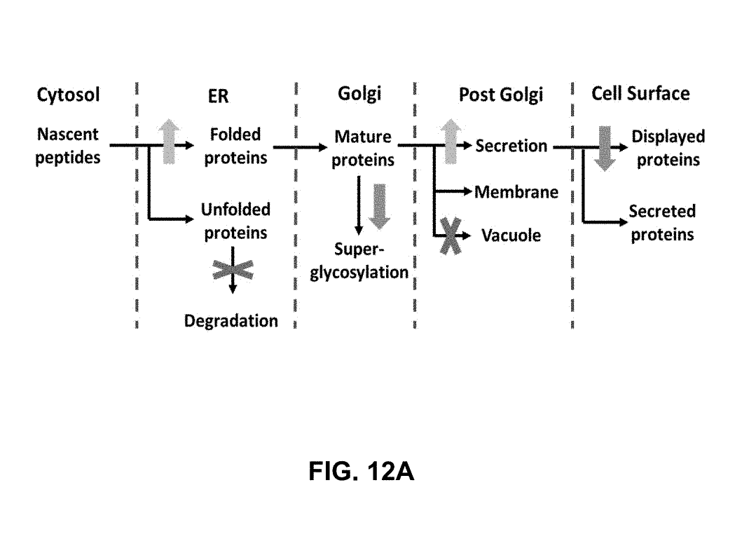

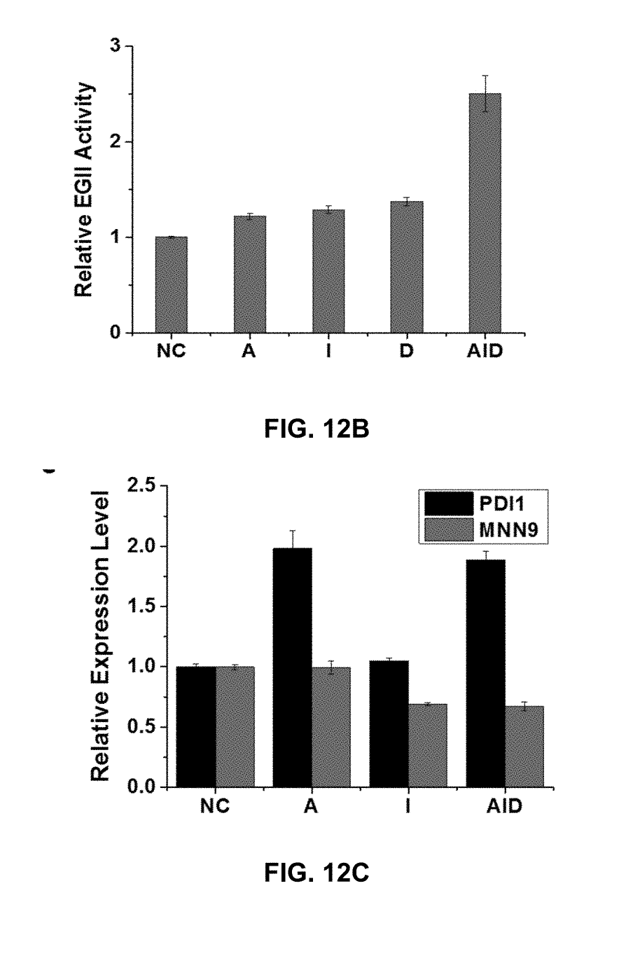

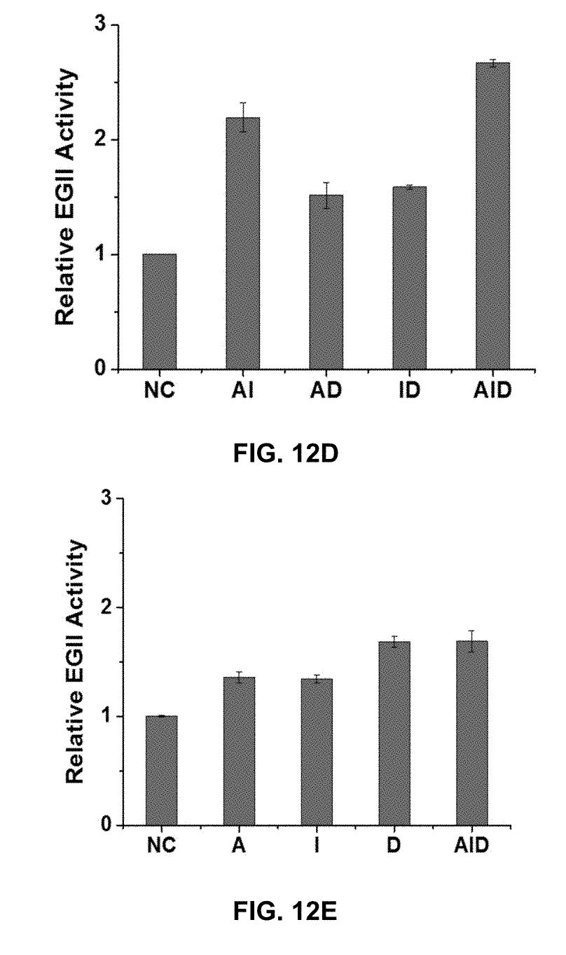

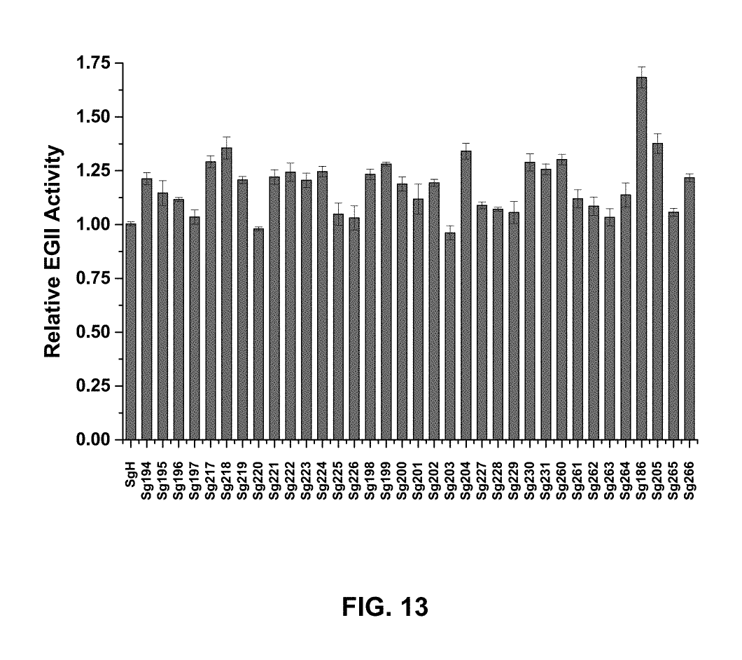

The recombinant protein expression via yeast surface display phenotype was selected because the entire biological process is very important but rather complicated: proteins are translated in the cytosol, folded in the ER, glycosylated in the Golgi, and sorted and secreted to different compartments, and finally attached to the yeast cell surface (FIG. 12A). Many engineering targets have been explored (Hou, J., et al., FEMS Yeast Res. 12:491-510 (2012)), including the up-regulation of the secretory pathway, and down-regulation of the protein degradation and competing pathways, although they have been mainly tested individually. Using CRISPR-AID, the gain-of-function and loss-of-function combinations that work synergistically to increase recombinant protein displaying levels can be determined. Here, Trichoderma reesei endoglucanase II (EGII) was selected as the protein of interest (Si, T., et al., Nat. Commun. 8:15187 (2017)), and 14 targets for CRISPRa, 17 targets for CRISPRi, and 5 targets for CRISPRd (Table 10), most of which increased EGII display levels when tested individually (FIG. 13).

TABLE 10
CRISPR-AID library for EGII display on yeast surface.

| CRISPRa | Target | CRISPRi | Target | CRISPRd | Target |
|---|---|---|---|---|---|
| Sg194 | PEX5 | Sg198 | SED1 | Sg186 | ROX1 |
| Sg195 | PEX5 | Sg199 | SED1 | Sg205 | PMR1 |
| Sg196 | PTI1 | Sg200 | SED1 | Sg265 | PEP4 |
| Sg197 | PTI1 | Sg201 | YCH1 | Sg266 | VPS8 |
| Sg217 | CCW12 | Sg202 | YCH1 | Sg267 | YPS1 |
| Sg218 | ERO1 | Sg203 | YCH1 | | |
| Sg219 | HAC1 | Sg204 | YMR1 | | |
| Sg220 | KAR2 | Sg227 | OCH1 | | |
| Sg221 | PDI1 | Sg228 | OCH1 | | |
| Sg222 | SEC1 | Sg229 | OCH1 | | |
| Sg223 | SLY1 | Sg230 | MNN9 | | |
| Sg224 | SSO1 | Sg231 | MNN9 | | |
| Sg225 | SSO2 | Sg260 | PMR1 | | |
| Sg226 | UBI4 | Sg261 | PMR1 | | |
| | | Sg262 | KEX2 | | |
| | | Sg263 | KEX2 | | |
| | | Sg264 | KEX2 | | |

The empty vector without gRNA sequences was also included in the library, and a library covered all the possible combinations (15*18*6=1620) was created.

A library consisting of all the possible combinations (15*18*6=1620) was generated. Genotyping of several randomly picked colonies indicated that all plasmids were assembled correctly and the library was representative (Table 11).

TABLE 11
Sequencing results of random clones of the combinatorial library for
EGII display on yeast surface.

| | A | I | D |
|---|---|---|---|
| EGII-Random 1 | Sg221 | Sg230 | Sg265 |
| EGII-Random 2 | Sg225 | Sg263 | Sg205 |
| EGII-Random 3 | Sg219 | Sg261 | Sg267 |
| EGII-Random 4 | Sg226 | Sg264 | Sg205 |
| EGII-Random 5 | Sg225 | Sg262 | SgH |
| EGII-Random 6 | SgH | Sg231 | Sg265 |

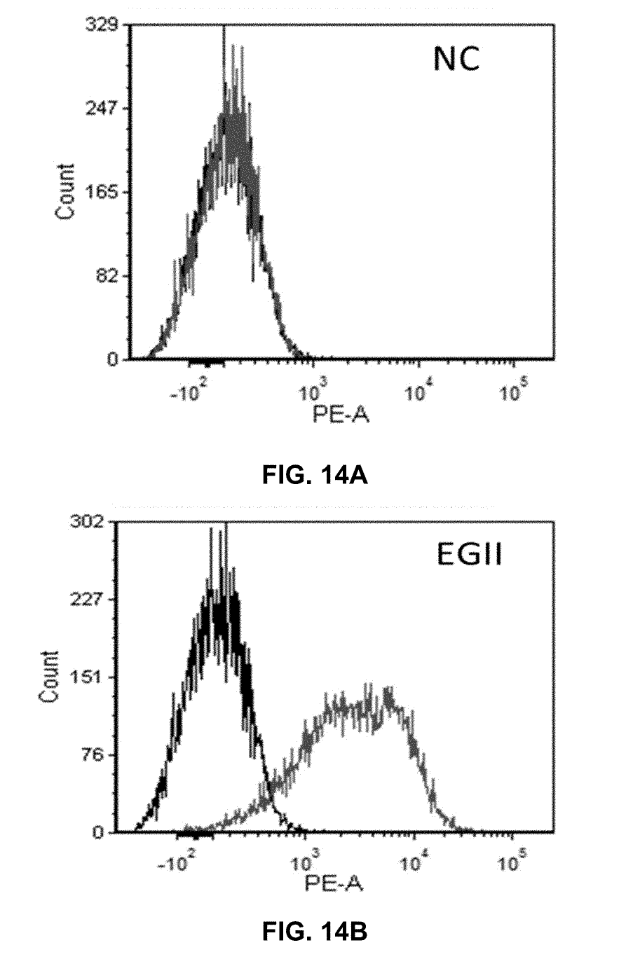

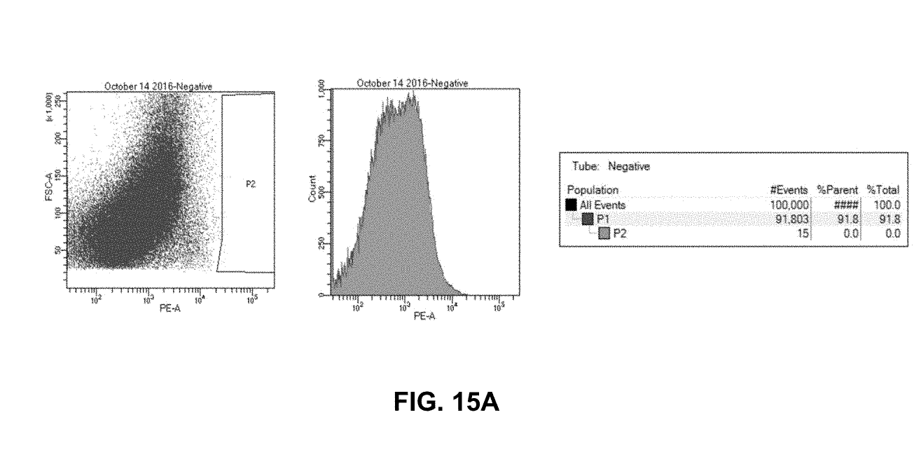

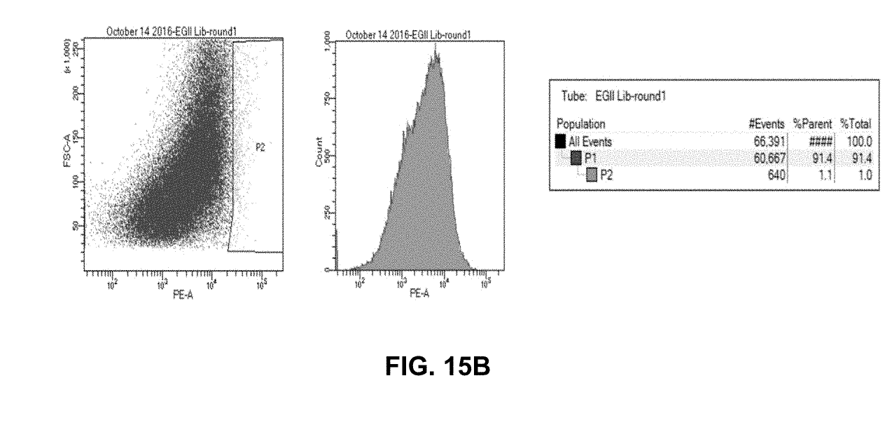

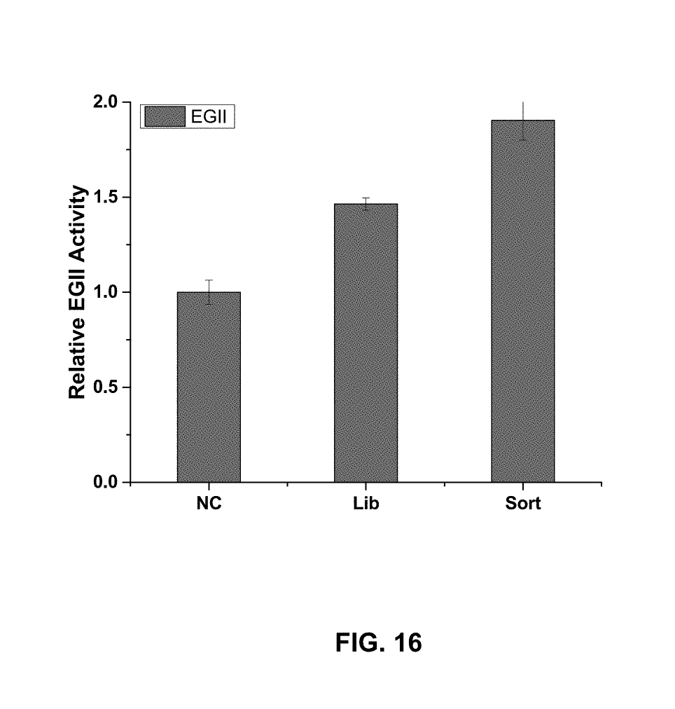

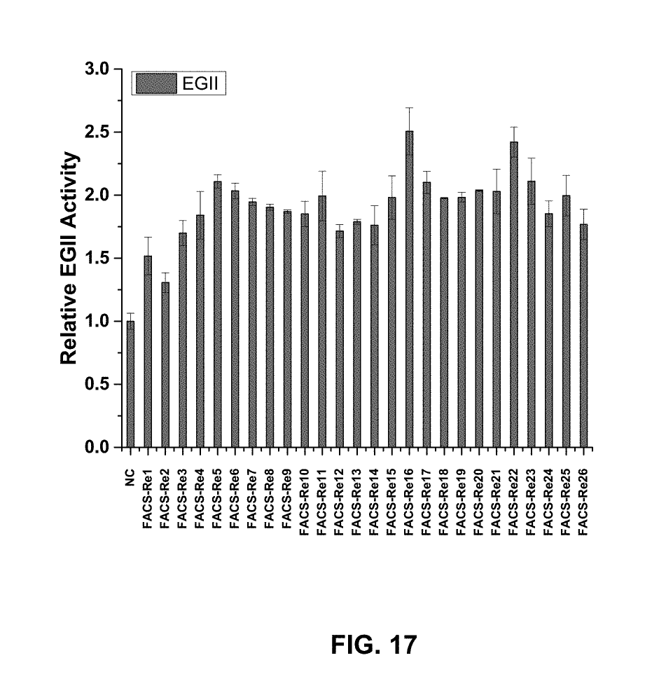

Since the proteins are expressed on the yeast surface, an antibody was used conjugated with a fluorescent dye to detect the epitope tag and convert protein expression levels to fluorescence signals (FIG. 14A-14B, FIG. 15A-15B). Increased EGII activity of the sorted library using FACS (Fluorescence Activated Cell Sorting) indicated that the protein display levels were positively correlated with the fluorescence intensities (FIG. 16). By enriching the highly fluorescent yeast cells, a few combinations that increased the protein expression levels and EGII activities significantly were obtained (FIG. 17). Through DNA sequencing, it was found that the interference and deletion targets were highly enriched, and the two clones showing the highest cellulase activity shared the same combination (Table 12).

Therefore, the combination of PDI1 up-regulation, MNN9 down-regulation, and PMR1 deletion increased EGII display levels and cellulase activity the most (FIG. 12B). The increased expression of PDI1 (CRISPRa) and decreased expression of MNN9 (CRISPRi) were further confirmed using qPCR (FIG. 12C), and the deletion of PMR1 (CRISPRd) at high efficiency was verified by diagnostic PCR (FIG. 11B).

TABLE 12
Sequencing results of top clones of the combinatorial library for EGII
display on yeast surface.

| | A | I | D |
|---|---|---|---|
| EGII-FACS5 | CCW12 | MNN9 | PMR1 |
| EGII-FACS11 | CCW12 | MNN9 | PMR1 |
| EGII-FACS16 | PDI1 | MNN9 | PMR1 |
| EGII-FACS17 | SEC1 | MNN9 | PMR1 |
| EGII-FACS22 | PDI1 | MNN9 | PMR1 |
| EGII-FACS23 | SLY1 | MNN9 | PEP4 |

The top clones were obtained by FACS sorting of the combinatorial library and cellulase activity assay verification.

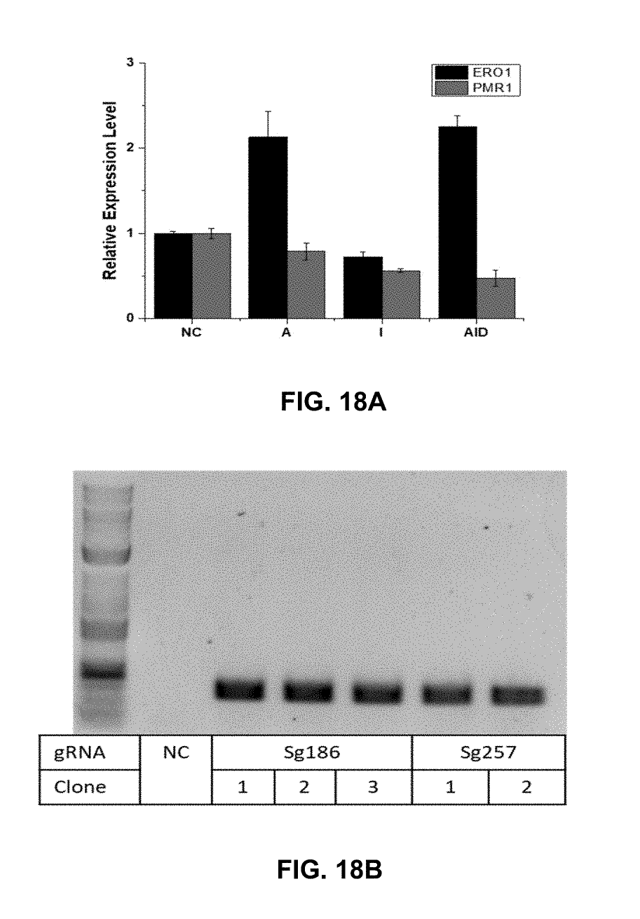

Interestingly, none of the components (PDI1 activation, MNN9 interference, and PMR1 deletion) of the best combination increased EGII display level the most in each category when tested individually, indicating possible synergistic interactions among these genomic modifications. To figure out the potential synergistic interactions, all the double mutants were constructed, including AI (PDI1 activation and MNN9 interference), AD (PDI1 activation and PMR1 deletion), and ID (MNN9 interference and PMR1 deletion), and measured their cellulase activities. As shown in FIG. 12D, a clear synergistic interaction between PDI1 activation and MNN9 interference to increase the protein display levels and EGII activities was observed, but not between the activation and interference targets and the deletion target. PDI1 encodes a protein disulfide isomerase, which is essential for disulfide bond formation in secretory and cell-surface proteins. MNN9 encodes a subunit of Golgi mannosyltransferase complex, which mediates elongation of the polysaccharide mannan backbone and involves in N-glycosylation of the native and recombinant proteins. A previous study (Tang, H., et al., Sci. Rep. 6:25654 (2016)) found that the deletion of MNN9 increased the expression of a couple of genes related to protein secretion, but did not induce the unfolded protein response, such as the expression of PDI1, which might explain the synergy between PDI1 overexpression and MNN9 down-regulation for recombinant protein secretion and display. Finally, combinatorial optimization was compared with the traditionally used single-factor optimization for metabolic engineering applications, where the top candidates from each category (ERO1 activation, PMR1 interference, and ROX1 deletion) were combined. As shown in FIG. 18A-18B, transcriptional regulation of ERO1 and PMR1 and genome editing of ROX1 were verified by qPCR (FIG. 18A) and diagnostic PCR, respectively (FIG. 18B). Unfortunately, no positive effects by combining these three metabolic engineering targets together was observed (FIG. 12E), indicating the significance of combinatorial optimization of cellular metabolism and the advantage of CRISPR-AID to explore the synergy of various metabolic engineering targets for microbial cell factory development.

Thus, CRISPR-AID was also demonstrated for combinatorial optimization of the metabolic engineering targets to enhance the expression and display of a recombinant protein on the yeast surface by 2.5-fold as well as exploring the synergistic interactions among these genomic modifications.

### Example 5: CRISPR-AID Design with Truncated gRNA

As mentioned above, although the CRISPR based genome engineering technology has grown exponentially in recent years, most of the current studies mainly focus on a mono-function CRISPR in a specific biological system.

The initial design of a tri-functional CRISPR system was to combine two strategies: truncated gRNA with the MS2 aptamer to recruit MS2-VP64 for transcriptional activation, truncated gRNA with the Com aptamer to recruit Com-MXI1 for transcriptional interference, and full-length gRNA for gene deletion. gRNAs with different length of targeting sequences were tested in catalytically active SpCas9 containing yeast strain. If the targeting sequences were longer than 16nt, no survival clones could be obtained, due to the introduction of a double strand break in the genome by the catalytically active Cas9. When the targeting sequences were between 16 and 12nt, efficient transcriptional regulation (CRISPRi in this case) could be achieved. If the targeting sequences were shorted than 12nt, CRISPRi efficiency was dramatically decreased.

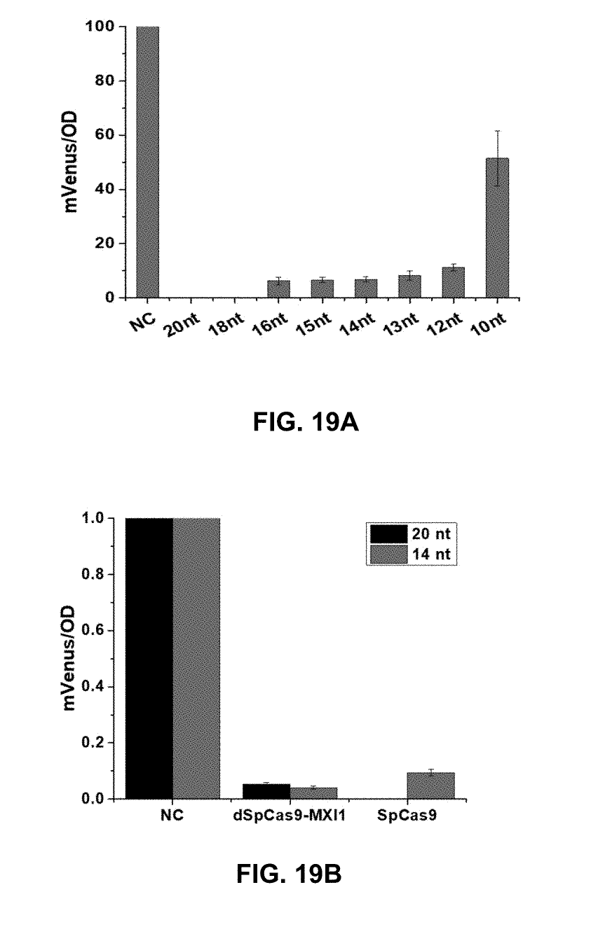

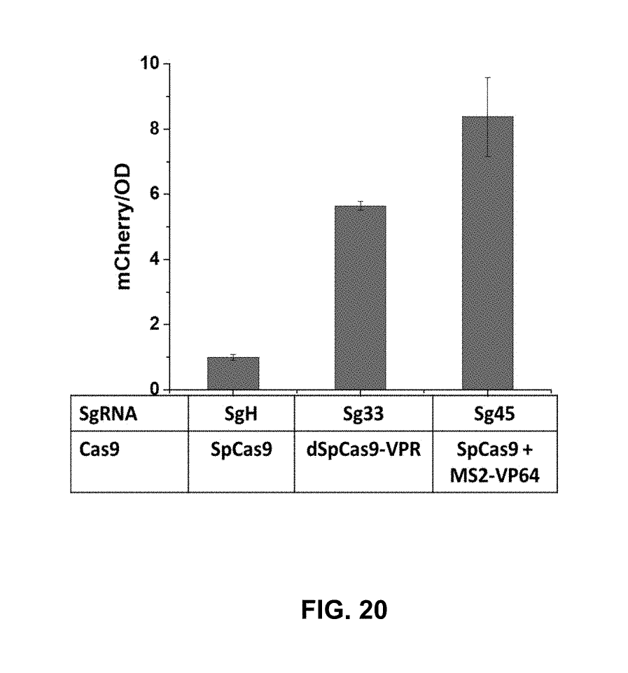

Thus, compared with that of the full-length gRNA, we found that truncated gRNAs (12-16 nt targeting sequences) resulted in comparable CRISPRi (FIG. 19A-19B) and CRISPRa (FIG. 20) efficiency in yeast.

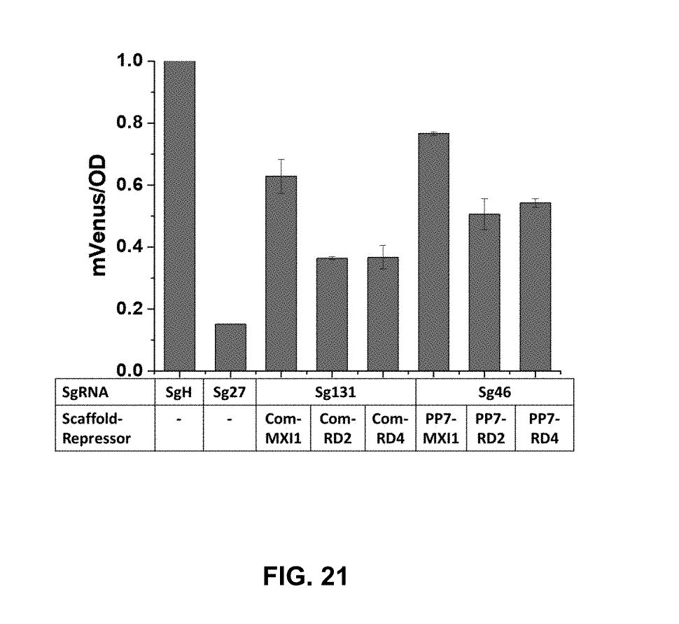

In addition, the use of truncated gRNA together with modular RNA scaffold engineering (SpCas9+Sg45+MS2-VP64) worked equally well as one of the optimal CRISPRa designs (dSpCas9-VPR+Sg33 or Sg6). Unfortunately, CRISPRi efficiency was dramatically decreased when an aptamer was added to the gRNA scaffold, which might result from lower binding affinity between Cas9 and the engineered gRNA. The change of repression domains and the use of another aptamer-RNA binding domain pair did not significantly improve CRISPRi efficiency either (FIG. 21). Interestingly, although orthogonal transcriptional regulation was developed using modular RNA scaffolds, the use of such a system for CRISPRi was only demonstrated in mammalian cells and un-modified gRNA (without aptamer and RNA binding protein to recruit a repression domain) was used for CRISPRi in yeast (Zalatan, J. G., et al., Cell 160:339-350 (2015)). A most recent study following a similar design (gRNA with the MS2 aptamer to recruit MS2-VPR for transcriptional activation and gRNA with the PP7 aptamer to recruit PCP-MXI1 for transcriptional interference) resulted in limited success in transcriptional reprogramming and metabolic engineering applications in yeast (Jensen, E. D., et al., Microb. Cell Fact. 16:46 (2017)). In both cases, gRNAs were modified to be independent of each other to enable a dual-functional CRISPR system, while the Cas9 protein remained intact (Zalatan, J. G., et al., Cell 160:339-350 (2015); Kiani, S., et al., Nat. Methods 12:1051-1054 (2015); Dahlman, J. E., et al., Nat. Biotechnol. 33:1159-1161 (2015)). In other words, they are not fully orthogonal CRISPR systems, since competition between different gRNAs may still occur. Overall, a simple combination of the modular RNA scaffold engineering and the gRNA truncation strategies did not work to develop a tri-functional CRISPR system. In this study, we developed a fully orthogonal tri-functional CRISPR-AID by using three independent CRISPR proteins, whose protospacer adjacent motif (PAM) sequences and gRNA scaffold sequences are different from each other.

CRISPR-AID was utilized for genome-scale engineering, with potential applications in both metabolic engineering and fundamental studies. Although yeast is one of the most well studied microorganisms, the whole metabolic and regulatory networks are still not clearly understood. In previous metabolic engineering efforts, it was often found that some unknown or unrelated targets resulted in the highest increase in the desired phenotype (Caspeta, L., et al., Science 346:75-78 (2014); Kim, S. R., et al., PLoS One 8:e57048 (2013)). Therefore, genome-scale metabolic engineering is needed to cover all the possible important targets. In the genome-scale CRISPR-AID system, a comprehensive library can be created that can control the expression of any single gene in the yeast genome to different levels (increased expression, decreased expression, and zero expression). Followed by high throughput screening and next generation sequencing, multiple hits that increase the desired phenotype can be obtained, and the process can be repeated iteratively until the construction of optimal microbial cell factories (see Example 6).

In summary, a tri-functional CRISPR-AID system was developed by combining transcriptional activation, transcriptional interference, and gene deletion in a single system, and applied CRISPR-AID for rational and combinatorial metabolic engineering. We also explored synergistic interactions among different genome modifications.

### Example 6: Design of CRISPR-MAGIC for a Multi-Functional Genome-Scale System

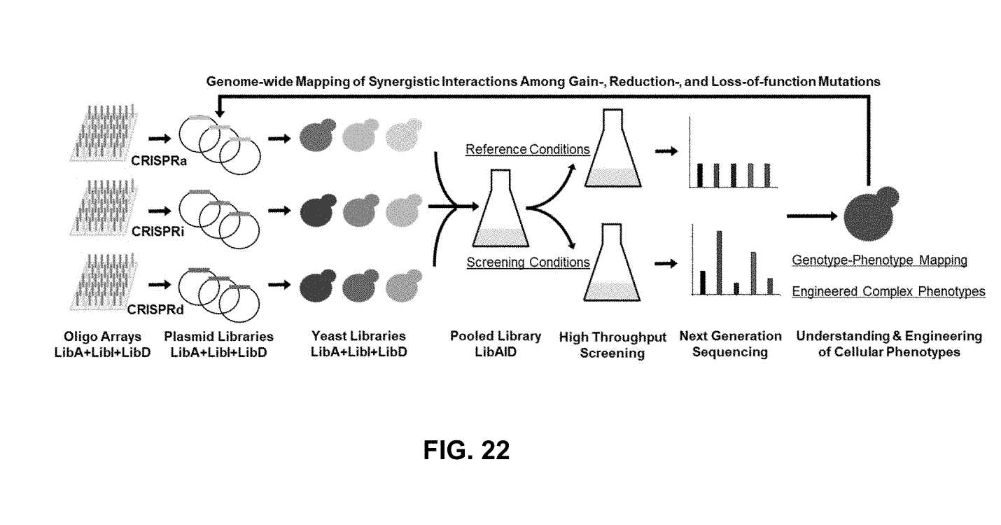

As described above, a tri-functional CRISPR system (CRISPR-AID) was constructed, where three orthogonal CRISPR proteins were integrated to achieve gene activation, interference, and deletion simultaneously (Lian, J., et al., Nat. Commun. 8:1688, (2017)). To further develop a multi-functional genome-wide CRISPR (MAGIC) system, three genome-scale gRNA expressing plasmid libraries from pools of array-synthesized oligos were designed and constructed, each for upregulating, downregulating, and deleting all the genes in the yeast genome, respectively (FIG. 22).

Strains, Media, and Cultivation Conditions.

Escherichia coli strain NEB10β (New England Biolabs, Ipswich, Mass.) was used to maintain and amplify plasmids and recombinant strains were cultured at 37° C. in Luria broth medium containing 100 μg/mL ampicillin. S. cerevisiae BY4742 was used as the host for genome-scale engineering of furfural tolerance and surface display of recombinant proteins. Yeast strains were cultivated in complex medium consisting of 2% peptone, 1% yeast extract, and 2% glucose (YPD) or synthetic complete medium consisting of 0.17% yeast nitrogen base, 0.1% mono-sodium glutamate, 0.077% CSM-URA, and 2% glucose (SED-URA) at 30° C., 250 rpm. When necessary, 200 μg/mL G418 (KSE Scientific, Durham, N.C.) was supplemented.

Plasmid and Strain Construction.

SNR52p-BsaI-BsaI-gRNA structural sequences-SUP4t (Lian, J., et al., Nat. Commun. 8:1688, (2017)) were cloned into BsaI-free pRS426 to construct gRNA expression plasmids, including p426*-LbSgH for CRISPRa, p426*-SpSgH for CRISPRi, and p426*-SaSgH for CRISPRd. Then the targeting sequences were synthesized as short oligos and cloned into the BsaI sites of the helper plasmids. Yeast plasmids were isolated using a Zymoprep Yeast Plasmid Miniprep II Kit (Zymo Research, Irvine, Calif.) and amplified in E. coli. All the recombinant plasmids and oligonucleotides used in this study were listed in Table 13 and Table 14, respectively.

TABLE 13
Plasmids constructed in this study.

| Name | Description | Applications |
|---|---|---|
| pAID6 | pRS41K-INT-[dLbCpf1-VP]-Csy4-[dSpCas9-RD1152]-SaCas9 | Integrate AID |
| p426*-LbSgH | SNR52p-Scaffold-BsaI-BsaI-SUP4t cloned into BsaI-free pRS426 | Helper |
| p426*-SpSgH | SNR52p-BsaI-BsaI-Scaffold-SUP4t cloned into BsaI-free pRS426 | plasmids for |
| p426*-SaSgH | SNR52p-BsaI-BsaI-Scaffold-SUP4t cloned into BsaI-free pRS426 | gRNA cloning |
| pSg482 | SPC97a guide sequences cloned into p426-LbSgH | SPC97a |
| pSg483 | BUD22a guide sequences cloned into p426-LbSgH | BUD22a |
| pSg486 | SIZ1i guide sequences cloned into p426-SpSgH | SIZ1i |
| pSg487 | SLX5i guide sequences cloned into p426-SpSgH | SLX5i |
| pSg488 | NUP133i guide sequences cloned into p426-SpSgH | NUP133i |
| pSg489 | GPI17i guide sequences cloned into p426-SpSgH | GPI17i |
| pSg490 | UME1i guide sequences cloned into p426-SpSgH | UME1i |
| pSg553 | MRPL32a guide sequences cloned into p426-LbSgH | MRPL32a |
| pSg554 | ASE1a guide sequences cloned into p426-LbSgH | ASE1a |
| pSg558 | RCF1a guide sequences cloned into p426-LbSgH | RCF1a |
| pSg591 | NAT1a guide sequences cloned into p426-LbSgH | NAT1a |
| pSg592 | NRT1a guide sequences cloned into p426-LbSgH | NRT1a |
| pSg593 | COQ4a guide sequences cloned into p426-LbSgH | COQ4a |
| pSg549 | NEO1i guide sequences cloned into p426-SpSgH | NEO1i |
| pSg587 | YNL146Wi guide sequences cloned into p426-SpSgH | YNL146Wi |
| pSg588 | tH(GUG)Ki guide sequences cloned into p426-SpSgH | tH(GUG)Ki |
| pSg589 | SNU66i guide sequences cloned into p426-SpSgH | SNU66i |
| pSg590 | DDL1i guide sequences cloned into p426-SpSgH | DDL1i |
| pSg615 | YNR064Ca guide sequences cloned into p426-LbSgH | YNR064Ca |
| pSg616 | MGR1a guide sequences cloned into p426-LbSgH | MGR1a |
| pSg617 | PEP7i guide sequences cloned into p426-SpSgH | PEP7i |
| pSg618 | VPS8i guide sequences cloned into p426-SpSgH | VPS8i |
| pSg619 | ZRT1i guide sequences cloned into p426-SpSgH | ZRT1i |
| pSg621 | WHI2i guide sequences cloned into p426-SpSgH | WHI2i |
| pSg622 | PDR1i guide sequences cloned into p426-SpSgH | PDR1i |
| pSg624 | MUK1i guide sequences cloned into p426-SpSgH | MUK1i |
| pFACS20 | 1^{st} round FACS isolated plasmid for HOC1 deletion | HOC1d |
| pFACS22 | 1^{st} round FACS isolated plasmid for UBP3 interference | UBP3i |
| pFACS23 | 1^{st} round FACS isolated plasmid for MNN9 interference | MNN9i |
| pFACS8 | 2^{nd} round FACS isolated plasmid for NUP157 interference | NUP157i |
| pFACS25 | 2^{nd} round FACS isolated plasmid for PDI1 activation | PDI1a |
| pSg334 | X2-targeting guide sequences cloned into p426-SaSgH | SaCas9 |
| pSg335 | X3-targeting guide sequences cloned into p426-SaSgH | mediated |
| pSg336 | X4-targeting guide sequences cloned into p426-SaSgH | marker-less |
| pSg337 | XI1-targeting guide sequences cloned into p426-SaSgH | genome |
| pSg338 | XI2-targeting guide sequences cloned into p426-SaSgH | integration |
| pSg339 | XI3-targeting guide sequences cloned into p426-SaSgH | |
| pSg340 | XI4-targeting guide sequences cloned into p426-SaSgH | |
| pSg341 | XII2-targeting guide sequences cloned into p426-SaSgH | |
| pSg342 | XII4-targeting guide sequences cloned into p426-SaSgH | |
| pSg343 | XII5-targeting guide sequences cloned into p426-SaSgH | |

For plasmids pAID6, p426*-LbSgH, p426*-SpSgH p426*-SaSgH, see Lian, J., et al., 2017, Nat. Commun. 8:1688.

TABLE 14
Primers used in this study.

| | | SEQ ID | |
|---|---|---|---|
| Names | Sequences (5′-3′) | NO: | Applications |
| X4-INT-T7F | ggtttccagccacagttgtagtcacgtgcgcgccatgctgtaatacgactcactataggg | 455 | Integrate EGII |
| X4-INT- | cttggtagttggagcgcaattagcgtatcctgtaccatacaattaaccctcactaaaggg | 456 | into X4 locus |
| T3R | | | |
| LibA-F | tccttaagtggtccgtgttcggacctaatc | 457 | Amplify |
| LibA-R | ccagctgccacctctaagaatggacgacgt | 458 | gRNA |
| LibI-F | cggagcagacattgtaaggctacgttcacc | 459 | libraries from |
| LibI-R | gtaggcctctcgtgctatcttcgttggacg | 460 | the oligo pools |
| LibD-F | gtatctcgcagccggtctccgatc | 461 | |
| LibD-R | cggttctctctcgtggtctcgaaac | 462 | |
| AID-NGS- | tcgtcggcagcgtcagatgtgtataagagacagcttctccgcagtgaaagataaatgatc | 463 | Amplify |
| F1 | | | gRNA |
| AID-NGS- | gtctcgtgggctcggagatgtgtataagagacagctttgagtgagctgataccgctcg | 464 | libraries for |
| R1 | | | NGS |
| pSg482F | agatttgttccgcgactaccaggggaa | 465 | gRNA primers |
| pSg482R | aaaattcccctggtagtcgcggaacaa | 466 | for SPC97a |
| pSg483F | agatatgagacgttttcttcattgatg | 467 | gRNA primers |
| pSg483R | aaaacatcaatgaagaaaacgtctcat | 468 | for BUD22a |
| pSg486F | gatccagcagttccatcagagtga | 469 | gRNA primers |
| pSg486R | aaactcactctgatggaactgctg | 470 | for SIZli |
| pSg487F | gatcagagcgtgtgttgcgttgat | 471 | gRNA primers |
| pSg487R | aaacatcaacgcaacacacgctct | 472 | for SLX5i |
| pSg488F | gatcaaccaaaacatacaccattt | 473 | gRNA primers |
| pSg488R | aaacaaatggtgtatgttttggtt | 474 | for NUP133i |
| pSg489F | gatcatacgtaacacagatttaac | 475 | gRNA primers |
| pSg489R | aaacgttaaatctgtgttacgtat | 476 | for GPI17i |
| pSg490F | gatctcaacgcctgagccaaagat | 477 | gRNA primers |
| pSg490R | aaacatctttggctcaggcgttga | 478 | for UME1i |
| pSg553F | agataggcaaagacaagaaaatacaag | 479 | gRNA primers |
| pSg553R | aaaacttgtattttcttgtctttgcct | 480 | for MRPL32a |
| pSg554F | agatactaaataaccgcccagaaaatc | 481 | gRNA primers |
| pSg554R | aaaagattttctgggcggttatttagt | 482 | for ASE1a |
| pSg558F | agatgatgcagacgtggccaagttggc | 483 | gRNA primers |
| pSg558R | aaaagccaacttggccacgtctgcatc | 484 | for RCF1a |
| pSg591F | agatgacgcggagcagggtaaaaagtg | 485 | gRNA primers |
| pSg591R | aaaacactttttaccctgctccgcgtc | 486 | for NAT1a |
| pSg592F | agatcccgaagaacaaatagcggtagc | 487 | gRNA primers |
| pSg592R | aaaagctaccgctatttgttcttcggg | 488 | for NRT1a |
| pSg593F | agataggatgccgtaaaagaatgctcc | 489 | gRNA primers |
| pSg593R | aaaaggagcattcttttacggcatcct | 490 | for COQ4a |
| pSg549F | gatcacagtgttatgcttactaag | 491 | gA primers |
| pSg549R | aaaccttagtaagcataacactgt | 492 | for NEO1i |
| pSg587F | gatcaattaagattgtagagggag | 493 | gRNA primers |
| pSg587R | aaacctccctctacaatcttaatt | 494 | for |
| | | | YNL146Wi |
| pSg588F | gatctacaacgtagaactgataaa | 495 | gRNA primers |
| pSg588R | aaactttatcagttctacgttgta | 496 | for |
| | | | tH(GUG)Ki |
| pSg589F | gatctgaatacctataactgctaa | 497 | gRNA primers |
| pSg589R | aaacttagcagttataggtattca | 498 | for SNU66i |
| pSg590F | gatctgtcgctttggaagaaaaag | 499 | gRNA primers |
| pSg590R | aaacctttttcttccaaagcgaca | 500 | for DDL1i |
| pSg615F | agataatgactatgttaataacaaagg | 501 | gRNA primers |
| pSg615R | aaaacctttgttattaacatagtcatt | 502 | for |
| | | | YNR064Ca |
| pSg616F | agattcattaaatagagatatataaga | 503 | gRNA primers |
| pSg616R | aaaatcttatatatctctatttaatga | 504 | for MGR1a |
| pSg617F | gatccctttaaaaaccatgagatc | 505 | gRNA primers |
| pSg617R | aaacgatctcatggtttttaaagg | 506 | for PEP7i |
| pSg618F | gatcggtgtaatgagtaatggtct | 507 | gRNA primers |
| pSg618R | aaacagaccattactcattacacc | 508 | for VPS8i |
| pSg619F | gatcagatcatgacagccgatacc | 509 | gRNA primers |
| pSg619R | aaacggtatcggctgtcatgatct | 510 | for ZRT1i |
| pSg621F | gatcctgttcttgtagaatcggag | 511 | gRNA primers |
| pSg621R | aaacctccgattctacaagaacag | 512 | for WHI2i |
| pSg622F | gatcgcggccatatagacattacc | 513 | gRNA primers |
| pSg622R | aaacggtaatgtctatatggccgc | 514 | for PDR1i |
| pSg624F | gatcgattgattagggtcaaacct | 515 | gRNA primers |
| pSg624R | aaacaggtttgaccctaatcaatc | 516 | for MUK1i |
| qSIZ1 F | aacaattgccgaacattctggg | 517 | Primers for |
| qSIZ1 R | tttcttggcgttggggatgata | 518 | qPCR analysis |
| qNAT1 F | atgatatcgagccatgcgtctt | 519 | |
| qNAT1 R | cgcgtctacaattgacccaat | 520 | |
| qPDR1 F | ttcgatatcatctgcagggagc | 521 | |
| qPDR1 R | aagggctgcggtaagtgattta | 522 | |
| qNUP157 | agtactagaaggggatgcaggt | 523 | |
| qNUP157 | taaaacgcctcttgactggtca | 524 | |
| R2 | | | |
| qACT1 F2 | ctgtcttcccatctatcgtcgg | 525 | |
| qACT1 R2 | agcttcatcaccaacgtaggag | 526 | |
| pSg334F | gatcagtaagttgagtgtaaggtgg | 527 | gRNA for X2 |
| pSg334R | aaacccaccttacactcaacttact | 528 | integration |
| pSg206F | gatcgtgattgttagttcagcgtaa | 529 | gRNA for X3 |
| pSg206R | aaacttacgctgaactaacaatcac | 530 | integration |
| pSg207F | gatcggcagccgtcgttgggcagaa | 531 | gRNA for X4 |
| pSg207R | aaacttctgcccaacgacggctgcc | 532 | integration |
| pSg337F | gatctgcatcgcgatgttagtttag | 533 | gRNA for XI1 |
| pSg337R | aaacctaaactaacatcgcgatgca | 534 | integration |
| pSg338F | gatcccttctgttcatgcgtgacgg | 535 | gRNA for XI2 |
| pSg338R | aaacccgtcacgcatgaacagaagg | 536 | integration |
| pSg339F | gatcggagaaaggaaagtagaaatg | 537 | gRNA for XI3 |
| pSg339R | aaaccatttctactttcctttctcc | 538 | integration |
| pSg340F | gatcgtcgctaagatcattgtaact | 539 | gRNA for |
| pSg340R | aaacagttacaatgatcttagcgac | 540 | XII1 |
| | | | integration |
| pSg341F | gatcaatagtctcacttactgggcg | 541 | gRNA for |
| pSg341R | aaaccgcccagtaagtgagactatt | 542 | XII2 |
| | | | integration |
| pSg342F | gatctactgccacgtatttaatgag | 543 | gRNA for |
| pSg342R | aaacctcattaaatacgtggcagta | 544 | XII4 |
| | | | integration |
| pSg343F | gatctctaccgtgagaaataaagca | 545 | gRNA for |
| pSg343R | aaactgctttatttctcacggtaga | 546 | XII5 |
| | | | integration |
| X2-INT-F | gccacccataatcggcgcttagtttcggagttcaatcatactttgaaaagataatgtatg | 547 | Donner for X2 |
| X2-INT-R | atatggggtcagtggcgatattatactataggagttaaagaggaaacagctatgaccatg | 548 | integration |
| X3-INT-F | atcaggcacgaaggcacactcgtatatgcatgttgttgaactttgaaaagataatgtatg | 549 | Donner for X3 |
| X3-INT-R | ttccatggggtcgcaacttttcccggtgacctctacatgtaggaaacagctatgaccatg | 550 | integration |
| X4-INT-F | cagccacagttgtagtcacgtgcgcgccatgctgactaatctttgaaaagataatgtatg | 551 | Donner for X4 |
| X4-INT-R | tggtagttggagcgcaattagcgtatcctgtaccatactaaggaaacagctatgaccatg | 552 | integration |
| XI1-INT-F | gcgccggttttcattttcttccacggaataccaagcccatctttgaaaagataatgtatg | 553 | Donner for |
| XI1-INT-R | ctgtacgcagcatttagcagagatttgccaatgccaagaaaggaaacagctatgaccatg | 554 | XI1 |
| | | | integration |
| XI2-INT-F | ttcacgcaagttaagtccaggaaggtgagcaaatgctcatctttgaaaagataatgtatg | 555 | Donner for |
| XI2-INT-R | aggcacggaaacggctgcacgggtacgccagataaggataaggaaacagctatgac | 556 | XI2 |
| | catg | | integration |
| XI3-INT-F | ccaatcaaagaagcatcggttcagatcgagcaaactgtagctagaaaagataatgtatg | 557 | Donner for |
| XI3-INT-R | tgacatccaaactacaaaaccgagattggacatatagcacaggaaacagctatgaccatg | 558 | XI3 |
| | | | integration |
| XII1-INT-F | atacaatagcacatctcattacccagttatgattgacgtcctagaaaagataatgtatg | 559 | Donner for |
| XII1-INT-R | cgaggaaaattagaattagtggagcaaataatgagcacagaggaaacagctatgacca | 560 | XII1 |
| | tg | | integration |
| XII2-INT-F | tgcgtctaacgcttttgccacttggatttctattataggactttgaaaagataatgtatg | 561 | Donner for |
| XII2-INT-R | aagaaattcttcctgtgcttcatcaaaacgcgaaaattcgaggaaacagctatgaccatg | 562 | XII2 |
| | | | integration |
| XII4-INT-F | agcgcttataaggttggggcaatactaaaactgtgatcttctttgaaaagataatgtatg | 563 | Donner for |
| XII4-INT-R | ttccgactctgttgtacctattgtactaatagggtacgaggaaacagctatgaccatg | 564 | XII4 |
| | | | integration |
| XII5-INT-F | tactaactcttctcacgctgcccctatctgttcttccgcctttgaaaagataatgtatg | 565 | Donner for |
| XII5-INT-R | ctagccttattgttttagttcagtgacagcgaactgccgtaggaaacagctatgaccatg | 566 | XII5 |
| | | | integration |

The CRISPR-AID strain (bAID) was constructed by integrating PmeI digested pAID6 (Lian, J., et al., Nat. Commun. 8:1688, (2017)) into the genome of BY4742 and selection for G418 resistance. The Trichoderma reesei endoglucanase II (EGII)-displaying strain (bAID-EG) was constructed by integrating the TEF1p-prepro-HisTag-EGII-AGA1-PGK1t cassette (Lian, J., et al., Nat. Commun. 8:1688, (2017); Si, T. et al., Nat. Commun. 8:15187, (2017)) into the X4 locus of bAID. The gRNA expression cassettes identified by MAGIC screening were integrated into the predefined loci (Table 15) in a CRISPR-assisted and marker-less manner.

TABLE 15
Characterization of the genomic loci for marker-less integration of
gRNA expression cassettes.

| Site | A | I | Sum of A and I | No donor | gRNA |
|---|---|---|---|---|---|
| X2 | 0/8 | 3/8 | 3/16 | Confluent | pSg334 |
| X3 | 7/8 | 8/8 | 15/16 | 4 | pSg335 |
| X4 | | | | 0 | pSg336 |
| XI1 | 8/8 | 8/8 | 16/16 | 30 | pSg337 |
| XI2 | 1/8 | 5/8 | 6/16 | Confluent | pSg338 |
| XI3 | 8/8 | 8/8 | 16/16 | 1 | pSg339 |
| XII1 | 0/8 | 0/8 | 0/16 | Confluent | pSg340 |
| XII2 | 7/8 | 8/8 | 15/16 | 20 | pSg341 |
| XII4 | 6/8 | 7/8 | 13/16 | 20 | pSg342 |
| XII5 | 8/8 | 8/8 | 16/16 | 1 | pSg343 |

The gRNA targeting efficiency was tested by transforming the gRNA plasmid without any donor to repair the double strand break, and efficient gRNA should result in no survived colonies. The integration efficiency and gRNA expression level were evaluated by co-transforming the reporter strain (bAID-RV) with gRNA plasmid as well as its corresponding linear donor fragment, which contained a gRNA expression cassette to activate the expression of mCherry or to repress the expression of mVenus. Eight colonies were randomly picked up to measure the change in fluorescence intensities. The corresponding results were shown in Example 10 below. The loci and the corresponding gRNAs chosen for CRISPR-assisted and marker-less genome integration are shown in bold in Table 15.

Recombinant yeast strains constructed in this study are listed in Table 16.

TABLE 16
Strains constructed in this study

| Name | Genotypes |
|---|---|
| BY4742 | MATα his3Δ1 leu2Δ0 lys2Δ0 ura3Δ0 |
| bAID | BY4742-Delta::KanMX-[dLbCpf1-VP]-[Csy4]- |
| | [dSpCas9-RD1152]-[SaCas9] |
| bAID-RV | bAID-X4::[CYC1p-mCherry-TEF1t]- |
| | [TEF1p-mVenus-PGK1t] |
| bAID-EG | bAID-X4::[TEF1p-prepro-HIS-EGII-AGA1-PGK1t] |
| R1 | bAID-X3::SIZ1i |
| R2 | bAID-X3::SIZ1i-X4::NAT1a |
| R3 | bAID-X3::SIZ1i-X4::NAT1a-XI1::PDR1i |
| T1 | Same as R1 |
| T2 | bAID-X4::NAT1a |
| T3 | bAID-XI1::PDR1i |
| T1 + T2 | Same as R2 |
| T1 + T3 | bAID-X3::SIZ1i-XI1::PDR1i |
| T2 + T3 | bAID-X4::NAT1a-XI1::PDR1i |
| T1 + T2 + T3 | Same as R3 |
| EG11 | bAID-EG-HOC1d |
| EG12 | bAID-EG-UBP3i |
| EG13 | bAID-EG-MNN9i |
| EG21 | bAID-EG-HOC1d-NUP157i |
| EG22 | bAID-EG-HOC1d-PDI1a |

Design and Synthesis of the MAGIC Library.

To create a MAGIC library, first all possible guide sequences targeting all ORFs and RNA genes (rRNAs, tRNAs, snRNAs, snoRNAs, and ncRNAs) were obtained and ranked using previously described criteria and empirical experiences (Bao, Z., et al., Nat. Biotechnol. 36:505-508, (2018); Lian, J., et al., Nat. Commun. 8:1688, (2017)) (Table 17). All ORF and RNA coding sequences and their promoter sequences were extracted from the Saccharomyces genome database (yeastgenome.org). The promoter sequences, entire sequences, and coding sequences were used for the design of activation, interference, and deletion guide sequences, respectively. The desired region sequences were given to the CHOPCHOP program to generate all possible guide sequences (Labun, K., et al., Nucleic Acids Res. 44:W272-276, (2016); Montague, T. G., et al., Nucleic Acids Res. 42:W401-407, (2014)).

Different from CRISPRd, the gRNA binding sites relative to the transcriptional starting sites can be equally important as the guide sequences for CRISPRa and CRISPRi (Gilbert, L. A., et al., Cell 159:647-661, (2014); Lian, J., et al.., Nat. Commun. 8:1688, (2017)). Therefore, the following criteria were included to rank the guide sequences, targeting efficiency, targeting positions, GC contents, and off-target effects. The guide sequences containing polyT, polyG, and BsaI sites were excluded. In addition, to make the genome-scale libraries more diversified, only the top ranked guides were kept if multiple guide sequences were clustered together. The ranking criteria were validated using the previously designed gRNAs (Lian, J., et al., Nat. Commun. 8:1688, (2017)) with high efficiency. The ranking criteria are detailed in Table 17 and validated by the previously designed gRNAs showing high efficiency (Table 18).

TABLE 17
Criteria for scoring of the guide sequences for the CRISPRa,
CRISPRi, and CRISPRd libraries

| | LibA | LibI | LibD |
|---|---|---|---|
| Efficiency score E¹ | 0 | CHOPCHOP | CHOPCHOP |
| Position score a² | a=\|X-250\|/250 | if X < 0, a = \|X+125\|/125; | if X/CDS < ⅓ a = 0 |
| | | if X >= 0 and from T a = 0.25 | if ⅓ =< X/CDS <= ⅔ a = 0.2 |
| | | if X >= 0 and from NT a = 1 | if X/CDS > ⅔ a = 0.5 |

| GC score b | if 40-60% | b=0 |
|---|---|---|
| | if 30-40% or 60-70% | b=0.2 |
| | if 20-30% or 70-80% | b=0.4 |
| | if 10-20% or 80-90% | b=0.6 |
| | if 0-10% or 90-100% | b=0.8 |
| Off-target score c³ | c=(SM+MM0+MM1+MM2+MM3)/20 | |
| PolyT score d⁴ | if ConsecutiveT < 4 | d=0 |
| | if ConsecutiveT > 4 | d= ConsecutiveT/10 |
| PolyG score e⁵ | if consecutiveG > 5 | e=0 |
| | else | e=1 |

| | | |
|---|---|---|
| BsaI score f⁶ | if BsaI | f=0 |
| | else | f=1 |

| | | |
|---|---|---|
| Diversity score g⁷ | if distance < 10 bp | g=0 |
| | else | g=1 |

| | |
|---|---|
| Total Score S | S=(3+E−a−b−c−d)*e*f*g |

¹Efficiency score is from COPCHOP (Labun, K., Montague, et al., E. Nucleic Acids Res. 44: W272-276, (2016)), and the computational program for the efficiency score of Cpf1 was not available when the library sequences were designed. Therefore, the highest scores for the activation, interference, and deletion gRNA libraries are 3, 4, and 4, respectively.
²X represents the gRNA binding site, with X = 0 presenting the start codon (ATG). Based on previous experience, CRIAPRa is the most active when binding to ~200 bp upstream of the transcription starting site (TSS) or ~250 bp upstream of the start codon); the efficiency of CRISPRi is the highest when targeting to the promoter region (~75 bp upstream of TSS or ~125 bp upstream of the start codon) and the template strand (T) of the coding sequences; for gene disruption, it is better to target the 5′-end of the coding sequences.
³SC and MM scores are from CHOPCHOP. SC, self-complementarity; MM0, no mismatche; MM1, 1 mismatch; MM2, 2 mismatches; MM3, 3 mismatches.
⁴PolyT may be read as a terminator by the Type III RNA polymerase.
⁵PolyG is difficult for DNA synthesis.
⁶BsaI is used for the cloning of the gRNA plasmid libraries.
⁷The gRNAs cluster together may have similar targeting efficiency and it may result in low library diversity.

TABLE 18
Validation of the gRNA ranking criteria.

| CRISPRa | gRNA | Ranking | CRISPRi | gRNA | Ranking | CRISPRd | gRNA | Ranking |
|---|---|---|---|---|---|---|---|---|
| CCW12 | Sg217 | 4 | CYS4 | Sg246 | 3 | ADE2 | Sg93 | 3 |
| ERO1 | Sg218 | Close to 1 | | Sg247 | 11 | | Sg94 | 5 |
| GAL11 | Sg242 | 3 | | Sg248 | Close to 5 | | Sg95 | 10 |
| | Sg243 | 5 | ERG9 | Sg170 | Close to 1 | PEP1 | Sg265 | 2 |
| HMG1 | Sg175 | 1 | | Sg171 | Close to 2 | ADO1 | Sg255 | 2 |
| | Sg176 | 3 | | Sg172 | 3 | ROX1 | Sg186 | 7 |
| | Sg177 | 6 | | Sg173 | 4 | VPS8 | Sg266 | 1 |
| MET6 | Sg252 | 1 | | Sg174 | 12 | YPS1 | Sg267 | 2 |
| | Sg253 | 2 | KEX2 | Sg262 | 9 | | | |
| | Sg254 | 3 | | Sg263 | 3 | | | |
| PEX5 | Sg194 | 2 | | Sg264 | 1 | | | |
| | Sg195 | Close to 4 | MNN9 | Sg230 | 4 | | | |
| PTI1 | Sg196 | Close to 3 | | Sg231 | Close to 5 | | | |
| | Sg197 | 5 | OCH1 | Sg227 | 1 | | | |
| SAM2 | Sg244 | Close to 1 | | Sg228 | 9 | | | |
| | Sg245 | 3 | | Sg229 | 4 | | | |
| SEC1 | Sg222 | 2 | PMR1 | Sg204 | 2 | | | |
| SSO1 | Sg224 | 1 | | Sg260 | 6 | | | |
| | | | | Sg261 | Close to 3 | | | |
| | | | SED1 | Sg198 | 1 | | | |
| | | | | Sg199 | 3 | | | |
| | | | | Sg200 | 2 | | | |
| | | | YCH1 | Sg201 | Close to 2 | | | |
| | | | | Sg202 | 1 | | | |
| | | | | Sg203 | 4 | | | |
| | | | TEF1 | Sg28 | 3 | | | |
| | | | | Sg27 | 10 | | | |

Most of the previously designed gRNAs (Lian, J., et al., Nat. Commun. 8:1688, (2017)) with high efficiency was found to be highly ranked in the designed genome-scale CRISPRa, CRISPRi, and CRISPRd libraries.

For each gene, the top-six, top-six, and top-four guide sequences with the highest scores were selected for CRISPRa, CRISPRi, and CRISPRd libraries, respectively. 100 non-targeting guide sequences were included in each library as negative controls. Adapters containing priming sequences and BsaI sites were added to both ends of each oligonucleotide for PCR amplification and Golden Gate assembly. The unique priming sequences allowed the construction of each library independently. The CRISPRa and CRISPRi oligonucleotide libraries were synthesized on a 92918 format chip, while the CRISPRd oligonucleotide library was synthesized on two 12472 format chips (CustomArray, Bothell, Wash.) and mixed at equal molar ratio.

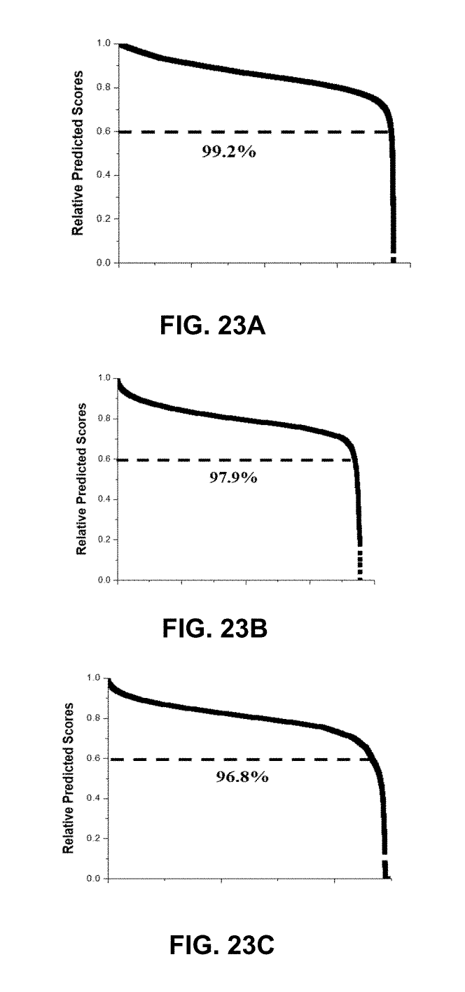

On average, ˜98% of the designed gRNAs showed high scores (FIG. 23A-23C). 100 randomly generated guide sequences were also included as negative controls in each library. Adapters were added to both ends of these oligos for cloning purposes (Table 19).

TABLE 19
Design of oligonucleotides for CRISPRa, CRISPRi,
and CRISPRd libraries.

| | Sequences (5′ to 3′) |
|---|---|
| LibA | 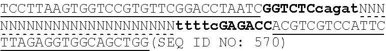 |
| LibI | 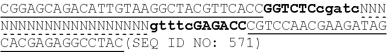 |
| LibD | 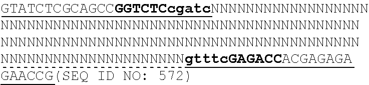 |

The priming sites are underlined, BsaI sites for golden-gate assembly are highlighted in bold, guide sequences are dotted underlines and the homology donor for HI-CRISPR gene deletion are plain capital letters.

In summary, 37817, 37870, and 24806 unique guide sequences were designed and synthesized for the CRISPRa, CRISPRi, and CRISPRd libraries, respectively (Table 20 and Table 21).

TABLE 20
Construction and Characterization of the MAGIC plasmid library

| | | |
|---|---|---|
| LibA | LibI | LibD |

Design and construction of MAGIC libraries

| | | | |
|---|---|---|---|
| CRISPR protein | dLbCpf1-VP | dSpCas9-RD1152 | SaCas9 |
| Length of gRNA¹ | 20 + 23 bp | 20 + 82 bp | 121 + 127 bp |
| No. of guides | 37817 | 37870 | 24806 |
| Fold coverage² | ~133x | ~106x | ~121x |

Characterization of MAGIC libraries

| | | | |
|---|---|---|---|
| Mapping ratio | ~87.7% | ~86.8% | ~72.6% |
| gRNA coverage | ~99.9% | 100% | ~88.9% |
| Gene coverage³ | 100% | 100% | ~98.3% |

¹The length of guide (underlined) and structural sequences.
²Calculated as estimated library size/No. of guide sequences.
³At least one guide for each gene.

TABLE 21
Guide sequence distribution of the designed
CRISPRa, CRISPRi, and CRISPRd libraries.

| | Genes Targeting | | | | | | Total | Total |
|---|---|---|---|---|---|---|---|---|
| No. of guides | 1 | 2 | 3 | 4 | 5 | 6 | genes | guides |
| No. of genes | 0 | 1 | 6 | 10 | 11 | 6267 | 6295 | 37717 |
| in LibA | | | | | | | | |
| No. of genes | 0 | 0 | 0 | 0 | 0 | 6295 | 6295 | 37770 |
| in LibI | | | | | | | | |
| No. of genes | 44 | 111 | 108 | 6029 | 0 | 0 | 6295 | 24706 |
| in LibD | | | | | | | | |

Notably, 100 randomly generated guide sequences in each library were not included in this table.

Exemplary guide sequences for the top-six activation guide sequences, the top-six interference guide sequences, and top-four deletion guide sequences for the ACS1, ADE1, AIM2, ATS1, and BDH1 genes are shown in Table 22. The full list of 37817, 37870, and 24806 unique guide sequences that were designed and synthesized for the CRISPRa, CRISPRi, and CRISPRd libraries are not shown for brevity.

TABLE 22
Exemplary guide sequences with scores
CRISPRa Library

| | Gene | | | SEQ ID |
|---|---|---|---|---|
| Number | Name | Score | Sequence | NO: |
| 0 | ACS1 | 2.796 | CCACGGCATGTCAACAGGTGAGT | 663 |
| 0 | ACS1 | 2.632 | CCACCGAGGAACTGTACCCCAAC | 664 |
| 0 | ACS1 | 2.588 | CTTTGGATCTTAGAGATAACAGA | 665 |
| 0 | ACS1 | 2.444 | TAGGGGATGGAGAGTGCTACGCC | 666 |
| 0 | ACS1 | 2.244 | CACAGCCGTACATACACGTGCCA | 667 |
| 0 | ACS1 | 2.124 | TATACAAAATGAAGGGAGAACTA | 668 |
| 1 | ADE1 | 2.7 | GAGTATGGCTACATGGATCAAGT | 669 |
| 1 | ADE1 | 2.684 | CTGAAGGTTGAAAAAGAATGCCA | 670 |
| 1 | ADE1 | 2.644 | AACCTTCAGGAAAAGTTTCAGAT | 671 |
| 1 | ADE1 | 2.452 | TTTACAGCACTTGATCCATGTAG | 672 |
| 1 | ADE1 | 2.424 | TGCTTTGCTATCGTGTAGAACTG | 673 |
| 1 | ADE1 | 2.368 | AGATGAGTTGAAATTTCGAGTAT | 674 |
| 2 | AIM2 | 2.914 | GGTCCACTGTTGGATTCGTAGCA | 675 |
| 2 | AIM2 | 2.74 | ATTAACGTAAAGGAACATAGTGC | 676 |
| 2 | AIM2 | 2.736 | GCTGCTGTTTCTTCTGGCAATCC | 677 |
| 2 | AIM2 | 2.6 | TGCCAGGATCAAGAGCAGCTTCT | 678 |
| 2 | AIM2 | 2.568 | TATGATATCTGGCCTAAGGCGGA | 679 |
| 2 | AIM2 | 2.312 | TCTGTAGTCGACATCTTTTGCTG | 680 |
| 3 | ATS1 | 2.96 | CGTTCCTTACTGTAGATAGTCGG | 681 |
| 3 | ATS1 | 2.826 | TTGCTACTGGTGGACACCCGACT | 682 |
| 3 | ATS1 | 2.528 | AGGGAGACGACGATGCTACCTTG | 683 |
| 3 | ATS1 | 2.42 | AGTTACGTGTTGCATTGCGAGAT | 684 |
| 3 | ATS1 | 2.3364 | TCTTGTTTACGTTCCTTACTGTA | 685 |
| 3 | ATS1 | 2.304 | TAGGATTAAAAGAGATCATGAGC | 686 |
| 4 | BDH1 | 2.976 | CTATCCTTGCCTATTCTTTCCTC | 687 |
| 4 | BDH1 | 2.92 | GACGGAGAGAAGAAACCGGTGTT | 688 |
| 4 | BDH1 | 2.896 | CTCCTTACGGGGTCCTAGCCTGT | 689 |
| 4 | BDH1 | 2.736 | ACATCAAGCCGGATTTGCTCACG | 690 |
| 4 | BDH1 | 2.582 | TCGAGCCAATCGAGGGCAGCAGT | 691 |
| 4 | BDH1 | 2.392 | TCTTGATATGATAATAGGTGGAA | 692 |

CRISPRi Library

| | | | | SEQ ID |
|---|---|---|---|---|
| Number | Name | Score | Sequence | NO: |
| 0 | ACS1 | 3.74 | CGTACTACCAGATAACCTAA | 693 |
| 0 | ACS1 | 3.42 | GTTGGGGTACAGTTCCTCGG | 694 |
| 0 | ACS1 | 3.27 | GGGAGAACTATTTGCCACCG | 695 |
| 0 | ACS1 | 3.17 | TACCCATTGAATAATGGCAT | 696 |
| 0 | ACS1 | 3.14 | CAGTTTATATACAAAATGAA | 697 |
| 0 | ACS1 | 3.11 | GTCCAAGTGTGGAGAATAGT | 698 |
| 1 | ADE1 | 3.53 | CCAGATTCTTTGAGGTAAGA | 699 |
| 1 | ADE1 | 3.27 | TCTGACTCTTGCGAGAGATG | 700 |
| 1 | ADE1 | 3.16 | GTATGTCTATATGTATTAGA | 701 |
| 1 | ADE1 | 3.08 | ACTTTACCTCTGGCCACCAA | 702 |
| 1 | ADE1 | 2.81 | ACTCTGACAGTTTGGTCAAT | 703 |
| 1 | ADE1 | 2.77 | GATTACGAACATCGTTGGAC | 704 |
| 2 | AIM2 | 3.51 | CTATGATATCTGGCCTAAGG | 705 |
| 2 | AIM2 | 3.51 | TCTGCTGTAGTTAGACGTAG | 706 |
| 2 | AIM2 | 3.41 | AGGTTTCTTGCAAATGAGCG | 707 |
| 2 | AIM2 | 3.14 | ATTTCTTCACGACGACCCTT | 708 |
| 2 | AIM2 | 3.05 | CCTTCAAAGCAACACTTGCC | 709 |
| 2 | AIM2 | 2.95 | ATGCCCAAATTTCTATATTA | 710 |
| 3 | ATS1 | 3.28 | TGAAAAATTTCGCGGCGACG | 711 |
| 3 | ATS1 | 3.26 | CTGCATTATCAAGGCTCAAA | 712 |
| 3 | ATS1 | 3.2 | ACATTCCATCACTTGCGCTT | 713 |
| 3 | ATS1 | 3.14 | TTACGTGTTGCATTGCGAGA | 714 |
| 3 | ATS1 | 3.09 | CATTTGTCAGCATCACGCTG | 715 |
| 3 | ATS1 | 3.06 | TGATCATTAAAGGCTATAAC | 716 |
| 4 | BDH1 | 3.25 | GCAGATACTTCGTGTGACAA | 717 |
| 4 | BDH1 | 3.1 | AAGGGCAACATCTGCCCAAA | 718 |
| 4 | BDH1 | 3.09 | ATGGCCAATTCAAGCCCTTT | 719 |
| 4 | BDH1 | 3.08 | CATATCAAGAGAAACAGGCT | 720 |
| 4 | BDH1 | 3.07 | AAACAGGCTAGGACCCCGTA | 721 |
| 4 | BDH1 | 3 | TCTCTTGATATGATAATAGG | 722 |

CRISPRd Library

| | | | | SEQ ID |
|---|---|---|---|---|
| Number | Name | Score | Sequence | NO: |
| 0 | ACS1 | 3.73 | TGGGATGAACACCTTATCGAATGGCTTAGA | 723 |
| | | | CCAGTTTAAAAATTGGGTAGTTCAATAGACT | |
| | | | CCTTGTGCAAGCGCTGATAGTCCTGCAACCC | |
| | | | GTCCAAGTCTTTAGAACCGAAGAACTTAG | |
| 0 | ACS1 | 3.48 | GATCGTGCCACAACGGCCCATCTCAGATAG | 724 |
| | | | ACTGCAGCCCGCAATTGCTAGCAGGACTAT | |
| | | | CAGCGCTTGCACAAGGAGTCTATTGAAGAC | |
| | | | CCTGCTAAGTCCCACTATTCTCCACACTTGG | |
| 0 | ACS1 | 3.39 | GATGACAACTTTAGAGTCCCCATCGTTGATA | 725 |
| | | | CGATCTCTCAAGGAGTTGGAATGGCACCGA | |
| | | | TACGGGAAATGGCCAACAAGGTTATGATTG | |
| | | | CTTCTGGGAAAGAAAACCCGGCAAAGACTA | |
| 0 | ACS1 | 3.27 | GTCAAGTGAAATTGACAAGTTGAAAGCAAA | 726 |
| | | | AATGTCCCAGTCTGCCGCCATGAACATTTGA | |
| | | | CTTCGGTCAAGATCGTGCCACAACGGCCCA | |
| | | | TCTCAGATACTGCGCAGCAGAAGAAGGAAC | |
| 1 | ADE1 | 3.47 | TCGTATCTCTGCATATGACGTTATTATGGAA | 727 |
| | | | AACAGCATTCCTGAAAAGGCTGGTTCAAGT | |
| | | | TCCTGTCCAACGATGTTCGTAATCATTTGGT | |
| | | | CGACATCGGGATCCTATTGACCAAACTGT | |
| 1 | ADE1 | 3.31 | GAACAAGGTGAACATGACGAAAACATCTCT | 728 |
| | | | CCTGCCCAGGCCGCTGAGCTGCAGAACTGG | |
| | | | CTGTAAAACTGTACTCCAAGTGCAAAGATT | |
| | | | ATGCTAAGGAGGTGGGTGAAGATTTGTCACG | |
| 1 | ADE1 | 3.29 | AGAAGACCGCTCTCTATTGGTTCACAAACAT | 729 |
| | | | AAACTAATTCCATTGGAAGTGCTTGGAAAG | |
| | | | AGTACGTAAAAACAGGTACTGTGCATGGTT | |
| | | | TGAAACAACTAATTGTCAGAGGCTACATCA | |
| 1 | ADE1 | 3.2 | ACGTTGCTGTTTGTTGCTACGGATCGTATCT | 730 |
| | | | CTGCATATGACGTTATTATCTATTGACCAAA | |
| | | | CTGTCAGAGTTCTGGTTCAAGTTCCTGTCCA | |
| | | | ACGATGTGGAAAACAGCATTCCTGAAAA | |
| 2 | AIM2 | 3.38 | ATTTCAATCAAATGGCATCTAATCAACCTGG | 731 |
| | | | CAAGTGTTGCTTTGAAGGAGTCGTGAAGAA | |
| | | | ATCTTCGGTTTAGATACTTATGCAGCAGGCT | |
| | | | CTACATCTGTTTGTCACGATGGAACACCC | |
| 2 | AIM2 | 3.36 | TGGTGACTTCAGGAGAATGTCTTTGAAACC | 732 |
| | | | AGGCATCACGATCAATTGGTAAATATCGGG | |
| | | | AACAAAGACCATGTACCCAGCACTAGCAAA | |
| | | | TTTGTCGGCCTTGTCCGATGAGATAGCATCG | |
| 2 | AIM2 | 3.21 | CATCTTTCGTCAGCATCGAGGAAATTGAAG | 733 |
| | | | CAATTGATAGCAAGAAACCAACATCTTTCC | |
| | | | GGCAAACTTAAGACACTTAACGGAGGAAAA | |
| | | | ATTAAAGGATATATTGATTTCAGCAGCGGAA | |
| 2 | AIM2 | 3.17 | ATCGGACAAACCAATTGATCGTGATGCCTG | 734 |
| | | | GTTTCAAAGACATTCTCCTGCATGAAGTTGT | |
| | | | TAAAACTTGAATATGACCCAAAGTTTATTGG | |
| | | | CGTTGTGGAAGTCACCAAGAAAATTGTTG | |
| 3 | ATS1 | 3.72 | CAGGAGATGATGGAGCAATAGTCAGGAAGA | 735 |
| | | | TAGCGTGCGGTGGGAACCACTGGTAGGATG | |
| | | | TGGAGATAACAGACGGGGAGAACTGGATAG | |
| | | | TGCGCAAGCAAGCGTGATGCTGACAAATGAC | |
| 3 | ATS1 | 3.63 | TTGTGGATGCTGATGGCCGTGTATGGCAGA | 736 |
| | | | GAGGAGGCGGTTGCTACGAGCCAACGATGA | |
| | | | GCGCATCGCAGTATACGGATGTTTCCAGAA | |
| | | | CTTTGTGGTGTTCACTCAGCAACATGTGCCA | |
| 3 | ATS1 | 3.55 | CCTTGCCCATGGCCACGTAGTCTACGGCCAC | 737 |
| | | | AGACCCGGTATCGTACACCTGGGCTCTTGCA | |
| | | | ATTGACA CTTTGTGTTGCTGC CC CAGCCGTA | |
| | | | TACTCGGAATACGGGCTCTTTCAGTGAT | |
| 3 | ATS1 | 3.54 | TGATAATGCAGGCAGATCCAAAGCGCAAGT | 738 |
| | | | GATGGAATGTGATCATTAAAGTGTGTATGC | |
| | | | GTTTGGGTCTAATGGGCAAAGGCAACTGGG | |
| | | | ACTGGGGCACGGCTATAACAGGCTTGTATCG | |
| 4 | BDH1 | 3.67 | TTGTCACTTTAGGACCAACCTTGGAAACAAT | 739 |
| | | | TCCTGACATCTCATGGCCCATTTATGGCACT | |
| | | | CTCCATCTTTAGGCATGAAGATTGGACCATC | |
| | | | CAAGTACATTGCCAGAGGTAAAGCAGCG | |
| 4 | BDH1 | 3.64 | TCCAAGTACTCGTGAAGATCCGAGCCACAA | 740 |
| | | | ATCCCACACCAAGAGACGTCTCTGGCCTAG | |
| | | | GGATATCATTAGTGAAGTGAATATCACCCTT | |
| | | | CTTGAAATAGATAATAACCTCATCGTCGGT | |
| 4 | BDH1 | 3.41 | TGAGGTGTTCAATCCCTCCAAGCACGGTCAT | 741 |
| | | | AAATCTATAGAGATACTACTGATTACAGTTA | |
| | | | TGATTGTTCTGGTATTCAAGTTACTTTCGAA | |
| | | | ACCTCTTGTGGTTTGACCAAGAGCCATG | |
| 4 | BDH1 | 3.22 | ATATCCCTAGGCCAGAAATCCAAACCGACG | 742 |
| | | | ATGAGGTTATTATCGACGTCTTCACGAGTAC | |
| | | | TTGGATGGTCCAATCTTCATGCCTAAAGATG | |
| | | | GAGAGTGCTCTTGGTGTGGGATTTGTGGC | |

Construction of the Plasmid Libraries.

10 ng oligonucleotide pool was used as template for PCR amplification with the corresponding primers (Table 14). 15 ng gel purified PCR products were assembled with 50 ng p426*-LbSgH, p426*-SpSgH, and p426*-SaSgH, respectively, using Golden Gate Assembly method (Bao, Z., et al., Nat. Biotechnol. 36:505-508, (2018); Bao, Z., et al., ACS Synth. Biol. 4:585-594, (2015)). The reaction mixture was transformed into NEB Turbo competent cells (New England Biolabs), yielding at least 5*10⁶ independent clones for each library, with ˜100-fold redundancy (Table 20). Each library was plated onto 25 LB/Amp agar plates and all the bacteria were collected to extract plasmids with a Qiagen Plasmid Maxi Kit.

Construction of the MAGIC Libraries.

The yeast mutant libraries were constructed by transforming 10 μg CRISPRa, 10 μg CRISPRi, and 20 μg CRISPRd plasmid libraries, respectively, into 10 OD₆₀₀ unit of CRISPR-AID strains using the LiAc/SS carrier DNA/PEG method (Gietz, R. D. & Schiestl, R. H., Nat. Protoc. 2:31-34, (2007)) with minor modification. After heat shock at 42° C. for 1 h, cells were resuspended in 4 mL YPD medium and recovered at 30° C. for ˜4h, which were then diluted 1000-fold and spread into SED-URA agar plates to evaluate the transformation efficiency. The remaining cells were cultured 50 mL SED-URA/G418 medium for ˜2 days. The independent clones for each library should be >10⁶, with at least 30-fold redundancy. The MAGIC libraries were constructed by pooling 1 OD unit cells from each library, which would be subject to growth enrichment under stressed conditions or high throughput screening.

Next Generation Sequencing.

NGS adapters were added to the extracted plasmid libraries using the Nextera Index Kit (Illumina, San Diego, Calif.) with a two-step PCR approach. The first step PCR added the Illumina overhang adapter sequences to all guide sequences (Table 23) using primers AID-NGS-F1 and AID-NGS-R1.

TABLE 23
NGS sequencing cassettes for CRISPRa, CRISPRi, and CRISPRd
libraries

| | Sequences (5′ to 3′) |
|---|---|
| LibA | TCGTCGGCAGCGTCAGATGTGTATAAGAGACAGcttctccgcagtgaaagataaatgatcAA |
| | TTTCTACTAAGTGTAGATNNNNNNNNNNNNNNNNNNNNNNNtttttttgttttttatgtct |
| | gagctccctgcaggcatgcaagcttggcgtaatcatggtcatagctgtttcctgtgtgaaattgttatccgctcacaattccacaca |
| | acatacgagccggaagcataaagtgtaaagcctggggtgcctaatgagtgagctaactcacattaattgcgttgcgctcactgc |
| | ccgctaccagtcgggaaacctgtcgtgccagctgcattaatgaatcggccaacgcgcggggagaggcggtttgcgtattggg |
| | cgctcttccgcttcctcgctcactgactcgctgcgctcggtcgttcggctgcggcgagcggtatcagctcactcaaagCTGT |
| | CTCTTATACACATCTCCGAGCCCACGAGAC (SEQ ID NO: 567) |
| LibI | TCGTCGGCAGCGTCAGATGTGTATAAGAGACAGcttctccgcagtgaaagataaatgatcNN |
| | NNNNNNNNNNNNNNNNNNGTTTTAGAGCTAGAAATAGCAAGTTAAAATAA |
| | GGCTAGTCCGTTATCAACTTGAAAAAGTGGCACCGAGTCGGTGCTTTTTTGA |
| | TCCtttttttgttttttatgtctgagctccctgcaggcatgcaagcttggcgtaatcatggtcatagctgtttcctgtgtgaaattgtta |
| | tccgctcacaattccacacaacatacgagccggaagcataaagtgtaaagcctggggtgcctaatgagtgagctaactcacatt |
| | aattgcgttgcgctcactgcccgctaccagtcgggaaacctgtcgtgccagctgcattaatgaatcggccaacgcgcggggag |
| | aggcggtttgcgtattgggcgctcttccgcttcctcgctcactgactcgctgcgctcggtcgttcggctgcggcgagcggtatca |
| | gctcactcaaagCTGTCTCTTATACACATCTCCGAGCCCACGAGAC (SEQ ID NO: 568) |
| LibD | TCGTCGGCAGCGTCAGATGTGTATAAGAGACAGcttctccgcagtgaaagataaatgatcNN |
| | NNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNN |
| | NNNNNNNNNNNNNNNNNNNNNGTTTTAGTACTCTGTAATTTTAGGTATGAG |
| | GTAGACGAAAATTGTACTTATACCTAAAATTACAGAATCTACTAAAACAAGG |
| | CAAAATGCCGTGTTTATCTCGTCAACTTGTTGGCGAGATTTTTTTGATCCtttttttg |
| | tttttttatgtctgagctccctgcaggcatgcaagcttggcgtaatcatggtcatagctgtttcctgtgtgaaattgttatccgctca |
| | caattccacacaacatacgagccggaagcataaagtgtaaagcctggggtgcctaatgagtgagctaactcacattaattgcgttgcg |
| | ctcactgcccgattccagtcgggaaacctgtcgtgccagctgcattaatgaatcggccaacgcgcggggagaggcggtttgc |
| | gtattgggcgctcttccgcttcctcgctcactgactcgctgcgctcggtcgttcggctgcggcgagcggtatcagctcactcaaa |
| | gCTGTCTCTTATACACATCTCCGAGCCCACGAGAC (SEQ ID NO: 569) |

The 3′-end of SNR52 promoter sequences, SUP4 terminator sequences, and part of the vector sequences are shown in lower case, the gRNA structural sequences are capitalized, the guide sequences are represented as N, and the Illumina overhang adapter sequences were underlined. The 43 bp region extracted from the NGS data for mapping into the reference sequences are shown in bold in Table 23.

The second step PCR attached Nextera indexes to each library, and the resultant products were gel purified and quantitated with Qubit (ThermoFisher). ˜60 ng of each library was pooled, followed by quantitation by qPCR and sequencing on one lane for 161 cycles from one end of the fragments on a HiSeq 2500 using a HiSeq SBS Sequencing Kit Version 4 (Illumina).

NGS Data Processing and Analysis.

Fastq files were generated and demultiplexed with the bcl2fastq v2.17.1.14 Conversion Software (Illumina). A bowtie index was prepared for all the designed 100,493 guide sequences and used as the reference sequences. An exemplary bowtie index for the guide sequences for the top-6 activation guide sequences, the top-6 interference guide sequences, and top-4 deletion guide sequences for the ACS1, ADE1, AIM2, ATS1, and BDH1 genes is shown in Table 24. The full list of 100,493 polynucleotide guide sequences is not shown for brevity.

TABLE 24
Exemplary bowtie index sequences

| Library gene | | SEQ ID |
|---|---|---|
| name | Guide sequence | NO: |
| 1_a_0ACS1 | AATTTCTACTAAGTGTAGATCCACGGCATGTCAACAGGTGAGT | 582 |
| 2_a_0ACS1 | AATTTCTACTAAGTGTAGATCCACCGAGGAACTGTACCCCAAC | 583 |
| 3_a_0ACS1 | AATTTCTACTAAGTGTAGATCTTTGGATCTTAGAGATAACAGA | 584 |
| 4_a_0ACS1 | AATTTCTACTAAGTGTAGATTAGGGGATGGAGAGTGCTACGCC | 585 |
| 5_a_0ACS1 | AATTTCTACTAAGTGTAGATCACAGCCGTACATACACGTGCCA | 586 |
| 6_a_0ACS1 | AATTTCTACTAAGTGTAGATTATACAAAATGAAGGGAGAACTA | 587 |
| 7_a_1ADE1 | AATTTCTACTAAGTGTAGATGAGTATGGCTACATGGATCAAGT | 588 |
| 8_a_1ADE1 | AATTTCTACTAAGTGTAGATCTGAAGGTTGAAAAAGAATGCCA | 589 |
| 9_a_1ADE1 | AATTTCTACTAAGTGTAGATAACCTTCAGGAAAAGTTTCAGAT | 590 |
| 10_a_1ADE1 | AATTTCTACTAAGTGTAGATTTTACAGCACTTGATCCATGTAG | 591 |
| 11_a_1ADE1 | AATTTCTACTAAGTGTAGATTGCTTTGCTATCGTGTAGAACTG | 592 |
| 12_a_1ADE1 | AATTTCTACTAAGTGTAGATAGATGAGTTGAAATTTCGAGTAT | 593 |
| 13_a_2AIM2 | AATTTCTACTAAGTGTAGATGGTCCACTGTTGGATTCGTAGCA | 594 |
| 14_a_2AIM2 | AATTTCTACTAAGTGTAGATATTAACGTAAAGGAACATAGTGC | 595 |
| 15_a_2AIM2 | AATTTCTACTAAGTGTAGATGCTGCTGTTTCTTCTGGCAATCC | 596 |
| 16_a_2AIM2 | AATTTCTACTAAGTGTAGATTGCCAGGATCAAGAGCAGCTTCT | 597 |
| 17_a_2AIM2 | AATTTCTACTAAGTGTAGATTATGATATCTGGCCTAAGGCGGA | 598 |
| 18_a_2AIM2 | AATTTCTACTAAGTGTAGATTCTGTAGTCGACATCTTTTGCTG | 599 |
| 19_a_3ATS1 | AATTTCTACTAAGTGTAGATCGTTCCTTACTGTAGATAGTCGG | 600 |
| 20_a_3ATS1 | AATTTCTACTAAGTGTAGATTTGCTACTGGTGGACACCCGACT | 601 |
| 21_a_3ATS1 | AATTTCTACTAAGTGTAGATAGGGAGACGACGATGCTACCTTG | 602 |
| 22_a_3ATS1 | AATTTCTACTAAGTGTAGATAGTTACGTGTTGCATTGCGAGAT | 603 |
| 23_a_3ATS1 | AATTTCTACTAAGTGTAGATTCTTGTTTACGTTCCTTACTGTA | 604 |
| 24_a_3ATS1 | AATTTCTACTAAGTGTAGATTAGGATTAAAAGAGATCATGAGC | 605 |
| 25_a_4BDH1 | AATTTCTACTAAGTGTAGATCTATCCTTGCCTATTCTTTCCTC | 606 |
| 26_a_4BDH1 | AATTTCTACTAAGTGTAGATGACGGAGAGAAGAAACCGGTGTT | 607 |
| 27_a_4BDH1 | AATTTCTACTAAGTGTAGATCTCCTTACGGGGTCCTAGCCTGT | 608 |
| 28_a_4BDH1 | AATTTCTACTAAGTGTAGATACATCAAGCCGGATTTGCTCACG | 609 |
| 29_a_4BDH1 | AATTTCTACTAAGTGTAGATTCGAGCCAATCGAGGGCAGCAGT | 610 |
| 30_a_4BDH1 | AATTTCTACTAAGTGTAGATTCTTGATATGATAATAGGTGGAA | 611 |
| 1_i_0ACS1 | CGTACTACCAGATAACCTAAGTTTTAGAGCTAGAAATAGCAAGT | 612 |
| 2_i_0ACS1 | GTTGGGGTACAGTTCCTCGGGTTTTAGAGCTAGAAATAGCAAGT | 613 |
| 3_i_0ACS1 | GGGAGAACTATTTGCCACCGGTTTTAGAGCTAGAAATAGCAAGT | 614 |
| 4_i_0ACS1 | TACCCATTGAATAATGGCATGTTTTAGAGCTAGAAATAGCAAGT | 615 |
| 5_i_0ACS1 | CAGTTTATATACAAAATGAAGTTTTAGAGCTAGAAATAGCAAGT | 616 |
| 6_i_0ACS1 | GTCCAAGTGTGGAGAATAGTGTTTTAGAGCTAGAAATAGCAAGT | 617 |
| 7_i_1ADE1 | CCAGATTCTTTGAGGTAAGAGTTTTAGAGCTAGAAATAGCAAGT | 618 |
| 8_i_1ADE1 | TCTGACTCTTGCGAGAGATGGTTTTAGAGCTAGAAATAGCAAGT | 619 |
| 9_i_1ADE1 | GTATGTCTATATGTATTAGAGTTTTAGAGCTAGAAATAGCAAGT | 620 |
| 10_i_1ADE1 | ACTTTACCTCTGGCCACCAAGTTTTAGAGCTAGAAATAGCAAGT | 621 |
| 11_i_1ADE1 | ACTCTGACAGTTTGGTCAATGTTTTAGAGCTAGAAATAGCAAGT | 622 |
| 12_i_1ADE1 | GATTACGAACATCGTTGGACGTTTTAGAGCTAGAAATAGCAAGT | 623 |
| 13_i_2AIM2 | CTATGATATCTGGCCTAAGGGTTTTAGAGCTAGAAATAGCAAGT | 624 |
| 14_i_2AIM2 | TCTGCTGTAGTTAGACGTAGGTTTTAGAGCTAGAAATAGCAAGT | 625 |
| 15_i_2AIM2 | AGGTTTCTTGCAAATGAGCGGTTTTAGAGCTAGAAATAGCAAGT | 626 |
| 16_i_2AIM2 | ATTTCTTCACGACGACCCTTGTTTTAGAGCTAGAAATAGCAAGT | 627 |
| 17_i_2AIM2 | CCTTCAAAGCAACACTTGCCGTTTTAGAGCTAGAAATAGCAAGT | 628 |
| 18_i_2AIM2 | ATGCCCAAATTTCTATATTAGTTTTAGAGCTAGAAATAGCAAGT | 629 |
| 19_i_3ATS1 | TGAAAAATTTCGCGGCGACGGTTTTAGAGCTAGAAATAGCAAGT | 630 |
| 20_i_3ATS1 | CTGCATTATCAAGGCTCAAAGTTTTAGAGCTAGAAATAGCAAGT | 631 |
| 21_i_3ATS1 | ACATTCCATCACTTGCGCTTGTTTTAGAGCTAGAAATAGCAAGT | 632 |
| 22_i_3ATS1 | TTACGTGTTGCATTGCGAGAGTTTTAGAGCTAGAAATAGCAAGT | 633 |
| 23_i_3ATS1 | CATTTGTCAGCATCACGCTGGTTTTAGAGCTAGAAATAGCAAGT | 634 |
| 24_i_3ATS1 | TGATCATTAAAGGCTATAACGTTTTAGAGCTAGAAATAGCAAGT | 635 |
| 25_i_4BDH1 | GCAGATACTTCGTGTGACAAGTTTTAGAGCTAGAAATAGCAAGT | 636 |
| 26_i_4BDH1 | AAGGGCAACATCTGCCCAAAGTTTTAGAGCTAGAAATAGCAAGT | 637 |
| 27_i_4BDH1 | ATGGCCAATTCAAGCCCTTTGTTTTAGAGCTAGAAATAGCAAGT | 638 |
| 28_i_4BDH1 | CATATCAAGAGAAACAGGCTGTTTTAGAGCTAGAAATAGCAAGT | 639 |
| 29_i_4BDH1 | AAACAGGCTAGGACCCCGTAGTTTTAGAGCTAGAAATAGCAAGT | 640 |
| 30_i_4BDH1 | TCTCTTGATATGATAATAGGGTTTTAGAGCTAGAAATAGCAAGT | 641 |
| 1_d_0ACS1 | TGGGATGAACACCTTATCGAATGGCTTAGACCAGTTTAAAAATT | 642 |
| 2_d_0ACS1 | GATCGTGCCACAACGGCCCATCTCAGATAGACTGCAGCCCGCAA | 643 |
| 3_d_0ACS1 | GATGACAACTTTAGAGTCCCCATCGTTGATACGATCTCTCAAGG | 644 |
| 4_d_0ACS1 | GTCAAGTGAAATTGACAAGTTGAAAGCAAAAATGTCCCAGTCTG | 645 |
| 5_d_1ADE1 | TCGTATCTCTGCATATGACGTTATTATGGAAAACAGCATTCCTG | 646 |
| 6_d_1ADE1 | GAACAAGGTGAACATGACGAAAACATCTCTCCTGCCCAGGCCGC | 647 |
| 7_d_1ADE1 | AGAAGACCGCTCTCTATTGGTTCACAAACATAAACTAATTCCAT | 648 |
| 8_d_1ADE1 | ACGTTGCTGTTTGTTGCTACGGATCGTATCTCTGCATATGACGT | 649 |
| 9_d_2AIM2 | ATTTCAATCAAATGGCATCTAATCAACCTGGCAAGTGTTGCTTT | 650 |
| 10_d_2AIM2 | TGGTGACTTCAGGAGAATGTCTTTGAAACCAGGCATCACGATCA | 651 |
| 11_d_2AIM2 | CATCTTTCGTCAGCATCGAGGAAATTGAAGCAATTGATAGCAAG | 652 |
| 12_d_2AIM2 | ATCGGACAAACCAATTGATCGTGATGCCTGGTTTCAAAGACATT | 653 |
| 13_d_3ATS1 | CAGGAGATGATGGAGCAATAGTCAGGAAGATAGCGTGCGGTGGG | 654 |
| 14_d_3ATS1 | TTGTGGATGCTGATGGCCGTGTATGGCAGAGAGGAGGCGGTTGC | 655 |
| 15_d_3ATS1 | CCTTGCCCATGGCCACGTAGTCTACGGCCACAGACCCGGTATCG | 656 |
| 16_d_3ATS1 | TGATAATGCAGGCAGATCCAAAGCGCAAGTGATGGAATGTGATC | 657 |
| 17_d_4BDH1 | TTGTCACTTTAGGACCAACCTTGGAAACAATTCCTGACATCTCA | 658 |
| 18_d_4BDH1 | TCCAAGTACTCGTGAAGATCCGAGCCACAAATCCCACACCAAGA | 659 |
| 19_d_4BDH1 | TGAGGTGTTCAATCCCTCCAAGCACGGTCATAAATCTATAGAGA | 660 |
| 20_d_4BDH1 | ATATCCCTAGGCCAGAAATCCAAACCGACGATGAGGTTATTATC | 661 |

From this point on, all the sequence manipulations were performed using commands on Galaxy (usegalaxy.org). The reads of 43 bp between SNR52p and SUP4t that contains a unique sequence in all three AID libraries (Table 23) were extracted from the NGS data using FASTQ Trimmer by column (Galaxy Version 1.0.0). Extracted guide sequences were then mapped to the bowtie index using Map with Bowtie for Illumina (Galaxy Version 1.1.2) with the default settings. Unmapped reads were removed and reads mapped to each unique guide sequence were counted. The raw guide count sequence was then mapped to the original reference file and the number of reads for each guide sequences was obtained. The number of reads per guide in each library was normalized to the total read counts of that library. A threshold of one read in all six libraries (biological triplicates for untreated and furfural stressed libraries) and 5-fold enrichment (Normalized No. of guide in the furfural stressed library/Normalized No. of guide in the untreated library) for each replicate was set to keep a guide sequence. The targets with the highest average folds of enrichment were chosen for further verification.

Quantitative PCR Analysis.

Mid-log phase yeast cells were collected to extract total RNAs using the RNeasy Mini Kit (QIAGEN, Valencia, Calif., USA) following the manufacturer's instructions. 2 μg of the RNA samples were then reversed transcribed into cDNA using the Transcriptor First Strand cDNA Synthesis Kit using oligo-dT primer (Roche, Indianapolis, Ind., USA). The qPCR experiments were carried out using SYBR Green-based method using the Roche LightCycler 480 System.

Results

Transforming the plasmid libraries into S. cerevisiae (Lian, J., et al., Nat. Commun. 8:1688, (2017)) resulted in the construction of the MAGIC library (FIG. 22), which represents the most comprehensive and diversified library ever reported. The unique guide sequence in each plasmid serves as a genetic barcode for high throughput phenotyping by next generation sequencing (NGS). Genotype-phenotype relationships can be mapped by tracking the enrichment or depletion of guide sequences, and the synergistic interactions among gain-, reduction-, and loss-of-function mutations can be identified in an iterative and genome-wide manner.

The pooled oligonucleotides were amplified by PCR and cloned into the corresponding gRNA expression plasmids. The plasmid libraries were sequenced and it was found that ˜87% of the CRISPRa and CRISPRi libraries and ˜73% of the CRISPRd libraries had the correct guide sequences. Lower mapping ratio of the CRISPRd library should result from higher synthesis error rate for longer oligos. As a result, nearly all gRNAs and genes were covered in the CRISPRa and CRISPRi plasmid libraries, while there was at least one gRNA for ˜98% of the yeast genes in the CRISPRd library (Table 20). The coverage of the genome-scale CRISPR-AID libraries were significantly higher than the previously reported cDNA based genome-scale libraries (Si, T., et al., Nat. Commun. 8:15187, (2017)).

### Example 7: MAGIC to Identify Genetic Determinants of Furfural Tolerance

Also described herein is a multi-functional genome-wide CRISPR (MAGIC) system for high throughput genotype-phenotype mapping. To determine if MAGIC could be used to identify genetic determinants of complex phenotypes, such as furfural tolerance, the MAGIC library was screened in the presence of 5 mM furfural and many enriched guide sequences were observed as compared to that under the reference conditions.

MAGIC Screening of Furfural Tolerance.

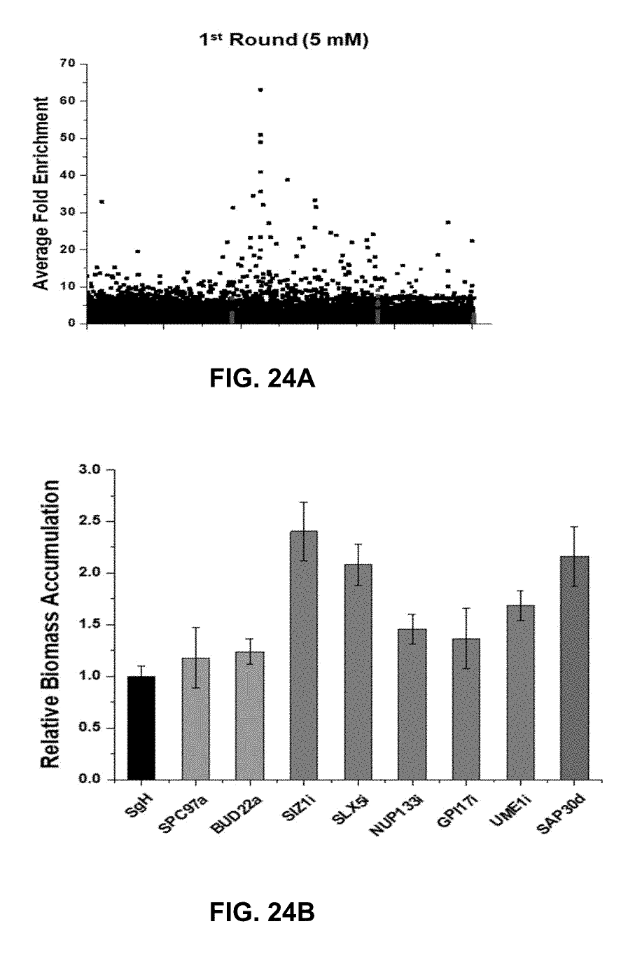

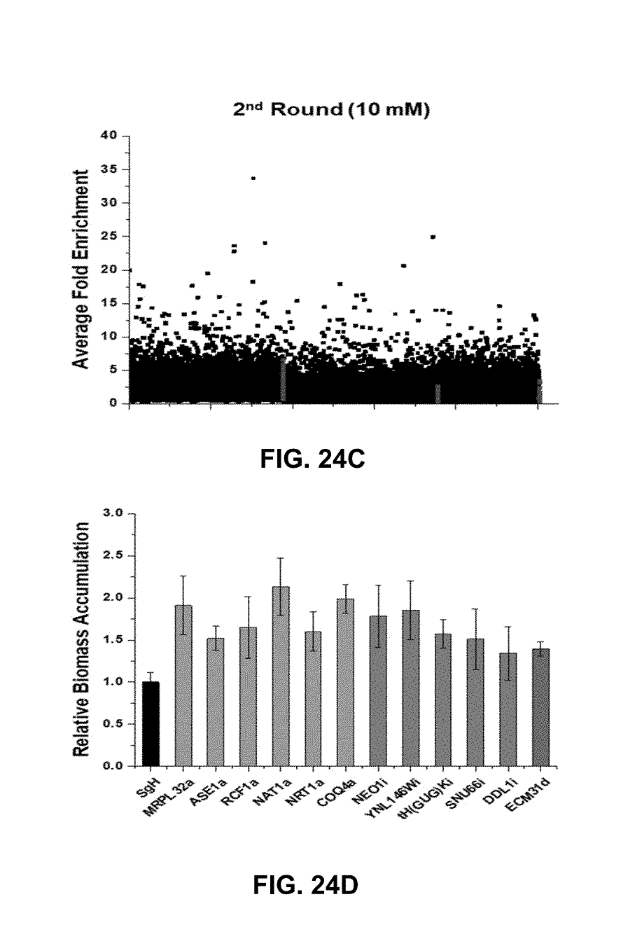

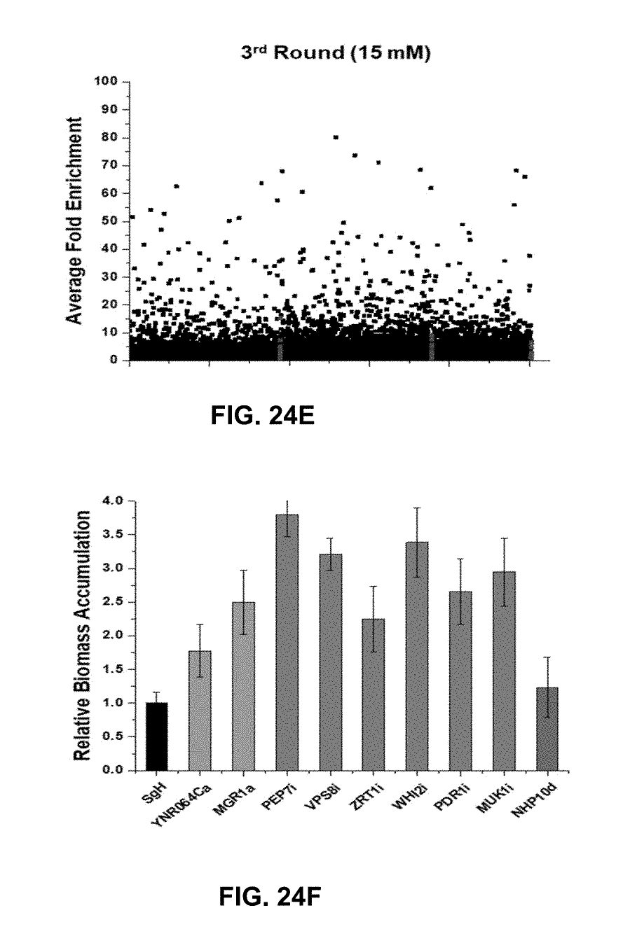

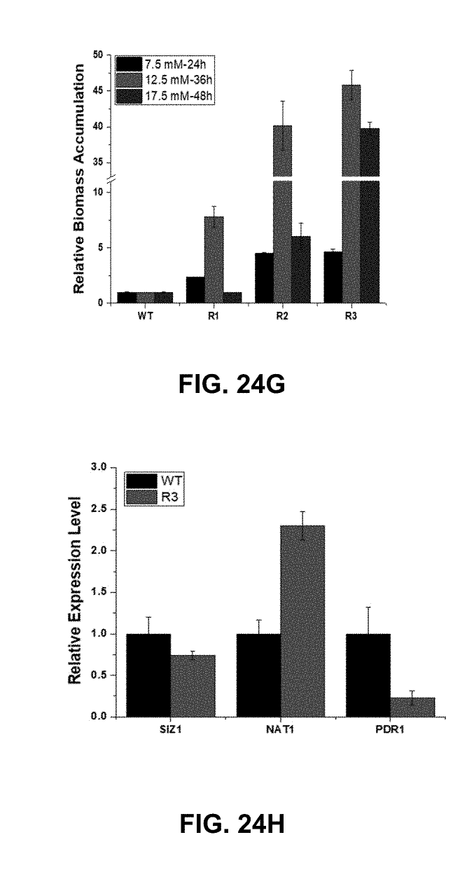

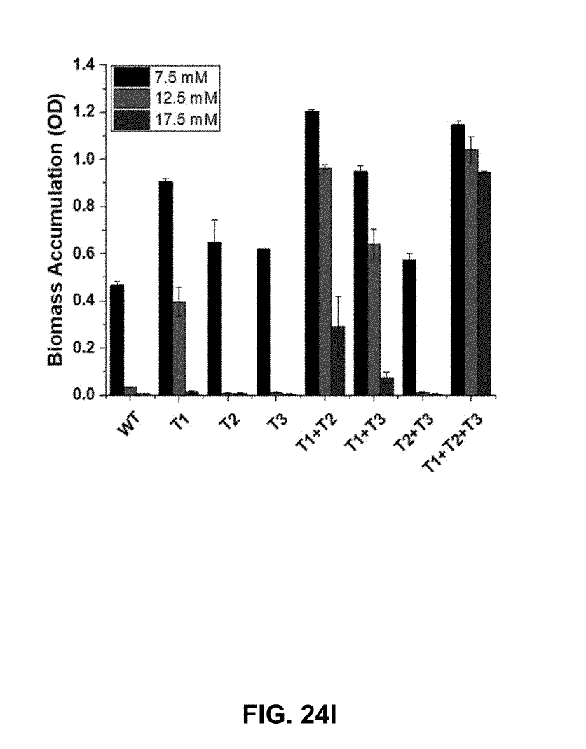

The MAGIC libraries in triplicates were inoculated into 50 mL SED-URA/G418 medium with or without furfural in a 250 mL baffled flask. 1 OD of the mid-log phase growing cells from each of the untreated and stressed libraries was collected and the plasmids were extracted for NGS analysis. 5 mM, 10 mM, and 15 mM furfural were used for the first, second, and third round of MAGIC screening, respectively. Single (T1, T2, and T3), double (T1+T2, T1+T3, and T2+T3), and triple (T1+T2+T3) mutants were constructed to investigate the synergistic interactions among SIZ1i, NAT1a, and/or PDR1i for enhanced tolerance against different concentrations (7.5, 12.5, and 17.5 mM) of furfural. Due to the lower metabolic burdens than the plasmid bearing strains, the integrated strains (i.e. R1, R2, and R3) were evaluated with a furfural concentration of 7.5 mM, 12.5 mM, and 17.5 mM, respectively (FIGS. 24A-24I).

Fermentation and HPLC Analysis.

A single colony of WT and R3 were inoculated into 3 mL SED/G418 medium and cultured until saturation, which was then transferred into 50 mL fresh SED/G418 medium with or without the supplementation of 17.5 mM furfural in a 250 mL un-baffled shaker flask with an initial OD of 0.05. Fermentation was performed under oxygen-limited conditions (30° C. and 100 rpm), and samples were taken every 24h and analyzed by HPLC. Cell growth was determined by measuring the absorbance at 600 nm using a Tecan Infinite M1000 PRO microplate reader (Tecan Trading AG, Switzerland). Glucose, ethanol, furfural, and furfuryl alcohol were quantified using a Shimadzu HPLC (Columbia, Md.) equipped with an Aminex HPX-87H column (Bio-Rad) and Shimadzu RID-10A refractive index detector. The column was kept at 65° C. with 0.5 mM sulfuric acid solution at a flow rate of 0.6 ml/min as the mobile phase.

Results

The MAGIC library was subject to iterative rounds of screening under gradually increased furfural concentration, 5 mM, 10 mM, and 15 mM for the first (FIG. 24A and FIG. 24B), second (FIG. 24C and FIG. 24D), and third (FIGS. 24E and 24F) round of MAGIC screening, respectively. The guide sequences of the enriched libraries were profiled (FIG. 24A, FIG. 24C, FIG. 24E) using next generation sequencing and the top hits were verified (FIG. 24B, FIG. 24D, FIG. 24F) under the corresponding screening condition. Notably, the control guide sequences were not enriched, indicating the association of the enriched guide sequences with furfural stress (FIG. 24A). Among those highly enriched guides, SIZ1i (refer to S/Z/interference) and SAP30d have been reported as furfural tolerance related targets via genome-wide screening in S. cerevisiae (Bao, Z., et al., Nat. Biotechnol. 36:505-508, (2018); Xiao, H. & Zhao, H., Biotechnol. Biofuels 7:78, (2014)), while SLX5i, NUP133i, GPI17i, and UME1i were newly identified targets (FIG. 24B). The identification of both known and novel genetic targets suggests the effectiveness and power of MAGIC for genome-wide profiling. Interestingly, SIZ1 and SLX5 are both involved in ubiquitin-mediated protein degradation; SAP30 and UME1 are both components of the RPD3L histone deacetylase complex (Table 25).

TABLE 25
Functional annotation of the MAGIC screening identified genetic
targets from SGD (Saccharomyces Genome Database, (yeastgenome.org).

| | AID | Function |
|---|---|---|
| SPC97 | A | Component of the microtubule-nucleating Tub4p (gamma-tubulin) complex; |
| | | interacts with Spc110p at the spindle pole body (SPB) inner plaque and with |
| | | Spc72p at the SPB outer plaque |
| BUD22 | A | Protein required for rRNA maturation and ribosomal subunit biogenesis; |
| | | required for 18S rRNA maturation; also required for small ribosomal |
| | | subunit biogenesis; cosediments with pre-ribosomal particles; mutation |
| | | decreases efficiency of +1 Ty1 frameshifting and transposition, and affects |
| | | budding pattern |
| SIZ1 | I | SUMO E3 ligase; promotes attachment of small ubiquitin-related modifier |
| | | sumo (Smt3p) to primarily cytoplasmic proteins; regulates Rsp5p ubiquitin |
| | | ligase activity and is in turn itself regulated by Rsp5p; required for |
| | | sumoylation of septins and histone H3 variant Cse4p, a prerequisite for |
| | | STUbL-mediated Ub-dependent degradation; localizes to the septin ring; |
| | | acts as an adapter between E2, Ubc9p and substrates; tends to compensate |
| | | for survival of DNA damage in absence of Nfi1p |
| SLX5 | I | Subunit of the Slx5-Slx8 SUMO-targeted Ub ligase (STUbL) complex; role |
| | | in Ub-mediated degradation of histone variant Cse4p preventing |
| | | mislocalization to euchromatin; role in proteolysis of spindle positioning |
| | | protein Kar9p, and DNA repair proteins Rad52p and Rad57p; forms SUMO- |
| | | dependent nuclear foci, including DNA repair centers; contains a RING |
| | | domain and two SIM motifs; associates with the centromere; required for |
| | | maintenance of genome integrity like human ortholog RNF4 |
| NUP133 | I | Subunit of Nup84p subcomplex of nuclear pore complex (NPC); contributes |
| | | to nucleocytoplasmic transport, NPC biogenesis; is involved in |
| | | establishment of a normal nucleocytoplasmic concentration gradient of |
| | | GTPase Gsp1p; also plays roles in several processes that may require |
| | | localization of genes or chromosomes at nuclear periphery, including |
| | | double-strand break repair, transcription and chromatin silencing; |
| | | relocalizes to cytosol in response to hypoxia; homolog of human NUP133 |
| GPI17 | I | Transmembrane protein; subunit of the glycosylphosphatidylinositol |
| | | transamidase complex that adds GPIs to newly synthesized proteins; human |
| | | PIG-S homolog |
| UME1 | I | Component of both the Rpd3S and Rpd3L histone deacetylase complexes; |
| | | negative regulator of meiosis; required for repression of a subset of meiotic |
| | | genes during vegetative growth, binding of histone deacetylase Rpd3p |
| | | required for activity, contains a NEE box and a WD repeat motif; |
| | | homologous with Wtm1p; UME1 has a paralog, WTM2, that arose from the |
| | | whole genome duplication |
| SAP30 | D | Component of Rpd3L histone deacetylase complex; involved in silencing at |
| | | telomeres, rDNA, and silent mating-type loci; involved in telomere |
| | | maintenance |
| MRPL32 | A | Mitochondrial ribosomal protein of the large subunit; protein abundance |
| | | increases in response to DNA replication stress |
| ASE1 | A | Mitotic spindle midzone-localized microtubule bundling protein; |
| | | microtubule-associated protein (MAP) family member; required for spindle |
| | | elongation and stabilization; undergoes cell cycle-regulated degradation by |
| | | anaphase promoting complex; potential Cdc28p substrate; relative |
| | | distribution to microtubules decreases upon DNA replication stress |
| RCF1 | A | Cytochrome c oxidase subunit; required for assembly of the Complex III- |
| | | Complex IV supercomplex, and for assembly of Cox13p and Rcf2p into |
| | | cytochrome c oxidase; similar to Rcf2p, and either Rcf1p or Rcf2p is |
| | | required for late-stage assembly of the Cox12p and Cox13p subunits and for |
| | | cytochrome c oxidase activity; required for growth under hypoxic |
| | | conditions; member of the hypoxia induced gene family; C. elegans and |
| | | human orthologs are functional in yeast |
| NAT1 | A | Subunit of protein N-terminal acetyltransferase NatA; NatA comprised of |
| | | Nat1p, Ard1p, and Nat5p; N-terminally acetylates many proteins to |
| | | influence multiple processes such as cell cycle progression, heat-shock |
| | | resistance, mating, sporulation, telomeric silencing and early stages of |
| | | mitophagy; orthologous to human NAA15; expression of both human |
| | | NAA10 and NAA15 functionally complements ard1 nat1 double mutant |
| | | although single mutations are not complemented by their orthologs |
| NRT1 | A | High-affinity nicotinamide riboside transporter; also transports thiamine |
| | | with low affinity; major transporter for 5-aminoimidazole-4-carboxamide-1- |
| | | beta-D-ribofuranoside (acadesine) uptake; shares sequence similarity with |
| | | Thi7p and Thi72p; proposed to be involved in 5-fluorocytosine sensitivity |
| COQ4 | A | Protein with a role in ubiquinone (Coenzyme Q) biosynthesis; possibly |
| | | functioning in stabilization of Coq7p; located on matrix face of |
| | | mitochondrial inner membrane; component of a mitochondrial ubiquinone- |
| | | synthesizing complex; human homolog COQ4 can complement yeast coq4 |
| | | null mutant |
| NEO1 | I | Phospholipid translocase (flippase), role in phospholipid asymmetry of |
| | | plasma membrane; involved in endocytosis, vacuolar biogenesis and Golgi |
| | | to ER vesicle-mediated transport; localizes to endosomes and the Golgi |
| | | apparatus |
| YNL146W | I | Putative protein of unknown function; green fluorescent protein (GFP)- |
| | | fusion protein localizes to the endoplasmic reticulum; YNL146W is not an |
| | | essential gene |
| tH(GUG)K | I | Histidine tRNA (tRNA-His) |
| SNU66 | I | Component of the U4/U6.U5 snRNP complex; involved in pre-mRNA |
| | | splicing via spliceosome; also required for pre-5S rRNA processing and |
| | | may act in concert with Rnh70p; has homology to human SART-1 |
| DDL1 | I | DDHD domain-containing phospholipase A1; mitochondrial matrix enzyme |
| | | with sn-1-specific activity, hydrolyzing cardiolipin, PE, PC, PG and PA; |
| | | implicated in remodeling of mitochondrial phospholipids; antagonistically |
| | | regulated by Aft1p and Aft2p; in humans, mutations in DDHD1 and |
| | | DDHD2 genes cause specific types of hereditary spastic paraplegia, while |
| | | DDL1-defective yeast share similar phenotypes such as mitochondrial |
| | | dysfunction and defects in lipid metabolism |
| ECM31 | D | Ketopantoate hydroxymethyltransferase; required for pantothenic acid |
| | | biosynthesis, converts 2-oxoisovalerate into 2-dehydropantoate |
| YNR064C | A | Epoxide hydrolase; member of the alpha/beta hydrolase fold family; may |
| | | have a role in detoxification of epoxides |
| MGR1 | A | Subunit of the mitochondrial (mt) i-AAA protease supercomplex; i-AAA |
| | | degrades misfolded mitochondrial proteins; forms a subcomplex with |
| | | Mgr3p that binds to substrates to facilitate proteolysis; required for growth |
| | | of cells lacking mtDNA |
| PEP7 | I | Adaptor protein involved in vesicle-mediated vacuolar protein sorting; |
| | | multivalent adaptor protein; facilitates vesicle-mediated vacuolar protein |
| | | sorting by ensuring high-fidelity vesicle docking and fusion, which are |
| | | essential for targeting of vesicles to the endosome; required for vacuole |
| | | inheritance |
| VPS8 | I | Membrane-binding component of the CORVET complex; involved in |
| | | endosomal vesicle tethering and fusion in the endosome to vacuole protein |
| | | targeting pathway; interacts with Vps21p; contains RING finger motif |
| ZRT1 | I | High-affinity zinc transporter of the plasma membrane; responsible for the |
| | | majority of zinc uptake; transcription is induced under low-zinc conditions |
| | | by the Zap1p transcription factor |
| WHI2 | I | Protein required for full activation of the general stress response; required |
| | | with binding partner Psr1p, possibly through Msn2p dephosphorylation; |
| | | regulates growth during the diauxic shift; negative regulator of G1 cyclin |
| | | expression; SWAT-GFP, seamless-GFP and mCherry fusion proteins |
| | | localize to the cell periphery |
| PDR1 | I | Transcription factor that regulates the pleiotropic drug response; zinc cluster |
| | | protein that is a master regulator involved in recruiting other zinc cluster |
| | | proteins to pleiotropic drug response elements (PDREs) to fine tune the |
| | | regulation of multidrug resistance genes; relocalizes to the cytosol in |
| | | response to hypoxia; PDR1 has a paralog, PDR3, that arose from the whole |
| | | genome duplication |
| MUK1 | I | Guanine nucleotide exchange factor (GEF); involved in vesicle-mediated |
| | | vacuolar transport, including Golgi-endosome trafficking and sorting |
| | | through the multivesicular body (MVB); specifically stimulates the intrinsic |
| | | guanine nucleotide exchange activity of Rab family members |
| | | (Vps21p/Ypt52p/Ypt53p); partially redundant with GEF VPS9; required for |
| | | localization of the CORVET complex to endosomes; contains a VPS9 |
| | | domain |
| NHP10 | D | Non-essential INO80 chromatin remodeling complex subunit; preferentially |
| | | binds DNA ends, protecting them from exonucleatic cleavage; deletion |
| | | affects telomere maintenance via recombination; related to mammalian high |
| | | mobility group proteins |

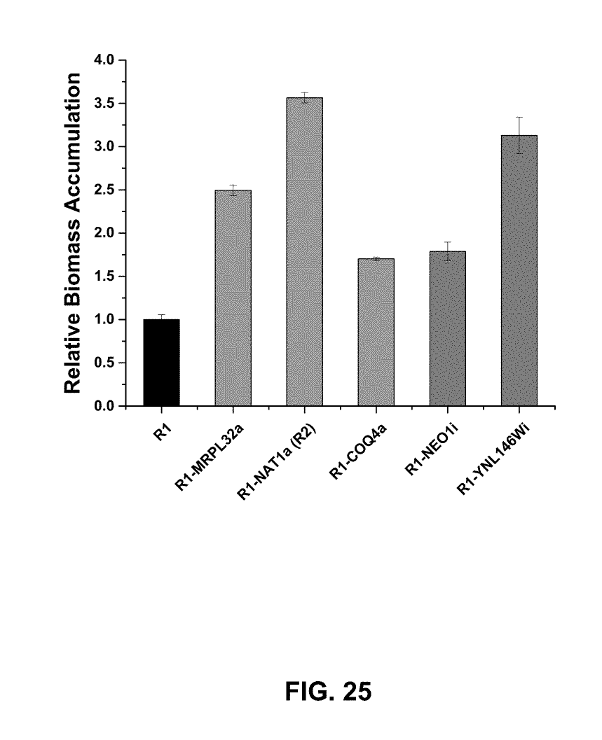

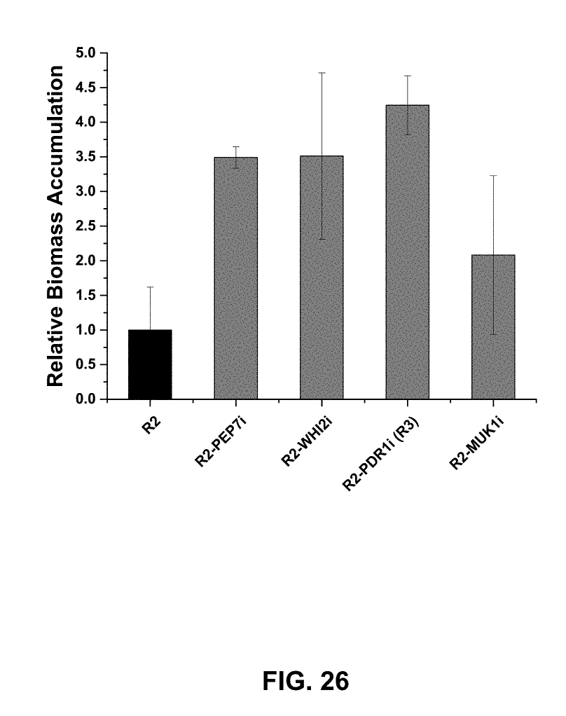

These results highlighted the roles of protein degradation and histone modification in furfural tolerance. As SIZ1i improved furfural tolerance the most, we constructed strain R1 by integrating the SIZ1i cassette into the X4 locus of the genome (Table 15). A second round of MAGIC screening was performed and enriched several new guide sequences, which could further increase the growth rate in the presence of 10 mM furfural (FIG. 24C). Interestingly, none of the targets have been ever reported to associate with furfural tolerance. Among those highly enriched guides, several targets related to mitochondrial functions, such as MRPL32, RCF1, COQ4, DDL1, and NAT1 were identified (Table 24). The supply of ATP should be beneficial to tackle furfural stress. Interestingly, the repression of an uncharacterized ORF (YNL146W) and two RNAs (SNU66 and a histidine tRNA) also improved furfural tolerance (FIG. 24D and FIG. 25). Then the NAT1a and SIZ1i integrated strain (R2) was used as the new parent strain for the third round of genome-wide screening, and continued to observe highly enriched guide sequences (FIG. 24E). PDR1i was the optimal hit to improve furfural tolerance when integrated into the chromosome together with SIZ1i and NAT1a (R3, FIG. 24F and FIG. 26). PDR1 is a transcriptional factor that negatively regulated the expression of pleiotropic drug resistance genes (i.e. PDR5) (Nishida-Aoki, N., et al., Curr. Genet. 61:153-164, (2015)). Thus, PDR1i could increase the expression of PDR5 to export furfural out of the cell, leading to improved furfural tolerance.

After 3 rounds of genome-scale engineering, not only were genetic determinants of furfural tolerance profiled, but also an engineered strain showing ready growth at high furfural concentrations was obtained. As shown in FIG. 24G, the engineered strains grew much faster than the control strain, with more significant effect observed at higher furfural concentrations. Quantitative PCR confirmed the desired genome modification, including the interference of SIZ1, activation of NAT1, and interference of PDR1 (FIG. 24H).

Finally, synergistic interactions among the genetic determinants identified in iterative rounds of MAGIC screening were identified. Using the engineered furfural tolerant strain R3 as an example, single (T1, T2, and T3), double (T1+T2, T1+T3, and T2+T3), and triple (T1+T2+T3) mutants were constructed and compared their tolerance against different concentrations of furfural. As shown in FIG. 24I, the 2^{nd} and 3^{rd} round hits, alone (T2 or T3) or in combination (T2+T3), marginally improved furfural tolerance in the reference strain. In other words, T2 and T3 only demonstrated furfural tolerant phenotypes when combined with T1, demonstrating a synergistic interaction between NAT1a and SIZ1i as well as PDR1i and SIZ1i. Notably, T1+T3 also endowed higher furfural tolerance than T1 and T3, particularly at high furfural concentrations. Therefore, there might be additive or synergistic effects between NAT1a and PDR1i in the SIZ1i background strain. These results highlighted the significance of iterative rounds of genome-wide screening in understanding and engineering of complex phenotypes.

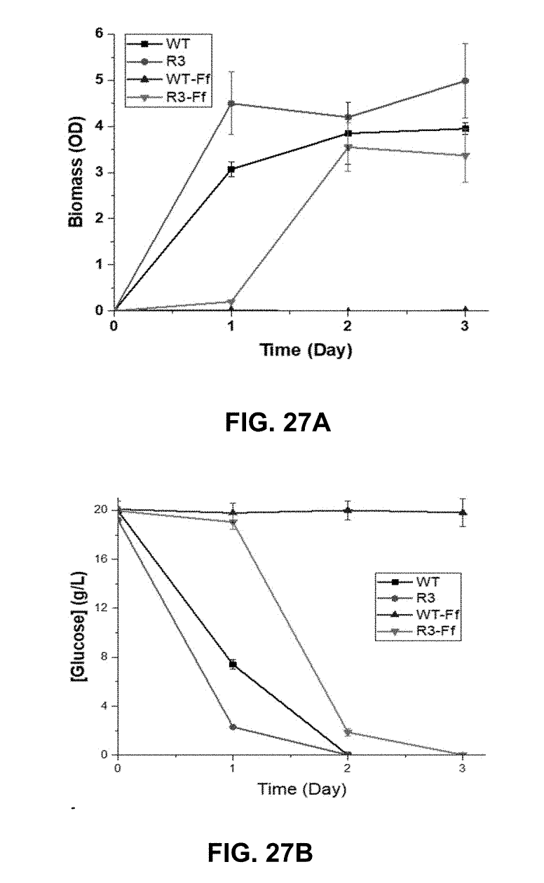

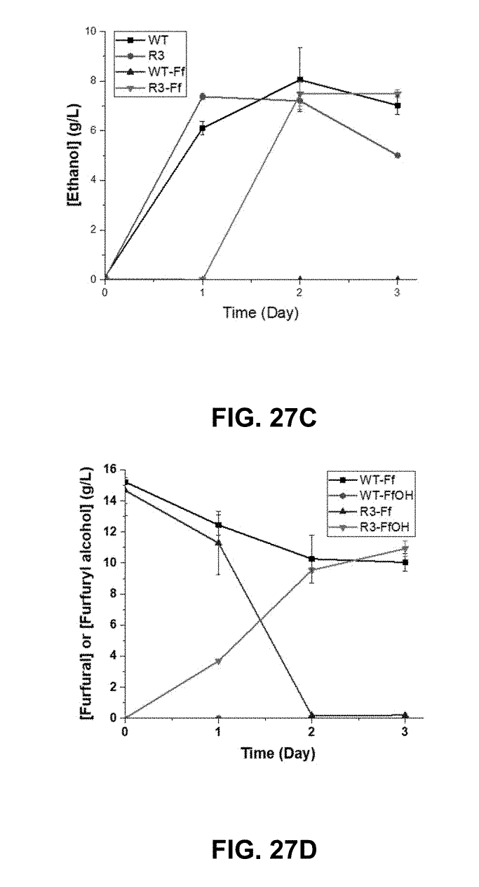

The fermentation performance of the wild-type (WT) and the engineered (R3) strain were also compared (FIG. 27A-27D). A single colony of WT and R3 were inoculated into 3 mL SED/G418 medium and cultured until saturation, which was then transferred into 50 mL fresh SED/G418 medium with or without the supplementation of 17.5 mM furfural in a 250 mL un-baffled shaker flask. In the absence of furfural, these strains showed comparable fermentation performance. On the contrary, when 17.5 mM furfural was supplemented, the control strain failed to grow after 6 days of culture, while R3 was able to consume most of glucose in 2 days. The decrease of furfural concentration in WT might result from evaporation, as no growth and furfuryl alcohol production were observed. More importantly, the final concentration of ethanol was comparable to the control strain under furfural-free conditions, indicating that the central metabolism of our engineered yeast strain was not significantly changed.

### Example 8: MAGIC to Identify Genetic Determinants of Yeast Surface Display of Recombinant Proteins

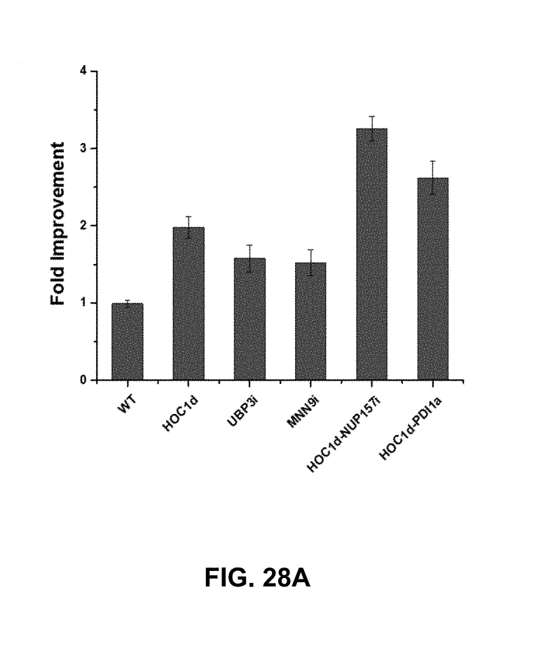

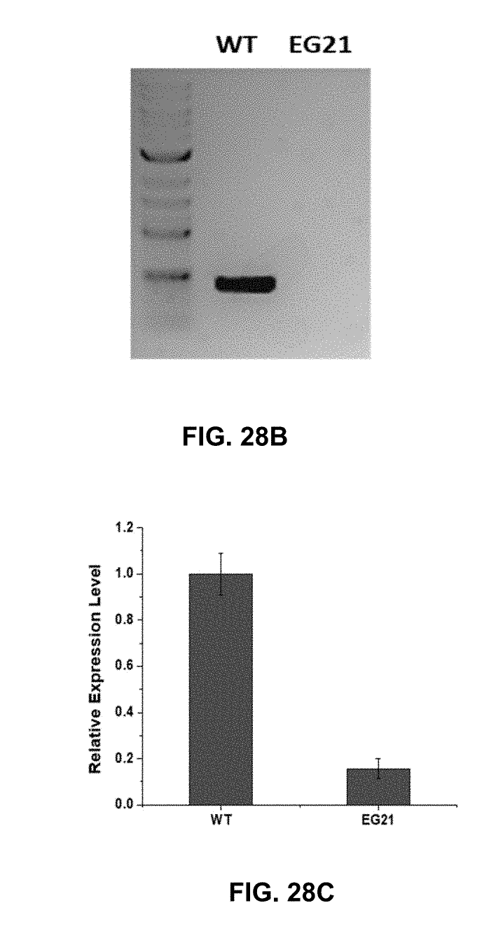

Besides furfural tolerance, the application of MAGIC for functional profiling of another complex phenotype, yeast surface display of recombinant proteins was also demonstrated (FIG. 28A-28C).

MAGIC Screening of Yeast Surface Display Mutants.

The MAGIC library was cultured at 30° C. for 2 days and then subject to immunostaining and fluorescence activated cell sorting (FACS), following a previously developed protocol (Lian, J., et al., Nat. Commun. 8:1688, (2017); Si, T., et al., Nat. Commun. 8:15187, (2017)). The primary and secondary antibodies were monoclonal mouse anti-histidine tag antibody (1:100 dilution, Bio-Rad, Raleigh, N.C., catalog # MCA1396GA) and goat anti-mouse IgG (H+L) secondary antibody, Biotin-XX conjugate (1:100 dilution, ThermoFisher Scientific, Rockford, Ill., catalog # B-2763), respectively. Streptavidin, R-phycoerythrin conjugate (1:100 dilution, ThermoFisher Scientific, catalog # S866) was used to quantify the amount of biotin on the yeast surface. BD FACS Aria III cell sorting system (BD Biosciences, San Jose, Calif.) was used for collecting the most fluorescent yeast mutants. In the first round of sorting, 30,000 cells representing the top 1% highest fluorescence were collected. The second round sorted 96 individual yeast cells with the highest fluorescence. Then the plasmids were extracted and retransformed into the bAID-EG strain, the resulting recombinant strains were further analyzed by the cellulase activity assay. Briefly, 400 μL yeast cells were washed twice with ddH₂O and resuspend in the same volume of 1% (w/v) carboxymethyl cellulose (CMC) solution (0.1 M sodium acetate, pH 5). After incubation at 30° C. for 16 h with vigorous shaking, the amount of reducing sugars in the supernatant was quantified by a modified DNS method (Lian, J., et al., Nat. Commun. 8:1688, (2017); Si, T., et al., Nat. Commun. 8:15187, (2017)). The gRNA plasmids enabling higher cellulase activity were sent for DNA sequencing.

Using the Trichoderma reesei endoglucanase (EGII) (Lian, J., et al., Nat. Commun. 8:1688, (2017); Si, T., et al., Nat. Commun. 8:15187, (2017)) as an example, HOCld was the highest enriched target to enhance protein secretion and surface display levels, followed by UBP3i and MNN9i. HOC1 and MNN9 are both subunits of the Golgi mannosyltransferase complex, the disruption of which minimized protein super-glycosylation and enhanced protein secretion (Tang, H., et al., Sci. Rep. 6:25654, (2016)) (FIG. 28A). UBP3 is thiol-dependent ubiquitin-specific protease and its downregulation should enable higher protein stability and abundance (Table 24). A second round of MAGIC screening identified NUP157i and PDI1a as the best targets (FIG. 28A). PDI1 (protein disulfide isomerase) is essential for disulfide bond formation in secretory proteins and its overexpression has been found to work synergistically with the downregulation of mannosyltransferase encoding genes (i.e. MNN9) (Lian, J., et al., Nat. Commun. 8:1688, (2017)), while the effect of NUP157i on protein secretion and display is less understood.

### Example 9: Comparison of MAGIC to Traditional Genome-Scale Engineering Strategies

Compared with the traditional genome-scale engineering strategies, such as cDNA overexpression libraries (Liu, H., et al., Genetics 132:665-673 (1992)) and knock out collections (Giaever, G., et al., Nature 418:387-391 (2002)), CRISPR based technology offers a more flexible alternative for constructing a genome-wide set of mutants under different strain backgrounds. Although there are prior CRISPR-enabled genome-scale engineering attempts, the genotypic diversity is only limited to the targets that share the same type of genomic alteration.

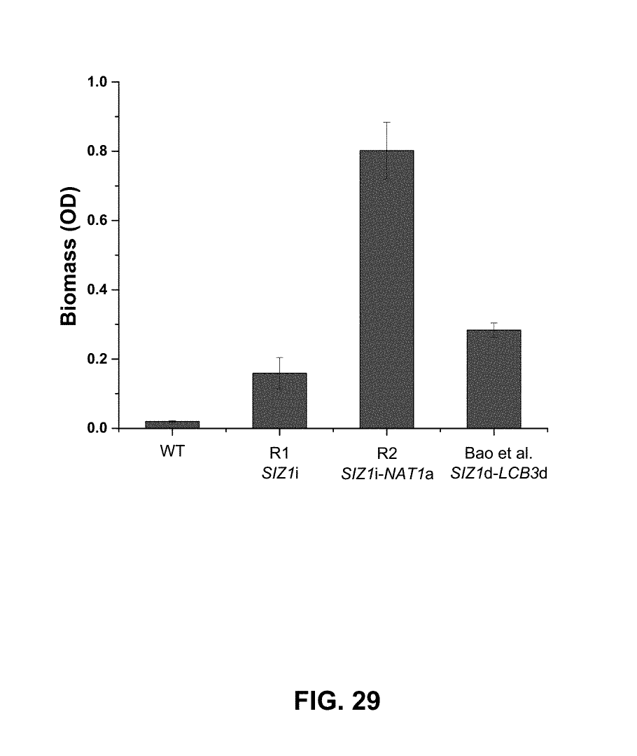

To address this limitation, MAGIC for mapping synergistic interactions among overexpression, repression, and deletion targets in a genome-wide manner in S. cerevisiae was developed. Taken the furfural tolerant phenotype for example, the genome-wide RNAi technology (RAGE) failed to identify new targets after one round screening with 5 mM furfural (Xiao, H. & Zhao, H., Biotechnol. Biofuels 7:78 (2014)), and another genome-scale CRISPRd system (CHAnGE) could not obtain enriched targets after two rounds of screening at 10 mM furfural (Bao, Z., et al., Nat. Biotechnol. 36:505-508 (2018)), while MAGIC continued to enrich novel genetic determinants even after 4 rounds of screening at 20 mM furfural (data not shown). In addition, although screened under the same conditions (10 mM furfural and two rounds of evolution), the MAGIC engineered strain (SIZ1i-NAT1a) performed much better than the CHAnGE modified strain (SIZ1d-LCB3d) (FIG. 29). In other words, MAGIC not only identified more genetic determinants of furfural tolerance, but also engineered more furfural tolerant strains. These results demonstrated the necessity of combinatorial optimization and the power of MAGIC. MAGIC can be adopted for genome-scale engineering of higher eukaryotic organisms. For example, several orthogonal CRISPR proteins have been functionally characterized (Esvelt, K. M., et al., Nat. Methods 10:1116-1121 (2013)) and genome-scale CRISPRa (Konermann, S., et al., Nature 517:583-588 (2015); Gilbert, L. A., et al., Cell 159:647-661, (2014)), CRISPRi (Gilbert, L. A., et al., Cell 159:647-661 (2014); Liu, S. J., et al., Science 355, (2017)), and CRISPRd (Shalem, O., et al., Science 343:84-87, (2014)) have been individually reported in mammalian cells.

Recently, cDNA overexpression and RNA interference (RNAi) was combined to achieve combinatorial genome-scale engineering of complex phenotypes in yeast (Lian, J., et al. Metab. Eng., (2018)). Both strategies enable the exploration of the gain- and loss-of-function combinations that work synergistically to improve the desired phenotypes. Nevertheless, MAGIC not only introduces a third mode of genome engineering (gene deletion), but also offers several advantages of the CRISPR system. Most importantly, MAGIC represents the most comprehensive library ever created, with an average of >99% coverage of all ORFs and RNA genes for genome-wide overexpression, repression, and deletion (Table 20); while the cDNA based library covers ˜92% of all ORFs (Lian, J., et al. Metab. Eng., (2018)), as not all genes will be expressed under a given condition and RNA genes will not be included. MAGIC is less biased than the cDNA library, as all the MAGIC cassettes have the same or similar size to minimize cloning and transformation bias. In addition, the regulation mechanisms are different, CRISPRi blocks transcription in the nucleus while RNAi affects mRNA stability and translation in the cytosol.

Thus, by combining the tri-functional CRISPR system and array-synthesized oligo pools, MAGIC was used to create the most diversified library and identify novel genetic determinants of complex phenotypes, particularly those with synergistic interactions when regulated to different expression levels. Overall, MAGIC represents a powerful and generally applicable strategy to investigate fundamental biological questions as well as engineer complex phenotypes for biotechnological applications in yeast and possibly higher eukaryotes.

### Example 10: Characterization of the Genomic Loci for SaCas9-Assisted and Marker-Less Integration of gRNA Expression Cassettes

Previously characterized integration loci (Mikkelsen, M. D. et al. Metab. Eng. 14:104-111, (2012)) were chosen, which were flanked by highly expressed essential genes to enable efficient and stable expression of heterologous genes and pathways. Ten gRNA plasmids based on SaCas9 were constructed to integrate heterologous cassettes into X2, X3, X4, XI1, XI2, XI3, XII1, XII2, XII4, and XII5 loci, respectively.

To characterize the integration and gRNA expression efficiency of the pre-selected genomic loci, the integration efficiency and gRNA expression level were evaluated by co-transforming the reporter strain (bAID-RV) with gRNA plasmid as well as its corresponding linear donor fragment, which contained a gRNA expression cassette to activate the expression of mCherry or to repress the expression of mVenus. The gRNA targeting efficiency was tested by transforming the gRNA plasmid without any donor to repair the double strand break, and efficient gRNA should result in no survived colonies.

Eight colonies were randomly picked up to measure the change in fluorescence intensities. The mVenus and mCherry fluorescence signals were measured at 514-528 nm and 587-610 nm, respectively, using a Tecan Infinite M1000 PRO multimode reader (Tecan Trading AG, Switzerland). The fluorescence intensity (relative fluorescence units; RFU) was normalized to cell density that was determined by measuring the absorbance at 600 nm using the same microplate reader. The higher activation or repression efficiency of the integrated gRNA than its plasmid counterpart might result from lower metabolic burdens.

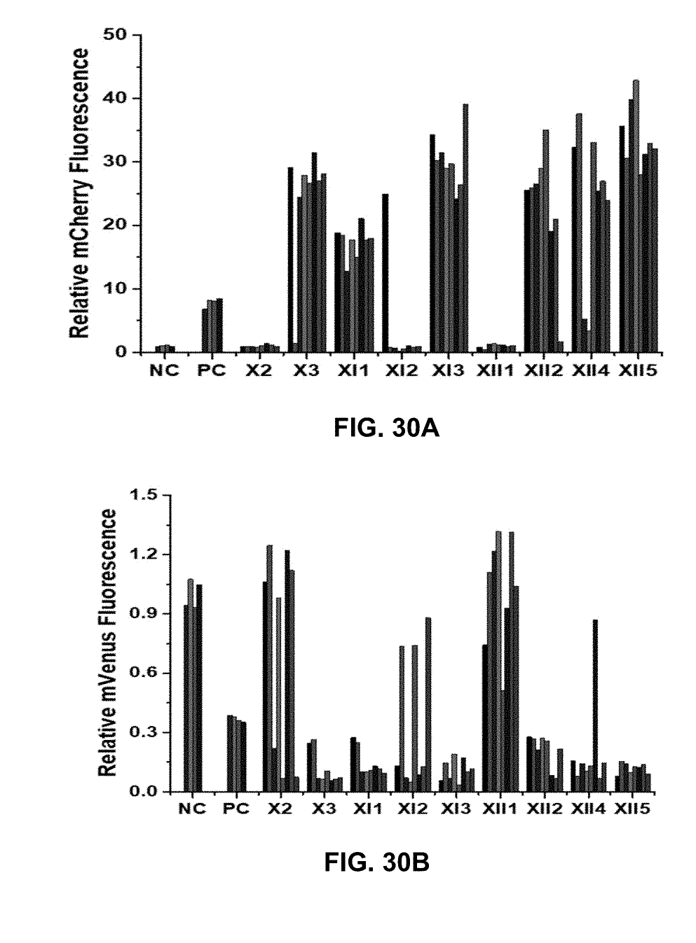

As shown in FIG. 30A-30B and Table 15, X3, X4, XI1, XI3, XII2, XII4, and XII5 together with their corresponding gRNAs were chosen for CRISPR-assisted and marker-less integration of gRNA expression cassettes.

We claim:

1. A system for targeted genome engineering, the system comprising one or more vectors comprising:
(i) a first single guide RNA (sgRNA) that is capable of binding a target nucleic acid and binding a first nuclease-deficient RNA-guided DNA endonuclease protein;
(ii) a second sgRNA that is capable of binding a target nucleic acid and binding a second nuclease-deficient RNA-guided DNA endonuclease protein;
(iii) a third sgRNA that is capable of binding a target nucleic acid and binding a catalytically-active RNA-guided DNA endonuclease protein;
(iv) a polynucleotide encoding a first nuclease-deficient RNA-guided DNA endonuclease protein that binds to the first sgRNA and causes transcriptional activation;
(v) a polynucleotide encoding a second nuclease-deficient RNA-guided DNA endonuclease protein that binds to the second sgRNA and causes transcriptional interference; and
(vi) a polynucleotide encoding a catalytically active RNA-guided DNA endonuclease protein that binds to the third sgRNA and causes a double-stranded nucleic acid break and causes gene deletion.

2. The system of claim 1, wherein components (i), (ii), (iiii), (iv), (v), and (vi) are located on the same or different vectors of the system.

3. The system of claim 1, wherein the catalytically active RNA-guided DNA endonuclease protein is CRISPR associated protein (Cas9).

4. The system of claim 3, wherein the Cas9 is a Cas9 from Streptococcus pyogenes (SpCas9), Neisseria meningitides (NmCas9), Streptococcus thermophiles (St1Cas9), or Staphylococcus aureus (SaCas9).

5. The system of claim 1, wherein the one or more vectors are plasmids or viral vectors.

6. The system of claim 1, wherein the first nuclease-deficient RNA-guided DNA endonuclease protein is functional only when bound to the first sgRNA.

7. The system of claim 1, wherein the second nuclease-deficient RNA-guided DNA endonuclease protein is functional only when bound to the second sgRNA.

8. The system of claim 1, wherein the catalytically active RNA-guided DNA endonuclease protein is functional only when bound to the third sgRNA.

9. The system of claim 1, wherein the system does not utilize synthetic CRISPR-repressible promoters or synthetic CRISPR-activatable promoters.

10. The system of claim 1, wherein all the sgRNAs are expressed in an expression cassette comprising a type II promoter or a type III promoter.

11. A polynucleotide comprising a nucleotide sequence encoding a Cpf1 nuclease-deficient RNA-guided DNA endonuclease protein operably linked to at least one VP64-p65AD (VP) activator domain.

12. The polynucleotide of claim 11, wherein the Cpf1 protein is from Lachnospiraceae bacterium or Acidaminococcus sp.

13. A polynucleotide comprising a nucleotide sequence encoding a Cas9 RNA-guided DNA endonuclease protein operably linked to more than one repression domain.

14. The polynucleotide of claim 13, wherein the Cas9 protein is from Streptococcus pyogenes, Neisseria meningitides, Streptococcus thermophiles, or Staphylococcus aureus.

15. The polynucleotide of claim 13, wherein the polynucleotide comprises a nucleotide sequence encoding a dSpCas9 protein operably linked to the C-terminal end to a RD11 repression domain, wherein a RD5 repression domain is operably linked to the C-terminal end of the RD11 domain, wherein a RD2 repression domain is operably linked to the C-terminal end of the RD5 domain.

16. The polynucleotide of claim 13, wherein the at least one repression domain is operably linked to the N-terminal and/or C-terminal ends of the nuclease-deficient RNA-guided DNA endonuclease protein, or operably linked in tandem at the C-terminal end of the nuclease-deficient RNA-guided DNA endonuclease protein.

17. A method of altering the expression of gene products, the method comprising:
introducing into a cell the system of claim 1,
wherein the expression of at least one gene product is increased, the expression of at least one gene product is decreased, and the expression of at least one gene product is deleted relative to a cell that has not been transformed with the system of claim 1.

18. The method of claim 17, wherein the method further comprises selecting for successfully transformed cells by applying selective pressure.

19. The method of claim 17, wherein the method occurs in vivo or in vitro.

20. The method of claim 17, wherein the cell is a eukaryotic cell.

21. The method of claim 24, wherein the cell is a yeast cell.

22. The method of claim 21, wherein the yeast cell is Saccharomyces cerevisiae.

23. The method of claim 17, further comprising increasing expression of a surface protein on the cell.

24. A method of identifying the genetic basis of one or more phenotypes of cells, the method comprising:
preparing three genome-scale sgRNA expressing plasmid libraries from oligonucleotides wherein the first genome-scale sgRNA expressing plasmid library is for upregulating genes of the cells, wherein the second genome-scale sgRNA expressing plasmid library is for downregulating genes of the cells, and the third genome-scale sgRNA expressing plasmid library is for deleting genes of the cells;
(ii) transforming the three genome-scale sgRNA expressing plasmid libraries into the cells;
(iii) introducing into the cells a polynucleotide encoding a first nuclease-deficient RNA-guided DNA endonuclease protein that binds to the sgRNA of the first genome-scale sgRNA expressing plasmid library and causes transcriptional activation of genes of the cells, a polynucleotide encoding a second nuclease-deficient RNA-guided DNA endonuclease protein that binds to the sgRNA of the second genome-scale sgRNA expressing plasmid library and causes transcriptional repression of genes of the cells, and a polynucleotide encoding a catalytically active RNA-guided DNA endonuclease protein that binds to a the sgRNA of the third genome-scale sgRNA expressing plasmid library and causes double-stranded nucleic acid breaks and gene deletion of genes of the cells;
(iv) isolating transformed cells with one or more phenotypes; and
(v) determining the genomic loci of the DNA molecule that causes the one or more phenotypes.

25. The method of claim 24, wherein the cell is a yeast cell.

26. The method of claim 24, wherein the cell is a eukaryotic cell.

27. The method of claim 24, wherein the phenotype is furfural tolerance or yeast surface display of recombinant proteins.


SEQUENCE LISTING (743 sequences)

SEQ ID NO: 1 - DNA, 20 bp, Artificial Sequence
  Synthetic oligonucleotide
ttgatatttaagttattaaa

SEQ ID NO: 2 - DNA, 20 bp, Artificial Sequence
  Synthetic oligonucleotide
actttagtgctgacacatac

SEQ ID NO: 3 - DNA, 20 bp, Artificial Sequence
  Synthetic oligonucleotide
gatatcaagaggattggaaa

SEQ ID NO: 4 - DNA, 20 bp, Artificial Sequence
  Synthetic oligonucleotide
acgtccctattgaatgttgg

SEQ ID NO: 5 - DNA, 20 bp, Artificial Sequence
  Synthetic oligonucleotide
aactctggacattataccat

SEQ ID NO: 6 - DNA, 20 bp, Artificial Sequence
  Synthetic oligonucleotide
aaaaatgggcaccatttact

SEQ ID NO: 7 - DNA, 20 bp, Artificial Sequence
  Synthetic oligonucleotide
ccaattgtagagactatcca

SEQ ID NO: 8 - DNA, 14 bp, Artificial Sequence
  Synthetic oligonucleotide
tttaagttattaaa

SEQ ID NO: 9 - DNA, 14 bp, Artificial Sequence
  Synthetic oligonucleotide
taaatatcaatggg

SEQ ID NO: 10 - DNA, 20 bp, Artificial Sequence
  Synthetic oligonucleotide
gaagctcatttgagatcaat

SEQ ID NO: 11 - DNA, 20 bp, Artificial Sequence
  Synthetic oligonucleotide
ggaagaggtaacttcgttgt

SEQ ID NO: 12 - DNA, 23 bp, Artificial Sequence
  Synthetic oligonucleotide
gcaagcatcaatggtataatgtc

SEQ ID NO: 13 - DNA, 24 bp, Artificial Sequence
  Synthetic oligonucleotide
gtaacttcgttgtaaagaataagg

SEQ ID NO: 14 - DNA, 14 bp, Artificial Sequence
  Synthetic oligonucleotide
gtgctgacacatac

SEQ ID NO: 15 - DNA, 18 bp, Artificial Sequence
  Synthetic oligonucleotide
gatatttaagttattaaa

SEQ ID NO: 16 - DNA, 16 bp, Artificial Sequence
  Synthetic oligonucleotide
tatttaagttattaaa

SEQ ID NO: 17 - DNA, 15 bp, Artificial Sequence
  Synthetic oligonucleotide
atttaagttattaaa

SEQ ID NO: 18 - DNA, 13 bp, Artificial Sequence
  Synthetic oligonucleotide
ttaagttattaaa

SEQ ID NO: 19 - DNA, 12 bp, Artificial Sequence
  Synthetic oligonucleotide
taagttattaaa

SEQ ID NO: 20 - DNA, 10 bp, Artificial Sequence
  Synthetic oligonucleotide
agttattaaa

SEQ ID NO: 21 - DNA, 14 bp, Artificial Sequence
  Synthetic oligonucleotide
gaaaaaagtagcta

SEQ ID NO: 22 - DNA, 14 bp, Artificial Sequence
  Synthetic oligonucleotide
ccgtaccataccct

SEQ ID NO: 23 - DNA, 20 bp, Artificial Sequence
  Synthetic oligonucleotide
ggatagtctctacaattggg

SEQ ID NO: 24 - DNA, 23 bp, Artificial Sequence
  Synthetic oligonucleotide
tccgccaggcgtgtatatatagc

SEQ ID NO: 25 - DNA, 23 bp, Artificial Sequence
  Synthetic oligonucleotide
aacgaagcaggaaatgagagaat

SEQ ID NO: 26 - DNA, 23 bp, Artificial Sequence
  Synthetic oligonucleotide
gatatcaagaggattggaaaagg

SEQ ID NO: 27 - DNA, 23 bp, Artificial Sequence
  Synthetic oligonucleotide
gatatcaagaggattggaaaagg

SEQ ID NO: 28 - DNA, 21 bp, Artificial Sequence
  Synthetic oligonucleotide
cgaagcaggaaatgagagaat

SEQ ID NO: 29 - DNA, 21 bp, Artificial Sequence
  Synthetic oligonucleotide
cttcgttcatttcgagtttcc

SEQ ID NO: 30 - DNA, 21 bp, Artificial Sequence
  Synthetic oligonucleotide
cagacctccctgcgagcgggc

SEQ ID NO: 31 - DNA, 21 bp, Artificial Sequence
  Synthetic oligonucleotide
cgccaggcgtgtatatatagc

SEQ ID NO: 32 - DNA, 21 bp, Artificial Sequence
  Synthetic oligonucleotide
tcatttggcgagcgttggttg

SEQ ID NO: 33 - DNA, 21 bp, Artificial Sequence
  Synthetic oligonucleotide
gatctttccggtctctttggc

SEQ ID NO: 34 - DNA, 21 bp, Artificial Sequence
  Synthetic oligonucleotide
ggcttgttccacaggaacact

SEQ ID NO: 35 - DNA, 21 bp, Artificial Sequence
  Synthetic oligonucleotide
gccaaagtcctcgacttcaag

SEQ ID NO: 36 - DNA, 21 bp, Artificial Sequence
  Synthetic oligonucleotide
acaacttcgccttaagttgaa

SEQ ID NO: 37 - DNA, 20 bp, Artificial Sequence
  Synthetic oligonucleotide
tctaagttttaattacaaaa

SEQ ID NO: 38 - DNA, 20 bp, Artificial Sequence
  Synthetic oligonucleotide
ggaattcgtgagcaagggcg

SEQ ID NO: 39 - DNA, 20 bp, Artificial Sequence
  Synthetic oligonucleotide
cgaggagctgttcaccgggg

SEQ ID NO: 40 - DNA, 20 bp, Artificial Sequence
  Synthetic oligonucleotide
gaccaggatgggcaccaccc

SEQ ID NO: 41 - DNA, 20 bp, Artificial Sequence
  Synthetic oligonucleotide
cgtcgccgtccagctcgacc

SEQ ID NO: 42 - DNA, 20 bp, Artificial Sequence
  Synthetic oligonucleotide
ggtggtgcagatcagcttca

SEQ ID NO: 43 - DNA, 20 bp, Artificial Sequence
  Synthetic oligonucleotide
caagtaatacatattcaaaa

SEQ ID NO: 44 - DNA, 20 bp, Artificial Sequence
  Synthetic oligonucleotide
gaatatgtattacttggtta

SEQ ID NO: 45 - DNA, 20 bp, Artificial Sequence
  Synthetic oligonucleotide
aagaacagaagaataacgca

SEQ ID NO: 46 - DNA, 20 bp, Artificial Sequence
  Synthetic oligonucleotide
ttatccctcatgttgtctaa

SEQ ID NO: 47 - DNA, 20 bp, Artificial Sequence
  Synthetic oligonucleotide
caatcaatacaataaaataa

SEQ ID NO: 48 - DNA, 20 bp, Artificial Sequence
  Synthetic oligonucleotide
tactcttttgaacaagatgt

SEQ ID NO: 49 - DNA, 20 bp, Artificial Sequence
  Synthetic oligonucleotide
ataagtatattaggatgagg

SEQ ID NO: 50 - DNA, 23 bp, Artificial Sequence
  Synthetic oligonucleotide
gtgtaggaacatcaacatgctca

SEQ ID NO: 51 - DNA, 23 bp, Artificial Sequence
  Synthetic oligonucleotide
cccttctccagaaacaatcagat

SEQ ID NO: 52 - DNA, 23 bp, Artificial Sequence
  Synthetic oligonucleotide
ccattcgtcttgaagtcgaggac

SEQ ID NO: 53 - DNA, 23 bp, Artificial Sequence
  Synthetic oligonucleotide
agttattaaatggtcttcaattt

SEQ ID NO: 54 - DNA, 23 bp, Artificial Sequence
  Synthetic oligonucleotide
ataacttaaatatcaatgggagg

SEQ ID NO: 55 - DNA, 20 bp, Artificial Sequence
  Synthetic oligonucleotide
aatgaacgaagcaggaaatg

SEQ ID NO: 56 - DNA, 20 bp, Artificial Sequence
  Synthetic oligonucleotide
gcgtgttgttgctgctgaca

SEQ ID NO: 57 - DNA, 23 bp, Artificial Sequence
  Synthetic oligonucleotide
cttcttgctcattagaaagaaag

SEQ ID NO: 58 - DNA, 23 bp, Artificial Sequence
  Synthetic oligonucleotide
taattaaaacttagattagattg

SEQ ID NO: 59 - DNA, 23 bp, Artificial Sequence
  Synthetic oligonucleotide
cgtcgccgtccagctcgaccagg

SEQ ID NO: 60 - DNA, 20 bp, Artificial Sequence
  Synthetic oligonucleotide
tttcttagcaaagcaaagga

SEQ ID NO: 61 - DNA, 20 bp, Artificial Sequence
  Synthetic oligonucleotide
ggaaactcgaaatgaacgaa

SEQ ID NO: 62 - DNA, 20 bp, Artificial Sequence
  Synthetic oligonucleotide
cttagcaaagcaaaggaggg

SEQ ID NO: 63 - DNA, 20 bp, Artificial Sequence
  Synthetic oligonucleotide
atagcggtagtgtttgcgcg

SEQ ID NO: 64 - DNA, 20 bp, Artificial Sequence
  Synthetic oligonucleotide
gtaaaccccggccaaagaga

SEQ ID NO: 65 - DNA, 20 bp, Artificial Sequence
  Synthetic oligonucleotide
acacgcctggcggatctgct

SEQ ID NO: 66 - DNA, 20 bp, Artificial Sequence
  Synthetic oligonucleotide
acctgaatctaaaattcccg

SEQ ID NO: 67 - DNA, 20 bp, Artificial Sequence
  Synthetic oligonucleotide
gccggggtttacggacgatg

SEQ ID NO: 68 - DNA, 20 bp, Artificial Sequence
  Synthetic oligonucleotide
gacggaaaaaagtagctaag

SEQ ID NO: 69 - DNA, 20 bp, Artificial Sequence
  Synthetic oligonucleotide
caaagcattcaattcaaatg

SEQ ID NO: 70 - DNA, 23 bp, Artificial Sequence
  Synthetic oligonucleotide
caagggtatggtacggtgctatc

SEQ ID NO: 71 - DNA, 23 bp, Artificial Sequence
  Synthetic oligonucleotide
tcagcagcaacaacacgctacgc

SEQ ID NO: 72 - DNA, 23 bp, Artificial Sequence
  Synthetic oligonucleotide
cggacgatggcagaagaccaaag

SEQ ID NO: 73 - DNA, 23 bp, Artificial Sequence
  Synthetic oligonucleotide
gcgagcgttggttggtggatcaa

SEQ ID NO: 74 - DNA, 23 bp, Artificial Sequence
  Synthetic oligonucleotide
gtgctgacacatacaggcatata

SEQ ID NO: 75 - DNA, 20 bp, Artificial Sequence
  Synthetic oligonucleotide
ataaatggaaagttaggaca

SEQ ID NO: 76 - DNA, 23 bp, Artificial Sequence
  Synthetic oligonucleotide
cggctatgaaaagctgttgttcg

SEQ ID NO: 77 - DNA, 21 bp, Artificial Sequence
  Synthetic oligonucleotide
actaccacaggatcttaatag

SEQ ID NO: 78 - DNA, 23 bp, Artificial Sequence
  Synthetic oligonucleotide
catattcgaagcttacaatcgag

SEQ ID NO: 79 - DNA, 23 bp, Artificial Sequence
  Synthetic oligonucleotide
taccagcaatcagctgactaaca

SEQ ID NO: 80 - DNA, 23 bp, Artificial Sequence
  Synthetic oligonucleotide
ttgctcttacccgactctgaaga

SEQ ID NO: 81 - DNA, 23 bp, Artificial Sequence
  Synthetic oligonucleotide
gcaagacctcaaacaatcgtact

SEQ ID NO: 82 - DNA, 20 bp, Artificial Sequence
  Synthetic oligonucleotide
gctggggtagaactagagta

SEQ ID NO: 83 - DNA, 20 bp, Artificial Sequence
  Synthetic oligonucleotide
ttatatgacagttcaaaaga

SEQ ID NO: 84 - DNA, 20 bp, Artificial Sequence
  Synthetic oligonucleotide
ggaagtggagatggaagagg

SEQ ID NO: 85 - DNA, 20 bp, Artificial Sequence
  Synthetic oligonucleotide
ctacatgcaaacgacaaata

SEQ ID NO: 86 - DNA, 20 bp, Artificial Sequence
  Synthetic oligonucleotide
gctgaaaactgtatgtgcgg

SEQ ID NO: 87 - DNA, 20 bp, Artificial Sequence
  Synthetic oligonucleotide
atccaacgatgcaattcagt

SEQ ID NO: 88 - DNA, 20 bp, Artificial Sequence
  Synthetic oligonucleotide
aaatgggaatggaaagaacg

SEQ ID NO: 89 - DNA, 21 bp, Artificial Sequence
  Synthetic oligonucleotide
atctctcagaaatcggtacaa

SEQ ID NO: 90 - DNA, 23 bp, Artificial Sequence
  Synthetic oligonucleotide
caacaactatctgcgataactca

SEQ ID NO: 91 - DNA, 23 bp, Artificial Sequence
  Synthetic oligonucleotide
cagggtcttctataagagaaacc

SEQ ID NO: 92 - DNA, 23 bp, Artificial Sequence
  Synthetic oligonucleotide
agccctacttaatgctgagccac

SEQ ID NO: 93 - DNA, 23 bp, Artificial Sequence
  Synthetic oligonucleotide
gctatgttagctgcaactttcta

SEQ ID NO: 94 - DNA, 23 bp, Artificial Sequence
  Synthetic oligonucleotide
gaaacacgtgtcctgaaaattat

SEQ ID NO: 95 - DNA, 23 bp, Artificial Sequence
  Synthetic oligonucleotide
aaaatcatcgaatagccgatcga

SEQ ID NO: 96 - DNA, 23 bp, Artificial Sequence
  Synthetic oligonucleotide
ccagtcactatcatcatcatcat

SEQ ID NO: 97 - DNA, 23 bp, Artificial Sequence
  Synthetic oligonucleotide
acgggcaaaaactggattctccc

SEQ ID NO: 98 - DNA, 23 bp, Artificial Sequence
  Synthetic oligonucleotide
tgtcttacgagccgggtaccaag

SEQ ID NO: 99 - DNA, 23 bp, Artificial Sequence
  Synthetic oligonucleotide
caggggcgatgccacttatcagt

SEQ ID NO: 100 - DNA, 20 bp, Artificial Sequence
  Synthetic oligonucleotide
ggattggcgagaaataatgt

SEQ ID NO: 101 - DNA, 20 bp, Artificial Sequence
  Synthetic oligonucleotide
gcagatggggagagagaatg

SEQ ID NO: 102 - DNA, 20 bp, Artificial Sequence
  Synthetic oligonucleotide
tttccttgtagcgatcaggt

SEQ ID NO: 103 - DNA, 20 bp, Artificial Sequence
  Synthetic oligonucleotide
gaaataacgggtcccaagag

SEQ ID NO: 104 - DNA, 20 bp, Artificial Sequence
  Synthetic oligonucleotide
cccacgggttctttcttagg

SEQ ID NO: 105 - DNA, 20 bp, Artificial Sequence
  Synthetic oligonucleotide
gcgagcaaacactattatga

SEQ ID NO: 106 - DNA, 20 bp, Artificial Sequence
  Synthetic oligonucleotide
agaagggcttggtttcgaaa

SEQ ID NO: 107 - DNA, 20 bp, Artificial Sequence
  Synthetic oligonucleotide
caaaacgggatatttaagcc

SEQ ID NO: 108 - DNA, 20 bp, Artificial Sequence
  Synthetic oligonucleotide
agccgaatgaatgaaatatg

SEQ ID NO: 109 - DNA, 20 bp, Artificial Sequence
  Synthetic oligonucleotide
ttgttgtgatgatacaagag

SEQ ID NO: 110 - DNA, 21 bp, Artificial Sequence
  Synthetic oligonucleotide
ttgaaggtatcggtttaggcg

SEQ ID NO: 111 - DNA, 21 bp, Artificial Sequence
  Synthetic oligonucleotide
tatgcatttggaacttgaacg

SEQ ID NO: 112 - DNA, 21 bp, Artificial Sequence
  Synthetic oligonucleotide
atacgtaataccctatcctgg

SEQ ID NO: 113 - DNA, 51 bp, Artificial Sequence
  Synthetic oligonucleotide
ctcactatagggcgaattgggtaccctcgagaatttttttggaaaaccaag

SEQ ID NO: 114 - DNA, 50 bp, Artificial Sequence
  Synthetic oligonucleotide
gttatcctcctcgcccttgctcaccattattaatttagtgtgtgtatttg

SEQ ID NO: 115 - DNA, 46 bp, Artificial Sequence
  Synthetic oligonucleotide
cacaaatacacacactaaattaataatggtgagcaagggcgaggag

SEQ ID NO: 116 - DNA, 47 bp, Artificial Sequence
  Synthetic oligonucleotide
gcctgttgctatcgataccgtcgacatagcgccgatcaaagtatttg

SEQ ID NO: 117 - DNA, 44 bp, Artificial Sequence
  Synthetic oligonucleotide
tcggcgctatgtcgacggtatcgatagcaacaggcgcgttggac

SEQ ID NO: 118 - DNA, 46 bp, Artificial Sequence
  Synthetic oligonucleotide
ctaaagggaacaaaagctggagctccaggaagaatacactatactg

SEQ ID NO: 119 - DNA, 37 bp, Artificial Sequence
  Synthetic oligonucleotide
cgctatgtcgactgggtcattacgtaaataatgatag

SEQ ID NO: 120 - DNA, 39 bp, Artificial Sequence
  Synthetic oligonucleotide
ctcacgaattccattttgaatatgtattacttggttatg

SEQ ID NO: 121 - DNA, 33 bp, Artificial Sequence
  Synthetic oligonucleotide
cgctatgtcgacgttttgacaccgagccatagc

SEQ ID NO: 122 - DNA, 36 bp, Artificial Sequence
  Synthetic oligonucleotide
ctcacgaattccattattttattgtattgattgttg

SEQ ID NO: 123 - DNA, 32 bp, Artificial Sequence
  Synthetic oligonucleotide
cgctatgtcgaccatccacatattttaatcac

SEQ ID NO: 124 - DNA, 35 bp, Artificial Sequence
  Synthetic oligonucleotide
ctcacgaattccatcgctggatatgcctagaaatg

SEQ ID NO: 125 - DNA, 35 bp, Artificial Sequence
  Synthetic oligonucleotide
cgctatgtcgacaactatgcgaaatccggagcaac

SEQ ID NO: 126 - DNA, 35 bp, Artificial Sequence
  Synthetic oligonucleotide
ctcacgaattccatggtaattggacaaataaatac

SEQ ID NO: 127 - DNA, 36 bp, Artificial Sequence
  Synthetic oligonucleotide
gttcgcggatccatggtgcctaagaagaagagaaag

SEQ ID NO: 128 - DNA, 35 bp, Artificial Sequence
  Synthetic oligonucleotide
cacccgctcgagttaatccagcttctttttcttcg

SEQ ID NO: 129 - DNA, 36 bp, Artificial Sequence
  Synthetic oligonucleotide
gttcgcggatccatgagcgacctggtgctgggcctg

SEQ ID NO: 130 - DNA, 35 bp, Artificial Sequence
  Synthetic oligonucleotide
cacccgctcgagtcacaccttcctcttcttcttgg

SEQ ID NO: 131 - DNA, 32 bp, Artificial Sequence
  Synthetic oligonucleotide
gacatgccatggggaaacggaactacatcctg

SEQ ID NO: 132 - DNA, 34 bp, Artificial Sequence
  Synthetic oligonucleotide
gaacgcgtcgacttacttgtcatcgtcatccttg

SEQ ID NO: 133 - DNA, 35 bp, Artificial Sequence
  Synthetic oligonucleotide
gttcgcggatccatgacacagttcgagggctttac

SEQ ID NO: 134 - DNA, 41 bp, Artificial Sequence
  Synthetic oligonucleotide
gttcgcggatccatgagcaagctggagaagtttacaaactg

SEQ ID NO: 135 - DNA, 36 bp, Artificial Sequence
  Synthetic oligonucleotide
cacccgctcgagtcactttttcttttttgcctggcc

SEQ ID NO: 136 - DNA, 33 bp, Artificial Sequence
  Synthetic oligonucleotide
ccactacgtgctcgagtctttgaaaagataatg

SEQ ID NO: 137 - DNA, 67 bp, Artificial Sequence
  Synthetic oligonucleotide
gcagggagctcagacataaaaaacaaaaaaaggagacctcggtctccgatcatttatctttcactgc

SEQ ID NO: 138 - DNA, 60 bp, Artificial Sequence
  Synthetic oligonucleotide
ctccgcagtgaaagataaatgatcggagaccgaggtctccgttttagagctagaaatagc

SEQ ID NO: 139 - DNA, 46 bp, Artificial Sequence
  Synthetic oligonucleotide
cagacataaaaaacaaaaaaaggatcaaaaaagcaccgactcggtg

SEQ ID NO: 140 - DNA, 60 bp, Artificial Sequence
  Synthetic oligonucleotide
ctccgcagtgaaagataaatgatcggagaccgaggtctccgttgtagctccctttctcat

SEQ ID NO: 141 - DNA, 46 bp, Artificial Sequence
  Synthetic oligonucleotide
cagacataaaaaacaaaaaaaggatctaaacgatgccccttaaagc

SEQ ID NO: 142 - DNA, 60 bp, Artificial Sequence
  Synthetic oligonucleotide
ctccgcagtgaaagataaatgatcggagaccgaggtctccgtttttgtactctcagaaat

SEQ ID NO: 143 - DNA, 50 bp, Artificial Sequence
  Synthetic oligonucleotide
cagacataaaaaacaaaaaaaggatcaaaaaaacaccctgccataaaatg

SEQ ID NO: 144 - DNA, 60 bp, Artificial Sequence
  Synthetic oligonucleotide
ctccgcagtgaaagataaatgatcggagaccgaggtctccgttttagtactctgtaattt

SEQ ID NO: 145 - DNA, 46 bp, Artificial Sequence
  Synthetic oligonucleotide
cagacataaaaaacaaaaaaaggatcaaaaaaatctcgccaacaag

SEQ ID NO: 146 - DNA, 45 bp, Artificial Sequence
  Synthetic oligonucleotide
gatccatgcctccaaaaaagaagagaaaggtcggtagtggttctg

SEQ ID NO: 147 - DNA, 45 bp, Artificial Sequence
  Synthetic oligonucleotide
gatccagaaccactaccgacctttctcttcttttttggaggcatg

SEQ ID NO: 148 - DNA, 45 bp, Artificial Sequence
  Synthetic oligonucleotide
catgggccctccaaaaaagaagagaaaggtcggtagtggttcttc

SEQ ID NO: 149 - DNA, 45 bp, Artificial Sequence
  Synthetic oligonucleotide
catggaagaaccactaccgacctttctcttcttttttggagggcc

SEQ ID NO: 150 - DNA, 60 bp, Artificial Sequence
  Synthetic oligonucleotide
atggattctagaacagttggtatattaggagggggacaatttcgtacgctgcaggtcgac

SEQ ID NO: 151 - DNA, 60 bp, Artificial Sequence
  Synthetic oligonucleotide
ttacttgttttctagataagcttcgtaaccgacagtttctgcataggccactagtggatc

SEQ ID NO: 152 - DNA, 37 bp, Artificial Sequence
  Synthetic oligonucleotide
gttggaagatctatgggtgatcattatctggatattc

SEQ ID NO: 153 - DNA, 32 bp, Artificial Sequence
  Synthetic oligonucleotide
cacccgctcgagttaaaaccagggcacgaaac

SEQ ID NO: 154 - DNA, 50 bp, Artificial Sequence
  Synthetic oligonucleotide
actttttacaacaaatataaaacagatggactacaaagaccatgacggtg

SEQ ID NO: 155 - DNA, 55 bp, Artificial Sequence
  Synthetic oligonucleotide
gaattaataaaagtgttcgcaaaggatctcacagcaaggctgagaaatccatatc

SEQ ID NO: 156 - DNA, 52 bp, Artificial Sequence
  Synthetic oligonucleotide
gaattaataaaagtgttcgcaaaggatctcataacatatcgagatcgaaatc

SEQ ID NO: 157 - DNA, 52 bp, Artificial Sequence
  Synthetic oligonucleotide
gaattaataaaagtgttcgcaaaggatctcaagaagcgtagtccggaacgtc

SEQ ID NO: 158 - DNA, 49 bp, Artificial Sequence
  Synthetic oligonucleotide
actttttacaacaaatataaaacagatggccccaaagaagaagcggaag

SEQ ID NO: 159 - DNA, 55 bp, Artificial Sequence
  Synthetic oligonucleotide
gaattaataaaagtgttcgcaaaggatccagcatgtccaggtcgaaatcatcaag

SEQ ID NO: 160 - DNA, 53 bp, Artificial Sequence
  Synthetic oligonucleotide
gaattaataaaagtgttcgcaaaggatctcaaaacagagatgtgtcgaagatg

SEQ ID NO: 161 - DNA, 35 bp, Artificial Sequence
  Synthetic oligonucleotide
ccgccaccatggctcctccaaaaaagaagagaaag

SEQ ID NO: 162 - DNA, 60 bp, Artificial Sequence
  Synthetic oligonucleotide
caccacgatatacagcagattgcgctcgcccctagcgatgccgatcacataggggttatc

SEQ ID NO: 163 - DNA, 60 bp, Artificial Sequence
  Synthetic oligonucleotide
ctgaagcacgacgataacccctatgtgatcggcatcgctaggggcgagcgcaatctgctg

SEQ ID NO: 164 - DNA, 35 bp, Artificial Sequence
  Synthetic oligonucleotide
ccgccgaagcttctttttcttttttgcctggccgg

SEQ ID NO: 165 - DNA, 53 bp, Artificial Sequence
  Synthetic oligonucleotide
agttccaagcttggcggcagcggcggcagcatgactgccagcgtttcgaatac

SEQ ID NO: 166 - DNA, 39 bp, Artificial Sequence
  Synthetic oligonucleotide
cacccgctcgagttaaggtggttgctgttgttgaagttg

SEQ ID NO: 167 - DNA, 51 bp, Artificial Sequence
  Synthetic oligonucleotide
agttccaagcttggcggcagcggcggcagctacgaagaagagatcaagcac

SEQ ID NO: 168 - DNA, 38 bp, Artificial Sequence
  Synthetic oligonucleotide
cacccgctcgagttacgcaactggaacagatgcagatg

SEQ ID NO: 169 - DNA, 51 bp, Artificial Sequence
  Synthetic oligonucleotide
agttccaagcttggcggcagcggcggcagcgctagtttgcaccaggatcac

SEQ ID NO: 170 - DNA, 36 bp, Artificial Sequence
  Synthetic oligonucleotide
cacccgctcgagttaagatttgtgtaactcaacgtc

SEQ ID NO: 171 - DNA, 51 bp, Artificial Sequence
  Synthetic oligonucleotide
agttccaagcttggcggcagcggcggcagcgattcacaagttcaagaactg

SEQ ID NO: 172 - DNA, 33 bp, Artificial Sequence
  Synthetic oligonucleotide
cacccgctcgagtcagtccatgtgtgggaaggg

SEQ ID NO: 173 - DNA, 51 bp, Artificial Sequence
  Synthetic oligonucleotide
agttccaagcttggcggcagcggcggcagcactagtggtacgaatttgcac

SEQ ID NO: 174 - DNA, 50 bp, Artificial Sequence
  Synthetic oligonucleotide
agttccaagcttggcggcagcggcggcagcatggtaatcttcaaagaacg

SEQ ID NO: 175 - DNA, 35 bp, Artificial Sequence
  Synthetic oligonucleotide
cacccgctcgagttagataagtggcggtaatattg

SEQ ID NO: 176 - DNA, 39 bp, Artificial Sequence
  Synthetic oligonucleotide
cacccgctcgagttaagatttgttattttctgcaatttg

SEQ ID NO: 177 - DNA, 54 bp, Artificial Sequence
  Synthetic oligonucleotide
agttccaagcttggcggcagcggcggcagcttctgtcaagttttcgtaacaaag

SEQ ID NO: 178 - DNA, 33 bp, Artificial Sequence
  Synthetic oligonucleotide
cacccgctcgagttaaacttttaggccattgac

SEQ ID NO: 179 - DNA, 51 bp, Artificial Sequence
  Synthetic oligonucleotide
agttccaagcttggcggcagcggcggcagctgtgtagtgaacttgcaaaac

SEQ ID NO: 180 - DNA, 36 bp, Artificial Sequence
  Synthetic oligonucleotide
cacccgctcgagttaatcacggaggtatctcaaccg

SEQ ID NO: 181 - DNA, 50 bp, Artificial Sequence
  Synthetic oligonucleotide
agttccaagcttggcggcagcggcggcagcaattctgcatcttcatctac

SEQ ID NO: 182 - DNA, 40 bp, Artificial Sequence
  Synthetic oligonucleotide
cacccgctcgagttatgtagaattgttgctttcgaaaatg

SEQ ID NO: 183 - DNA, 36 bp, Artificial Sequence
  Synthetic oligonucleotide
ccgccaccatggctcccaagaaaaagcgcaaggtag

SEQ ID NO: 184 - DNA, 36 bp, Artificial Sequence
  Synthetic oligonucleotide
gaggagccatggacgcaactggaacagatgcagatg

SEQ ID NO: 185 - DNA, 36 bp, Artificial Sequence
  Synthetic oligonucleotide
gaggagccatggagtccatgtgtgggaagggcaacg

SEQ ID NO: 186 - DNA, 37 bp, Artificial Sequence
  Synthetic oligonucleotide
  misc_feature (1)..(5): n can be a, g, c, or t
nnnnnggtctccggactctttgaaaagataatgtatg

SEQ ID NO: 187 - DNA, 38 bp, Artificial Sequence
  Synthetic oligonucleotide
  misc_feature (1)..(5): n can be a, g, c, or t
nnnnnggtctcccggacttgcatgcctgcagggagctc

SEQ ID NO: 188 - DNA, 37 bp, Artificial Sequence
  Synthetic oligonucleotide
  misc_feature (1)..(5): n can be a, g, c, or t
nnnnnggtctcctccgtctttgaaaagataatgtatg

SEQ ID NO: 189 - DNA, 38 bp, Artificial Sequence
  Synthetic oligonucleotide
  misc_feature (1)..(5): n can be a, g, c, or t
nnnnnggtctccctggcttgcatgcctgcagggagctc

SEQ ID NO: 190 - DNA, 37 bp, Artificial Sequence
  Synthetic oligonucleotide
  misc_feature (1)..(5): n can be a, g, c, or t
nnnnnggtctccccagtctttgaaaagataatgtatg

SEQ ID NO: 191 - DNA, 38 bp, Artificial Sequence
  Synthetic oligonucleotide
  misc_feature (1)..(5): n can be a, g, c, or t
nnnnnggtctcccaaccttgcatgcctgcagggagctc

SEQ ID NO: 192 - DNA, 33 bp, Artificial Sequence
  Synthetic oligonucleotide
ggttgagtgttgttccagtttggaacaagagtc

SEQ ID NO: 193 - DNA, 29 bp, Artificial Sequence
  Synthetic oligonucleotide
catgccggtagaggtgtggtcaataagag

SEQ ID NO: 194 - DNA, 28 bp, Artificial Sequence
  Synthetic oligonucleotide
agctttggacttcttcgccagaggtttg

SEQ ID NO: 195 - DNA, 28 bp, Artificial Sequence
  Synthetic oligonucleotide
gcttggtgccacttgtcacatacaattc

SEQ ID NO: 196 - DNA, 33 bp, Artificial Sequence
  Synthetic oligonucleotide
cctgcagggtgtcgacgctgcgggtatagaaag

SEQ ID NO: 197 - DNA, 34 bp, Artificial Sequence
  Synthetic oligonucleotide
ctgccctttatattccctgttacagcagccgagc

SEQ ID NO: 198 - DNA, 32 bp, Artificial Sequence
  Synthetic oligonucleotide
gcggccgctatatctaggaacccatcaggttg

SEQ ID NO: 199 - DNA, 33 bp, Artificial Sequence
  Synthetic oligonucleotide
gattgctatgctttctttctaatgagcaagaag

SEQ ID NO: 200 - DNA, 29 bp, Artificial Sequence
  Synthetic oligonucleotide
ccgcggatagcttcaaaatgtttctactc

SEQ ID NO: 201 - DNA, 28 bp, Artificial Sequence
  Synthetic oligonucleotide
gggtttcgccacctctgacttgagcgtc

SEQ ID NO: 202 - DNA, 47 bp, Artificial Sequence
  Synthetic oligonucleotide
cagacataaaaaacaaaaaaaggatcgggaagactccccagtgactg

SEQ ID NO: 203 - DNA, 49 bp, Artificial Sequence
  Synthetic oligonucleotide
cagacataaaaaacaaaaaaaggatcgggaactgctgcgtaagggtttc

SEQ ID NO: 204 - DNA, 60 bp, Artificial Sequence
  Synthetic oligonucleotide
cagacataaaaaacaaaaaaaggatcgatgctcgcaggcattcaggcaccgactcggtgc

SEQ ID NO: 205 - DNA, 36 bp, Artificial Sequence
  Synthetic oligonucleotide
gttcgcggatccatgcccaaaaagaaaagaaaagtg

SEQ ID NO: 206 - DNA, 33 bp, Artificial Sequence
  Synthetic oligonucleotide
tcttgggagctccctcggagccacggcccagcg

SEQ ID NO: 207 - DNA, 36 bp, Artificial Sequence
  Synthetic oligonucleotide
gttggaagatctatgcccaaaaagaaaagaaaagtg

SEQ ID NO: 208 - DNA, 36 bp, Artificial Sequence
  Synthetic oligonucleotide
cacccgctcgagtcagttgatgagcatgtccagatc

SEQ ID NO: 209 - DNA, 21 bp, Artificial Sequence
  Synthetic oligonucleotide
tattctgttcagacagggacc

SEQ ID NO: 210 - DNA, 22 bp, Artificial Sequence
  Synthetic oligonucleotide
gatagctgttcgagcttgacac

SEQ ID NO: 211 - DNA, 22 bp, Artificial Sequence
  Synthetic oligonucleotide
catctaacgaggccaacaatag

SEQ ID NO: 212 - DNA, 22 bp, Artificial Sequence
  Synthetic oligonucleotide
atataagctatacaagaggctg

SEQ ID NO: 213 - DNA, 22 bp, Artificial Sequence
  Synthetic oligonucleotide
cgatcatgaagcttcatcaagc

SEQ ID NO: 214 - DNA, 22 bp, Artificial Sequence
  Synthetic oligonucleotide
ctctccaattcggcgacttgac

SEQ ID NO: 215 - DNA, 22 bp, Artificial Sequence
  Synthetic oligonucleotide
acgagaccggaaatatagagtg

SEQ ID NO: 216 - DNA, 22 bp, Artificial Sequence
  Synthetic oligonucleotide
caggagaatggctagcggactg

SEQ ID NO: 217 - DNA, 22 bp, Artificial Sequence
  Synthetic oligonucleotide
cgacttgaacgttaccgggttg

SEQ ID NO: 218 - DNA, 22 bp, Artificial Sequence
  Synthetic oligonucleotide
tcagatggacagtccattgcgc

SEQ ID NO: 219 - DNA, 21 bp, Artificial Sequence
  Synthetic oligonucleotide
agaagtggacggtgatttgag

SEQ ID NO: 220 - DNA, 20 bp, Artificial Sequence
  Synthetic oligonucleotide
catggcaccttgtggttcta

SEQ ID NO: 221 - DNA, 20 bp, Artificial Sequence
  Synthetic oligonucleotide
cttctggcccaaggaaatct

SEQ ID NO: 222 - DNA, 22 bp, Artificial Sequence
  Synthetic oligonucleotide
gacgaggtggtttatacagtcc

SEQ ID NO: 223 - DNA, 20 bp, Artificial Sequence
  Synthetic oligonucleotide
gtcaacgacccaaagaagga

SEQ ID NO: 224 - DNA, 20 bp, Artificial Sequence
  Synthetic oligonucleotide
tggcgtaggtatcagctagt

SEQ ID NO: 225 - DNA, 22 bp, Artificial Sequence
  Synthetic oligonucleotide
ggagaaggaaagacacgcttta

SEQ ID NO: 226 - DNA, 22 bp, Artificial Sequence
  Synthetic oligonucleotide
ccaagaagtgtgaggtcctatg

SEQ ID NO: 227 - DNA, 22 bp, Artificial Sequence
  Synthetic oligonucleotide
ttgctctgttgatgtcgtagag

SEQ ID NO: 228 - DNA, 22 bp, Artificial Sequence
  Synthetic oligonucleotide
tcatccgcttccttcattgtat

SEQ ID NO: 229 - DNA, 20 bp, Artificial Sequence
  Synthetic oligonucleotide
ccttagcggttgctgctatt

SEQ ID NO: 230 - DNA, 21 bp, Artificial Sequence
  Synthetic oligonucleotide
accttctcacgatggctttac

SEQ ID NO: 231 - DNA, 21 bp, Artificial Sequence
  Synthetic oligonucleotide
ccgttgccatgttgttgtatg

SEQ ID NO: 232 - DNA, 22 bp, Artificial Sequence
  Synthetic oligonucleotide
gccaggaaattgtacgctaaac

SEQ ID NO: 233 - DNA, 24 bp, Artificial Sequence
  Synthetic oligonucleotide
gatcttgatatttaagttattaaa

SEQ ID NO: 234 - DNA, 24 bp, Artificial Sequence
  Synthetic oligonucleotide
aaactttaataacttaaatatcaa

SEQ ID NO: 235 - DNA, 24 bp, Artificial Sequence
  Synthetic oligonucleotide
gatcactttagtgctgacacatac

SEQ ID NO: 236 - DNA, 24 bp, Artificial Sequence
  Synthetic oligonucleotide
aaacgtatgtgtcagcactaaagt

SEQ ID NO: 237 - DNA, 24 bp, Artificial Sequence
  Synthetic oligonucleotide
gatcgatatcaagaggattggaaa

SEQ ID NO: 238 - DNA, 24 bp, Artificial Sequence
  Synthetic oligonucleotide
aaactttccaatcctcttgatatc

SEQ ID NO: 239 - DNA, 24 bp, Artificial Sequence
  Synthetic oligonucleotide
gatcacgtccctattgaatgttgg

SEQ ID NO: 240 - DNA, 24 bp, Artificial Sequence
  Synthetic oligonucleotide
caacccaacattcaatagggacgt

SEQ ID NO: 241 - DNA, 24 bp, Artificial Sequence
  Synthetic oligonucleotide
gatcaactctggacattataccat

SEQ ID NO: 242 - DNA, 24 bp, Artificial Sequence
  Synthetic oligonucleotide
caacatggtataatgtccagagtt

SEQ ID NO: 243 - DNA, 24 bp, Artificial Sequence
  Synthetic oligonucleotide
gatcaaaaatgggcaccatttact

SEQ ID NO: 244 - DNA, 24 bp, Artificial Sequence
  Synthetic oligonucleotide
aaacagtaaatggtgcccattttt

SEQ ID NO: 245 - DNA, 24 bp, Artificial Sequence
  Synthetic oligonucleotide
gatcccaattgtagagactatcca

SEQ ID NO: 246 - DNA, 24 bp, Artificial Sequence
  Synthetic oligonucleotide
aaactggatagtctctacaattgg

SEQ ID NO: 247 - DNA, 18 bp, Artificial Sequence
  Synthetic oligonucleotide
gatctttaagttattaaa

SEQ ID NO: 248 - DNA, 18 bp, Artificial Sequence
  Synthetic oligonucleotide
aaactttaataacttaaa

SEQ ID NO: 249 - DNA, 18 bp, Artificial Sequence
  Synthetic oligonucleotide
gatctaaatatcaatggg

SEQ ID NO: 250 - DNA, 18 bp, Artificial Sequence
  Synthetic oligonucleotide
aaaccccattgatattta

SEQ ID NO: 251 - DNA, 24 bp, Artificial Sequence
  Synthetic oligonucleotide
gatcgaagctcatttgagatcaat

SEQ ID NO: 252 - DNA, 24 bp, Artificial Sequence
  Synthetic oligonucleotide
caacattgatctcaaatgagcttc

SEQ ID NO: 253 - DNA, 24 bp, Artificial Sequence
  Synthetic oligonucleotide
gatcggaagaggtaacttcgttgt

SEQ ID NO: 254 - DNA, 24 bp, Artificial Sequence
  Synthetic oligonucleotide
aaacacaacgaagttacctcttcc

SEQ ID NO: 255 - DNA, 27 bp, Artificial Sequence
  Synthetic oligonucleotide
gatcgcaagcatcaatggtataatgtc

SEQ ID NO: 256 - DNA, 27 bp, Artificial Sequence
  Synthetic oligonucleotide
aaacgacattataccattgatgcttgc

SEQ ID NO: 257 - DNA, 28 bp, Artificial Sequence
  Synthetic oligonucleotide
gatcgtaacttcgttgtaaagaataagg

SEQ ID NO: 258 - DNA, 28 bp, Artificial Sequence
  Synthetic oligonucleotide
caacccttattctttacaacgaagttac

SEQ ID NO: 259 - DNA, 18 bp, Artificial Sequence
  Synthetic oligonucleotide
gatcgtgctgacacatac

SEQ ID NO: 260 - DNA, 18 bp, Artificial Sequence
  Synthetic oligonucleotide
aaacgtatgtgtcagcac

SEQ ID NO: 261 - DNA, 22 bp, Artificial Sequence
  Synthetic oligonucleotide
gatcgatatttaagttattaaa

SEQ ID NO: 262 - DNA, 18 bp, Artificial Sequence
  Synthetic oligonucleotide
tttaataacttaaatatc

SEQ ID NO: 263 - DNA, 20 bp, Artificial Sequence
  Synthetic oligonucleotide
gatctatttaagttattaaa

SEQ ID NO: 264 - DNA, 20 bp, Artificial Sequence
  Synthetic oligonucleotide
aaactttaataacttaaata

SEQ ID NO: 265 - DNA, 19 bp, Artificial Sequence
  Synthetic oligonucleotide
gatcatttaagttattaaa

SEQ ID NO: 266 - DNA, 19 bp, Artificial Sequence
  Synthetic oligonucleotide
aaactttaataacttaaat

SEQ ID NO: 267 - DNA, 17 bp, Artificial Sequence
  Synthetic oligonucleotide
gatcttaagttattaaa

SEQ ID NO: 268 - DNA, 17 bp, Artificial Sequence
  Synthetic oligonucleotide
aaactttaataacttaa

SEQ ID NO: 269 - DNA, 16 bp, Artificial Sequence
  Synthetic oligonucleotide
gatctaagttattaaa

SEQ ID NO: 270 - DNA, 16 bp, Artificial Sequence
  Synthetic oligonucleotide
aaactttaataactta

SEQ ID NO: 271 - DNA, 14 bp, Artificial Sequence
  Synthetic oligonucleotide
gatcagttattaaa

SEQ ID NO: 272 - DNA, 14 bp, Artificial Sequence
  Synthetic oligonucleotide
aaactttaataact

SEQ ID NO: 273 - DNA, 18 bp, Artificial Sequence
  Synthetic oligonucleotide
gatcgaaaaaagtagcta

SEQ ID NO: 274 - DNA, 18 bp, Artificial Sequence
  Synthetic oligonucleotide
aaactagctacttttttc

SEQ ID NO: 275 - DNA, 18 bp, Artificial Sequence
  Synthetic oligonucleotide
gatcccgtaccataccct

SEQ ID NO: 276 - DNA, 18 bp, Artificial Sequence
  Synthetic oligonucleotide
aaacagggtatggtacgg

SEQ ID NO: 277 - DNA, 24 bp, Artificial Sequence
  Synthetic oligonucleotide
gatcggatagtctctacaattggg

SEQ ID NO: 278 - DNA, 24 bp, Artificial Sequence
  Synthetic oligonucleotide
aaaccccaattgtagagactatcc

SEQ ID NO: 279 - DNA, 27 bp, Artificial Sequence
  Synthetic oligonucleotide
gatctccgccaggcgtgtatatatagc

SEQ ID NO: 280 - DNA, 27 bp, Artificial Sequence
  Synthetic oligonucleotide
aaacgctatatatacacgcctggcgga

SEQ ID NO: 281 - DNA, 27 bp, Artificial Sequence
  Synthetic oligonucleotide
gatcaacgaagcaggaaatgagagaat

SEQ ID NO: 282 - DNA, 27 bp, Artificial Sequence
  Synthetic oligonucleotide
aaacattctctcatttcctgcttcgtt

SEQ ID NO: 283 - DNA, 47 bp, Artificial Sequence
  Synthetic oligonucleotide
gatctaatttctactcttgtagatgatatcaagaggattggaaaagg

SEQ ID NO: 284 - DNA, 47 bp, Artificial Sequence
  Synthetic oligonucleotide
aaaaccttttccaatcctcttgatatcatctacaagagtagaaatta

SEQ ID NO: 285 - DNA, 47 bp, Artificial Sequence
  Synthetic oligonucleotide
gatcaatttctactaagtgtagatgatatcaagaggattggaaaagg

SEQ ID NO: 286 - DNA, 47 bp, Artificial Sequence
  Synthetic oligonucleotide
aaaaccttttccaatcctcttgatatcatctacacttagtagaaatt

SEQ ID NO: 287 - DNA, 25 bp, Artificial Sequence
  Synthetic oligonucleotide
gatccgaagcaggaaatgagagaat

SEQ ID NO: 288 - DNA, 25 bp, Artificial Sequence
  Synthetic oligonucleotide
aaacattctctcatttcctgcttcg

SEQ ID NO: 289 - DNA, 25 bp, Artificial Sequence
  Synthetic oligonucleotide
gatccttcgttcatttcgagtttcc

SEQ ID NO: 290 - DNA, 25 bp, Artificial Sequence
  Synthetic oligonucleotide
aaacggaaactcgaaatgaacgaag

SEQ ID NO: 291 - DNA, 25 bp, Artificial Sequence
  Synthetic oligonucleotide
gatccagacctccctgcgagcgggc

SEQ ID NO: 292 - DNA, 25 bp, Artificial Sequence
  Synthetic oligonucleotide
aaacgcccgctcgcagggaggtctg

SEQ ID NO: 293 - DNA, 25 bp, Artificial Sequence
  Synthetic oligonucleotide
gatccgccaggcgtgtatatatagc

SEQ ID NO: 294 - DNA, 25 bp, Artificial Sequence
  Synthetic oligonucleotide
aaacgctatatatacacgcctggcg

SEQ ID NO: 295 - DNA, 25 bp, Artificial Sequence
  Synthetic oligonucleotide
gatctcatttggcgagcgttggttg

SEQ ID NO: 296 - DNA, 25 bp, Artificial Sequence
  Synthetic oligonucleotide
aaaccaaccaacgctcgccaaatga

SEQ ID NO: 297 - DNA, 25 bp, Artificial Sequence
  Synthetic oligonucleotide
gatcgatctttccggtctctttggc

SEQ ID NO: 298 - DNA, 25 bp, Artificial Sequence
  Synthetic oligonucleotide
aaacgccaaagagaccggaaagatc

SEQ ID NO: 299 - DNA, 25 bp, Artificial Sequence
  Synthetic oligonucleotide
gatcggcttgttccacaggaacact

SEQ ID NO: 300 - DNA, 25 bp, Artificial Sequence
  Synthetic oligonucleotide
aaacagtgttcctgtggaacaagcc

SEQ ID NO: 301 - DNA, 25 bp, Artificial Sequence
  Synthetic oligonucleotide
gatcgccaaagtcctcgacttcaag

SEQ ID NO: 302 - DNA, 25 bp, Artificial Sequence
  Synthetic oligonucleotide
aaaccttgaagtcgaggactttggc

SEQ ID NO: 303 - DNA, 25 bp, Artificial Sequence
  Synthetic oligonucleotide
gatcacaacttcgccttaagttgaa

SEQ ID NO: 304 - DNA, 25 bp, Artificial Sequence
  Synthetic oligonucleotide
aaacttcaacttaaggcgaagttgt

SEQ ID NO: 305 - DNA, 24 bp, Artificial Sequence
  Synthetic oligonucleotide
gatctctaagttttaattacaaaa

SEQ ID NO: 306 - DNA, 24 bp, Artificial Sequence
  Synthetic oligonucleotide
aaacttttgtaattaaaacttaga

SEQ ID NO: 307 - DNA, 24 bp, Artificial Sequence
  Synthetic oligonucleotide
gatcggaattcgtgagcaagggcg

SEQ ID NO: 308 - DNA, 24 bp, Artificial Sequence
  Synthetic oligonucleotide
aaaccgcccttgctcacgaattcc

SEQ ID NO: 309 - DNA, 24 bp, Artificial Sequence
  Synthetic oligonucleotide
gatccgaggagctgttcaccgggg

SEQ ID NO: 310 - DNA, 24 bp, Artificial Sequence
  Synthetic oligonucleotide
aaacccccggtgaacagctcctcg

SEQ ID NO: 311 - DNA, 24 bp, Artificial Sequence
  Synthetic oligonucleotide
gatcgaccaggatgggcaccaccc

SEQ ID NO: 312 - DNA, 24 bp, Artificial Sequence
  Synthetic oligonucleotide
aaacgggtggtgcccatcctggtc

SEQ ID NO: 313 - DNA, 24 bp, Artificial Sequence
  Synthetic oligonucleotide
gatccgtcgccgtccagctcgacc

SEQ ID NO: 314 - DNA, 24 bp, Artificial Sequence
  Synthetic oligonucleotide
aaacggtcgagctggacggcgacg

SEQ ID NO: 315 - DNA, 24 bp, Artificial Sequence
  Synthetic oligonucleotide
gatcggtggtgcagatcagcttca

SEQ ID NO: 316 - DNA, 24 bp, Artificial Sequence
  Synthetic oligonucleotide
aaactgaagctgatctgcaccacc

SEQ ID NO: 317 - DNA, 24 bp, Artificial Sequence
  Synthetic oligonucleotide
gatccaagtaatacatattcaaaa

SEQ ID NO: 318 - DNA, 24 bp, Artificial Sequence
  Synthetic oligonucleotide
aaacttttgaatatgtattacttg

SEQ ID NO: 319 - DNA, 24 bp, Artificial Sequence
  Synthetic oligonucleotide
gatcgaatatgtattacttggtta

SEQ ID NO: 320 - DNA, 24 bp, Artificial Sequence
  Synthetic oligonucleotide
aaactaaccaagtaatacatattc

SEQ ID NO: 321 - DNA, 24 bp, Artificial Sequence
  Synthetic oligonucleotide
gatcaagaacagaagaataacgca

SEQ ID NO: 322 - DNA, 24 bp, Artificial Sequence
  Synthetic oligonucleotide
aaactgcgttattcttctgttctt

SEQ ID NO: 323 - DNA, 24 bp, Artificial Sequence
  Synthetic oligonucleotide
gatcttatccctcatgttgtctaa

SEQ ID NO: 324 - DNA, 24 bp, Artificial Sequence
  Synthetic oligonucleotide
aaacttagacaacatgagggataa

SEQ ID NO: 325 - DNA, 24 bp, Artificial Sequence
  Synthetic oligonucleotide
gatccaatcaatacaataaaataa

SEQ ID NO: 326 - DNA, 24 bp, Artificial Sequence
  Synthetic oligonucleotide
aaacttattttattgtattgattg

SEQ ID NO: 327 - DNA, 24 bp, Artificial Sequence
  Synthetic oligonucleotide
gatctactcttttgaacaagatgt

SEQ ID NO: 328 - DNA, 24 bp, Artificial Sequence
  Synthetic oligonucleotide
aaacacatcttgttcaaaagagta

SEQ ID NO: 329 - DNA, 24 bp, Artificial Sequence
  Synthetic oligonucleotide
gatcataagtatattaggatgagg

SEQ ID NO: 330 - DNA, 24 bp, Artificial Sequence
  Synthetic oligonucleotide
aaaccctcatcctaatatacttat

SEQ ID NO: 331 - DNA, 47 bp, Artificial Sequence
  Synthetic oligonucleotide
gatcaatttctactaagtgtagatgtgtaggaacatcaacatgctca

SEQ ID NO: 332 - DNA, 47 bp, Artificial Sequence
  Synthetic oligonucleotide
aaaatgagcatgttgatgttcctacacatctacacttagtagaaatt

SEQ ID NO: 333 - DNA, 47 bp, Artificial Sequence
  Synthetic oligonucleotide
gatcaatttctactaagtgtagatcccttctccagaaacaatcagat

SEQ ID NO: 334 - DNA, 47 bp, Artificial Sequence
  Synthetic oligonucleotide
aaaaatctgattgtttctggagaagggatctacacttagtagaaatt

SEQ ID NO: 335 - DNA, 47 bp, Artificial Sequence
  Synthetic oligonucleotide
gatcaatttctactaagtgtagatccattcgtcttgaagtcgaggac

SEQ ID NO: 336 - DNA, 47 bp, Artificial Sequence
  Synthetic oligonucleotide
aaaagtcctcgacttcaagacgaatggatctacacttagtagaaatt

SEQ ID NO: 337 - DNA, 47 bp, Artificial Sequence
  Synthetic oligonucleotide
gatcaatttctactaagtgtagatagttattaaatggtcttcaattt

SEQ ID NO: 338 - DNA, 47 bp, Artificial Sequence
  Synthetic oligonucleotide
aaaaaaattgaagaccatttaataactatctacacttagtagaaatt

SEQ ID NO: 339 - DNA, 47 bp, Artificial Sequence
  Synthetic oligonucleotide
gatcaatttctactaagtgtagatataacttaaatatcaatgggagg

SEQ ID NO: 340 - DNA, 47 bp, Artificial Sequence
  Synthetic oligonucleotide
aaaacctcccattgatatttaagttatatctacacttagtagaaatt

SEQ ID NO: 341 - DNA, 24 bp, Artificial Sequence
  Synthetic oligonucleotide
gatcaatgaacgaagcaggaaatg

SEQ ID NO: 342 - DNA, 24 bp, Artificial Sequence
  Synthetic oligonucleotide
aaaccatttcctgcttcgttcatt

SEQ ID NO: 343 - DNA, 24 bp, Artificial Sequence
  Synthetic oligonucleotide
gatcgcgtgttgttgctgctgaca

SEQ ID NO: 344 - DNA, 24 bp, Artificial Sequence
  Synthetic oligonucleotide
aaactgtcagcagcaacaacacgc

SEQ ID NO: 345 - DNA, 47 bp, Artificial Sequence
  Synthetic oligonucleotide
gatcaatttctactaagtgtagatcttcttgctcattagaaagaaag

SEQ ID NO: 346 - DNA, 47 bp, Artificial Sequence
  Synthetic oligonucleotide
aaaactttctttctaatgagcaagaagatctacacttagtagaaatt

SEQ ID NO: 347 - DNA, 47 bp, Artificial Sequence
  Synthetic oligonucleotide
gatcaatttctactaagtgtagattaattaaaacttagattagattg

SEQ ID NO: 348 - DNA, 47 bp, Artificial Sequence
  Synthetic oligonucleotide
aaaacaatctaatctaagttttaattaatctacacttagtagaaatt

SEQ ID NO: 349 - DNA, 47 bp, Artificial Sequence
  Synthetic oligonucleotide
gatcaatttctactaagtgtagatcgtcgccgtccagctcgaccagg

SEQ ID NO: 350 - DNA, 47 bp, Artificial Sequence
  Synthetic oligonucleotide
aaaacctggtcgagctggacggcgacgatctacacttagtagaaatt

SEQ ID NO: 351 - DNA, 24 bp, Artificial Sequence
  Synthetic oligonucleotide
gatctttcttagcaaagcaaagga

SEQ ID NO: 352 - DNA, 24 bp, Artificial Sequence
  Synthetic oligonucleotide
aaactcctttgctttgctaagaaa

SEQ ID NO: 353 - DNA, 24 bp, Artificial Sequence
  Synthetic oligonucleotide
gatcggaaactcgaaatgaacgaa

SEQ ID NO: 354 - DNA, 24 bp, Artificial Sequence
  Synthetic oligonucleotide
aaacttcgttcatttcgagtttcc

SEQ ID NO: 355 - DNA, 24 bp, Artificial Sequence
  Synthetic oligonucleotide
gatccttagcaaagcaaaggaggg

SEQ ID NO: 356 - DNA, 24 bp, Artificial Sequence
  Synthetic oligonucleotide
aaacccctcctttgctttgctaag

SEQ ID NO: 357 - DNA, 24 bp, Artificial Sequence
  Synthetic oligonucleotide
gatcatagcggtagtgtttgcgcg

SEQ ID NO: 358 - DNA, 24 bp, Artificial Sequence
  Synthetic oligonucleotide
aaaccgcgcaaacactaccgctat

SEQ ID NO: 359 - DNA, 24 bp, Artificial Sequence
  Synthetic oligonucleotide
gatcgtaaaccccggccaaagaga

SEQ ID NO: 360 - DNA, 24 bp, Artificial Sequence
  Synthetic oligonucleotide
aaactctctttggccggggtttac

SEQ ID NO: 361 - DNA, 24 bp, Artificial Sequence
  Synthetic oligonucleotide
gatcacacgcctggcggatctgct

SEQ ID NO: 362 - DNA, 24 bp, Artificial Sequence
  Synthetic oligonucleotide
aaacagcagatccgccaggcgtgt

SEQ ID NO: 363 - DNA, 24 bp, Artificial Sequence
  Synthetic oligonucleotide
gatcacctgaatctaaaattcccg

SEQ ID NO: 364 - DNA, 24 bp, Artificial Sequence
  Synthetic oligonucleotide
aaaccgggaattttagattcaggt

SEQ ID NO: 365 - DNA, 24 bp, Artificial Sequence
  Synthetic oligonucleotide
gatcgccggggtttacggacgatg

SEQ ID NO: 366 - DNA, 24 bp, Artificial Sequence
  Synthetic oligonucleotide
aaaccatcgtccgtaaaccccggc

SEQ ID NO: 367 - DNA, 24 bp, Artificial Sequence
  Synthetic oligonucleotide
gatcgacggaaaaaagtagctaag

SEQ ID NO: 368 - DNA, 24 bp, Artificial Sequence
  Synthetic oligonucleotide
aaaccttagctacttttttccgtc

SEQ ID NO: 369 - DNA, 24 bp, Artificial Sequence
  Synthetic oligonucleotide
gatccaaagcattcaattcaaatg

SEQ ID NO: 370 - DNA, 24 bp, Artificial Sequence
  Synthetic oligonucleotide
aaaccatttgaattgaatgctttg

SEQ ID NO: 371 - DNA, 47 bp, Artificial Sequence
  Synthetic oligonucleotide
gatcaatttctactaagtgtagatcaagggtatggtacggtgctatc

SEQ ID NO: 372 - DNA, 47 bp, Artificial Sequence
  Synthetic oligonucleotide
aaaagatagcaccgtaccatacccttgatctacacttagtagaaatt

SEQ ID NO: 373 - DNA, 47 bp, Artificial Sequence
  Synthetic oligonucleotide
gatcaatttctactaagtgtagattcagcagcaacaacacgctacgc

SEQ ID NO: 374 - DNA, 47 bp, Artificial Sequence
  Synthetic oligonucleotide
aaaagcgtagcgtgttgttgctgctgaatctacacttagtagaaatt

SEQ ID NO: 375 - DNA, 47 bp, Artificial Sequence
  Synthetic oligonucleotide
gatcaatttctactaagtgtagatcggacgatggcagaagaccaaag

SEQ ID NO: 376 - DNA, 47 bp, Artificial Sequence
  Synthetic oligonucleotide
aaaactttggtcttctgccatcgtccgatctacacttagtagaaatt

SEQ ID NO: 377 - DNA, 47 bp, Artificial Sequence
  Synthetic oligonucleotide
gatcaatttctactaagtgtagatgcgagcgttggttggtggatcaa

SEQ ID NO: 378 - DNA, 47 bp, Artificial Sequence
  Synthetic oligonucleotide
aaaattgatccaccaaccaacgctcgcatctacacttagtagaaatt

SEQ ID NO: 379 - DNA, 47 bp, Artificial Sequence
  Synthetic oligonucleotide
gatcaatttctactaagtgtagatgtgctgacacatacaggcatata

SEQ ID NO: 380 - DNA, 47 bp, Artificial Sequence
  Synthetic oligonucleotide
aaaatatatgcctgtatgtgtcagcacatctacacttagtagaaatt

SEQ ID NO: 381 - DNA, 24 bp, Artificial Sequence
  Synthetic oligonucleotide
gatcataaatggaaagttaggaca

SEQ ID NO: 382 - DNA, 24 bp, Artificial Sequence
  Synthetic oligonucleotide
aaactgtcctaactttccatttat

SEQ ID NO: 383 - DNA, 47 bp, Artificial Sequence
  Synthetic oligonucleotide
gatcaatttctactaagtgtagatcggctatgaaaagctgttgttcg

SEQ ID NO: 384 - DNA, 47 bp, Artificial Sequence
  Synthetic oligonucleotide
aaaacgaacaacagcttttcatagccgatctacacttagtagaaatt

SEQ ID NO: 385 - DNA, 47 bp, Artificial Sequence
  Synthetic oligonucleotide
gatcaatttctactaagtgtagatcatattcgaagcttacaatcgag

SEQ ID NO: 386 - DNA, 47 bp, Artificial Sequence
  Synthetic oligonucleotide
aaaactcgattgtaagcttcgaatatgatctacacttagtagaaatt

SEQ ID NO: 387 - DNA, 47 bp, Artificial Sequence
  Synthetic oligonucleotide
gatcaatttctactaagtgtagattaccagcaatcagctgactaaca

SEQ ID NO: 388 - DNA, 47 bp, Artificial Sequence
  Synthetic oligonucleotide
aaaatgttagtcagctgattgctggtaatctacacttagtagaaatt

SEQ ID NO: 389 - DNA, 47 bp, Artificial Sequence
  Synthetic oligonucleotide
gatcaatttctactaagtgtagatttgctcttacccgactctgaaga

SEQ ID NO: 390 - DNA, 47 bp, Artificial Sequence
  Synthetic oligonucleotide
aaaatcttcagagtcgggtaagagcaaatctacacttagtagaaatt

SEQ ID NO: 391 - DNA, 47 bp, Artificial Sequence
  Synthetic oligonucleotide
gatcaatttctactaagtgtagatgcaagacctcaaacaatcgtact

SEQ ID NO: 392 - DNA, 47 bp, Artificial Sequence
  Synthetic oligonucleotide
aaaaagtacgattgtttgaggtcttgcatctacacttagtagaaatt

SEQ ID NO: 393 - DNA, 24 bp, Artificial Sequence
  Synthetic oligonucleotide
gatcgctggggtagaactagagta

SEQ ID NO: 394 - DNA, 24 bp, Artificial Sequence
  Synthetic oligonucleotide
aaactactctagttctaccccagc

SEQ ID NO: 395 - DNA, 24 bp, Artificial Sequence
  Synthetic oligonucleotide
gatcttatatgacagttcaaaaga

SEQ ID NO: 396 - DNA, 24 bp, Artificial Sequence
  Synthetic oligonucleotide
aaactcttttgaactgtcatataa

SEQ ID NO: 397 - DNA, 24 bp, Artificial Sequence
  Synthetic oligonucleotide
gatcggaagtggagatggaagagg

SEQ ID NO: 398 - DNA, 24 bp, Artificial Sequence
  Synthetic oligonucleotide
aaaccctcttccatctccacttcc

SEQ ID NO: 399 - DNA, 24 bp, Artificial Sequence
  Synthetic oligonucleotide
gatcctacatgcaaacgacaaata

SEQ ID NO: 400 - DNA, 24 bp, Artificial Sequence
  Synthetic oligonucleotide
aaactatttgtcgtttgcatgtag

SEQ ID NO: 401 - DNA, 24 bp, Artificial Sequence
  Synthetic oligonucleotide
gatcgctgaaaactgtatgtgcgg

SEQ ID NO: 402 - DNA, 24 bp, Artificial Sequence
  Synthetic oligonucleotide
aaacccgcacatacagttttcagc

SEQ ID NO: 403 - DNA, 24 bp, Artificial Sequence
  Synthetic oligonucleotide
gatcatccaacgatgcaattcagt

SEQ ID NO: 404 - DNA, 24 bp, Artificial Sequence
  Synthetic oligonucleotide
aaacactgaattgcatcgttggat

SEQ ID NO: 405 - DNA, 24 bp, Artificial Sequence
  Synthetic oligonucleotide
gatcaaatgggaatggaaagaacg

SEQ ID NO: 406 - DNA, 24 bp, Artificial Sequence
  Synthetic oligonucleotide
aaaccgttctttccattcccattt

SEQ ID NO: 407 - DNA, 47 bp, Artificial Sequence
  Synthetic oligonucleotide
gatcaatttctactaagtgtagatcaacaactatctgcgataactca

SEQ ID NO: 408 - DNA, 47 bp, Artificial Sequence
  Synthetic oligonucleotide
aaaatgagttatcgcagatagttgttgatctacacttagtagaaatt

SEQ ID NO: 409 - DNA, 47 bp, Artificial Sequence
  Synthetic oligonucleotide
gatcaatttctactaagtgtagatcagggtcttctataagagaaacc

SEQ ID NO: 410 - DNA, 47 bp, Artificial Sequence
  Synthetic oligonucleotide
aaaaggtttctcttatagaagaccctgatctacacttagtagaaatt

SEQ ID NO: 411 - DNA, 47 bp, Artificial Sequence
  Synthetic oligonucleotide
gatcaatttctactaagtgtagatagccctacttaatgctgagccac

SEQ ID NO: 412 - DNA, 47 bp, Artificial Sequence
  Synthetic oligonucleotide
aaaagtggctcagcattaagtagggctatctacacttagtagaaatt

SEQ ID NO: 413 - DNA, 47 bp, Artificial Sequence
  Synthetic oligonucleotide
gatcaatttctactaagtgtagatgctatgttagctgcaactttcta

SEQ ID NO: 414 - DNA, 47 bp, Artificial Sequence
  Synthetic oligonucleotide
aaaatagaaagttgcagctaacatagcatctacacttagtagaaatt

SEQ ID NO: 415 - DNA, 47 bp, Artificial Sequence
  Synthetic oligonucleotide
gatcaatttctactaagtgtagatgaaacacgtgtcctgaaaattat

SEQ ID NO: 416 - DNA, 47 bp, Artificial Sequence
  Synthetic oligonucleotide
aaaaataattttcaggacacgtgtttcatctacacttagtagaaatt

SEQ ID NO: 417 - DNA, 47 bp, Artificial Sequence
  Synthetic oligonucleotide
gatcaatttctactaagtgtagataaaatcatcgaatagccgatcga

SEQ ID NO: 418 - DNA, 47 bp, Artificial Sequence
  Synthetic oligonucleotide
aaaatcgatcggctattcgatgattttatctacacttagtagaaatt

SEQ ID NO: 419 - DNA, 47 bp, Artificial Sequence
  Synthetic oligonucleotide
gatcaatttctactaagtgtagatccagtcactatcatcatcatcat

SEQ ID NO: 420 - DNA, 47 bp, Artificial Sequence
  Synthetic oligonucleotide
aaaaatgatgatgatgatagtgactggatctacacttagtagaaatt

SEQ ID NO: 421 - DNA, 47 bp, Artificial Sequence
  Synthetic oligonucleotide
gatcaatttctactaagtgtagatacgggcaaaaactggattctccc

SEQ ID NO: 422 - DNA, 47 bp, Artificial Sequence
  Synthetic oligonucleotide
aaaagggagaatccagtttttgcccgtatctacacttagtagaaatt

SEQ ID NO: 423 - DNA, 47 bp, Artificial Sequence
  Synthetic oligonucleotide
gatcaatttctactaagtgtagattgtcttacgagccgggtaccaag

SEQ ID NO: 424 - DNA, 47 bp, Artificial Sequence
  Synthetic oligonucleotide
aaaacttggtacccggctcgtaagacaatctacacttagtagaaatt

SEQ ID NO: 425 - DNA, 47 bp, Artificial Sequence
  Synthetic oligonucleotide
gatcaatttctactaagtgtagatcaggggcgatgccacttatcagt

SEQ ID NO: 426 - DNA, 47 bp, Artificial Sequence
  Synthetic oligonucleotide
aaaaactgataagtggcatcgcccctgatctacacttagtagaaatt

SEQ ID NO: 427 - DNA, 24 bp, Artificial Sequence
  Synthetic oligonucleotide
gatcggattggcgagaaataatgt

SEQ ID NO: 428 - DNA, 24 bp, Artificial Sequence
  Synthetic oligonucleotide
aaacacattatttctcgccaatcc

SEQ ID NO: 429 - DNA, 24 bp, Artificial Sequence
  Synthetic oligonucleotide
gatcgcagatggggagagagaatg

SEQ ID NO: 430 - DNA, 24 bp, Artificial Sequence
  Synthetic oligonucleotide
aaaccattctctctccccatctgc

SEQ ID NO: 431 - DNA, 24 bp, Artificial Sequence
  Synthetic oligonucleotide
gatctttccttgtagcgatcaggt

SEQ ID NO: 432 - DNA, 24 bp, Artificial Sequence
  Synthetic oligonucleotide
aaacacctgatcgctacaaggaaa

SEQ ID NO: 433 - DNA, 24 bp, Artificial Sequence
  Synthetic oligonucleotide
gatcgaaataacgggtcccaagag

SEQ ID NO: 434 - DNA, 24 bp, Artificial Sequence
  Synthetic oligonucleotide
aaacctcttgggacccgttatttc

SEQ ID NO: 435 - DNA, 24 bp, Artificial Sequence
  Synthetic oligonucleotide
gatccccacgggttctttcttagg

SEQ ID NO: 436 - DNA, 24 bp, Artificial Sequence
  Synthetic oligonucleotide
aaaccctaagaaagaacccgtggg

SEQ ID NO: 437 - DNA, 24 bp, Artificial Sequence
  Synthetic oligonucleotide
gatcgcgagcaaacactattatga

SEQ ID NO: 438 - DNA, 24 bp, Artificial Sequence
  Synthetic oligonucleotide
aaactcataatagtgtttgctcgc

SEQ ID NO: 439 - DNA, 24 bp, Artificial Sequence
  Synthetic oligonucleotide
gatcagaagggcttggtttcgaaa

SEQ ID NO: 440 - DNA, 24 bp, Artificial Sequence
  Synthetic oligonucleotide
aaactttcgaaaccaagcccttct

SEQ ID NO: 441 - DNA, 24 bp, Artificial Sequence
  Synthetic oligonucleotide
gatccaaaacgggatatttaagcc

SEQ ID NO: 442 - DNA, 24 bp, Artificial Sequence
  Synthetic oligonucleotide
aaacggcttaaatatcccgttttg

SEQ ID NO: 443 - DNA, 24 bp, Artificial Sequence
  Synthetic oligonucleotide
gatcagccgaatgaatgaaatatg

SEQ ID NO: 444 - DNA, 24 bp, Artificial Sequence
  Synthetic oligonucleotide
aaaccatatttcattcattcggct

SEQ ID NO: 445 - DNA, 24 bp, Artificial Sequence
  Synthetic oligonucleotide
gatcttgttgtgatgatacaagag

SEQ ID NO: 446 - DNA, 24 bp, Artificial Sequence
  Synthetic oligonucleotide
aaacctcttgtatcatcacaacaa

SEQ ID NO: 447 - DNA, 150 bp, Artificial Sequence
  Synthetic oligonucleotide
ctttggtctccgatcaaattctcctgccaaacaaataagcaactccaatgaccacgttaatggctaatcctcttgatatcgaaaaactagctgaaaaatgtgatgtgctaacgatgatatcaagaggattggaaagtttggagacctttc

SEQ ID NO: 448 - DNA, 151 bp, Artificial Sequence
  Synthetic oligonucleotide
ctttggtctccgatctccacaaggacaatatttgtgacttatgttatgcgcctgctagagttccgggcagaaaatgcaatcaaatcttttcccggttgtggtatatttggtgtggacaacttcgccttaagttgaagtttggagacctttc

SEQ ID NO: 449 - DNA, 440 bp, Artificial Sequence
  Synthetic oligonucleotide
gttcgcggatccgttcactgccgtataggcagaatttctactaagtgtagatgcgagcgttggttggtggatcaagttcactgccgtataggcaggaccaggatgggcaccacccgttttagagctagaaatagcaagttaaaataaggctagtccgttatcaacttgaaaaagtggcaccgagtcggtgcgttcactgccgtataggcagtccacaaggacaatatttgtgacttatgttatgcgcctgctagagttccgggcagaaaatgcaatcaaatcttttcccggttgtggtatatttggtgtggacaacttcgccttaagttgaagttttagtactctggaaacagaatctactaaaacaaggcaaaatgccgtgtttatctcgtcaacttgttggcgagagttcactgccgtataggcagctcgagcgggtg

SEQ ID NO: 450 - DNA, 151 bp, Artificial Sequence
  Synthetic oligonucleotide
ctttggtctccgatcctacacctaagattccaagacccaagaacgcatttattctgttcagacagggaccgctcaaggtgtggaaataccccataattcaaacatttctaaaattactaccacaggatcttaataggtttggagacctttc

SEQ ID NO: 451 - DNA, 151 bp, Artificial Sequence
  Synthetic oligonucleotide
ctttggtctccgatctacataaaacctcacaaacgatcgaaaaatcttcctttaacgatcagcctcttgtatagcttatatgggtacattagtcaaggaaggtcatggtaagggtatctctcagaaatcggtacaagtttggagacctttc

SEQ ID NO: 452 - DNA, 151 bp, Artificial Sequence
  Synthetic oligonucleotide
ctttggtctccgatccgatatcacttggttacctgttcgtcgtaaggcttactgggaagtcaagtcgccgaattggagagccatggtgccgccatcgatactggtacttctttgattgaaggtatcggtttaggcggtttggagacctttc

SEQ ID NO: 453 - DNA, 151 bp, Artificial Sequence
  Synthetic oligonucleotide
ctttggtctccgatccatatgttccgatggtactcatgtagctgcctcataccagaccggaaatatagagtgaaacccacttctgaaccaacaaatggtatgaccccaacgcctgtatgcatttggaacttgaacggtttggagacctttc

SEQ ID NO: 454 - DNA, 151 bp, Artificial Sequence
  Synthetic oligonucleotide
ctttggtctccgatcttgcgcacctagttcagtagcgatcatacttaccactgtttgaggtaaataccaccaaaatcgaacactatttccatactatcatcagatggacagtccaatacgtaataccctatcctgggtttggagacctttc

SEQ ID NO: 455 - DNA, 60 bp, Artificial Sequence
  Synthetic oligonucleotide
ggtttccagccacagttgtagtcacgtgcgcgccatgctgtaatacgactcactataggg

SEQ ID NO: 456 - DNA, 60 bp, Artificial Sequence
  Synthetic oligonucleotide
cttggtagttggagcgcaattagcgtatcctgtaccatacaattaaccctcactaaaggg

SEQ ID NO: 457 - DNA, 30 bp, Artificial Sequence
  Synthetic oligonucleotide
tccttaagtggtccgtgttcggacctaatc

SEQ ID NO: 458 - DNA, 30 bp, Artificial Sequence
  Synthetic oligonucleotide
ccagctgccacctctaagaatggacgacgt

SEQ ID NO: 459 - DNA, 30 bp, Artificial Sequence
  Synthetic oligonucleotide
cggagcagacattgtaaggctacgttcacc

SEQ ID NO: 460 - DNA, 30 bp, Artificial Sequence
  Synthetic oligonucleotide
gtaggcctctcgtgctatcttcgttggacg

SEQ ID NO: 461 - DNA, 24 bp, Artificial Sequence
  Synthetic oligonucleotide
gtatctcgcagccggtctccgatc

SEQ ID NO: 462 - DNA, 25 bp, Artificial Sequence
  Synthetic oligonucleotide
cggttctctctcgtggtctcgaaac

SEQ ID NO: 463 - DNA, 60 bp, Artificial Sequence
  Synthetic oligonucleotide
tcgtcggcagcgtcagatgtgtataagagacagcttctccgcagtgaaagataaatgatc

SEQ ID NO: 464 - DNA, 58 bp, Artificial Sequence
  Synthetic oligonucleotide
gtctcgtgggctcggagatgtgtataagagacagctttgagtgagctgataccgctcg

SEQ ID NO: 465 - DNA, 27 bp, Artificial Sequence
  Synthetic oligonucleotide
agatttgttccgcgactaccaggggaa

SEQ ID NO: 466 - DNA, 27 bp, Artificial Sequence
  Synthetic oligonucleotide
aaaattcccctggtagtcgcggaacaa

SEQ ID NO: 467 - DNA, 27 bp, Artificial Sequence
  Synthetic oligonucleotide
agatatgagacgttttcttcattgatg

SEQ ID NO: 468 - DNA, 27 bp, Artificial Sequence
  Synthetic oligonucleotide
aaaacatcaatgaagaaaacgtctcat

SEQ ID NO: 469 - DNA, 24 bp, Artificial Sequence
  Synthetic oligonucleotide
gatccagcagttccatcagagtga

SEQ ID NO: 470 - DNA, 24 bp, Artificial Sequence
  Synthetic oligonucleotide
aaactcactctgatggaactgctg

SEQ ID NO: 471 - DNA, 24 bp, Artificial Sequence
  Synthetic oligonucleotide
gatcagagcgtgtgttgcgttgat

SEQ ID NO: 472 - DNA, 24 bp, Artificial Sequence
  Synthetic oligonucleotide
aaacatcaacgcaacacacgctct

SEQ ID NO: 473 - DNA, 24 bp, Artificial Sequence
  Synthetic oligonucleotide
gatcaaccaaaacatacaccattt

SEQ ID NO: 474 - DNA, 24 bp, Artificial Sequence
  Synthetic oligonucleotide
aaacaaatggtgtatgttttggtt

SEQ ID NO: 475 - DNA, 24 bp, Artificial Sequence
  Synthetic oligonucleotide
gatcatacgtaacacagatttaac

SEQ ID NO: 476 - DNA, 24 bp, Artificial Sequence
  Synthetic oligonucleotide
aaacgttaaatctgtgttacgtat

SEQ ID NO: 477 - DNA, 24 bp, Artificial Sequence
  Synthetic oligonucleotide
gatctcaacgcctgagccaaagat

SEQ ID NO: 478 - DNA, 24 bp, Artificial Sequence
  Synthetic oligonucleotide
aaacatctttggctcaggcgttga

SEQ ID NO: 479 - DNA, 27 bp, Artificial Sequence
  Synthetic oligonucleotide
agataggcaaagacaagaaaatacaag

SEQ ID NO: 480 - DNA, 27 bp, Artificial Sequence
  Synthetic oligonucleotide
aaaacttgtattttcttgtctttgcct

SEQ ID NO: 481 - DNA, 27 bp, Artificial Sequence
  Synthetic oligonucleotide
agatactaaataaccgcccagaaaatc

SEQ ID NO: 482 - DNA, 27 bp, Artificial Sequence
  Synthetic oligonucleotide
aaaagattttctgggcggttatttagt

SEQ ID NO: 483 - DNA, 27 bp, Artificial Sequence
  Synthetic oligonucleotide
agatgatgcagacgtggccaagttggc

SEQ ID NO: 484 - DNA, 27 bp, Artificial Sequence
  Synthetic oligonucleotide
aaaagccaacttggccacgtctgcatc

SEQ ID NO: 485 - DNA, 27 bp, Artificial Sequence
  Synthetic oligonucleotide
agatgacgcggagcagggtaaaaagtg

SEQ ID NO: 486 - DNA, 27 bp, Artificial Sequence
  Synthetic oligonucleotide
aaaacactttttaccctgctccgcgtc

SEQ ID NO: 487 - DNA, 27 bp, Artificial Sequence
  Synthetic oligonucleotide
agatcccgaagaacaaatagcggtagc

SEQ ID NO: 488 - DNA, 27 bp, Artificial Sequence
  Synthetic oligonucleotide
aaaagctaccgctatttgttcttcggg

SEQ ID NO: 489 - DNA, 27 bp, Artificial Sequence
  Synthetic oligonucleotide
agataggatgccgtaaaagaatgctcc

SEQ ID NO: 490 - DNA, 27 bp, Artificial Sequence
  Synthetic oligonucleotide
aaaaggagcattcttttacggcatcct

SEQ ID NO: 491 - DNA, 24 bp, Artificial Sequence
  Synthetic oligonucleotide
gatcacagtgttatgcttactaag

SEQ ID NO: 492 - DNA, 24 bp, Artificial Sequence
  Synthetic oligonucleotide
aaaccttagtaagcataacactgt

SEQ ID NO: 493 - DNA, 24 bp, Artificial Sequence
  Synthetic oligonucleotide
gatcaattaagattgtagagggag

SEQ ID NO: 494 - DNA, 24 bp, Artificial Sequence
  Synthetic oligonucleotide
aaacctccctctacaatcttaatt

SEQ ID NO: 495 - DNA, 24 bp, Artificial Sequence
  Synthetic oligonucleotide
gatctacaacgtagaactgataaa

SEQ ID NO: 496 - DNA, 24 bp, Artificial Sequence
  Synthetic oligonucleotide
aaactttatcagttctacgttgta

SEQ ID NO: 497 - DNA, 24 bp, Artificial Sequence
  Synthetic oligonucleotide
gatctgaatacctataactgctaa

SEQ ID NO: 498 - DNA, 24 bp, Artificial Sequence
  Synthetic oligonucleotide
aaacttagcagttataggtattca

SEQ ID NO: 499 - DNA, 24 bp, Artificial Sequence
  Synthetic oligonucleotide
gatctgtcgctttggaagaaaaag

SEQ ID NO: 500 - DNA, 24 bp, Artificial Sequence
  Synthetic oligonucleotide
aaacctttttcttccaaagcgaca

SEQ ID NO: 501 - DNA, 27 bp, Artificial Sequence
  Synthetic oligonucleotide
agataatgactatgttaataacaaagg

SEQ ID NO: 502 - DNA, 27 bp, Artificial Sequence
  Synthetic oligonucleotide
aaaacctttgttattaacatagtcatt

SEQ ID NO: 503 - DNA, 27 bp, Artificial Sequence
  Synthetic oligonucleotide
agattcattaaatagagatatataaga

SEQ ID NO: 504 - DNA, 27 bp, Artificial Sequence
  Synthetic oligonucleotide
aaaatcttatatatctctatttaatga

SEQ ID NO: 505 - DNA, 24 bp, Artificial Sequence
  Synthetic oligonucleotide
gatccctttaaaaaccatgagatc

SEQ ID NO: 506 - DNA, 24 bp, Artificial Sequence
  Synthetic oligonucleotide
aaacgatctcatggtttttaaagg

SEQ ID NO: 507 - DNA, 24 bp, Artificial Sequence
  Synthetic oligonucleotide
gatcggtgtaatgagtaatggtct

SEQ ID NO: 508 - DNA, 24 bp, Artificial Sequence
  Synthetic oligonucleotide
aaacagaccattactcattacacc

SEQ ID NO: 509 - DNA, 24 bp, Artificial Sequence
  Synthetic oligonucleotide
gatcagatcatgacagccgatacc

SEQ ID NO: 510 - DNA, 24 bp, Artificial Sequence
  Synthetic oligonucleotide
aaacggtatcggctgtcatgatct

SEQ ID NO: 511 - DNA, 24 bp, Artificial Sequence
  Synthetic oligonucleotide
gatcctgttcttgtagaatcggag

SEQ ID NO: 512 - DNA, 24 bp, Artificial Sequence
  Synthetic oligonucleotide
aaacctccgattctacaagaacag

SEQ ID NO: 513 - DNA, 24 bp, Artificial Sequence
  Synthetic oligonucleotide
gatcgcggccatatagacattacc

SEQ ID NO: 514 - DNA, 24 bp, Artificial Sequence
  Synthetic oligonucleotide
aaacggtaatgtctatatggccgc

SEQ ID NO: 515 - DNA, 24 bp, Artificial Sequence
  Synthetic oligonucleotide
gatcgattgattagggtcaaacct

SEQ ID NO: 516 - DNA, 24 bp, Artificial Sequence
  Synthetic oligonucleotide
aaacaggtttgaccctaatcaatc

SEQ ID NO: 517 - DNA, 22 bp, Artificial Sequence
  Synthetic oligonucleotide
aacaattgccgaacattctggg

SEQ ID NO: 518 - DNA, 22 bp, Artificial Sequence
  Synthetic oligonucleotide
tttcttggcgttggggatgata

SEQ ID NO: 519 - DNA, 22 bp, Artificial Sequence
  Synthetic oligonucleotide
atgatatcgagccatgcgtctt

SEQ ID NO: 520 - DNA, 22 bp, Artificial Sequence
  Synthetic oligonucleotide
cgcgtctttcaattgacccaat

SEQ ID NO: 521 - DNA, 22 bp, Artificial Sequence
  Synthetic oligonucleotide
ttcgatatcatctgcagggagc

SEQ ID NO: 522 - DNA, 22 bp, Artificial Sequence
  Synthetic oligonucleotide
aagggctgcggtaagtgattta

SEQ ID NO: 523 - DNA, 22 bp, Artificial Sequence
  Synthetic oligonucleotide
agtactagaaggggatgcaggt

SEQ ID NO: 524 - DNA, 22 bp, Artificial Sequence
  Synthetic oligonucleotide
taaaacgcctcttgactggtca

SEQ ID NO: 525 - DNA, 22 bp, Artificial Sequence
  Synthetic oligonucleotide
ctgtcttcccatctatcgtcgg

SEQ ID NO: 526 - DNA, 22 bp, Artificial Sequence
  Synthetic oligonucleotide
agcttcatcaccaacgtaggag

SEQ ID NO: 527 - DNA, 25 bp, Artificial Sequence
  Synthetic oligonucleotide
gatcagtaagttgagtgtaaggtgg

SEQ ID NO: 528 - DNA, 25 bp, Artificial Sequence
  Synthetic oligonucleotide
aaacccaccttacactcaacttact

SEQ ID NO: 529 - DNA, 25 bp, Artificial Sequence
  Synthetic oligonucleotide
gatcgtgattgttagttcagcgtaa

SEQ ID NO: 530 - DNA, 25 bp, Artificial Sequence
  Synthetic oligonucleotide
aaacttacgctgaactaacaatcac

SEQ ID NO: 531 - DNA, 25 bp, Artificial Sequence
  Synthetic oligonucleotide
gatcggcagccgtcgttgggcagaa

SEQ ID NO: 532 - DNA, 25 bp, Artificial Sequence
  Synthetic oligonucleotide
aaacttctgcccaacgacggctgcc

SEQ ID NO: 533 - DNA, 25 bp, Artificial Sequence
  Synthetic oligonucleotide
gatctgcatcgcgatgttagtttag

SEQ ID NO: 534 - DNA, 25 bp, Artificial Sequence
  Synthetic oligonucleotide
aaacctaaactaacatcgcgatgca

SEQ ID NO: 535 - DNA, 25 bp, Artificial Sequence
  Synthetic oligonucleotide
gatcccttctgttcatgcgtgacgg

SEQ ID NO: 536 - DNA, 25 bp, Artificial Sequence
  Synthetic oligonucleotide
aaacccgtcacgcatgaacagaagg

SEQ ID NO: 537 - DNA, 25 bp, Artificial Sequence
  Synthetic oligonucleotide
gatcggagaaaggaaagtagaaatg

SEQ ID NO: 538 - DNA, 25 bp, Artificial Sequence
  Synthetic oligonucleotide
aaaccatttctactttcctttctcc

SEQ ID NO: 539 - DNA, 25 bp, Artificial Sequence
  Synthetic oligonucleotide
gatcgtcgctaagatcattgtaact

SEQ ID NO: 540 - DNA, 25 bp, Artificial Sequence
  Synthetic oligonucleotide
aaacagttacaatgatcttagcgac

SEQ ID NO: 541 - DNA, 25 bp, Artificial Sequence
  Synthetic oligonucleotide
gatcaatagtctcacttactgggcg

SEQ ID NO: 542 - DNA, 25 bp, Artificial Sequence
  Synthetic oligonucleotide
aaaccgcccagtaagtgagactatt

SEQ ID NO: 543 - DNA, 25 bp, Artificial Sequence
  Synthetic oligonucleotide
gatctactgccacgtatttaatgag

SEQ ID NO: 544 - DNA, 25 bp, Artificial Sequence
  Synthetic oligonucleotide
aaacctcattaaatacgtggcagta

SEQ ID NO: 545 - DNA, 25 bp, Artificial Sequence
  Synthetic oligonucleotide
gatctctaccgtgagaaataaagca

SEQ ID NO: 546 - DNA, 25 bp, Artificial Sequence
  Synthetic oligonucleotide
aaactgctttatttctcacggtaga

SEQ ID NO: 547 - DNA, 60 bp, Artificial Sequence
  Synthetic oligonucleotide
gccacccataatcggcgcttagtttcggagttcaatcatactttgaaaagataatgtatg

SEQ ID NO: 548 - DNA, 60 bp, Artificial Sequence
  Synthetic oligonucleotide
atatggggtcagtggcgatattatactataggagttaaagaggaaacagctatgaccatg

SEQ ID NO: 549 - DNA, 60 bp, Artificial Sequence
  Synthetic oligonucleotide
atcaggcacgaaggcacactcgtatatgcatgttgttgaactttgaaaagataatgtatg

SEQ ID NO: 550 - DNA, 60 bp, Artificial Sequence
  Synthetic oligonucleotide
ttccatggggtcgcaacttttcccggtgacctctacatgtaggaaacagctatgaccatg

SEQ ID NO: 551 - DNA, 60 bp, Artificial Sequence
  Synthetic oligonucleotide
cagccacagttgtagtcacgtgcgcgccatgctgactaatctttgaaaagataatgtatg

SEQ ID NO: 552 - DNA, 60 bp, Artificial Sequence
  Synthetic oligonucleotide
tggtagttggagcgcaattagcgtatcctgtaccatactaaggaaacagctatgaccatg

SEQ ID NO: 553 - DNA, 60 bp, Artificial Sequence
  Synthetic oligonucleotide
gcgccggttttcattttcttccacggaataccaagcccatctttgaaaagataatgtatg

SEQ ID NO: 554 - DNA, 60 bp, Artificial Sequence
  Synthetic oligonucleotide
ctgtacgcagcatttagcagagatttgccaatgccaagaaaggaaacagctatgaccatg

SEQ ID NO: 555 - DNA, 60 bp, Artificial Sequence
  Synthetic oligonucleotide
ttcacgcaagttaagtccaggaaggtgagcaaatgctcatctttgaaaagataatgtatg

SEQ ID NO: 556 - DNA, 60 bp, Artificial Sequence
  Synthetic oligonucleotide
aggcacggaaacggctgcacgggtacgccagataaggataaggaaacagctatgaccatg

SEQ ID NO: 557 - DNA, 60 bp, Artificial Sequence
  Synthetic oligonucleotide
ccaatcaaagaagcatcggttcagatcgagcaaactgtagctttgaaaagataatgtatg

SEQ ID NO: 558 - DNA, 60 bp, Artificial Sequence
  Synthetic oligonucleotide
tgacatccaaactacaaaaccgagattggacatatagcacaggaaacagctatgaccatg

SEQ ID NO: 559 - DNA, 60 bp, Artificial Sequence
  Synthetic oligonucleotide
atacaatagcacatctcattacccagttatgattgacgtcctttgaaaagataatgtatg

SEQ ID NO: 560 - DNA, 60 bp, Artificial Sequence
  Synthetic oligonucleotide
cgaggaaaattagaattagtggagcaaataatgagcacagaggaaacagctatgaccatg

SEQ ID NO: 561 - DNA, 60 bp, Artificial Sequence
  Synthetic oligonucleotide
tgcgtctaacgcttttgccacttggatttctattataggactttgaaaagataatgtatg

SEQ ID NO: 562 - DNA, 60 bp, Artificial Sequence
  Synthetic oligonucleotide
aagaaattcttcctgtgcttcatcaaaacgcgaaaattcgaggaaacagctatgaccatg

SEQ ID NO: 563 - DNA, 60 bp, Artificial Sequence
  Synthetic oligonucleotide
agcgcttataaggttggggcaatactaaaactgtgatcttctttgaaaagataatgtatg

SEQ ID NO: 564 - DNA, 60 bp, Artificial Sequence
  Synthetic oligonucleotide
ttccgactctgttgtttcctattgtttctaatagggtacgaggaaacagctatgaccatg

SEQ ID NO: 565 - DNA, 60 bp, Artificial Sequence
  Synthetic oligonucleotide
tttctaactcttctcacgctgcccctatctgttcttccgcctttgaaaagataatgtatg

SEQ ID NO: 566 - DNA, 60 bp, Artificial Sequence
  Synthetic oligonucleotide
ctagccttattgttttagttcagtgacagcgaactgccgtaggaaacagctatgaccatg

SEQ ID NO: 567 - DNA, 489 bp, Artificial Sequence
  Synthetic oligonucleotide
  misc_feature (81)..(103): Guide sequence; n can be any nucleotide.
tcgtcggcagcgtcagatgtgtataagagacagcttctccgcagtgaaagataaatgatcaatttctactaagtgtagatnnnnnnnnnnnnnnnnnnnnnnntttttttgttttttatgtctgagctccctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaagctgtctcttatacacatctccgagcccacgagac

SEQ ID NO: 568 - DNA, 553 bp, Artificial Sequence
  Synthetic oligonucleotide
  misc_feature (61)..(80): Guide sequence; n can be any nucleotide.
tcgtcggcagcgtcagatgtgtataagagacagcttctccgcagtgaaagataaatgatcnnnnnnnnnnnnnnnnnnnngttttagagctagaaatagcaagttaaaataaggctagtccgttatcaacttgaaaaagtggcaccgagtcggtgcttttttgatcctttttttgttttttatgtctgagctccctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaagctgtctcttatacacatctccgagcccacgagac

SEQ ID NO: 569 - DNA, 699 bp, Artificial Sequence
  Synthetic oligonucleotide
  misc_feature (61)..(181): Guide sequence; n can be any nucleotide.
tcgtcggcagcgtcagatgtgtataagagacagcttctccgcagtgaaagataaatgatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttttagtactctgtaattttaggtatgaggtagacgaaaattgtacttatacctaaaattacagaatctactaaaacaaggcaaaatgccgtgtttatctcgtcaacttgttggcgagatttttttgatcctttttttgttttttatgtctgagctccctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaagctgtctcttatacacatctccgagcccacgagac

SEQ ID NO: 570 - DNA, 105 bp, Artificial Sequence
  Synthetic oligonucleotide
  misc_feature (42)..(64): Guide sequence; n can be any nucleotide.
tccttaagtggtccgtgttcggacctaatcggtctccagatnnnnnnnnnnnnnnnnnnnnnnnttttcgagaccacgtcgtccattcttagaggtggcagctgg

SEQ ID NO: 571 - DNA, 102 bp, Artificial Sequence
  Synthetic oligonucleotide
  misc_feature (42)..(61): Guide sequence; n can be any nucleotide.
cggagcagacattgtaaggctacgttcaccggtctccgatcnnnnnnnnnnnnnnnnnnnngtttcgagacccgtccaacgaagatagcacgagaggcctac

SEQ ID NO: 572 - DNA, 170 bp, Artificial Sequence
  Synthetic oligonucleotide
  misc_feature (25)..(145): Guide sequence; n can be any nucleotide.
gtatctcgcagccggtctccgatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtttcgagaccacgagagagaaccg

SEQ ID NO: 573 - protein, 1262 aa, Artificial Sequence
  Synthetic peptide
  misc (4)..(10): NLS sequence
  misc (1245)..(1261): NLS sequence
MAPPKKKRKVGSGSGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKKL

SEQ ID NO: 574 - protein, 1613 aa, Artificial Sequence
  Synthetic peptide
  MISC_FEATURE (3)..(10): NLS sequence
  MISC_FEATURE (1244)..(1261): NLS sequence
MAPPKKKRKVGSGSGSMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKKLGGSGGSPKKKRKVGRAEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 575 - protein, 1389 aa, Artificial Sequence
  Synthetic peptide
  MISC_FEATURE (3)..(10): NLS sequence
  MISC_FEATURE (1383)..(1389): NLS sequence
MDVPKKKRKVGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGAPKKKRKV

SEQ ID NO: 576 - protein, 1600 aa, Artificial Sequence
  Synthetic peptide
  MISC_FEATURE (3)..(10): NLS sequence
  MISC_FEATURE (1383)..(1389): NLS sequence
  MISC_FEATURE (1390)..(1404): Linker sequence
  MISC_FEATURE (1493)..(1501): Linker sequence
  MISC_FEATURE (1527)..(1543): Linker sequence
MDVPKKKRKVGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGAPKKKRKVGSSKLSGGGSGGSGSNSASSSTKLDDDLGTAAAVLSNMRSSPYRTHDKPISNVNDMNNTNALGVPASRPHSSSFPSKGVLRPILLRIHNSEQQPIFESNNSTSGGGSGGSGSDSQVQELETLPPIRSLPLPFPHMDSGGGSGGSGSKLGGSGGSYEEEIKHLKLGLEQRDHQIASLTVQQQRQQQQQQQVQQHLQQQQQQLAAASASVPVA

SEQ ID NO: 577 - protein, 7 aa, Artificial Sequence
  nuclear localization sequence (NLS)
PKKKRKV

SEQ ID NO: 578 - protein, 17 aa, Artificial Sequence
  nuclear localization sequence (NLS)
KRPAATKKAGQAKKKKK

SEQ ID NO: 579 - protein, 15 aa, Artificial Sequence
  Linker
GSSKLSGGGSGGSGS

SEQ ID NO: 580 - protein, 9 aa, Artificial Sequence
  Linker
GGGSGGSGS

SEQ ID NO: 581 - protein, 17 aa, Artificial Sequence
  Linker
GGGSGGSGSKLGGSGGS

SEQ ID NO: 582 - DNA, 43 bp, Artificial Sequence
  Synthetic oligonucleotide
aatttctactaagtgtagatccacggcatgtcaacaggtgagt

SEQ ID NO: 583 - DNA, 43 bp, Artificial Sequence
  Synthetic oligonucleotide
aatttctactaagtgtagatccaccgaggaactgtaccccaac

SEQ ID NO: 584 - DNA, 43 bp, Artificial Sequence
  Synthetic oligonucleotide
aatttctactaagtgtagatctttggatcttagagataacaga

SEQ ID NO: 585 - DNA, 43 bp, Artificial Sequence
  Synthetic oligonucleotide
aatttctactaagtgtagattaggggatggagagtgctacgcc

SEQ ID NO: 586 - DNA, 43 bp, Artificial Sequence
  Synthetic oligonucleotide
aatttctactaagtgtagatcacagccgtacatacacgtgcca

SEQ ID NO: 587 - DNA, 43 bp, Artificial Sequence
  Synthetic oligonucleotide
aatttctactaagtgtagattatacaaaatgaagggagaacta

SEQ ID NO: 588 - DNA, 43 bp, Artificial Sequence
  Synthetic oligonucleotide
aatttctactaagtgtagatgagtatggctacatggatcaagt

SEQ ID NO: 589 - DNA, 43 bp, Artificial Sequence
  Synthetic oligonucleotide
aatttctactaagtgtagatctgaaggttgaaaaagaatgcca

SEQ ID NO: 590 - DNA, 43 bp, Artificial Sequence
  Synthetic oligonucleotide
aatttctactaagtgtagataaccttcaggaaaagtttcagat

SEQ ID NO: 591 - DNA, 43 bp, Artificial Sequence
  Synthetic oligonucleotide
aatttctactaagtgtagattttacagcacttgatccatgtag

SEQ ID NO: 592 - DNA, 43 bp, Artificial Sequence
  Synthetic oligonucleotide
aatttctactaagtgtagattgctttgctatcgtgtagaactg

SEQ ID NO: 593 - DNA, 43 bp, Artificial Sequence
  Synthetic oligonucleotide
aatttctactaagtgtagatagatgagttgaaatttcgagtat

SEQ ID NO: 594 - DNA, 43 bp, Artificial Sequence
  Synthetic oligonucleotide
aatttctactaagtgtagatggtccactgttggattcgtagca

SEQ ID NO: 595 - DNA, 43 bp, Artificial Sequence
  Synthetic oligonucleotide
aatttctactaagtgtagatattaacgtaaaggaacatagtgc

SEQ ID NO: 596 - DNA, 43 bp, Artificial Sequence
  Synthetic oligonucleotide
aatttctactaagtgtagatgctgctgtttcttctggcaatcc

SEQ ID NO: 597 - DNA, 43 bp, Artificial Sequence
  Synthetic oligonucleotide
aatttctactaagtgtagattgccaggatcaagagcagcttct

SEQ ID NO: 598 - DNA, 43 bp, Artificial Sequence
  Synthetic oligonucleotide
aatttctactaagtgtagattatgatatctggcctaaggcgga

SEQ ID NO: 599 - DNA, 43 bp, Artificial Sequence
  Synthetic oligonucleotide
aatttctactaagtgtagattctgtagtcgacatcttttgctg

SEQ ID NO: 600 - DNA, 43 bp, Artificial Sequence
  Synthetic oligonucletide
aatttctactaagtgtagatcgttccttactgtagatagtcgg

SEQ ID NO: 601 - DNA, 43 bp, Artificial Sequence
  Synthetic oligonucleotide
aatttctactaagtgtagatttgctactggtggacacccgact

SEQ ID NO: 602 - DNA, 43 bp, Artificial Sequence
  Synthetic oligonucleotide
aatttctactaagtgtagatagggagacgacgatgctaccttg

SEQ ID NO: 603 - DNA, 43 bp, Artificial Sequence
  Synthetic oligonucleotide
aatttctactaagtgtagatagttacgtgttgcattgcgagat

SEQ ID NO: 604 - DNA, 43 bp, Artificial Sequence
  Synthetic oligonucleotide
aatttctactaagtgtagattcttgtttacgttccttactgta

SEQ ID NO: 605 - DNA, 43 bp, Artificial Sequence
  Synthetic oligonucleotide
aatttctactaagtgtagattaggattaaaagagatcatgagc

SEQ ID NO: 606 - DNA, 43 bp, Artificial Sequence
  Synthetic oligonucleotide
aatttctactaagtgtagatctatccttgcctattctttcctc

SEQ ID NO: 607 - DNA, 43 bp, Artificial Sequence
  Synthetic oligonucleotide
aatttctactaagtgtagatgacggagagaagaaaccggtgtt

SEQ ID NO: 608 - DNA, 43 bp, Artificial Sequence
  Synthetic oligonucleotide
aatttctactaagtgtagatctccttacggggtcctagcctgt

SEQ ID NO: 609 - DNA, 43 bp, Artificial Sequence
  Synthetic oligonucleotide
aatttctactaagtgtagatacatcaagccggatttgctcacg

SEQ ID NO: 610 - DNA, 43 bp, Artificial Sequence
  Synthetic oligonucleotide
aatttctactaagtgtagattcgagccaatcgagggcagcagt

SEQ ID NO: 611 - DNA, 43 bp, Artificial Sequence
  Synthetic oligonucleotide
aatttctactaagtgtagattcttgatatgataataggtggaa

SEQ ID NO: 612 - DNA, 44 bp, Artificial Sequence
  Synthetic oligonucleotide
cgtactaccagataacctaagttttagagctagaaatagcaagt

SEQ ID NO: 613 - DNA, 44 bp, Artificial Sequence
  Synthetic oligonucleotide
gttggggtacagttcctcgggttttagagctagaaatagcaagt

SEQ ID NO: 614 - DNA, 44 bp, Artificial Sequence
  Synthetic oligonucleotide
gggagaactatttgccaccggttttagagctagaaatagcaagt

SEQ ID NO: 615 - DNA, 44 bp, Artificial Sequence
  Synthetic oligonucleotide
tacccattgaataatggcatgttttagagctagaaatagcaagt

SEQ ID NO: 616 - DNA, 44 bp, Artificial Sequence
  Synthetic oligonucleotide
cagtttatatacaaaatgaagttttagagctagaaatagcaagt

SEQ ID NO: 617 - DNA, 44 bp, Artificial Sequence
  Synthetic oligonucleotide
gtccaagtgtggagaatagtgttttagagctagaaatagcaagt

SEQ ID NO: 618 - DNA, 44 bp, Artificial Sequence
  Synthetic oligonucleotide
ccagattctttgaggtaagagttttagagctagaaatagcaagt

SEQ ID NO: 619 - DNA, 44 bp, Artificial Sequence
  Synthetic oligonucleotide
tctgactcttgcgagagatggttttagagctagaaatagcaagt

SEQ ID NO: 620 - DNA, 44 bp, Artificial Sequence
  Synthetic oligonucleotide
gtatgtctatatgtattagagttttagagctagaaatagcaagt

SEQ ID NO: 621 - DNA, 44 bp, Artificial Sequence
  Synthetic oligonucleotide
actttacctctggccaccaagttttagagctagaaatagcaagt

SEQ ID NO: 622 - DNA, 44 bp, Artificial Sequence
  Synthetic oligonucleotide
actctgacagtttggtcaatgttttagagctagaaatagcaagt

SEQ ID NO: 623 - DNA, 44 bp, Artificial Sequence
  Synthetic oligonucleotide
gattacgaacatcgttggacgttttagagctagaaatagcaagt

SEQ ID NO: 624 - DNA, 44 bp, Artificial Sequence
  Synthetic oligonucleotide
ctatgatatctggcctaagggttttagagctagaaatagcaagt

SEQ ID NO: 625 - DNA, 44 bp, Artificial Sequence
  Synthetic oligonucleotide
tctgctgtagttagacgtaggttttagagctagaaatagcaagt

SEQ ID NO: 626 - DNA, 44 bp, Artificial Sequence
  Synthetic oligonucleotide
aggtttcttgcaaatgagcggttttagagctagaaatagcaagt

SEQ ID NO: 627 - DNA, 44 bp, Artificial Sequence
  Synthetic oligonucleotide
atttcttcacgacgacccttgttttagagctagaaatagcaagt

SEQ ID NO: 628 - DNA, 44 bp, Artificial Sequence
  Synthetic oligonucleotide
ccttcaaagcaacacttgccgttttagagctagaaatagcaagt

SEQ ID NO: 629 - DNA, 44 bp, Artificial Sequence
  Synthetic oligonucleotide
atgcccaaatttctatattagttttagagctagaaatagcaagt

SEQ ID NO: 630 - DNA, 44 bp, Artificial Sequence
  Synthetic oligonucleotide
tgaaaaatttcgcggcgacggttttagagctagaaatagcaagt

SEQ ID NO: 631 - DNA, 44 bp, Artificial Sequence
  Synthetic oligonucleotide
ctgcattatcaaggctcaaagttttagagctagaaatagcaagt

SEQ ID NO: 632 - DNA, 44 bp, Artificial Sequence
  Synthetic oligonucleotide
acattccatcacttgcgcttgttttagagctagaaatagcaagt

SEQ ID NO: 633 - DNA, 44 bp, Artificial Sequence
  Synthetic oligonucleotide
ttacgtgttgcattgcgagagttttagagctagaaatagcaagt

SEQ ID NO: 634 - DNA, 44 bp, Artificial Sequence
  Synthetic oligonucleotide
catttgtcagcatcacgctggttttagagctagaaatagcaagt

SEQ ID NO: 635 - DNA, 44 bp, Artificial Sequence
  Synthetic oligonucleotide
tgatcattaaaggctataacgttttagagctagaaatagcaagt

SEQ ID NO: 636 - DNA, 44 bp, Artificial Sequence
  Synthetic oligonucleotide
gcagatacttcgtgtgacaagttttagagctagaaatagcaagt

SEQ ID NO: 637 - DNA, 44 bp, Artificial Sequence
  Synthetic oligonucleotide
aagggcaacatctgcccaaagttttagagctagaaatagcaagt

SEQ ID NO: 638 - DNA, 44 bp, Artificial Sequence
  Synthetic oligonucleotide
atggccaattcaagccctttgttttagagctagaaatagcaagt

SEQ ID NO: 639 - DNA, 44 bp, Artificial Sequence
  Synthetic oligonucleotide
catatcaagagaaacaggctgttttagagctagaaatagcaagt

SEQ ID NO: 640 - DNA, 44 bp, Artificial Sequence
  Synthetic oligonucleotide
aaacaggctaggaccccgtagttttagagctagaaatagcaagt

SEQ ID NO: 641 - DNA, 44 bp, Artificial Sequence
  Synthetic oligonucleotide
tctcttgatatgataatagggttttagagctagaaatagcaagt

SEQ ID NO: 642 - DNA, 44 bp, Artificial Sequence
  Synthetic oligonucleotide
tgggatgaacaccttatcgaatggcttagaccagtttaaaaatt

SEQ ID NO: 643 - DNA, 44 bp, Artificial Sequence
  Synthetic oligonucleotide
gatcgtgccacaacggcccatctcagatagactgcagcccgcaa

SEQ ID NO: 644 - DNA, 44 bp, Artificial Sequence
  Synthetic oligonucleotide
gatgacaactttagagtccccatcgttgatacgatctctcaagg

SEQ ID NO: 645 - DNA, 44 bp, Artificial Sequence
  Synthetic oligonucleotide
gtcaagtgaaattgacaagttgaaagcaaaaatgtcccagtctg

SEQ ID NO: 646 - DNA, 44 bp, Artificial Sequence
  Synthetic oligonucleotide
tcgtatctctgcatatgacgttattatggaaaacagcattcctg

SEQ ID NO: 647 - DNA, 44 bp, Artificial Sequence
  Synthetic oligonucleotide
gaacaaggtgaacatgacgaaaacatctctcctgcccaggccgc

SEQ ID NO: 648 - DNA, 44 bp, Artificial Sequence
  Synthetic oligonucleotide
agaagaccgctctctattggttcacaaacataaactaattccat

SEQ ID NO: 649 - DNA, 44 bp, Artificial Sequence
  Synthetic oligonucleotide
acgttgctgtttgttgctacggatcgtatctctgcatatgacgt

SEQ ID NO: 650 - DNA, 44 bp, Artificial Sequence
  Synthetic oligonucleotide
atttcaatcaaatggcatctaatcaacctggcaagtgttgcttt

SEQ ID NO: 651 - DNA, 44 bp, Artificial Sequence
  Synthetic oligonucleotide
tggtgacttcaggagaatgtctttgaaaccaggcatcacgatca

SEQ ID NO: 652 - DNA, 44 bp, Artificial Sequence
  Synthetic oligonucleotide
catctttcgtcagcatcgaggaaattgaagcaattgatagcaag

SEQ ID NO: 653 - DNA, 44 bp, Artificial Sequence
  Synthetic oligonucleotide
atcggacaaaccaattgatcgtgatgcctggtttcaaagacatt

SEQ ID NO: 654 - DNA, 44 bp, Artificial Sequence
  Synthetic oligonucleotide
caggagatgatggagcaatagtcaggaagatagcgtgcggtggg

SEQ ID NO: 655 - DNA, 44 bp, Artificial Sequence
  Synthetic oligonucleotide
ttgtggatgctgatggccgtgtatggcagagaggaggcggttgc

SEQ ID NO: 656 - DNA, 44 bp, Artificial Sequence
  Synthetic oligonucleotide
ccttgcccatggccacgtagtctacggccacagacccggtatcg

SEQ ID NO: 657 - DNA, 44 bp, Artificial Sequence
  Synthetic oligonucleotide
tgataatgcaggcagatccaaagcgcaagtgatggaatgtgatc

SEQ ID NO: 658 - DNA, 44 bp, Artificial Sequence
  Synthetic oligonucleotide
ttgtcactttaggaccaaccttggaaacaattcctgacatctca

SEQ ID NO: 659 - DNA, 44 bp, Artificial Sequence
  Synthetic oligonucleotide
tccaagtactcgtgaagatccgagccacaaatcccacaccaaga

SEQ ID NO: 660 - DNA, 44 bp, Artificial Sequence
  Synthetic oligonucleotide
tgaggtgttcaatccctccaagcacggtcataaatctatagaga

SEQ ID NO: 661 - DNA, 44 bp, Artificial Sequence
  Synthetic oligonucleotide
atatccctaggccagaaatccaaaccgacgatgaggttattatc

SEQ ID NO: 662 - DNA, 4842 bp, Artificial Sequence
  dLbCpf1-VP
atggctcctccaaaaaagaagagaaaggtcggtagtggttctggatccatgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgctaggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcacaaaaggccggcggccacgaaaaaggccggccaggcaaaaaagaaaaagaagcttggcggcagcggcggcagcccgaaaaagaaacgcaaagttgggcgcgccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgtga

SEQ ID NO: 663 - DNA, 23 bp, Artificial Sequence
  Synthetic oligonucleotide
ccacggcatgtcaacaggtgagt

SEQ ID NO: 664 - DNA, 23 bp, Artificial Sequence
  Synthetic oligonucleotide
ccaccgaggaactgtaccccaac

SEQ ID NO: 665 - DNA, 23 bp, Artificial Sequence
  Synthetic oligonucleotide
ctttggatcttagagataacaga

SEQ ID NO: 666 - DNA, 23 bp, Artificial Sequence
  Synthetic oligonucleotide
taggggatggagagtgctacgcc

SEQ ID NO: 667 - DNA, 23 bp, Artificial Sequence
  Synthetic oligonucleotide
cacagccgtacatacacgtgcca

SEQ ID NO: 668 - DNA, 23 bp, Artificial Sequence
  Synthetic oligonucleotide
tatacaaaatgaagggagaacta

SEQ ID NO: 669 - DNA, 23 bp, Artificial Sequence
  Synthetic oligonucleotide
gagtatggctacatggatcaagt

SEQ ID NO: 670 - DNA, 23 bp, Artificial Sequence
  Synthetic oligonucleotide
ctgaaggttgaaaaagaatgcca

SEQ ID NO: 671 - DNA, 23 bp, Artificial Sequence
  Synthetic oligonucleotide
aaccttcaggaaaagtttcagat

SEQ ID NO: 672 - DNA, 23 bp, Artificial Sequence
  Synthetic oligonucleotide
tttacagcacttgatccatgtag

SEQ ID NO: 673 - DNA, 23 bp, Artificial Sequence
  Synthetic oligonucleotide
tgctttgctatcgtgtagaactg

SEQ ID NO: 674 - DNA, 23 bp, Artificial Sequence
  Synthetic oligonucleotide
agatgagttgaaatttcgagtat

SEQ ID NO: 675 - DNA, 23 bp, Artificial Sequence
  Synthetic oligonucleotide
ggtccactgttggattcgtagca

SEQ ID NO: 676 - DNA, 23 bp, Artificial Sequence
  Synthetic oligonucleotide
attaacgtaaaggaacatagtgc

SEQ ID NO: 677 - DNA, 23 bp, Artificial Sequence
  Synthetic oligonucleotide
gctgctgtttcttctggcaatcc

SEQ ID NO: 678 - DNA, 23 bp, Artificial Sequence
  Synthetic oligonucleotide
tgccaggatcaagagcagcttct

SEQ ID NO: 679 - DNA, 23 bp, Artificial Sequence
  Synthetic oligonucleotide
tatgatatctggcctaaggcgga

SEQ ID NO: 680 - DNA, 23 bp, Artificial Sequence
  Synthetic oligonucleotide
tctgtagtcgacatcttttgctg

SEQ ID NO: 681 - DNA, 23 bp, Artificial Sequence
  Synthetic oligonucleotide
cgttccttactgtagatagtcgg

SEQ ID NO: 682 - DNA, 23 bp, Artificial Sequence
  Synthetic oligonucleotide
ttgctactggtggacacccgact

SEQ ID NO: 683 - DNA, 23 bp, Artificial Sequence
  Synthetic oligonucleotide
agggagacgacgatgctaccttg

SEQ ID NO: 684 - DNA, 23 bp, Artificial Sequence
  Synthetic oligonucleotide
agttacgtgttgcattgcgagat

SEQ ID NO: 685 - DNA, 23 bp, Artificial Sequence
  Synthetic oligonucleotide
tcttgtttacgttccttactgta

SEQ ID NO: 686 - DNA, 23 bp, Artificial Sequence
  Synthetic oligonucleotide
taggattaaaagagatcatgagc

SEQ ID NO: 687 - DNA, 23 bp, Artificial Sequence
  Synthetic oligonucleotide
ctatccttgcctattctttcctc

SEQ ID NO: 688 - DNA, 23 bp, Artificial Sequence
  Synthetic oligonucleotide
gacggagagaagaaaccggtgtt

SEQ ID NO: 689 - DNA, 23 bp, Artificial Sequence
  Synthetic oligonucleotide
ctccttacggggtcctagcctgt

SEQ ID NO: 690 - DNA, 23 bp, Artificial Sequence
  Synthetic oligonucleotide
acatcaagccggatttgctcacg

SEQ ID NO: 691 - DNA, 23 bp, Artificial Sequence
  Synthetic oligonucleotide
tcgagccaatcgagggcagcagt

SEQ ID NO: 692 - DNA, 23 bp, Artificial Sequence
  Synthetic oligonucleotide
tcttgatatgataataggtggaa

SEQ ID NO: 693 - DNA, 20 bp, Artificial Sequence
  Synthetic oligonucleotide
cgtactaccagataacctaa

SEQ ID NO: 694 - DNA, 20 bp, Artificial Sequence
  Synthetic oligonucleotide
gttggggtacagttcctcgg

SEQ ID NO: 695 - DNA, 20 bp, Artificial Sequence
  Synthetic oligonucleotide
gggagaactatttgccaccg

SEQ ID NO: 696 - DNA, 20 bp, Artificial Sequence
  Synthetic oligonucleotide
tacccattgaataatggcat

SEQ ID NO: 697 - DNA, 20 bp, Artificial Sequence
  Synthetic oligonucleotide
cagtttatatacaaaatgaa

SEQ ID NO: 698 - DNA, 20 bp, Artificial Sequence
  Synthetic oligonucleotide
gtccaagtgtggagaatagt

SEQ ID NO: 699 - DNA, 20 bp, Artificial Sequence
  Synthetic oligonucleotide
ccagattctttgaggtaaga

SEQ ID NO: 700 - DNA, 20 bp, Artificial Sequence
  Synthetic oligonucleotide
tctgactcttgcgagagatg

SEQ ID NO: 701 - DNA, 20 bp, Artificial Sequence
  Synthetic oligonucleotide
gtatgtctatatgtattaga

SEQ ID NO: 702 - DNA, 20 bp, Artificial Sequence
  Synthetic oligonucleotide
actttacctctggccaccaa

SEQ ID NO: 703 - DNA, 20 bp, Artificial Sequence
  Synthetic oligonucleotide
actctgacagtttggtcaat

SEQ ID NO: 704 - DNA, 20 bp, Artificial Sequence
  Synthetic oligonucleotide
gattacgaacatcgttggac

SEQ ID NO: 705 - DNA, 20 bp, Artificial Sequence
  Synthetic oligonucleotide
ctatgatatctggcctaagg

SEQ ID NO: 706 - DNA, 20 bp, Artificial Sequence
  Synthetic oligonucleotide
tctgctgtagttagacgtag

SEQ ID NO: 707 - DNA, 20 bp, Artificial Sequence
  Synthetic oligonucleotide
aggtttcttgcaaatgagcg

SEQ ID NO: 708 - DNA, 20 bp, Artificial Sequence
  Synthetic oligonucleotide
atttcttcacgacgaccctt

SEQ ID NO: 709 - DNA, 20 bp, Artificial Sequence
  Synthetic oligonucleotide
ccttcaaagcaacacttgcc

SEQ ID NO: 710 - DNA, 20 bp, Artificial Sequence
  Synthetic oligonucleotide
atgcccaaatttctatatta

SEQ ID NO: 711 - DNA, 20 bp, Artificial Sequence
  Synthetic oligonucleotide
tgaaaaatttcgcggcgacg

SEQ ID NO: 712 - DNA, 20 bp, Artificial Sequence
  Synthetic oligonucleotide
ctgcattatcaaggctcaaa

SEQ ID NO: 713 - DNA, 20 bp, Artificial Sequence
  Synthetic oligonucleotide
acattccatcacttgcgctt

SEQ ID NO: 714 - DNA, 20 bp, Artificial Sequence
  Synthetic oligonucleotide
ttacgtgttgcattgcgaga

SEQ ID NO: 715 - DNA, 20 bp, Artificial Sequence
  Synthetic oligonucleotide
catttgtcagcatcacgctg

SEQ ID NO: 716 - DNA, 20 bp, Artificial Sequence
  Synthetic oligonucleotide
tgatcattaaaggctataac

SEQ ID NO: 717 - DNA, 20 bp, Artificial Sequence
  Synthetic oligonucleotide
gcagatacttcgtgtgacaa

SEQ ID NO: 718 - DNA, 20 bp, Artificial Sequence
  Synthetic oligonucleotide
aagggcaacatctgcccaaa

SEQ ID NO: 719 - DNA, 20 bp, Artificial Sequence
  Synthetic oligonucleotide
atggccaattcaagcccttt

SEQ ID NO: 720 - DNA, 20 bp, Artificial Sequence
  Synthetic oligonucleotide
catatcaagagaaacaggct

SEQ ID NO: 721 - DNA, 20 bp, Artificial Sequence
  Synthetic oligonucleotide
aaacaggctaggaccccgta

SEQ ID NO: 722 - DNA, 20 bp, Artificial Sequence
  Synthetic oligonucleotide
tctcttgatatgataatagg

SEQ ID NO: 723 - DNA, 121 bp, Artificial Sequence
  Synthetic oligonucleotide
tgggatgaacaccttatcgaatggcttagaccagtttaaaaattgggtagttcaatagactccttgtgcaagcgctgatagtcctgcaacccgtccaagtctttagaaccgaagaacttag

SEQ ID NO: 724 - DNA, 121 bp, Artificial Sequence
  Synthetic oligonucleotide
gatcgtgccacaacggcccatctcagatagactgcagcccgcaattgctagcaggactatcagcgcttgcacaaggagtctattgaagaccctgctaagtcccactattctccacacttgg

SEQ ID NO: 725 - DNA, 121 bp, Artificial Sequence
  Synthetic oligonucleotide
gatgacaactttagagtccccatcgttgatacgatctctcaaggagttggaatggcaccgatacgggaaatggccaacaaggttatgattgcttctgggaaagaaaacccggcaaagacta

SEQ ID NO: 726 - DNA, 121 bp, Artificial Sequence
  Synthetic oligonucleotide
gtcaagtgaaattgacaagttgaaagcaaaaatgtcccagtctgccgccatgaacatttgacttcggtcaagatcgtgccacaacggcccatctcagatactgcgcagcagaagaaggaac

SEQ ID NO: 727 - DNA, 121 bp, Artificial Sequence
  Synthetic oligonucleotide
tcgtatctctgcatatgacgttattatggaaaacagcattcctgaaaaggctggttcaagttcctgtccaacgatgttcgtaatcatttggtcgacatcgggatcctattgaccaaactgt

SEQ ID NO: 728 - DNA, 121 bp, Artificial Sequence
  Synthetic oligonucleotide
gaacaaggtgaacatgacgaaaacatctctcctgcccaggccgctgagctgcagaactggctgtaaaactgtactccaagtgcaaagattatgctaaggaggtgggtgaagatttgtcacg

SEQ ID NO: 729 - DNA, 121 bp, Artificial Sequence
  Synthetic oligonucleotide
agaagaccgctctctattggttcacaaacataaactaattccattggaagtgcttggaaagagtacgtaaaaacaggtactgtgcatggtttgaaacaactaattgtcagaggctacatca

SEQ ID NO: 730 - DNA, 121 bp, Artificial Sequence
  Synthetic oligonucleotide
acgttgctgtttgttgctacggatcgtatctctgcatatgacgttattatctattgaccaaactgtcagagttctggttcaagttcctgtccaacgatgtggaaaacagcattcctgaaaa

SEQ ID NO: 731 - DNA, 121 bp, Artificial Sequence
  Synthetic oligonucleotide
atttcaatcaaatggcatctaatcaacctggcaagtgttgctttgaaggagtcgtgaagaaatcttcggtttagatacttatgcagcaggctctacatctgtttgtcacgatggaacaccc

SEQ ID NO: 732 - DNA, 121 bp, Artificial Sequence
  Synthetic oligonucleotide
tggtgacttcaggagaatgtctttgaaaccaggcatcacgatcaattggtaaatatcgggaacaaagaccatgtacccagcactagcaaatttgtcggccttgtccgatgagatagcatcg

SEQ ID NO: 733 - DNA, 121 bp, Artificial Sequence
  Synthetic oligonucleotide
catctttcgtcagcatcgaggaaattgaagcaattgatagcaagaaaccaacatctttccggcaaacttaagacacttaacggaggaaaaattaaaggatatattgatttcagcagcggaa

SEQ ID NO: 734 - DNA, 121 bp, Artificial Sequence
  Synthetic oligonucleotide
atcggacaaaccaattgatcgtgatgcctggtttcaaagacattctcctgcatgaagttgttaaaacttgaatatgacccaaagtttattggcgttgtggaagtcaccaagaaaattgttg

SEQ ID NO: 735 - DNA, 121 bp, Artificial Sequence
  Synthetic oligonucleotide
caggagatgatggagcaatagtcaggaagatagcgtgcggtgggaaccactggtaggatgtggagataacagacggggagaactggatagtgcgcaagcaagcgtgatgctgacaaatgac

SEQ ID NO: 736 - DNA, 121 bp, Artificial Sequence
  Synthetic oligonucleotide
ttgtggatgctgatggccgtgtatggcagagaggaggcggttgctacgagccaacgatgagcgcatcgcagtatacggatgtttccagaactttgtggtgttcactcagcaacatgtgcca

SEQ ID NO: 737 - DNA, 121 bp, Artificial Sequence
  Synthetic oligonucleotide
ccttgcccatggccacgtagtctacggccacagacccggtatcgtacacctgggctcttgcaattgacactttgtgttgctgccccagccgtatactcggaatacgggctctttcagtgat

SEQ ID NO: 738 - DNA, 121 bp, Artificial Sequence
  Synthetic oligonucleotide
tgataatgcaggcagatccaaagcgcaagtgatggaatgtgatcattaaagtgtgtatgcgtttgggtctaatgggcaaaggcaactgggactggggcacggctataacaggcttgtatcg

SEQ ID NO: 739 - DNA, 121 bp, Artificial Sequence
  Synthetic oligonucleotide
ttgtcactttaggaccaaccttggaaacaattcctgacatctcatggcccatttatggcactctccatctttaggcatgaagattggaccatccaagtacattgccagaggtaaagcagcg

SEQ ID NO: 740 - DNA, 121 bp, Artificial Sequence
  Synthetic oligonucleotide
tccaagtactcgtgaagatccgagccacaaatcccacaccaagagacgtctctggcctagggatatcattagtgaagtgaatatcacccttcttgaaatagataataacctcatcgtcggt

SEQ ID NO: 741 - DNA, 121 bp, Artificial Sequence
  Synthetic oligonucleotide
tgaggtgttcaatccctccaagcacggtcataaatctatagagatactactgattacagttatgattgttctggtattcaagttactttcgaaacctcttgtggtttgaccaagagccatg

SEQ ID NO: 742 - DNA, 121 bp, Artificial Sequence
  Synthetic oligonucleotide
atatccctaggccagaaatccaaaccgacgatgaggttattatcgacgtcttcacgagtacttggatggtccaatcttcatgcctaaagatggagagtgctcttggtgtgggatttgtggc

SEQ ID NO: 743 - DNA, 4803 bp, Artificial Sequence
  dSpCas9-RD1152
atggacgtcccaaagaagaagagaaaggtaggcagcgacaagaagtattctatcggactggccatcgggactaatagcgtcgggtgggccgtgatcactgacgagtacaaggtgccctctaagaagttcaaggtgctcgggaacaccgaccggcattccatcaagaaaaatctgatcggagctctcctctttgattcaggggagaccgctgaagcaacccgcctcaagcggactgctagacggcggtacaccaggaggaagaaccggatttgttaccttcaagagatattctccaacgaaatggcaaaggtcgacgacagcttcttccataggctggaagaatcattcctcgtggaagaggataagaagcatgaacggcatcccatcttcggtaatatcgtcgacgaggtggcctatcacgagaaatacccaaccatctaccatcttcgcaaaaagctggtggactcaaccgacaaggcagacctccggcttatctacctggccctggcccacatgatcaagttcagaggccacttcctgatcgagggcgacctcaatcctgacaatagcgatgtggataaactgttcatccagctggtgcagacttacaaccagctctttgaagagaaccccatcaatgcaagcggagtcgatgccaaggccattctgtcagcccggctgtcaaagagccgcagacttgagaatcttatcgctcagctgccgggtgaaaagaaaaatggactgttcgggaacctgattgctctttcacttgggctgactcccaatttcaagtctaatttcgacctggcagaggatgccaagctgcaactgtccaaggacacctatgatgacgatctcgacaacctcctggcccagatcggtgaccaatacgccgaccttttccttgctgctaagaatctttctgacgccatcctgctgtctgacattctccgcgtgaacactgaaatcaccaaggcccctctttcagcttcaatgattaagcggtatgatgagcaccaccaggacctgaccctgcttaaggcactcgtccggcagcagcttccggagaagtacaaggaaatcttctttgaccagtcaaagaatggatacgccggctacatcgacggaggtgcctcccaagaggaattttataagtttatcaaacctatccttgagaagatggacggcaccgaagagctcctcgtgaaactgaatcgggaggatctgctgcggaagcagcgcactttcgacaatgggagcattccccaccagatccatcttggggagcttcacgccatccttcggcgccaagaggacttctacccctttcttaaggacaacagggagaagattgagaaaattctcactttccgcatcccctactacgtgggacccctcgccagaggaaatagccggtttgcttggatgaccagaaagtcagaagaaactatcactccctggaacttcgaagaggtggtggacaagggagccagcgctcagtcattcatcgaacggatgactaacttcgataagaacctccccaatgagaaggtcctgccgaaacattccctgctctacgagtactttaccgtgtacaacgagctgaccaaggtgaaatatgtcaccgaagggatgaggaagcccgcattcctgtcaggcgaacaaaagaaggcaattgtggaccttctgttcaagaccaatagaaaggtgaccgtgaagcagctgaaggaggactatttcaagaaaattgaatgcttcgactctgtggagattagcggggtcgaagatcggttcaacgcaagcctgggtacctaccatgatctgcttaagatcatcaaggacaaggattttctggacaatgaggagaacgaggacatccttgaggacattgtcctgactctcactctgttcgaggaccgggaaatgatcgaggagaggcttaagacctacgcccatctgttcgacgataaagtgatgaagcaacttaaacggagaagatataccggatggggacgccttagccgcaaactcatcaacggaatccgggacaaacagagcggaaagaccattcttgatttccttaagagcgacggattcgctaatcgcaacttcatgcaacttatccatgatgattccctgacctttaaggaggacatccagaaggcccaagtgtctggacaaggtgactcactgcacgagcatatcgcaaatctggctggttcacccgctattaagaagggtattctccagaccgtgaaagtcgtggacgagctggtcaaggtgatgggtcgccataaaccagagaacattgtcatcgagatggccagggaaaaccagactacccagaagggacagaagaacagcagggagcggatgaaaagaattgaggaagggattaaggagctcgggtcacagatccttaaagagcacccggtggaaaacacccagcttcagaatgagaagctctatctgtactaccttcaaaatggacgcgatatgtatgtggaccaagagcttgatatcaacaggctctcagactacgacgtggacgccatcgtccctcagagcttcctcaaagacgactcaattgacaataaggtgctgactcgctcagacaagaaccggggaaagtcagataacgtgccctcagaggaagtcgtgaaaaagatgaagaactattggcgccagcttctgaacgcaaagctgatcactcagcggaagttcgacaatctcactaaggctgagaggggcggactgagcgaactggacaaagcaggattcattaaacggcaacttgtggagactcggcagattactaaacatgtcgcccaaatccttgactcacgcatgaataccaagtacgacgaaaacgacaaacttatccgcgaggtgaaggtgattaccctgaagtccaagctggtcagcgatttcagaaaggactttcaattctacaaagtgcgggagatcaataactatcatcatgctcatgacgcatatctgaatgccgtggtgggaaccgccctgatcaagaagtacccaaagctggaaagcgagttcgtgtacggagactacaaggtctacgacgtgcgcaagatgattgccaaatctgagcaggagatcggaaaggccaccgcaaagtacttcttctacagcaacatcatgaatttcttcaagaccgaaatcacccttgcaaacggtgagatccggaagaggccgctcatcgagactaatggggagactggcgaaatcgtgtgggacaagggcagagatttcgctaccgtgcgcaaagtgctttctatgcctcaagtgaacatcgtgaagaaaaccgaggtgcaaaccggaggcttttctaaggaatcaatcctccccaagcgcaactccgacaagctcattgcaaggaagaaggattgggaccctaagaagtacggcggattcgattcaccaactgtggcttattctgtcctggtcgtggctaaggtggaaaaaggaaagtctaagaagctcaagagcgtgaaggaactgctgggtatcaccattatggagcgcagctccttcgagaagaacccaattgactttctcgaagccaaaggttacaaggaagtcaagaaggaccttatcatcaagctcccaaagtatagcctgttcgaactggagaatgggcggaagcggatgctcgcctccgctggcgaacttcagaagggtaatgagctggctctcccctccaagtacgtgaatttcctctaccttgcaagccattacgagaagctgaaggggagccccgaggacaacgagcaaaagcaactgtttgtggagcagcataagcattatctggacgagatcattgagcagatttccgagttttctaaacgcgtcattctcgctgatgccaacctcgataaagtccttagcgcatacaataagcacagagacaaaccaattcgggagcaggctgagaatatcatccacctgttcaccctcaccaatcttggtgcccctgccgcattcaagtacttcgacaccaccatcgaccggaaacgctatacctccaccaaagaagtgctggacgccaccctcatccaccagagcatcaccggactttacgaaactcggattgacctctcacagctcggaggggatgagggagctcccaagaaaaagcgcaaggtaggtagttccaagcttagtggtggaggaagtggcgggtcagggtcgaattctgcatcttcatctaccaaactagacgacgacttgggtacagcagcagcagtgctatcaaacatgagatcatccccatatagaactcatgataaacccatttccaatgtcaatgacatgaataacacaaatgcgctcggtgtgccggctagtaggcctcattcgtcatcttttccatcaaagggtgtcttaagaccaattctgttacgtatccataattccgaacaacaacccattttcgaaagcaacaattctacatcgggaggtggttcgggtggctctggatcagattcacaagttcaagaactggaaacattaccacccataagaagtttaccgttgcccttcccacacatggactcaggcggtggtagtggtgggagcggtagtaagcttggcggcagcggcggcagctacgaagaagagatcaagcacttgaaactagggctggagcaaagagaccatcaaattgcatctttgaccgtccagcaacagcggcaacagcaacagcagcaacaggtccagcagcatttacaacagcaacagcagcagctagccgctgcatctgcatctgttccagttgcgtaa